SEQUENCE LISTING (308 sequences)

SEQ ID NO: 1 - DNA, 26 bp, Artificial
  Oligonucleotide
gaattcaaggtcgggcaggaagaggg

SEQ ID NO: 2 - DNA, 42 bp, Artificial
  Oligonucleotide
aagcttagatctcgtctcacggtgtttcgtcctttccacaag

SEQ ID NO: 3 - DNA, 31 bp, Artificial
  Oligonucleotide
accgtgtgcaccggcacagacattcaagaga

SEQ ID NO: 4 - DNA, 19 bp, Artificial
  Oligonucleotide
gcaatgatcttgatcttca

SEQ ID NO: 5 - DNA, 29 bp, Artificial
  Oligonucleotide
gcaatgatcttgatcttcatttttggaaa

SEQ ID NO: 6 - DNA, 42 bp, Artificial
  Oligonucleotide
agcttttccaaaaatgaagatcaagatcattgctctcttgaa

SEQ ID NO: 7 - DNA, 29 bp, Artificial
  Oligonucleotide
cttgaagcaatgatcttgatcttcacggt

SEQ ID NO: 8 - DNA, 20 bp, Artificial
  Oligonucleotide
ggtgtttcgtcctttccaca

SEQ ID NO: 9 - DNA, 27 bp, Artificial
  Oligonucleotide
tttttggaaaagcttatcgataccgtc

SEQ ID NO: 10 - DNA, 58 bp, Artificial
  Oligonucleotide
ctcttgaacgctctctctccaacttccgtttctcatccggtgtttcgtcctttccaca

SEQ ID NO: 11 - DNA, 58 bp, Artificial
  Oligonucleotide
acgctctctctccaacttccgtttctcatcctttttggaaaagcttatcgataccgtc

SEQ ID NO: 12 - DNA, 62 bp, Artificial
  Oligonucleotide
gagaacatggttaactggttaagtcatgtcgtcccacaggagcgcaccatcttcttcaagga

SEQ ID NO: 13 - DNA, 66 bp, Artificial
  Oligonucleotide
tgaacatgagaagggctggccactctccacctcctgtactcacctggacgtagccttcgggcatgg

SEQ ID NO: 14 - DNA, 27 bp, Artificial
  Oligonucleotide
tcttggacgtgggtgtttcgtcctttc

SEQ ID NO: 15 - DNA, 31 bp, Artificial
  Oligonucleotide
tcttggaatgcttttttggaaaagcttatcg

SEQ ID NO: 16 - DNA, 42 bp, Artificial
  Oligonucleotide
acacaaagtaggagtagtgaaaggccggtgtttcgtcctttc

SEQ ID NO: 17 - DNA, 41 bp, Artificial
  Oligonucleotide
aggtaggagtagtgaaaggccttttttggaaaagcttatcg

SEQ ID NO: 18 - DNA, 64 bp, Artificial
  Oligonucleotide
acacaaagcaatgatcttgatcttcataagtaggagtagtgaaaggccggtgtttcgtcctttc

SEQ ID NO: 19 - DNA, 63 bp, Artificial
  Oligonucleotide
aggcaatgatcttgatcttcattggtaggagtagtgaaaggccttttttggaaaagcttatcg

SEQ ID NO: 20 - DNA, 45 bp, Artificial
  Oligonucleotide
accggcctttcactactcctacttagtgaagatcaagatcattgc

SEQ ID NO: 21 - DNA, 32 bp, Artificial
  Oligonucleotide
ttgatcttcactaagtaggagtagtgaaaggc

SEQ ID NO: 22 - DNA, 29 bp, Artificial
  Oligonucleotide
tttgtgtaggcaatgatcttgatcttcat

SEQ ID NO: 23 - DNA, 27 bp, Artificial
  Oligonucleotide
gatcattgcctacacaaagcaatgatc

SEQ ID NO: 24 - DNA, 33 bp, Artificial
  Oligonucleotide
tgagtaggagtagtgaaaggccttttttggaaa

SEQ ID NO: 25 - DNA, 48 bp, Artificial
  Oligonucleotide
agcttttccaaaaaaggcctttcactactcctactcaatgaagatcaa

SEQ ID NO: 26 - DNA, 65 bp, Artificial
  Oligonucleotide
acacaaagtaggagtagtgaaaggcctaagcaatgatcttgatcttcacggtgtttcgtcctttc

SEQ ID NO: 27 - DNA, 63 bp, Artificial
  Oligonucleotide
aggtaggagtagtgaaaggccttggcaatgatcttgatcttcattttttggaaaagcttatcg

SEQ ID NO: 28 - DNA, 65 bp, Artificial
  Oligonucleotide
acacaaagtaggagtagtgaaaggccctaagcaatgatcttgatcttcaggtgtttcgtcctttc

SEQ ID NO: 29 - DNA, 64 bp, Artificial
  Oligonucleotide
aggtaggagtagtgaaaggccttgagcaatgatcttgatcttcattttttggaaaagcttatcg

SEQ ID NO: 30 - DNA, 86 bp, Artificial
  Oligonucleotide
acaaatgaacaggtggtttcagtcctaagcaatgatcttgatcttcactaagtaggagtagtgaaaggccggtgtttcgtcctttc

SEQ ID NO: 31 - DNA, 89 bp, Artificial
  Oligonucleotide
gtagtgaacaggtggtttcagtcttgagcaatgatcttgatcttcattgagtaggagtagtgaaaggccttttttggaaaagcttatcg

SEQ ID NO: 32 - DNA, 89 bp, Artificial
  Oligonucleotide
cacaaatgaacaggtggtttcagtcctaagtaggagtagtgaaaggccctaagcaatgatcttgatcttcaccggtgtttcgtcctttc

SEQ ID NO: 33 - DNA, 88 bp, Artificial
  Oligonucleotide
tagtgaacaggtggtttcagtcttgagtaggagtagtgaaaggccttgagcaatgatcttgatcttcattttttggaaaagcttatcg

SEQ ID NO: 34 - DNA, 88 bp, Artificial
  Oligonucleotide
cacaaagtaggagtagtgaaaggccctaatgaacaggtggtttcagtcctaagcaatgatcttgatcttcacggtgtttcgtcctttc

SEQ ID NO: 35 - DNA, 88 bp, Artificial
  Oligonucleotide
taggtaggagtagtgaaaggccttgatgaacaggtggtttcagtcttgagcaatgatcttgatcttcattttttggaaaagcttatcg

SEQ ID NO: 36 - DNA, 88 bp, Artificial
  Oligonucleotide
cacaaagatgaacttcagggtcagcctaatgaacaggtggtttcagtcctaagcaatgatcttgatcttcacggtgtttcgtcctttc

SEQ ID NO: 37 - DNA, 88 bp, Artificial
  Oligonucleotide
taggatgaacttcagggtcagcttgatgaacaggtggtttcagtcttgagcaatgatcttgatcttcattttttggaaaagcttatcg

SEQ ID NO: 38 - DNA, 83 bp, Artificial
  Oligonucleotide
gttcatcaagctgaccctgaagttcatcctacacaaagatgaacttcagggtcagcctaatgaacaggtggtttcagtcctaa

SEQ ID NO: 39 - DNA, 80 bp, Artificial
  Oligonucleotide
aggtggtttcagtcttgagcaatgatcttgatcttcattgagtaggagtagtgaaaggccttttttggaaaagcttatcg

SEQ ID NO: 40 - DNA, 83 bp, Artificial; identical to SEQ ID NO: 38
  Oligonucleotide

SEQ ID NO: 41 - DNA, 80 bp, Artificial
  Oligonucleotide
aggtggtttcagtcttgagtaggagtagtgaaaggccttgagcaatgatcttgatcttcattttttggaaaagcttatcg

SEQ ID NO: 42 - DNA, 82 bp, Artificial
  Oligonucleotide
ctactcaagctgaccctgaagttcatcctacacaaagatgaacttcagggtcagcctaagtaggagtagtgaaaggccctaa

SEQ ID NO: 43 - DNA, 81 bp, Artificial
  Oligonucleotide
gagtagtgaaaggccttgatgaacaggtggtttcagtcttgagcaatgatcttgatcttcattttttggaaaagcttatcg

SEQ ID NO: 44 - DNA, 38 bp, Artificial
  Oligonucleotide
accgtgaagatcaagatcattgcttaggactgaaacca

SEQ ID NO: 45 - DNA, 43 bp, Artificial
  Oligonucleotide
atgaacaggtggtttcagtcctaagcaatgatcttgatcttca

SEQ ID NO: 46 - DNA, 35 bp, Artificial
  Oligonucleotide
cctgttcattaggctgaccctgaagttcatcttag

SEQ ID NO: 47 - DNA, 35 bp, Artificial
  Oligonucleotide
tgaaaggccctaagatgaacttcagggtcagccta

SEQ ID NO: 48 - DNA, 47 bp, Artificial
  Oligonucleotide
ggcctttcactactcctactttgtgtaggtaggagtagtgaaaggcc

SEQ ID NO: 49 - DNA, 47 bp, Artificial
  Oligonucleotide
tcatctcaaggcctttcactactcctacctacacaaagtaggagtag

SEQ ID NO: 50 - DNA, 46 bp, Artificial
  Oligonucleotide
ttgagatgaacttcagggtcagcttgatgaacaggtggtttcagtc

SEQ ID NO: 51 - DNA, 28 bp, Artificial
  Oligonucleotide
cacctgttcatcaagctgaccctgaagt

SEQ ID NO: 52 - DNA, 34 bp, Artificial
  Oligonucleotide
ttgagcaatgatcttgatcttcattttttggaaa

SEQ ID NO: 53 - DNA, 47 bp, Artificial
  Oligonucleotide
agcttttccaaaaaatgaagatcaagatcattgctcaagactgaaac

SEQ ID NO: 54 - DNA, 82 bp, Artificial
  Oligonucleotide
catctcaagtgtgcaccggcacagacactacacaaatgtctgtgccggtgcacacctaagatgaacttcagggtcagcctaa

SEQ ID NO: 55 - DNA, 81 bp, Artificial
  Oligonucleotide
aacttcagggtcagcttgatgaacaggtggtttcagtcttgagcaatgatcttgatcttcattttttggaaaagcttatcg

SEQ ID NO: 56 - DNA, 78 bp, Artificial
  Oligonucleotide
tcaagtgtgcaccggcacagacactacacaaatgtctgtgccggtgcacacctaagatgaacttcagggtcagcctaa

SEQ ID NO: 57 - DNA, 108 bp, Artificial
  Oligonucleotide
gatgaacttcagggtcagcttgatgaacaggtggtttcagtcttgagcaatgatcttgatcttcattgagtaggagtagtgaaaggccttttttggaaaagcttatcg

SEQ ID NO: 58 - DNA, 78 bp, Artificial; identical to SEQ ID NO: 56
  Oligonucleotide

SEQ ID NO: 59 - DNA, 108 bp, Artificial
  Oligonucleotide
gatgaacttcagggtcagcttgatgaacaggtggtttcagtcttgagtaggagtagtgaaaggccttgagcaatgatcttgatcttcattttttggaaaagcttatcg

SEQ ID NO: 60 - DNA, 78 bp, Artificial; identical to SEQ ID NO: 56
  Oligonucleotide

SEQ ID NO: 61 - DNA, 108 bp, Artificial
  Primer sequence
gatgaacttcagggtcagcttgagtaggagtagtgaaaggccttgatgaacaggtggtttcagtcttgagcaatgatcttgatcttcattttttggaaaagcttatcg

SEQ ID NO: 62 - DNA, 78 bp, Artificial
  Oligonucleotide
tcaagtgtgcaccggcacagacactacacaaatgtctgtgccggtgcacacctaagtaggagtagtgaaaggccctaa

SEQ ID NO: 63 - DNA, 108 bp, Artificial
  Primer sequence
gtaggagtagtgaaaggccttgagatgaacttcagggtcagcttgatgaacaggtggtttcagtcttgagcaatgatcttgatcttcattttttggaaaagcttatcg

SEQ ID NO: 64 - DNA, 38 bp, Artificial; identical to SEQ ID NO: 44
  Oligonucleotide

SEQ ID NO: 65 - DNA, 43 bp, Artificial; identical to SEQ ID NO: 45
  Oligonucleotide

SEQ ID NO: 66 - DNA, 35 bp, Artificial; identical to SEQ ID NO: 46
  Oligonucleotide

SEQ ID NO: 67 - DNA, 35 bp, Artificial
  Oligonucleotide
ggtgcacacctaagatgaacttcagggtcagccta

SEQ ID NO: 68 - DNA, 47 bp, Artificial
  Oligonucleotide
gtgtgcaccggcacagacattagggcctttcactactcctactttgt

SEQ ID NO: 69 - DNA, 47 bp, Artificial
  Oligonucleotide
cctacctacacaaagtaggagtagtgaaaggccctaatgtctgtgcc

SEQ ID NO: 70 - DNA, 46 bp, Artificial
  Oligonucleotide
gtaggtaggagtagtgaaaggccttgatgtctgtgccggtgcacac

SEQ ID NO: 71 - DNA, 46 bp, Artificial
  Oligonucleotide
tcatctcaagtgtgcaccggcacagacatcaaggcctttcactact

SEQ ID NO: 72 - DNA, 46 bp, Artificial; identical to SEQ ID NO: 50
  Oligonucleotide

SEQ ID NO: 73 - DNA, 28 bp, Artificial
  Oligonucleotide
cacctgttcatcaagctgaccctgaagt

SEQ ID NO: 74 - DNA, 34 bp, Artificial; identical to SEQ ID NO: 52
  Oligonucleotide

SEQ ID NO: 75 - DNA, 47 bp, Artificial; identical to SEQ ID NO: 53
  Oligonucleotide

SEQ ID NO: 76 - DNA, 78 bp, Artificial
  Oligonucleotide
tcaactggacttccagaagaacactacacaaatgttcttctggaagtccagctaatgtctgtgccggtgcacacctaa

SEQ ID NO: 77 - DNA, 108 bp, Artificial
  Oligonucleotide
tgtctgtgccggtgcacacttgagatgaacttcagggtcagcttgatgaacaggtggtttcagtcttgagcaatgatcttgatcttcattttttggaaaagcttatcg

SEQ ID NO: 78 - DNA, 65 bp, Artificial
  Oligonucleotide
cacaaagaggcgctcgtggtcctggctaacagcttctcgtggtcctggcggtgtttcgtcctttc

SEQ ID NO: 79 - DNA, 65 bp, Artificial
  Oligonucleotide
taggaggcgctcgtggtcctggttgacagcttctcgtggtcctggttttttggaaaagcttatcg

SEQ ID NO: 80 - DNA, 42 bp, Artificial
  Oligonucleotide
cacaaaggtgcccttgccgaggagtcggtgtttcgtcctttc

SEQ ID NO: 81 - DNA, 42 bp, Artificial
  Oligonucleotide
taggaggcgctcgtggtcctggttttttggaaaagcttatcg

SEQ ID NO: 82 - DNA, 42 bp, Artificial
  Oligonucleotide
cacaaagaggcgctcgtggtcctggcggtgtttcgtcctttc

SEQ ID NO: 83 - DNA, 41 bp, Artificial
  Oligonucleotide
tagggtgcccttgccgaggagttttttggaaaagcttatcg

SEQ ID NO: 84 - DNA, 65 bp, Artificial
  Oligonucleotide
cacaaagaggcgctcgtggtcctggctaaggtgcccttgccgaggagtcggtgtttcgtcctttc

SEQ ID NO: 85 - DNA, 64 bp, Artificial
  Oligonucleotide
taggaggcgctcgtggtcctggttgaggtgcccttgccgaggagttttttggaaaagcttatcg

SEQ ID NO: 86 - DNA, 32 bp, Artificial
  Oligonucleotide
accgtgaagatcaagatcattgctcctcctga

SEQ ID NO: 87 - DNA, 18 bp, Artificial
  Oligonucleotide
caatgatcttgatcttca

SEQ ID NO: 88 - DNA, 28 bp, Artificial
  Oligonucleotide
gcgcaagtactccgtgtggttcaagaga

SEQ ID NO: 89 - DNA, 29 bp, Artificial
  Oligonucleotide
ccacacggagtacttgcgctcaggaggag

SEQ ID NO: 90 - DNA, 31 bp, Artificial
  Oligonucleotide
ccacacggagtacttgcgctcaggaggagca

SEQ ID NO: 91 - DNA, 31 bp, Artificial
  Oligonucleotide
tgagcgcaagtactccgtgtggtctcttgaa

SEQ ID NO: 92 - DNA, 27 bp, Artificial
  Oligonucleotide
aatgatcttgatcttcatttttggaaa

SEQ ID NO: 93 - DNA, 39 bp, Artificial
  Oligonucleotide
agcttttccaaaaatgaagatcaagatcattgctcctcc

SEQ ID NO: 94 - DNA, 48 bp, Artificial
  Oligonucleotide
agagatgaacaggtggtttcagtctttttggaaaagcttatcgatacc

SEQ ID NO: 95 - DNA, 45 bp, Artificial
  Oligonucleotide
tgaatgaacaggtggtttcagtcggtgtttcgtcctttccacaag

SEQ ID NO: 96 - DNA, 48 bp, Artificial
  Oligonucleotide
agagaggctgtgaacagacgcgcctttttggaaaagcttatcgatacc

SEQ ID NO: 97 - DNA, 45 bp, Artificial
  Oligonucleotide
tgaaggctgtgaacagacgcgccggtgtttcgtcctttccacaag

SEQ ID NO: 98 - DNA, 50 bp, Artificial
  Oligonucleotide
agagaagtgctgctggaactcatgctttttggaaaagcttatcgataccg

SEQ ID NO: 99 - DNA, 46 bp, Artificial
  Oligonucleotide
tgaaagtgctgctggaactcatgcggtgtttcgtcctttccacaag

SEQ ID NO: 100 - DNA, 50 bp, Artificial
  Oligonucleotide
agagattatttctgcatggcagtcatttttggaaaagcttatcgataccg

SEQ ID NO: 101 - DNA, 45 bp, Artificial
  Oligonucleotide
tgaattatttctgcatggcagtcggtgtttcgtcctttccacaag

SEQ ID NO: 102 - DNA, 58 bp, Artificial
  Oligonucleotide
agagaggctgtgaacagacgcgcctttgaacaggtggtttcagtctttttggaaaagc

SEQ ID NO: 103 - DNA, 54 bp, Artificial
  Oligonucleotide
tgaaggctgtgaacagacgcgccaatgaacaggtggtttcagtcggtgtttcgt

SEQ ID NO: 104 - DNA, 59 bp, Artificial
  Oligonucleotide
agagaggctgtgaacagacgcgccttgtgaacaggtggtttcagtctttttggaaaagc

SEQ ID NO: 105 - DNA, 55 bp, Artificial
  Oligonucleotide
tgaaggctgtgaacagacgcgcctaatgaacaggtggtttcagtcggtgtttcgt

SEQ ID NO: 106 - DNA, 62 bp, Artificial
  Oligonucleotide
agagaggctgtgaacagacgcgccttgtaatgaacaggtggtttcagtctttttggaaaagc

SEQ ID NO: 107 - DNA, 57 bp, Artificial
  Oligonucleotide
tgaaggctgtgaacagacgcgccttgtaatgaacaggtggtttcagtcggtgtttcg

SEQ ID NO: 108 - DNA, 29 bp, Artificial
  Oligonucleotide
aatgactgccatgcagaaatacctgtctc

SEQ ID NO: 109 - DNA, 29 bp, Artificial
  Oligonucleotide
aatatttctgcatggcagtcacctgtctc

SEQ ID NO: 110 - DNA, 29 bp, Artificial
  Oligonucleotide
aatgaagatcaagatcattgccctgtctc

SEQ ID NO: 111 - DNA, 29 bp, Artificial
  Oligonucleotide
aagcaatgatcttgatcttcacctgtctc

SEQ ID NO: 112 - DNA, 50 bp, Artificial
  Oligonucleotide
ctgaaatctcttgaatttcagtctaagcaatgatcttgatcttcacggtg

SEQ ID NO: 113 - DNA, 51 bp, Artificial
  Oligonucleotide
tcttggcaatgatcttgatcttcatttttggaaaagcttatcgataccgtc

SEQ ID NO: 114 - DNA, 60 bp, Artificial
  Oligonucleotide
tcattgctctcttgaagcaatgatcttgatcttcaaatgaacaggtggtttcagtcggtg

SEQ ID NO: 115 - DNA, 60 bp, Artificial
  Oligonucleotide
tcttgatcttcatttgaacaggtggtttcagtctttttggaaaagcttatcgataccgtc

SEQ ID NO: 116 - DNA, 61 bp, Artificial
  Oligonucleotide
tcattgctctcttgaagcaatgatcttgatcttcataatgaacaggtggtttcagtcggtg

SEQ ID NO: 117 - DNA, 61 bp, Artificial
  Oligonucleotide
tcttgatcttcattgtgaacaggtggtttcagtctttttggaaaagcttatcgataccgtc

SEQ ID NO: 118 - DNA, 61 bp, Artificial
  Oligonucleotide
cattgctctcttgaagcaatgatcttgatcttcactaatgaacaggtggtttcagtcggtg

SEQ ID NO: 119 - DNA, 63 bp, Artificial
  Oligonucleotide
atcttgatcttcattgatgaacaggtggtttcagtctttttggaaaagcttatcgataccgtc

SEQ ID NO: 120 - DNA, 63 bp, Artificial
  Oligonucleotide
cattgctctcttgaagcaatgatcttgatcttcattgtaatgaacaggtggtttcagtcggtg

SEQ ID NO: 121 - DNA, 65 bp, Artificial
  Oligonucleotide
atcttgatcttcattgtaatgaacaggtggtttcagtctttttggaaaagcttatcgataccgtc

SEQ ID NO: 122 - DNA, 61 bp, Artificial
  Oligonucleotide
tgttcatctcttgaatgaacaggtggtttcagtctaagcaatgatcttgatcttcacggtg

SEQ ID NO: 123 - DNA, 62 bp, Artificial
  Oligonucleotide
ggtggtttcagtcttggcaatgatcttgatcttcatttttggaaaagcttatcgataccgtc

SEQ ID NO: 124 - DNA, 32 bp, Artificial
  Oligonucleotide
accgctgaccctgaagttcatcctggcctttc

SEQ ID NO: 125 - DNA, 36 bp, Artificial
  Oligonucleotide
ggagtagtgaaaggccaggatgaacttcagggtcag

SEQ ID NO: 126 - DNA, 22 bp, Artificial
  Oligonucleotide
actactcctactttgtgtaggt

SEQ ID NO: 127 - DNA, 23 bp, Artificial
  Oligonucleotide
actactcctacctacacaaagta

SEQ ID NO: 128 - DNA, 29 bp, Artificial
  Oligonucleotide
aggagtagtgaaaggccaggatgaacttc

SEQ ID NO: 129 - DNA, 28 bp, Artificial
  Oligonucleotide
ctgaccctgaagttcatcctggcctttc

SEQ ID NO: 130 - DNA, 20 bp, Artificial
  Oligonucleotide
agggtcagcttttttggaaa

SEQ ID NO: 131 - DNA, 16 bp, Artificial
  Oligonucleotide
agcttttccaaaaaag

SEQ ID NO: 132 - DNA, 32 bp, Artificial
  Oligonucleotide
accgctgaccctgaagttcatccnngcctttc

SEQ ID NO: 133 - DNA, 36 bp, Artificial
  Oligonucleotide
ggagtagtgaaaggcnnggatgaacttcagggtcag

SEQ ID NO: 134 - DNA, 22 bp, Artificial
  Oligonucleotide
actactcctactttcagtaggt

SEQ ID NO: 135 - DNA, 23 bp, Artificial
  Oligonucleotide
actactcctacctacacaaagta

SEQ ID NO: 136 - DNA, 29 bp, Artificial
  Oligonucleotide
aggagtagtgaaaggccaggatgaacttc

SEQ ID NO: 137 - DNA, 28 bp, Artificial
  Oligonucleotide
ctgaccctgaagttcatcctggcctttc

SEQ ID NO: 138 - DNA, 20 bp, Artificial
  Oligonucleotide
agggtcagcttttttggaaa

SEQ ID NO: 139 - DNA, 16 bp, Artificial
  Oligonucleotide
agcttttccaaaaaag

SEQ ID NO: 140 - DNA, 32 bp, Artificial; identical to SEQ ID NO: 124
  Oligonucleotide

SEQ ID NO: 141 - DNA, 36 bp, Artificial; identical to SEQ ID NO: 133
  Oligonucleotide

SEQ ID NO: 142 - DNA, 22 bp, Artificial
  Oligonucleotide
actactcctactttgtgtaggt

SEQ ID NO: 143 - DNA, 23 bp, Artificial
  Oligonucleotide
actactcctacctacacaaagta

SEQ ID NO: 144 - DNA, 29 bp, Artificial
  Oligonucleotide
aggagtagtgaaaggccannatgaacttc

SEQ ID NO: 145 - DNA, 28 bp, Artificial
  Oligonucleotide
ctgaccctgaagttcatnntggcctttc

SEQ ID NO: 146 - DNA, 20 bp, Artificial
  Oligonucleotide
agggtcagcttttttggaaa

SEQ ID NO: 147 - DNA, 16 bp, Artificial
  Oligonucleotide
agcttttccaaaaaag

SEQ ID NO: 148 - DNA, 32 bp, Artificial; identical to SEQ ID NO: 132
  Oligonucleotide

SEQ ID NO: 149 - DNA, 36 bp, Artificial; identical to SEQ ID NO: 133
  Oligonucleotide

SEQ ID NO: 150 - DNA, 22 bp, Artificial
  Oligonucleotide
actactcctactttgtgtaggt

SEQ ID NO: 151 - DNA, 23 bp, Artificial
  Oligonucleotide
actactcctacctacacaaagta

SEQ ID NO: 152 - DNA, 29 bp, Artificial
  Oligonucleotide
aggagtagtgaaaggccannatgaacttc

SEQ ID NO: 153 - DNA, 28 bp, Artificial
  Oligonucleotide
ctgaccctgaagttcatnntggcctttc

SEQ ID NO: 154 - DNA, 20 bp, Artificial
  Oligonucleotide
agggtcagcttttttggaaa

SEQ ID NO: 155 - DNA, 16 bp, Artificial
  Oligonucleotide
agcttttccaaaaaag

SEQ ID NO: 156 - DNA, 17 bp, Artificial
  Oligonucleotide
accgctgaccctgaagt

SEQ ID NO: 157 - DNA, 32 bp, Artificial
  Oligonucleotide
agtgaaaggddbhagatgaacttcagggtcag

SEQ ID NO: 158 - DNA, 33 bp, Artificial
  Oligonucleotide
tcatctdvhhcctttcactactcctactttgtg

SEQ ID NO: 159 - DNA, 24 bp, Artificial
  Oligonucleotide
ctcctacctacacaaagtaggagt

SEQ ID NO: 160 - DNA, 31 bp, Artificial
  Oligonucleotide
taggtaggagtagtgaaaggddbhagatgaa

SEQ ID NO: 161 - DNA, 30 bp, Artificial
  Oligonucleotide
accctgaagttcatctdvhhcctttcacta

SEQ ID NO: 162 - DNA, 32 bp, Artificial
  Oligonucleotide
agtgaaaggnttnagatgaacttcagggtcag

SEQ ID NO: 163 - DNA, 33 bp, Artificial
  Oligonucleotide
tcatctnaancctttcactactcctactttgtg

SEQ ID NO: 164 - DNA, 31 bp, Artificial
  Oligonucleotide
taggtaggagtagtgaaaggnaanagatgaa

SEQ ID NO: 165 - DNA, 30 bp, Artificial
  Oligonucleotide
accctgaagttcatctnttncctttcacta

SEQ ID NO: 166 - DNA, 62 bp, Artificial
  Oligonucleotide
cacaaagtaggagtagtgaaaggddbhgatgaacttcagggtcagcggtgtttcgtcctttc

SEQ ID NO: 167 - DNA, 63 bp, Artificial
  Oligonucleotide
taggtaggagtagtgaaaggccbhhbtgaacttcagggtcagcttttttggaaaagcttatcg

SEQ ID NO: 168 - DNA, 19 bp, Artificial
  Oligonucleotide
accgctgaccctgaagttc

SEQ ID NO: 169 - DNA, 24 bp, Artificial
  Oligonucleotide
gaaaggccagaacttcagggtcag

SEQ ID NO: 170 - DNA, 26 bp, Artificial
  Oligonucleotide
tggcctttcactactcctactttgtg

SEQ ID NO: 171 - DNA, 27 bp, Artificial
  Oligonucleotide
ctcctacctacacaaagtaggagtagt

SEQ ID NO: 172 - DNA, 26 bp, Artificial
  Oligonucleotide
taggtaggagtagtgaaaggccagaa

SEQ ID NO: 173 - DNA, 24 bp, Artificial
  Oligonucleotide
accctgaagttctggcctttcact

SEQ ID NO: 174 - DNA, 24 bp, Artificial
  Oligonucleotide
cttcagggtcagcttttttggaaa

SEQ ID NO: 175 - DNA, 19 bp, Artificial
  Oligonucleotide
agcttttccaaaaaagctg

SEQ ID NO: 176 - DNA, 25 bp, Artificial
  Oligonucleotide
gaaaggccatgaacttcagggtcag

SEQ ID NO: 177 - DNA, 27 bp, Artificial
  Oligonucleotide
atggcctttcactactcctactttgtg

SEQ ID NO: 178 - DNA, 27 bp, Artificial
  Oligonucleotide
taggtaggagtagtgaaaggccatgaa

SEQ ID NO: 179 - DNA, 26 bp, Artificial
  Oligonucleotide
accctgaagttcatggcctttcacta

SEQ ID NO: 180 - DNA, 26 bp, Artificial
  Oligonucleotide
gaaaggccaatgaacttcagggtcag

SEQ ID NO: 181 - DNA, 28 bp, Artificial
  Oligonucleotide
attggcctttcactactcctactttgtg

SEQ ID NO: 182 - DNA, 28 bp, Artificial
  Oligonucleotide
taggtaggagtagtgaaaggccagtgaa

SEQ ID NO: 183 - DNA, 27 bp, Artificial
  Oligonucleotide
accctgaagttcactggcctttcacta

SEQ ID NO: 184 - DNA, 27 bp, Artificial
  Oligonucleotide
gaaaggccagatgaacttcagggtcag

SEQ ID NO: 185 - DNA, 29 bp, Artificial
  Oligonucleotide
atctggcctttcactactcctactttgtg

SEQ ID NO: 186 - DNA, 29 bp, Artificial
  Oligonucleotide
taggtaggagtagtgaaaggccagatgaa

SEQ ID NO: 187 - DNA, 28 bp, Artificial
  Oligonucleotide
accctgaagttcatctggcctttcacta

SEQ ID NO: 188 - DNA, 28 bp, Artificial
  Oligonucleotide
gaaaggccaggatgaacttcagggtcag

SEQ ID NO: 189 - DNA, 30 bp, Artificial
  Oligonucleotide
atcctggcctttcactactcctactttgtg

SEQ ID NO: 190 - DNA, 30 bp, Artificial
  Primer sequence
taggtaggagtagtgaaaggccaggatgaa

SEQ ID NO: 191 - DNA, 29 bp, Artificial
  Oligonucleotide
accctgaagttcatcctggcctttcacta

SEQ ID NO: 192 - DNA, 29 bp, Artificial
  Oligonucleotide
gaaaggccagagatgaacttcagggtcag

SEQ ID NO: 193 - DNA, 31 bp, Artificial
  Oligonucleotide
atctctggcctttcactactcctactttgtg

SEQ ID NO: 194 - DNA, 31 bp, Artificial
  Oligonucleotide
taggtaggagtagtgaaaggccagagatgaa

SEQ ID NO: 195 - DNA, 31 bp, Artificial
  Oligonucleotide
accctgaagttcatctgctggcctttcacta

SEQ ID NO: 196 - DNA, 30 bp, Artificial
  Oligonucleotide
gaaaggccagcagatgaacttcagggtcag

SEQ ID NO: 197 - DNA, 32 bp, Artificial
  Oligonucleotide
atctgctggcctttcactactcctactttgtg

SEQ ID NO: 198 - DNA, 32 bp, Artificial
  Oligonucleotide
taggtaggagtagtgaaaggccagcagatgaa

SEQ ID NO: 199 - DNA, 31 bp, Artificial; identical to SEQ ID NO: 195
  Oligonucleotide

SEQ ID NO: 200 - DNA, 31 bp, Artificial
  Oligonucleotide
gaaaggccaggcagatgaacttcagggtcag

SEQ ID NO: 201 - DNA, 33 bp, Artificial
  Oligonucleotide
atctgcctggcctttcactactcctactttgtg

SEQ ID NO: 202 - DNA, 33 bp, Artificial
  Oligonucleotide
taggtaggagtagtgaaaggccaggcagatgaa

SEQ ID NO: 203 - DNA, 32 bp, Artificial
  Oligonucleotide
accctgaagttcatctgcctggcctttcacta

SEQ ID NO: 204 - DNA, 32 bp, Artificial
  Oligonucleotide
gaaaggccagtgcagatgaacttcagggtcag

SEQ ID NO: 205 - DNA, 34 bp, Artificial
  Oligonucleotide
atctgcactggcctttcactactcctactttgtg

SEQ ID NO: 206 - DNA, 34 bp, Artificial
  Oligonucleotide
taggtaggagtagtgaaaggccagtgcagatgaa

SEQ ID NO: 207 - DNA, 33 bp, Artificial
  Oligonucleotide
accctgaagttcatctgcactggcctttcacta

SEQ ID NO: 208 - DNA, 33 bp, Artificial
  Oligonucleotide
gaaaggccaggtgcagatgaacttcagggtcag

SEQ ID NO: 209 - DNA, 35 bp, Artificial
  Oligonucleotide
atctgcacctggcctttcactactcctactttgtg

SEQ ID NO: 210 - DNA, 35 bp, Artificial
  Oligonucleotide
taggtaggagtagtgaaaggccaggtgcagatgaa

SEQ ID NO: 211 - DNA, 34 bp, Artificial
  Oligonucleotide
accctgaagttcatctgcacctggcctttcacta

SEQ ID NO: 212 - DNA, 42 bp, Artificial
  Oligonucleotide
cacaaacagcttctcgtggtcctggcggtgtttcgtcctttc

SEQ ID NO: 213 - DNA, 42 bp, Artificial
  Oligonucleotide
tagcagcttctcgtggtcctggttttttggaaaagcttatcg

SEQ ID NO: 214 - DNA, 26 bp, Artificial
  Oligonucleotide
accggagagccatagtggtctggaaa

SEQ ID NO: 215 - DNA, 32 bp, Artificial
  Oligonucleotide
accggttccgtttccagaccactatggctctc

SEQ ID NO: 216 - DNA, 25 bp, Artificial
  Oligonucleotide
cggaaccggtgagtacacgaaaagg

SEQ ID NO: 217 - DNA, 30 bp, Artificial
  Oligonucleotide
gcacggtctacgagaccttttcgtgtactc

SEQ ID NO: 218 - DNA, 24 bp, Artificial
  Oligonucleotide
tctcgtagaccgtgcatttgtgta

SEQ ID NO: 219 - DNA, 20 bp, Artificial
  Oligonucleotide
agaccgtgcactacacaaat

SEQ ID NO: 220 - DNA, 27 bp, Artificial
  Oligonucleotide
gtgcacggtctacgagacctcaaggtg

SEQ ID NO: 221 - DNA, 24 bp, Artificial
  Oligonucleotide
ggtgagtacaccttgaggtctcgt

SEQ ID NO: 222 - DNA, 27 bp, Artificial
  Oligonucleotide
tactcaccggttccgcaagcagaccac

SEQ ID NO: 223 - DNA, 28 bp, Artificial
  Oligonucleotide
agagccatagtggtctgcttgcggaacc

SEQ ID NO: 224 - DNA, 22 bp, Artificial
  Oligonucleotide
tatggctctccttttttggaaa

SEQ ID NO: 225 - DNA, 17 bp, Artificial
  Oligonucleotide
agcttttccaaaaaagg

SEQ ID NO: 226 - RNA, 49 nt, Artificial
  Hairpin sequence
ugaagaucaagaucauugcuucaagagagcaaugaucuugaucuucauu

SEQ ID NO: 227 - DNA, 39 bp, Artificial
  Oligonucleotide
tataggcgcgccagagagcaatgatcttgatcttcattt

SEQ ID NO: 228 - DNA, 67 bp, Artificial
  Hairpin sequence
aaacaccgtgaagatcaagatcattgctttcaagagagagcaatgatcttgatcttcatttttggaa

SEQ ID NO: 229 - DNA, 30 bp, Artificial
  Oligonucleotide
cttgaaagcaatgatcttgatcttcacggt

SEQ ID NO: 230 - DNA, 58 bp, Artificial; identical to SEQ ID NO: 11
  Oligonucleotide

SEQ ID NO: 231 - DNA, 58 bp, Artificial
  Plasmid sequence
atatatcttgtggaaaggacgaaacacctttttggaaaagcttatcgataccgtcgac

SEQ ID NO: 232 - DNA, 58 bp, Artificial; identical to SEQ ID NO: 10
  Oligonucleotide

SEQ ID NO: 233 - DNA, 49 bp, Artificial
  Oligonucleotide
cagggcacgggcagcttgccggtggtgcagatgaacttcagggtcagcc

SEQ ID NO: 234 - DNA, 60 bp, Artificial
  Plasmid sequence
accgctgaccctgaagttcatcttcaagagagatgaacttcagggtcagctttttggaaa

SEQ ID NO: 235 - DNA, 49 bp, Artificial
  Oligonucleotide
cagggcacgggcagcttgccggtggtgcaagatgaacttcagggtcagc

SEQ ID NO: 236 - DNA, 62 bp, Artificial; identical to SEQ ID NO: 12
  Oligonucleotide

SEQ ID NO: 237 - DNA, 55 bp, Artificial
  Primer sequence
ccgccatgcccgaaggctacgtccaggagcgcaccatcttcttcaaggacgacgg

SEQ ID NO: 238 - DNA, 66 bp, Artificial; identical to SEQ ID NO: 13
  Oligonucleotide

SEQ ID NO: 239 - RNA, 49 nt, Artificial
  Hairpin sequence
ggccuuucacuacuccuacuuuguguagguaggaguagugaaaggccuu

SEQ ID NO: 240 - RNA, 93 nt, Artificial
  Hairpin sequence
ggccuuucacuacuccuacuuaugaagaucaagaucauugcuuguuguaggcaaugaucuugaucuucauugguaggaguagugaaaggccuu

SEQ ID NO: 241 - RNA, 95 nt, Artificial
  Hairpin sequence
ggccuuucacuacuccuacuuagugaagaucaagaucauugcuuguuguaggcaaugaucuugaucuucauugaguaggaguagugaaaggccuu

SEQ ID NO: 242 - RNA, 95 nt, Artificial
  Hairpin sequence
gugaagaucaagaucauugcuuaggccuuucacuacuccuacuuuguuguagguaggaguagugaaaggccuuggcaaugaucuugaucuucauu

SEQ ID NO: 243 - RNA, 97 nt, Artificial
  Hairpin sequence
gugaagaucaagaucauugcuuagggccuuucacuacuccuacuuuguuguagguaggaguagugaaaggccuugagcaaugaucuugaucuucauu

SEQ ID NO: 244 - RNA, 96 nt, Artificial
  Hairpin sequence
gugaagaucaagaucauugcuuaggacugaaaccaccuguucauuuguguagugaacaggugguuucagucuugagcaaugaucuugaucuucauu

SEQ ID NO: 245 - RNA, 142 nt, Artificial
  Hairpin sequence
ggccuuucacuacuccuacuuagugaagaucaagaucauugcuuaggacugaaaccaccuguucauuuguuguagugaacaggugguuucagucuugagcaaugaucuugaucuucauugaguaggaguaguguuuggccuu

SEQ ID NO: 246 - RNA, 143 nt, Artificial
  Hairpin sequence
gugaagaucaagaucauugcuuagggccuuucacuacuccuacuuaggacugaaaccaccuguucauuuguuguagugaacaggugguuucagucuugaguaggaguagugaaaggccuugagcaaugaucuugaucuucauu

SEQ ID NO: 247 - RNA, 143 nt, Artificial
  Hairpin sequence
gugaagaucaagaucauugcuuaggacugaaaccaccuguucauuagggccuuucacuacuccuacuuuguuguagguaggaguagugaaaggccuugaugaacaggugguuucagucuugagcaaugaucuugaucuucauu

SEQ ID NO: 248 - RNA, 143 nt, Artificial
  Hairpin Sequence
gugaagaucaagaucauugcuuaggacugaaaccaccuguucauuaggcugacccugaaguucaucuuuguuguaggaugaacuucagggucagcuugaugaacaggugguuucagucuugagcaaugaucuugaucuucauu

SEQ ID NO: 249 - RNA, 186 nt, Artificial
  Hairpin sequence
ggccuucacuacuccuacuuagugaagaucaagaucauugcuuaggacugaaaccaccuguucauuaggcugacccugaaguucaucuuuguuguaggaugaacuucagggucagcuugaugaacaggugguuucagucuugagcaaugaucuugaucuucauugaguaggaguagugaaggccuu

SEQ ID NO: 250 - RNA, 188 nt, Artificial
  Hairpin sequence
gugaagaucaagaucauugcuuagggccuuucacuacuccuacuuaggacugaaaccaccuguucauuaggcugacccugaaguucaucuuuguguaggaugaacuucagggucagcuugaugaacaggugguuucagucuugaguaggaguagugaaaggccuugagcaaugaucuugaucuucauu

SEQ ID NO: 251 - RNA, 188 nt, Artificial
  Hairpin sequence
gugaagaucaagaucauugcuuaggacugaaaccaccuguucauuagggccuuucacuacuccuacuuaggcugacccugaaguucaucuuuguguaggaugaacuucagggucagcuugaguaggaguagugaaaggccuugaugaacaggugguuucagucuugagcaaugaucuugaucuucauu

SEQ ID NO: 252 - RNA, 188 nt, Artificial
  Hairpin sequence
gugaagaucaagaucauugcuuaggacugaaaccaccuguucauuaggcugacccugaaguucaucuuagggccuuucacuacuccuacuuuguguagguaggaguagugaaaggccuugagaugaacuucagggucagcuugaugaacaggugguuucagucuugagcaaugaucuugaucuucauu

SEQ ID NO: 253 - RNA, 188 nt, Artificial
  Hairpin sequence
gugaagaucaagaucauugcuuaggacugaaaccaccuguucauuaggcugacccugaaguucaucuuaggugugcaccggcacagacauuuguguagugucugugccggugcacacuugagaugaacuucagggucagcuugaugaacaggugguuucagucuugagcaaugaucuugaucuucauu

SEQ ID NO: 254 - RNA, 233 nt, Artificial
  Hairpin sequence
ggccuuucacuacuccuacuuagugaagaucaagaucauugcuuaggacugaaaccaccuguucauuaggcugacccugaaguucaucuuaggugugcaccggcacagacauuuguguagugucugugccggugcacacuugagaugaacuucagggucagcuugaugaacaggugguuucagucuugagcaaugaucuugaucuucauugaguaggaguagugaaaggccuu

SEQ ID NO: 255 - RNA, 234 nt, Artificial
  Hairpin sequence
gugaagaucaagaucauugcuuagggccuuucacuacuccuacuuaggacugaaaccaccuguucauuaggcugacccugaaguucaucuuaggugugcaccggcacagacauuuguguagugucugugccggugcacacuugagaugaacuucagggucagcuugaugaacaggugguuucagucuugaguaggaguagugaaaggccuugagcaaugaucuugaucuucauu

SEQ ID NO: 256 - RNA, 234 nt, Artificial
  Hairpin sequence
gugaagaucaagaucauugcuuaggacugaaaccaccuguucauuagggccuuucacuacuccuacuuaggcugacccugaaguucaucuuaggugugcaccggcacagacauuuguguagugucugugccggugcacacuugagaugaacuucagggucagcuugaguaggaguagugaaaggccuugaugaacaggugguuucagucuugagcaaugaucuugaucuucauu

SEQ ID NO: 257 - RNA, 234 nt, Artificial
  Hairpin sequence
gugaagaucaagaucauugcuuaggacugaaaccaccuguucauuaggcugacccugaaguucaucuuagggccuuucacuacuccuacuuaggugugcaccggcacagacauuuguguagugucugugccggugcacacuugaguaggaguagugaaaggccuugagaugaacuucagggucagcuugaugaacaggugguuucagucuugagcaaugaucuugaucuucauu

SEQ ID NO: 258 - RNA, 234 nt, Artificial
  Hairpin sequence
gugaagaucaagaucauugcuuaggacugaaaccaccuguucauuaggcugacccugaaguucaucuuaggugugcaccggcacagacauuagggccuuucacuacuccuacuuuguguagguaggaguagugaaaggccuugaugucugugccggugcacacuugagaugaacuucagggucagcuugaugaacaggugguuucagucuugagcaaugaucuugaucuucauu

SEQ ID NO: 259 - RNA, 234 nt, Artificial
  Hairpin sequence
gugaagaucaagaucauugcuuaggacugaaaccaccuguucauuaggcugacccugaaguucaucuuaggugugcaccggcacagacauuagcuggacuuccagaagaacauuuguguaguguucuucuggaaguccaguugaugucugugccggugcacacuugagaugaacuucagggucagcuugaugaacaggugguuucagucuugagcaaugaucuugaucuucauu

SEQ ID NO: 260 - RNA, 96 nt, Artificial
  Hairpin sequence
gccaggaccacgagaagcuguuagccaggaccacgagcgccucuuuguguaggaggcgcucgugguccugguugacagcuucucgugguccugguu

SEQ ID NO: 261 - RNA, 50 nt, Artificial
  Hairpin sequence
gacuccucggcaagggcaccuuuguguagggugcccuugccgaggaguuu

SEQ ID NO: 262 - RNA, 50 nt, Artificial
  Hairpin sequence
gccaggaccacgagcgccucuuuguguaggaggcgcucgugguccugguu

SEQ ID NO: 263 - RNA, 95 nt, Artificial
  Hairpin sequence
gacuccucggcaagggcaccuuagccaggaccacgagcgccucuuuguguaggaggcgcucgugguccugguugaggugcccuugccgaggaguu

SEQ ID NO: 264 - RNA, 106 nt, Artificial
  Hairpin sequence
gugaagaucaagaucauugcuccuccugagcgcaaguacuccgugugguucaagagaccacacggaguacuugcgcucaggaggagcaaugaucuugaucuucauu

SEQ ID NO: 265 - RNA, 49 nt, Artificial
  Hairpin sequence
gacugaaaccaccuguucauucaagagaugaacaggugguuucagucuu

SEQ ID NO: 266 - RNA, 51 nt, Artificial
  Hairpin sequence
gcaugaguuccagcagcacuuucaagagaagugcugcuggaacucaugcuu

SEQ ID NO: 267 - RNA, 49 nt, Artificial
  Hairpin sequence
ggcgcgucuguucacagccuucaagagaggcugugaacagacgcgccuu

SEQ ID NO: 268 - RNA, 50 nt, Artificial
  Hairpin sequence
gacugccaugcagaaauaauucaagagauuauuucugcauggcagucauu

SEQ ID NO: 269 - RNA, 91 nt, Artificial
  Hairpin sequence
gacugaaaccaccuguucauuggcgcgucuguucacagccuucaagagaggcugugaacagacgcgccuuugaacaggugguuucagucuu

SEQ ID NO: 270 - RNA, 93 nt, Artificial
  Hairpin sequence
gacugaaaccaccuguucauuaggcgcgucuguucacagccuucaagagaggcugugaacagacgcgccuugugaacaggugguuucagucuu

SEQ ID NO: 271 - RNA, 99 nt, Artificial
  Hairpin sequence
gacugaaaccaccuguucauuacaaggcgcgucuguucacagccuucaagagaggcugugaacagacgcgccuuguaaugaacaggugguuucagucuu

SEQ ID NO: 272 - RNA, 70 nt, Artificial
  Hairpin sequence
ugaagaucaagaucauugcuuagacugaauucaagagauuucagucuuggcaaugaucuugaucuucauu

SEQ ID NO: 273 - RNA, 91 nt, Artificial
  Hairpin sequence
gacugaaaccaccuguucauuugaagaucaagaucauugcuucaagagagcaaugaucuugaucuucauuugaacaggugguuucagucuu

SEQ ID NO: 274 - RNA, 92 nt, Artificial
  Hairpin sequence
gacugaaaccaccuguucauuaugaagaucaagaucauugcuucaagagagcaaugaucuugaucuucauuugaacaggugguuucagucuu

SEQ ID NO: 275 - RNA, 95 nt, Artificial
  Hairpin sequence
gacugaaaccaccuguucauuagugaagaucaagaucauugcuucaagagagcaaugaucuugaucuucauugaugaacaggugguuucagucuu

SEQ ID NO: 276 - RNA, 99 nt, Artificial
  Hairpin sequence
gacugaaaccaccuguucauuacaaugaagaucaagaucauugcuucaagagagcaaugaucuugaucuucauuguugugaacaggugguuucagucuu

SEQ ID NO: 277 - RNA, 94 nt, Artificial
  Hairpin sequence
gugaagaucaagaucauugcuuagacugaaaccaccuguucauucaagagaugaacaggugguuucagucuuggcaaugaucuugaucuucauu

SEQ ID NO: 278 - RNA, 91 nt, Artificial
  Hairpin sequence
gcugacccugaaguucauccuggccuuucacuacuccuacuuuguguagguaggaguagugaaaggccaggaugaacuucagggucagcuu

SEQ ID NO: 279 - RNA, 91 nt, Artificial
  Hairpin sequence
gcugacccugaaguucauccnngccuuucacuacuccuacuuuguguagguaggaguagugaaaggccaggaugaacuucagggucagcuu

SEQ ID NO: 280 - RNA, 93 nt, Artificial
  Hairpin sequence
gcugacccugaaguucauccuggccuuucacuacuccuacuuuguguagaaguaggaguagugaaaggcnnggaugaacuucagggucagcuu

SEQ ID NO: 281 - RNA, 93 nt, Artificial
  Hairpin sequence
gcugacccugaaguucauccnngccuuucacuacuccuacuuuguguagaaguaggaguagugaaaggcnnggaugaacuucagggucagcuu

SEQ ID NO: 282 - RNA, 93 nt, Artificial
  Hairpin sequence
gcugacccugaaguucaucudvhhccuuucacuacuccuacuuuguguagguaggaguagugaaaggccagagaugaacuucagggucagcuu

SEQ ID NO: 283 - RNA, 93 nt, Artificial
  Hairpin sequence
gcugacccugaaguucaucucuggccuuucacuacuccuacuuuguguagguaggaguagugaaaggddbhagaugaacuucagggucagcuu

SEQ ID NO: 284 - RNA, 93 nt, Artificial
  Hairpin sequence
gcugacccugaaguucaucunaanccuuucacuacuccuacuuuguguagguaggaguagugaaaggccagagaugaacuucagggucagcuu

SEQ ID NO: 285 - RNA, 94 nt, Artificial
  Hairpin sequence
gcugacccugaaguucaucucuggccuuucacuacuccuacuuuguguagguaggaguagugaaaggnaangagaugaacuucagggucagcuu

SEQ ID NO: 286 - RNA, 93 nt, Artificial
  Hairpin sequence
gcugacccugaaguucaucdvhhccuuucacuacuccuacuuuguguagaaguaggaguagugaaaggbhhbgaugaacuucagggucagcuu

SEQ ID NO: 287 - RNA, 83 nt, Artificial
  Hairpin sequence
gcugacccugaaguucaggccuuucacuacuccuacuuuguguagguaggaguagugaaaggccugaacuucagggucagcuu

SEQ ID NO: 288 - RNA, 85 nt, Artificial
  Hairpin sequence
gcugacccugaaguucauggccuuucacuacuccuacuuuguguagguaggaguagugaaaggccaugaacuucagggucagcuu

SEQ ID NO: 289 - RNA, 87 nt, Artificial
  Hairpin sequence
gcugacccugaaguucaucggccuuucacuacuccuacuuuguguagguaggaguagugaaaggccgaugaacuucagggucagcuu

SEQ ID NO: 290 - RNA, 89 nt, Artificial
  Hairpin sequence
gcugacccugaaguucauccggccuuucacuacuccuacuuuguguagguaggaguagugaaaggccagaugaacuucagggucagcuu

SEQ ID NO: 291 - RNA, 91 nt, Artificial; identical to SEQ ID NO: 278
  Hairpin sequence

SEQ ID NO: 292 - RNA, 93 nt, Artificial
  Hairpin sequence
gcugacccugaaguucaucucuggccuuucacuacuccuacuuuguguagguaggaguagugaaaggccagagaugaacuucagggucagcuu

SEQ ID NO: 293 - RNA, 95 nt, Artificial
  Hairpin sequence
gcugacccugaaguucaucugcuggccuuucacuacuccuacuuuguguagguaggaguagugaaaggccagcagaugaacuucagggucagcuu

SEQ ID NO: 294 - RNA, 97 nt, Artificial
  Hairpin sequence
gcugacccugaaguucaucugccuggccuuucacuacuccuacuuuguguagguaggaguagugaaaggccaggcagaugaacuucagggucagcuu

SEQ ID NO: 295 - RNA, 99 nt, Artificial
  Hairpin sequence
gcugacccugaaguucaucugcacuggccuuucacuacuccuacuuuguguagguaggaguagugaaaggccagugcagaugaacuucagggucagcuu

SEQ ID NO: 296 - RNA, 101 nt, Artificial
  Hairpin sequence
gcugacccugaaguucaucugcaccuggccuuucacuacuccuacuuuguguagguaggaguagugaaaggccaggugcagaugaacuucagggucagcuu

SEQ ID NO: 297 - RNA, 50 nt, Artificial
  Hairpin sequence
gccaggaccacgagaagcuguuuguguagcagcuucucgugguccugguu

SEQ ID NO: 298 - RNA, 139 nt, Artificial
  Hairpin sequence
ggagagccauaguggucuggaaacggaaccggugaguacacgaaaaggucucguagaccgugcauuuguguagugcacggucuacgagaccucaagguguacucaccgguuccgcaagcagaccacuauggcucuccuu

SEQ ID NO: 299 - DNA, 38 bp, Artificial
  Oligonucleotide
tgagatgaacttcagggtcagcggtgtttcgtcctttc

SEQ ID NO: 300 - DNA, 41 bp, Artificial
  Oligonucleotide
agcctttcactactcctacttttttttggaaaagcttatcg

SEQ ID NO: 301 - DNA, 39 bp, Artificial
  Oligonucleotide
ctggcctttcactactcctacctggtgtttcgtcctttc

SEQ ID NO: 302 - DNA, 40 bp, Artificial
  Oligonucleotide
agatgaacttcagggtcagcttttttggaaaagcttatcg

SEQ ID NO: 303 - DNA, 51 bp, Artificial
  Oligonucleotide
gctgaccctgaagttcatctcaagcctttcactactcctacttcctgtctc

SEQ ID NO: 304 - DNA, 51 bp, Artificial
  Oligonucleotide
aaagtaggagtagtgaaaggccagagatgaacttcagggtcagcctgtctc

SEQ ID NO: 305 - RNA, 43 nt, Artificial
  Sense sequence
gcugacccugaaguucaucucaagccuuucacuacuccuacuu

SEQ ID NO: 306 - RNA, 46 nt, Artificial
  Antisense sequence
gagguaggaguagugaaaggccagagaugaacuucagggucagcuu

SEQ ID NO: 307 - RNA, 42 nt, Artificial
  Sense sequence
guaggaguagugaaaggcuugagaugaacuucagggucaguu

SEQ ID NO: 308 - RNA, 43 nt, Artificial
  Antisense sequence
cugacccugaaguucaucucuggccuuucacuacuccuaccuu

(19) United States
(12) Patent Application Publication
(10) Pub. No.: US 2011/0117608 A1
(43) Pub. Date: May 19, 2011

(54) DOUBLE-STRANDED NUCLEIC ACID

(75) Inventors: MICHAEL WAYNE GRAHAM, JINDALEE (AU); KENNETH CLIFFORD REED, ST. LUCIA (AU); ROBERT NORMAN RICE, SINNAMON PARK (AU); BRUCE THOMAS HARRISON, EASTERN HEIGHTS (AU); PETRUS ROELVINK, CAMPBELL, CA (US); DAVID SUHY, SAN RAMON, CA (US); ALEXANDER KOLYKHALOV, MOUNTAIN VIEW, CA (US)

(73) Assignee: BENITEC AUSTRALIA, LTD., MELBOURNE (AU)

(21) Appl. No.: 12/914,893

(22) Filed: Oct. 28, 2010

Related U.S. Application Data
(63) Continuation of application No. 10/861,191, filed on Jun. 3, 2004, now abandoned.
(60) Provisional application No. 60/475,827, filed on Jun. 3, 2003.
(60) Provisional application No. 60/479,616, filed on Jun. 17, 2003.
(60) Provisional application No. 60/550,504, filed on Mar. 5, 2004.
(60) Provisional application No. 60/553,920, filed on Mar. 17, 2004.

(30) Foreign Application Priority Data
Nov. 14, 2003 (AU) 2003906281
Mar. 10, 2004 (AU) 2004901258
Apr. 30, 2004 (AU) 2004902279

(51) Int. Cl. C12P 19/34; C07H 21/02; C12N 15/63; C12N 5/071; C12N 9/10

(52) U.S. Cl. 435/91.2; 536/24.5; 435/320.1; 435/325; 435/193

(57) ABSTRACT

The invention is directed towards constructs for RNAi techniques. The invention provides a ribonucleic acid (RNA) for use as interfering RNA in gene silencing techniques to silence a target gene comprising in a 5′ to 3′ direction at least a first effector sequence, a second effector sequence, a sequence substantially complementary to the second effector sequence and a sequence substantially complementary to the first effector sequence, wherein the complementary sequences are capable of forming double stranded regions with their respective effector sequences and wherein at least one of these sequences is substantially identical to the predicted transcript of a region of the target gene, and a nucleic acid construct encoding such an RNA.

### CROSS-REFERENCE TO RELATED APPLICATIONS

This application claims benefit of U.S. application Ser. No. 10/861,191, filed Jun. 3, 2004, which claims benefit to U.S. provisional patent application Ser. No. 60/475,827, filed Jun. 3, 2003; U.S. provisional patent application Ser. No. 60/479,616, filed Jun. 17, 2003; Australian patent application serial number 2003906281, filed Nov. 14, 2003; U.S. provisional patent application Ser. No. 60/550,504, filed Mar. 5, 2004; Australian patent application serial number 2004901258, filed Mar. 10, 2004; U.S. provisional patent application Ser. No. 60/553,920, filed Mar. 17, 2004; and Australian patent application serial number 2004902279, filed Apr. 30, 2004; which are herein incorporated by reference.

The present invention relates to a nucleic acid containing complementary sequences which may form multiple double stranded regions. The present invention also relates to sequences and constructs encoding such a nucleic acid and the uses of such a nucleic acid or construct to modify gene expression, particularly to reduce or inhibit gene expression.

### BACKGROUND OF THE INVENTION

Certain single stranded nucleic acid molecules are able to form a self-complementary double stranded region where part of the nucleotide sequence is able to interact with another part of the sequence by Watson-Crick base pairing between inverted repeats of the sequence. Where the repeated regions are adjacent or in close proximity to each other, the double stranded regions may form structures known as hairpin structures. The hairpin structure forms with an unpaired “loop” of nucleotides at one end of the hairpin structure, with the inverted repeat sequence annealed. The loop may also facilitate the folding of the nucleic acid chain.

Hairpin RNA sequences have become a powerful tool for basic and applied research. In particular these sequences have been used in interfering RNA and gene silencing technologies. Such techniques are described in the specification of PCT/AU99/00195 (U.S. patent application Ser. No. 09/646,807 and U.S. Pat. No. 6,573,099) and PCT/AU01/00297, the contents of which are herein incorporated by reference. In summary, RNA interference (RNAi) hairpin RNA sequences may be synthesised within a cell from DNA constructs coding these sequences, hereafter termed “hairpin DNA constructs”.

While many hairpin DNA constructs have proved effective in gene silencing, other DNA constructs only show partial gene silencing activity. Increasing the degree of gene inactivation produced by RNAi hairpin RNA would be advantageous, for example in gene therapy. Furthermore, in many situations, it would be advantageous to be able to silence two or more separate genes or gene regions simultaneously, particularly in respect of gene therapy applications.

Reference to any prior art in this specification is not, and should not be taken as, an acknowledgment or any form of suggestion that this prior art forms part of the common general knowledge of one skilled in the art.

There is a need for improved RNA hairpin sequences to be used in interfering RNA and gene silencing technology. Furthermore, there is a need for a DNA construct that is capable of producing hairpin RNA transcripts with an improved gene silencing activity and a need for a DNA construct encoding hairpin RNA capable of inactivating two or more separate genes. There is further a need for improved methods for the synthesis of such DNA constructs. It is an object of the present invention to overcome, or at least alleviate, one or more of these needs in light of the prior art.

### BRIEF DESCRIPTION OF THE DRAWINGS

FIG. 1 shows maps of the construct pU6.cass. A shows a map of a region of the construct. The human U6 promoter is shown as a grey arrow, binding sites of the U6FR1 and U6 R T5 Xba primers are shown below this. The positions of Eco RI, Bsm BI and Hind III restriction sites are shown. B shows a map of the entire plasmid which was constructed by inserting the Eco RI/Hind III fragment shown in A into the vector pBluescript II SK+ (Stratagene).

FIG. 2 shows maps of the construct pU6.ACTB-A hp. This construct was used as a negative control in some experiments. A Map of the plasmid is shown as in FIG. 1B. The relative positions of elements within the hairpin DNA transcription unit, namely the transcription start site, the ACTB-A sense, loop, ACTB-A antisense and pol III terminator sequences are shown, as are the positions of the Eco RI and Hind III restriction sites. B shows a map of a portion of the U6 transcriptional unit. Elements within the hairpin DNA transcription unit are shown; the sense and antisense regions of the hairpin are shown as arrows, the loop sequence is denoted as a stippled arrow and the terminator as a line below the map. C shows the predicted hairpin RNA produced from this construct which targets the ACTB-A site of β actin mRNA. The 5′ G ribonucleotide of the predicted transcript is required for U6 promoter activity, the pol III terminator is predicted to incorporate the 3′ sequence UU which is also not based paired in the hairpin transcript. The transcript is predicted to produce a 19 nt double-stranded RNA structure homologous to β actin mRNA, where the vertically aligned sequences denote potential base pairing. The loop sequence is 9 bases, the first and second bases can potentially pair with the eight and ninth bases, but for clarity this is not shown. In addition, the 5′ G might potentially base pair with the second-to-last 3′ U residue, but this is also not shown for clarity. The convention that all unpaired sequences are shown in this way is used throughout this specification.

FIG. 3 shows the general approach of using long-range PCR to modify plasmids A. Either circular or linear DNA can be used as amplification templates, although the latter is preferred. DNA is amplified with oligonucleotide primers (LRPCR primers) containing “clamp” sequences that can hybridize to the templates (thin lines) and sequences corresponding to roughly half of the desired inserts (thick lines). When combined these will form the insert, typically an hpRNA encoding insert. B. Template DNA is amplified using conditions suitable for long range PCR reactions. The favoured polymerase is PfuUltra (Stratagene), due to its low error rate, although other polymerases or mixtures can be used. C. The amplified DNA fragment is then circularised via an intramolecular ligation using T4 DNA ligase. For this step 5′ phosphorylation of at least one end is required, which can be achieved using phosphorylated oligonucleotides for the amplification, or by post-amplification treatment with T4 polynucleotide kinase. Flush ends are also required for efficient circularisation, Pfu polymerase produces flush ends, alternatively ends might be polished by post-amplification treatment with T4 DNA polymerase.

FIG. 4 shows the insertion of an Asc I restriction site into a plasmid. The oval lines at the top represent the plasmid used for insertion. The binding position and orientation of the LRPCR primers are also shown (diagrammatically, not to scale) around the point of sequence insertion is also shown. The sequence (SEQ ID NO: 228) of a region of the plasmid is shown below this, as are the sequences of the LRPCR primers (SEQ ID NOS: 227 and 229), the Asc I restriction site is shown as a bold underline.

FIG. 5 shows the insertion of a hp DNA sequence, containing inverted repeat and loop sequences into a plasmid as in FIG. 4. Partial sequence (SEQ ID NO: 231) of the insert and primers (SEQ ID NOS: 230 and 232) is also shown as in FIG. 4; antisense and sense hp sequences are shown as bold underline.

FIG. 6 shows the method of increasing the length of an inverted repeat in a plasmid. The Figure is shown as in FIG. 4, except only 1 primer is used. Partial sequences (SEQ ID NO: 234) of the inserts and primers (SEQ ID NOS: 233 and 235) is also shown as in FIG. 4.

FIG. 7 shows the insertion of a mouse IgE3 intron into a cloned insert in a plasmid as in FIG. 4.

FIG. 8 shows a map of the plasmid pU6.cass lin. A shows a map of a region of the construct corresponding to the U6 promoter and pol III terminator sequences. The positions of Bmg BI, Bgl II and Bsm I restriction sites are shown. B shows a map of the entire plasmid.

FIG. 9 shows maps of the construct pU6.Rluc hp; this targets humanised Renilla luciferase mRNA (Accession Number U47298) for degradation. A shows a map of a portion of the U6 transcriptional unit. Elements within the hairpin DNA transcription unit are shown as in FIG. 2B. B shows the predicted hairpin RNA (SEQ ID NO: 239) produced from this construct as in FIG. 2C.

FIG. 10 shows a map of pU6.Rluc/ACTB TTA. A shows a map of the hairpin DNA transcriptional unit. The position of “bubble” and “loop” sequences within the transcriptional unit are shown as stippled arrows. In this instance “stem” sequences, derived from β Actin (ACTB) and Renilla luciferase (Rluc) have been incorporated into the construct. B shows the predicted hairpin RNA (SEQ ID NO: 240) produced from this construct as in FIG. 2C. In this and other examples the bubble sequences, which are not capable of conventional base pairing, are shown above and below those potentially base paired sequences in the transcript. The convention of showing no base pairing between sequences in the bubbles regardless of the potential of the bases in these sequences to form Watson-Crick or non-Watson-Crick base pairs, is used throughout this specification. Sequences at the base of the hairpin target Renilla luciferase mRNA, sequences nearer the loop target β actin mRNA.

FIG. 11 shows a map of pU6.Rluc/ACTB TTAG. A shows a map of the hairpin DNA transcriptional unit as for FIG. 10A. B shows the predicted hairpin RNA (SEQ ID NO: 241) produced from this construct as in FIG. 10B.

FIG. 12 shows a map of pU6.ACTB/Rluc-TTA. A shows a map of the hairpin DNA transcriptional unit as for FIG. 10A B shows the predicted hairpin RNA (SEQ ID NO: 242) produced from this construct as in FIG. 10B.

FIG. 13 shows a map of pU6.ACTB/Rluc TTAG. A shows a map of the hairpin DNA transcriptional unit as for FIG. 10A. B shows the predicted hairpin RNA (SEQ ID NO: 243) produced from this construct as in FIG. 10B.

FIG. 14 shows a map of pU6.ACTB/AD1 hp. This construct was used as a negative control for some experiments. A shows a map of the hairpin DNA transcriptional unit as for Figure A. B shows the predicted hairpin RNA (SEQ ID NO: 244) produced from this construct as in FIG. 10B.

FIG. 15 shows a map of pU6 Rluc/ACTB/AD1 hp. A shows a map of the hairpin DNA transcriptional unit as for FIG. 10A. In this instance sequences, targeting Renilla luciferase (Rluc), β Actin (ACTB), and ADAR-1 (AD-1) have been incorporated into the construct. B shows the predicted hairpin RNA (SEQ ID NO: 245) produced from this construct as in FIG. 10B.

FIG. 16 shows a map of pU6 ACTB/Rluc/AD1. A shows a map of the hairpin DNA transcriptional unit as for FIG. 10A. B shows the predicted hairpin RNA (SEQ ID NO: 246) produced from this construct as in FIG. 10B.

FIG. 17 shows a map of pU6 ACTB/ADAR/Rluc hp. A shows a map of the hairpin DNA transcriptional unit as for FIG. 10A. B shows the predicted hairpin RNA (SEQ ID NO: 247) produced from this construct as in FIG. 10B.

FIG. 18 shows a map of pU6 ACTB/ADAR/GFP hp. This construct was used as a negative control for some experiments. A shows a map of the hairpin DNA transcriptional unit as for FIG. 10A. B shows the predicted hairpin RNA (SEQ ID NO: 248) produced from this construct as in FIG. 10B.

FIG. 19 shows a map of pU6.Rluc/ACTB/AD1/GFP hp. A shows a map of the hairpin DNA transcriptional unit as for FIG. 10A. B shows the predicted hairpin RNA (SEQ ID NO: 249) produced from this construct as in FIG. 10B.

FIG. 20 shows a map of pU6.ACTB/Rluc/AD1/GFP hp. In this and the following examples sequences, derived from β Actin (ACTB), Renilla luciferase (Rluc), ADAR1 (AD1) and GFP have been incorporated into the construct. A shows a map of the hairpin DNA transcriptional unit as for FIG. 10A. B shows the predicted hairpin RNA (SEQ ID NO: 250) produced from this construct as in FIG. 10B.

FIG. 21 shows a map of pU6.ACTB/AD1/Rluc/GFP hp. A shows a map of the hairpin DNA transcriptional unit as for FIG. 10A. B shows the predicted hairpin RNA (SEQ ID NO: 251) produced from this construct as in FIG. 10B.

FIG. 22 shows a map of pU6.ACTB/AD1/GFP/Rluc hp. A shows a map of the hairpin DNA transcriptional unit as for FIG. 10A. B shows the predicted hairpin RNA (SEQ ID NO: 252) produced from this construct as in FIG. 10B.

FIG. 23 shows a map of pU6.ACTB/AD1/GFP/HER2 hp. A shows a map of the hairpin DNA transcriptional unit as for FIG. 10A. B shows the predicted hairpin RNA (SEQ ID NO: 253) produced from this construct as in FIG. 10B.

FIG. 24 shows a map of pU6.Rluc/ACTB/AD1/GFP/HER2 hp. A shows a map of the hairpin DNA transcriptional unit as for FIG. 10A. In this instance sequences, derived from β Actin (ACTB), ADAR1 (AD1), Renilla luciferase (Rluc), HER2 and GFP have been incorporated into the construct. B shows the predicted hairpin RNA (SEQ ID NO: 254) produced from this construct as in FIG. 10B.

FIG. 25 show a map of pU6.ACTB/Rluc/AD1/GFP/HER2 hp. A shows a map of the hairpin DNA transcriptional unit as for FIG. 10A. B shows the predicted hairpin RNA (SEQ ID NO: 255) produced from this construct as in FIG. 10B.

FIG. 26 shows a map of pU6.ACTB/AD1/Rluc/GFP/HER2 hp. A shows a map of the hairpin DNA transcriptional unit as for FIG. 10A. B shows the predicted hairpin RNA (SEQ ID NO: 256) produced from this construct as in FIG. 10B.

FIG. 27 shows a map of pU6.ACTB/AD1/GFP/Rluc/HER2 hp. A shows a map of the hairpin DNA transcriptional unit as for FIG. 10A. B shows the predicted hairpin RNA (SEQ ID NO: 257) produced from this construct as in FIG. 10B.

FIG. 28 shows a map of pU6.ACTB/AD1/GFP/HER2/Rluc hp. A shows a map of the hairpin DNA transcriptional unit as for FIG. 10A. B shows the predicted hairpin RNA (SEQ ID NO: 258) produced from this construct as in FIG. 10B.

FIG. 29 shows a map of pU6.ACTB/AD1/GFP/HER2/LAM hp. A shows a map of the hairpin DNA transcriptional unit as for FIG. 10A. In this instance, sequences derived from lamin A/C (LAM) have been incorporated into the construct. B shows the predicted hairpin RNA (SEQ ID NO: 259) produced from this construct as in FIG. 10B.

FIG. 30 shows a graph describing the activity of double hairpin constructs targeting Renilla luciferase. Data are shown corrected to the relative Renilla luciferase activity in transgenic cells transfected with the construct pU6.cass (n=5, ±SD). The white bars denote activities of negative control constructs, namely pU6.cass, pU6.ACTB-A hp and pU6.ACTB/AD1 hp. The black bars denote activities of constructs targeting Rluc, namely pU6.Rluc hp and the double hairpin constructs pU6.Rluc/ACTB TTA, pU6.Rluc/ACTB TTAG, pU6.ACTB/Rluc TTA and pU6.ACTB/Rluc TTAG.

FIG. 31 shows the activity of triple hairpin constructs targeting Renilla luciferase as in FIG. 30. In this experiment the negative controls, shown as white bars, were pU6.cass, pU6.ACTB-A hp and pU6.ACTB/AD1/GFP hp; the test constructs, shown as black bars, were pU6.Rluc hp and the triple hairpin constructs pU6.Rluc/ACTB/AD1 hp, pU6.ACTB/Rluc/AD1 hp and pU6.ACTB/AD1/Rluc hp.

FIG. 32 shows the activity of constructs targeting 4 and 5 genes, with Renilla luciferase at position 4 or 5, adjacent to the loop as in FIG. 30. In this experiment the negative controls, shown as white bars, were pU6.cass, pU6.ACTB-A hp and pU6.ACTB/AD1/GFP hp; the test constructs, shown as black bars, were pU6.Rluc hp, pU6.ACTB/AD1/GFP/Rluc and pU6.ACTB/AD1/GFP/HER2/Rluc.

FIG. 33 shows a map of pU6.GF-2 which targets both the Akt1 and Akt2 genes for inactivation. A shows a map of the hairpin DNA transcriptional unit as for FIG. 10A. B shows the predicted hairpin RNA (SEQ ID NO: 260) produced from this construct as in FIG. 10B.

FIG. 34 shows Western blots demonstrating reductions in Akt1 and Akt2 proteins in cells transfected with the double hairpin construct pU6.GF-2. The Western blots were probed with antibodies specific to Sec5, which acts as a loading control, and antibodies specific to the targets, either Akt1 or Akt2. Lanes probed (kr) were control, non-transfected C2C12 and cells transfected with pU6.GF-2, both lanes probed with Sec5 and Akt1 antibodies and non-transfected C2C12 cells and cells transfected with pU6.GF-2, both lanes probed with Sec5 and Akt2 antibodies.

FIG. 35 shows a map of pU6.GG-2 which targets the Akt 2a site. A shows a map of the hairpin DNA transcriptional unit as for FIG. 10A. B shows the predicted hairpin RNA (SEQ ID NO: 261) produced from this construct as in FIG. 10B.

FIG. 36 shows a map of pU6.GG-3. A shows a map of the hairpin DNA transcriptional unit as for FIG. 10A. B shows the predicted hairpin RNA (SEQ ID NO: 262) produced from this construct as in FIG. 10B.

FIG. 37 shows a map of pU6.GG-4 which targets both the Akt 2a and Akt 2b sites of Akt 2. A shows a map of the hairpin DNA transcriptional unit as for FIG. 10A. B shows the predicted hairpin RNA (SEQ ID NO: 263) produced from this construct as in FIG. 10B.

FIG. 38 Western blots showing enhanced reductions in protein in cells transfected with pU6.GF-2. The Western blot was probed with antibodies specific to Sec 5, which acts as a loading control, and antibodies specific to the target Akt2. Lanes probed (l-r) were control non-transfected C2C12 cells, and cells transfected with the constructs pU6.GG-2, pU6.GG-3 and pU6.GG-4.

FIG. 39 shows maps of the construct pU6.ACTB-A48 hp. A shows a map of the hairpin DNA transcriptional unit as in FIG. 2B. B shows the predicted hairpin RNA (SEQ ID NO: 264) produced from this construct as in FIG. 2C. This hairpin RNA potentially targets the ACTB-A site of β actin mRNA as well as the next 29 nts of the mRNA. The transcript is predicted to produce a 48 nt double-stranded RNA.

FIG. 40 shows maps of the plasmid pU6.AD1-A. A shows a map of the hairpin DNA transcriptional unit as in FIG. 2B. B shows the predicted hairpin RNA (SEQ ID NO: 265) produced from this construct as in FIG. 2C. This transcript potentially targets the ADAR 1-A site of ADAR 1 mRNA.

FIG. 41 shows maps of the plasmid pU6.AD2-C. A shows a map of the hairpin DNA transcriptional unit as in FIG. 2B. B shows the predicted hairpin RNA (SEQ ID NO: 266) produced from this construct as in FIG. 2C. This transcript potentially targets the ADAR 2-C site of ADAR 2 mRNA.

FIG. 42 shows maps of the plasmid pU6.AD2-A. A shows a map of the hairpin DNA transcriptional unit as in FIG. 2B. B shows the predicted hairpin RNA (SEQ ID NO: 267) produced from this construct as in FIG. 2C. This transcript potentially targets the ADAR 2-A site of ADAR 2 mRNA.

FIG. 43 shows maps of the plasmid pU6.AD1/2-B. A shows a map of the hairpin DNA transcriptional unit as in FIG. 2B. B shows the predicted hairpin RNA (SEQ ID NO: 268) produced from this construct as in FIG. 2C. This transcript potentially targets both the ADAR 1-B site of ADAR 1 mRNA and the ADAR 2-B site of ADAR 2 mRNA.

FIG. 44 shows maps of the plasmid pU6.AD1&2-A/UU. A shows a map of the hairpin DNA transcriptional unit as in FIG. 10A, the position of “bubble” and loop sequences within the transcriptional unit are shown as stippled arrows. B shows the predicted hairpin RNA (SEQ ID NO: 269) produced from this construct as in FIG. 10B. Sequences at the base of the hairpin target the ADAR 1-A site of ADAR 1 mRNA, sequences nearer the loop target the ADAR 2-A site of ADAR 2 mRNA.

FIG. 45 shows maps of the plasmid pU6.AD1&2-A/UUA. A shows a map of the hairpin DNA transcriptional unit as in FIG. 10A. B shows the predicted hairpin RNA (SEQ ID NO: 270) produced from this construct as in FIG. 10B.

FIG. 46 shows maps of the plasmid pU6.AD1&2-A/UUACAA. A shows a map of the hairpin DNA transcriptional unit as in FIG. 10A. B shows the predicted hairpin RNA (SEQ ID NO: 271) produced from this construct as in FIG. 10B.

FIG. 47 shows a comparison showing the predicted transcripts produced by the constructs pU6.AD1&2-A/UU (SEQ ID NO: 269) (A), pU6.AD1&2-A/UUA (SEQ ID NO: 270) (B) and pU6.AD1&2-A/UUACAA (SEQ ID NO: 271) (C). Predicted structures are shown as in FIG. 10B.

FIG. 48 shows maps of the plasmid pU6.ACTB-A/UUA. A shows a map of the hairpin DNA transcriptional unit as in FIG. 10A; in this instance a “stem” sequence, derived from the first seven nucleotides of the ADAR 1-A target has been incorporated into the construct. Without being bound by any theory or mode of action, it is believed that this sequence is too short to target ADAR 1 mRNA, but can act by maintaining the structure of the bubble sequence in the construct. B shows the predicted hairpin RNA (SEQ ID NO: 272) produced from this construct as in FIG. 10B.

FIG. 49 shows maps of the plasmid pU6.AD1-A&ACTB-A/UU. A shows a map of the hairpin DNA transcriptional unit as in FIG. 10A. B shows the predicted hairpin RNA (SEQ ID NO: 273) produced from this construct as in FIG. 10B.

FIG. 50 shows maps of the plasmid pU6.AD1-A&ACTB-A/UUA. A shows a map of the hairpin DNA transcriptional unit as in FIG. 10A. B shows the predicted hairpin RNA (SEQ ID NO: 274) produced from this construct as in FIG. 10B.

FIG. 51 shows maps of the plasmid pU6.AD1-A&ACTB-A/UUAG. A shows a map of the hairpin DNA transcriptional unit as in FIG. 10A. B shows the predicted hairpin RNA (SEQ ID NO: 275) produced from this construct as in FIG. 10B.

FIG. 52 shows maps of the plasmid pU6.AD1-A&ACTB-A/UUACAA. A shows a map of the hairpin DNA transcriptional unit as in FIG. 10A. B shows the predicted hairpin RNA (SEQ ID NO: 276) produced from this construct as in FIG. 10B.

FIG. 53 shows maps of the plasmid pU6.ACTB-A&AD1-A/UUA. A shows a map of the hairpin DNA transcriptional unit as in FIG. 10A. B shows the predicted hairpin RNA (SEQ ID NO: 277) produced from this construct as in FIG. 10B.

FIG. 54 shows a comparison showing the encoded transcripts produced by the constructs pU6.ACTB-A/UUA (SEQ ID NO: 272) (A), pU6.AD1-A&ACTB-A/UU (SEQ ID NO: 273) (B), pU6.AD1-A&ACTB-A/UUA (SEQ ID NO: 274) (C), pU6.AD1-A&ACTB-A/UUAG (SEQ ID NO: 275) (D), pU6.AD1-A&ACTB-A/UUACAA (SEQ ID NO: 276) (E), pU6.ACTB-A&AD1-A/UUA (SEQ ID NO: 277) (F) as in FIG. 10B.

FIG. 55 shows the activity of double hairpin constructs targeting ADAR 1 and shows the enhanced activity of some bubble constructs compared to a single hairpin construct.

FIG. 56 shows the activity of double hairpin constructs targeting ADAR 2.

FIG. 57 shows the activity of double hairpin constructs targeting ADAR 1.

FIG. 58 shows the activity of double hairpin constructs targeting β actin.

FIG. 59 shows a map of pU6.GR-21 hp, which targets GFP and Rluc for inactivation. A shows a map of the hairpin DNA transcriptional unit as for FIG. 10A. B shows the predicted hairpin RNA (SEQ ID NO: 278) produced from this construct as in FIG. 10B.

FIG. 60 shows a map of the library construct pU6.GR-21-1-2N, which targets GFP and Rluc for inactivation. A shows a map of the hairpin DNA transcriptional unit as for FIG. 10A. The position of randomised sequences within the construct is shown as a stippled arrow below the map. B shows the predicted hairpin RNA (SEQ ID NO: 279) produced from this construct as in FIG. 10B, in this instance N represents any ribonucleotide (ie A, C, U or G).

FIG. 61 shows a map of the library construct pU6.GR-21-4-2N, which targets GFP and Rluc for inactivation. A shows a map of the hairpin DNA transcriptional unit as for FIG. 10A. B shows the predicted hairpin RNA (SEQ ID NO: 280) produced from this construct as in FIG. 10B.

FIG. 62 shows a map of the library construct pU6.GR-21-1&4-2N, which targets GFP and Rluc for inactivation. A shows a map of the hairpin DNA transcriptional unit as for FIG. 10A. B shows the predicted hairpin RNA (SEQ ID NO: 281) produced from this construct as in FIG. 10B.

FIG. 63 shows maps and sequences of the library construct series pU6.GR-22-1-4N and pU6.GR-22-4-4N, both target GFP and Rluc for inactivation. A shows a map of the hairpin DNA transcriptional unit of pU6.GR-22-1-4N as for FIG. 10A. B shows the predicted hairpin RNA (SEQ ID NO: 282) produced from this construct as in FIG. 10B; in this instance D represents the ribonucleotides A, G or U; V represents the ribonucleotides A, C or G; and H represents the ribonucleotides A, C or U. C shows a map of the hairpin DNA transcriptional unit of pU6.GR-22-4-4N as for

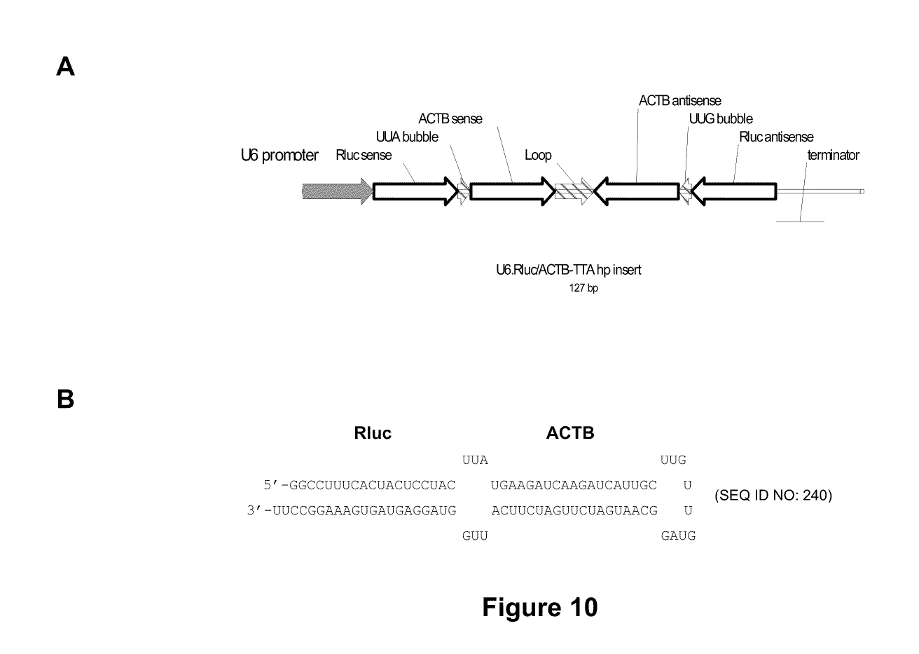

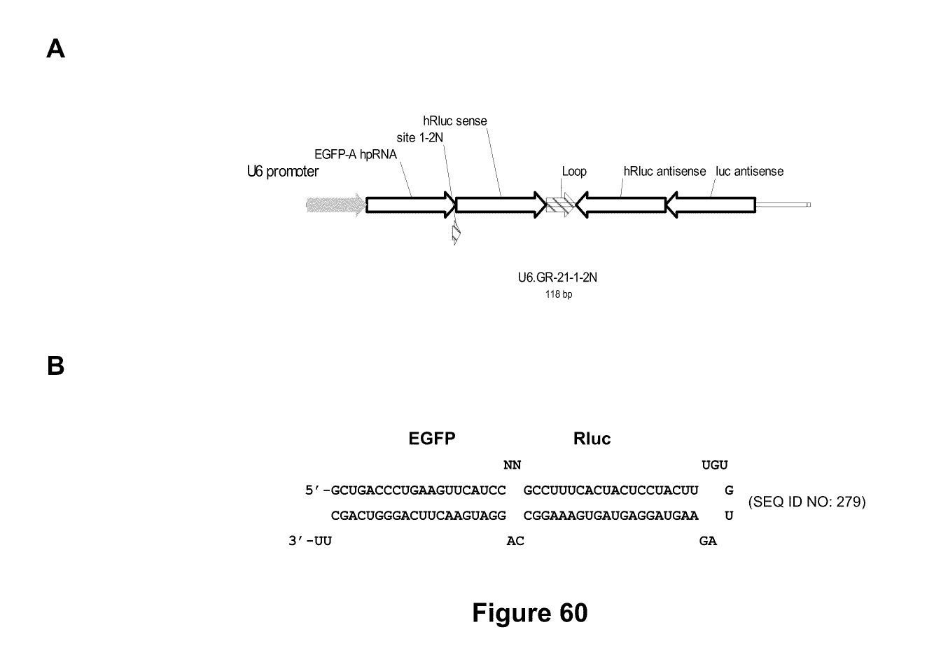

FIG. 60A. D shows the predicted hairpin RNA (SEQ ID NO: 283) produced from this construct as in FIG. 10B; in this instance H represents the ribonucleotides A, C or U; B represents the ribonucleotides C, G or U and D represents the ribonucleotides A, G or U.

FIG. 64 shows maps and sequences of the library construct pU6.GR-22-1-NAAN and pU6.GR-22-4-NAAN, both target GFP and Rluc for inactivation. A shows a map of the hairpin DNA transcriptional unit of pU6.GR-22-1-NAAN as for FIG. 10A. B shows the predicted hairpin RNA (SEQ ID NO: 284) produced from this construct as in FIG. 10B; in this instance N represents any ribonucleotide. C shows a map of the hairpin DNA transcriptional unit of pU6.GR-22-4-NAAN as for FIG. 60A. D shows the predicted hairpin RNA (SEQ ID NO: 285) produced from this construct as in FIG. 10B; in this instance in this instance N represents any ribonucleotide.

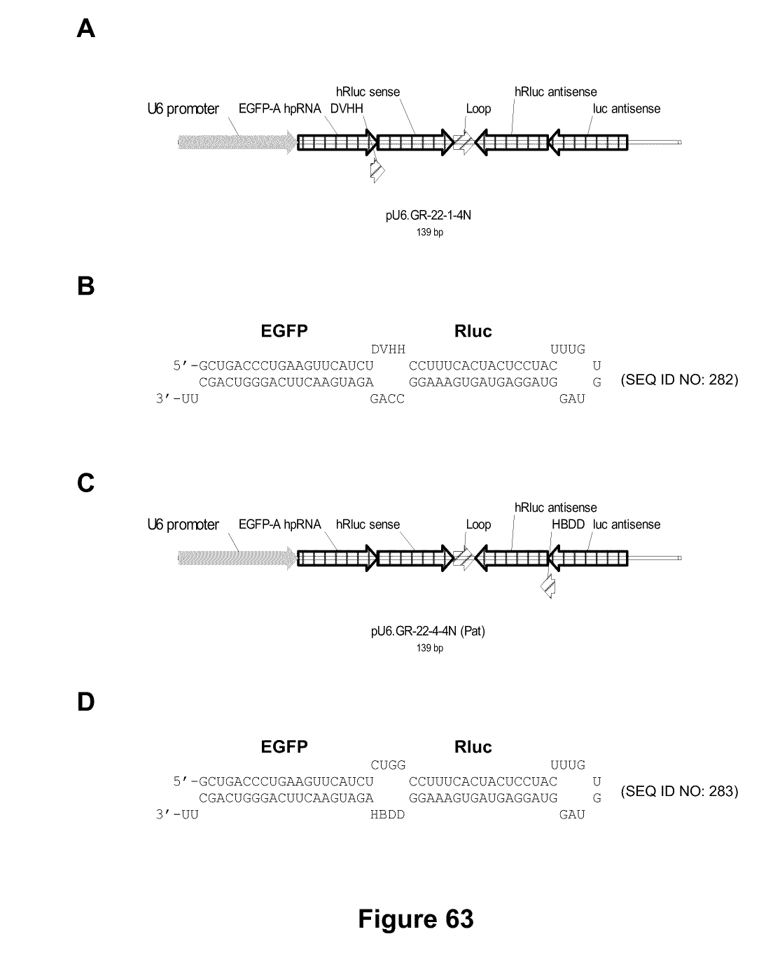

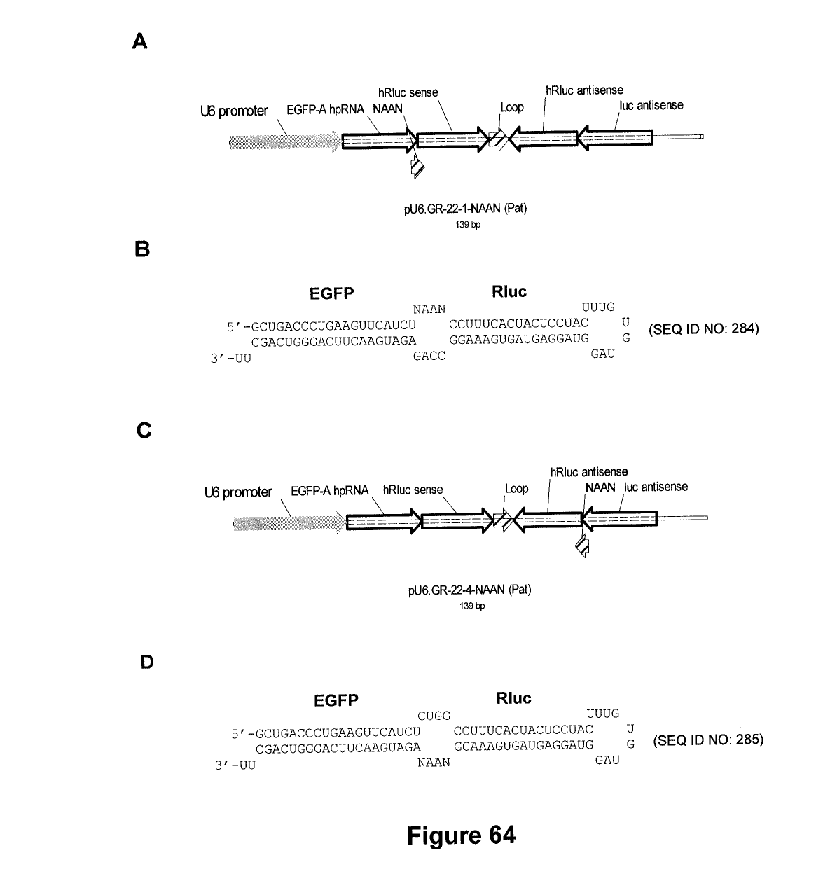

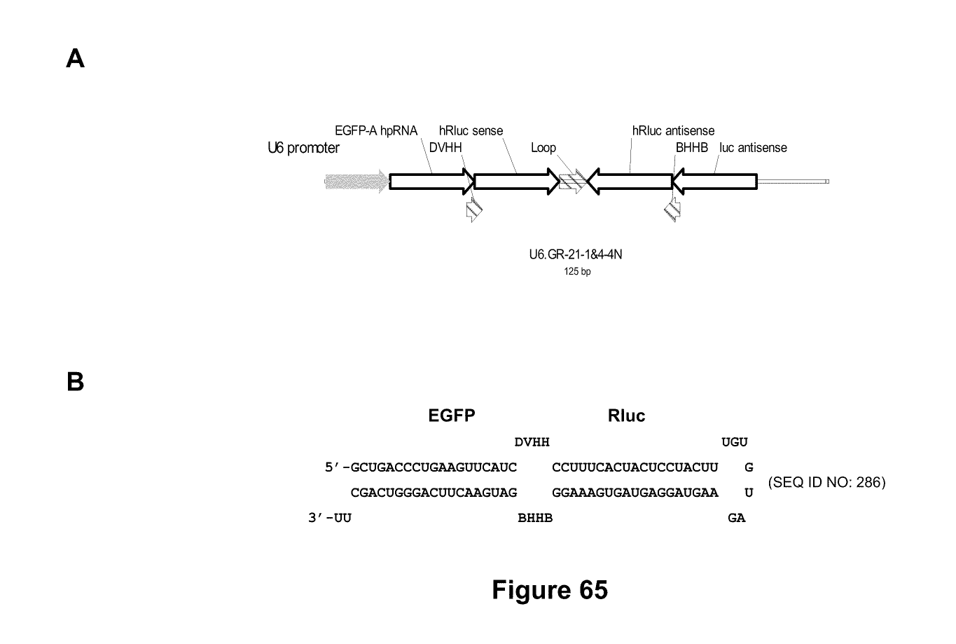

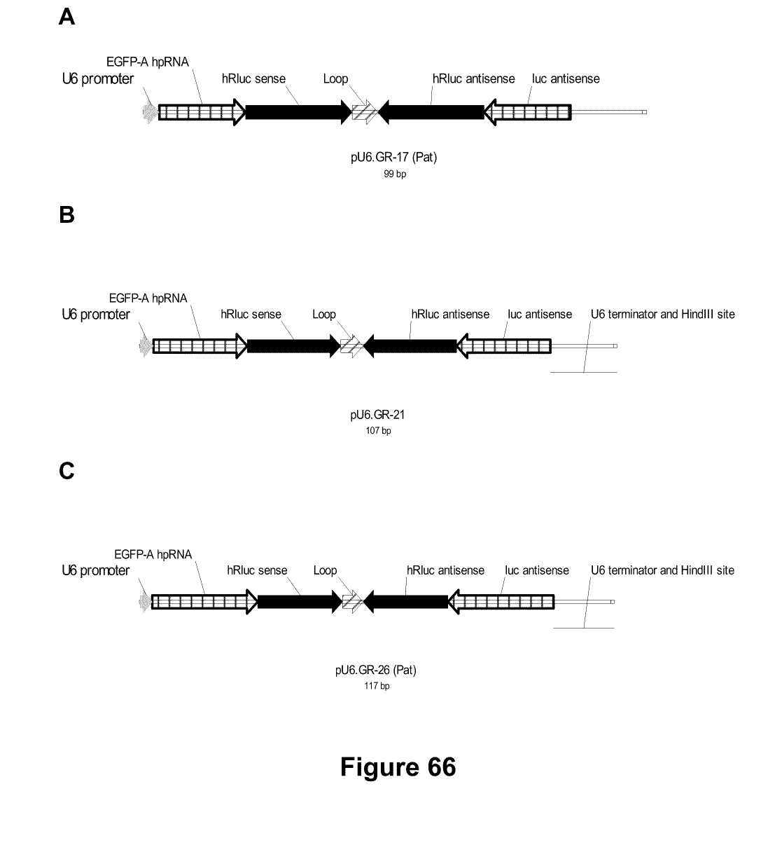

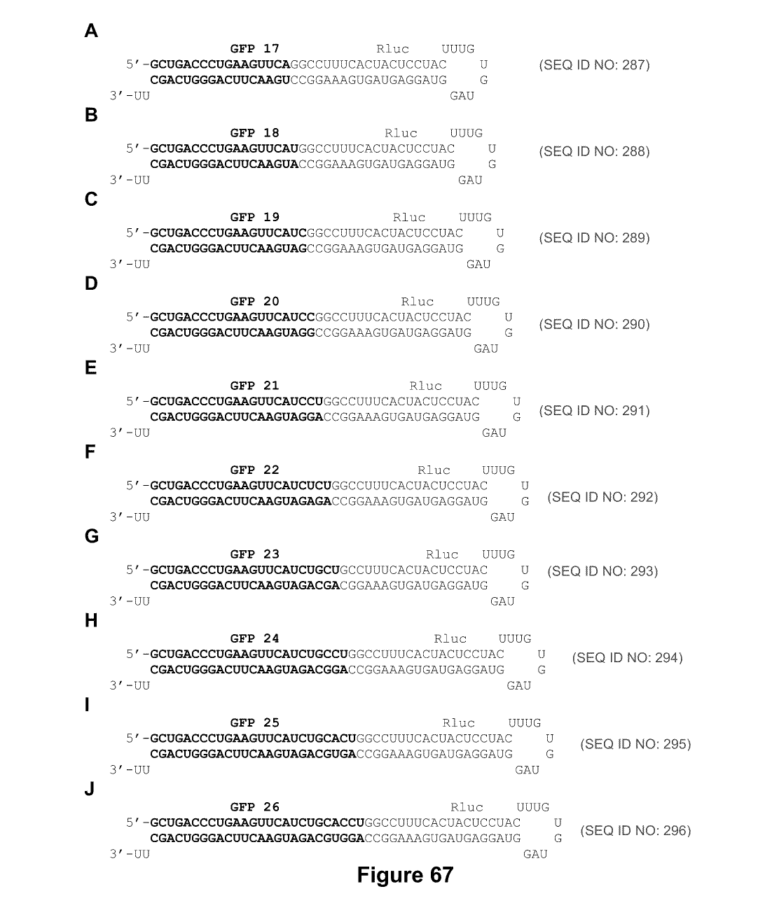

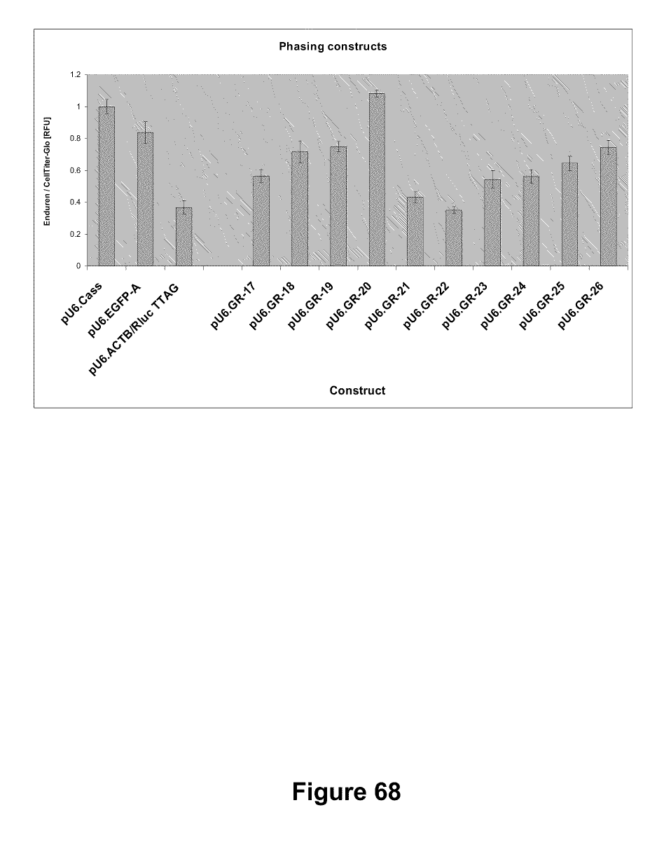

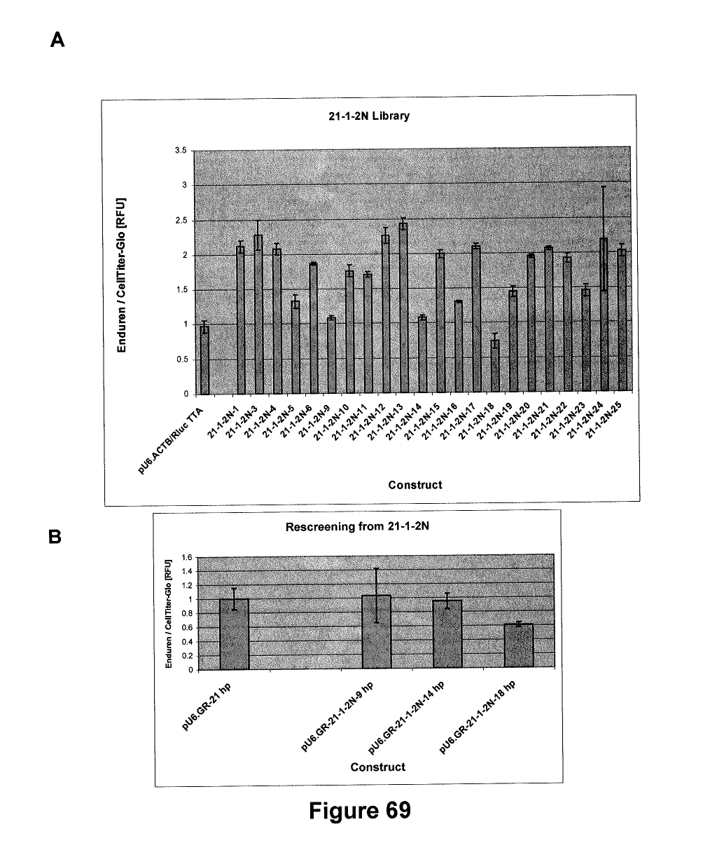

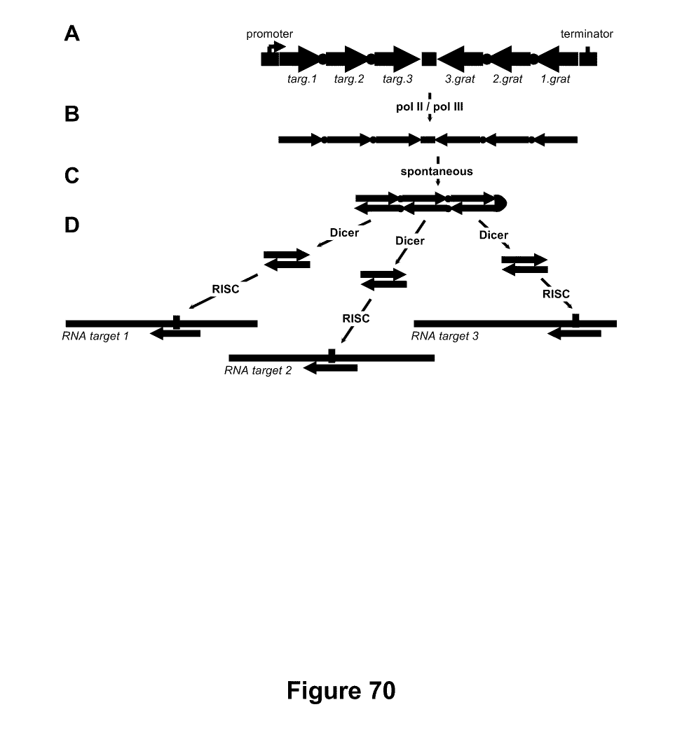

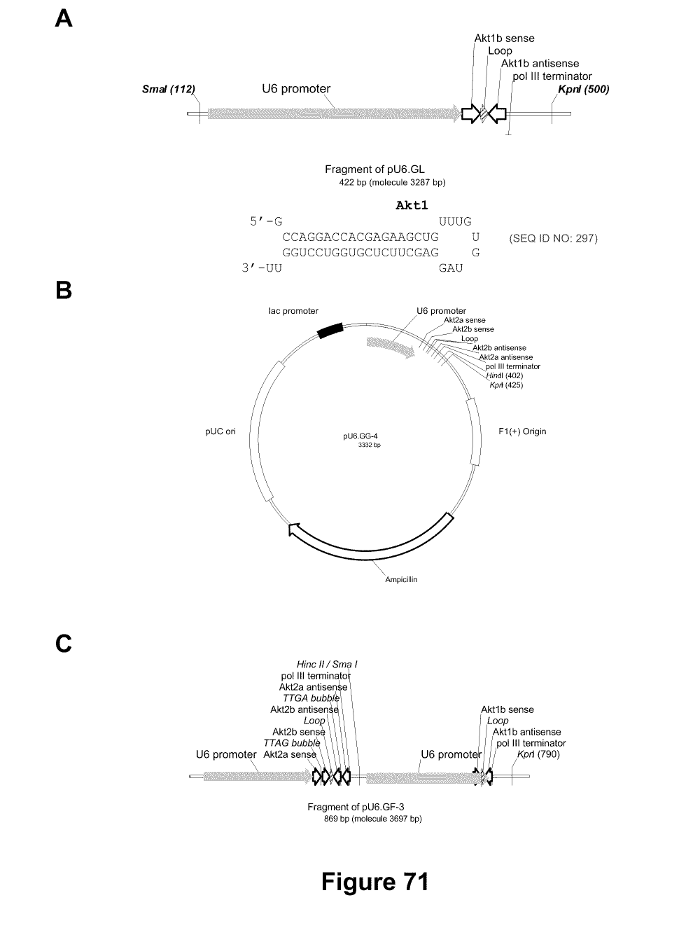

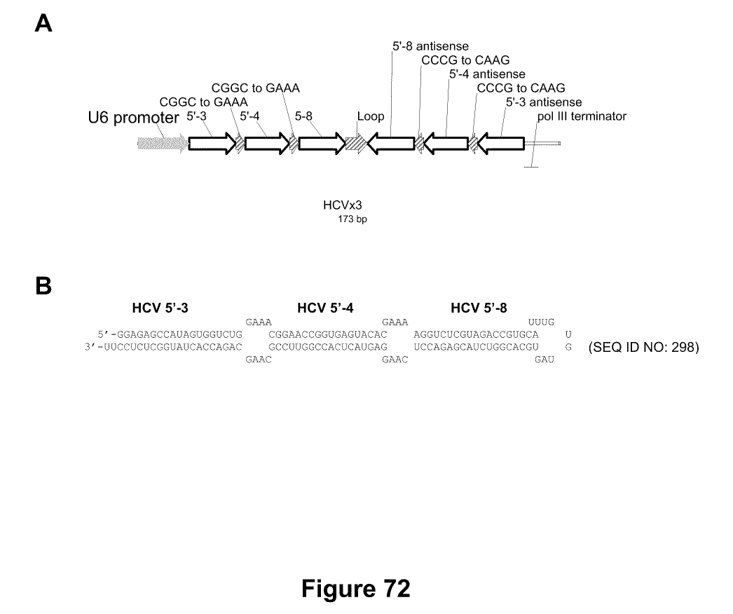

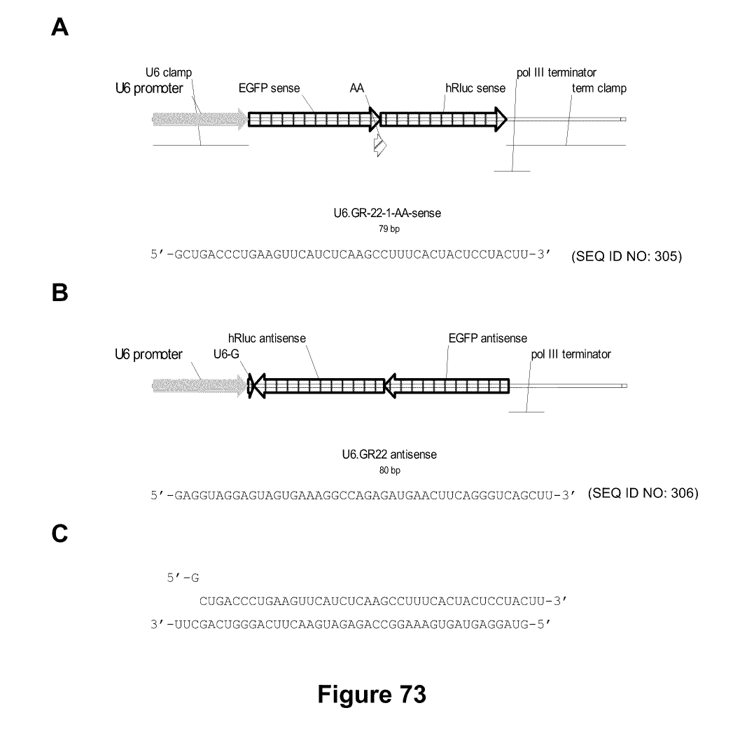

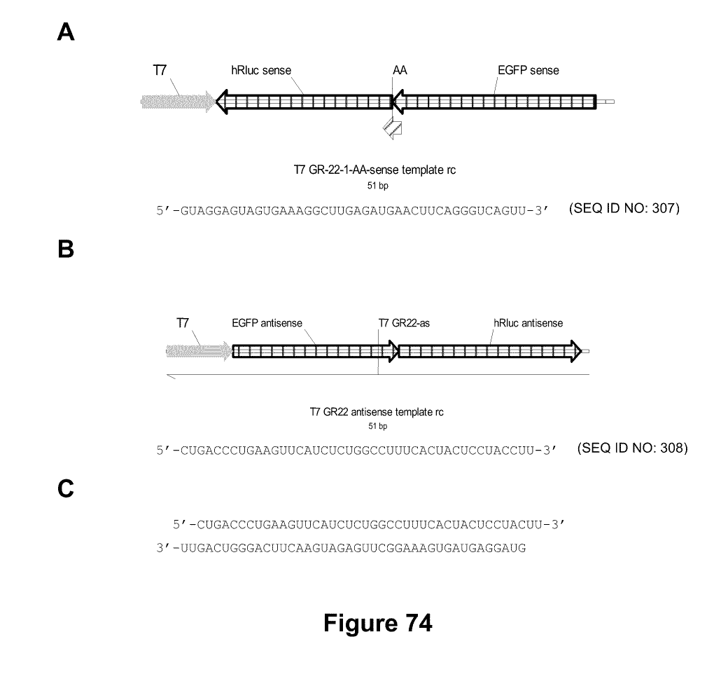

FIG. 65 shows a map of the library construct pU6.GR-21-1&4-4N, which targets GFP and Rluc for inactivation. A shows a map of the hairpin DNA transcriptional unit as for FIG. 63. B shows the predicted hairpin RNA (SEQ ID NO: 286) produced from this construct as in FIGS. 63 and 64.

FIG. 66 shows selected examples of 3 phasing constructs, namely pU6.GR-17 hp (A), pU6.GR-21 hp (B) and pU6.GR-26 hp (C). In these examples the grey bar represents a small region of the human U6 promoter; the open arrow represents the EGFP-A effector sequences which range from 17 to 26 nts; the black arrows represent the Rluc targeting sequences, which are constant in these constructs.

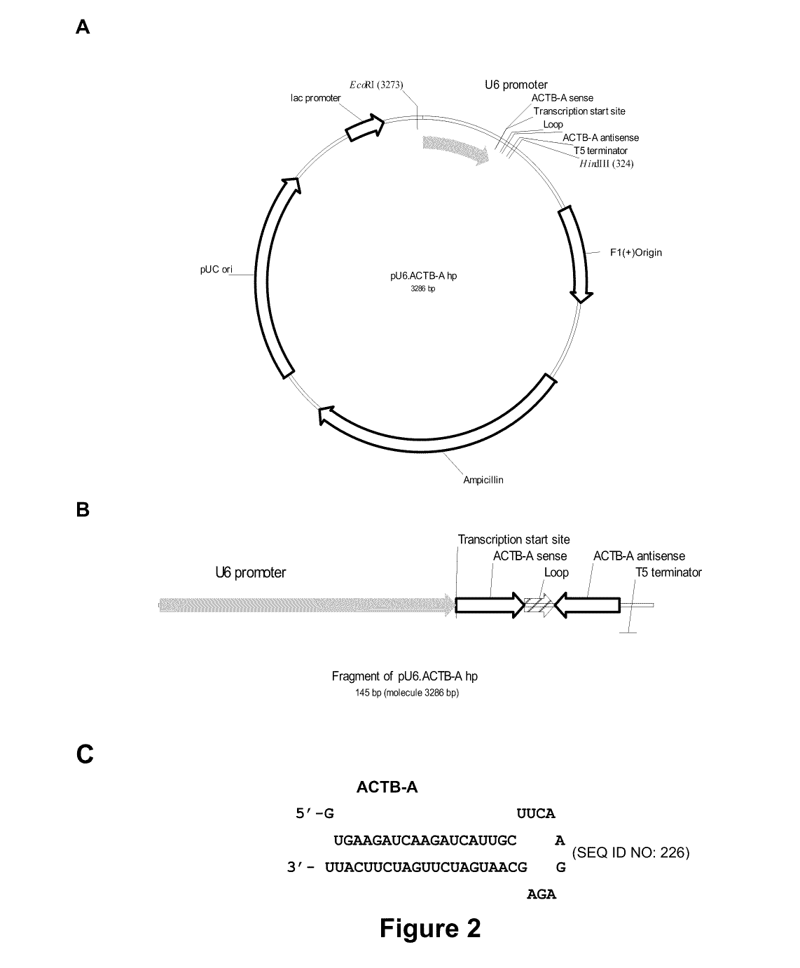

FIG. 67 shows the predicted transcripts (SEQ ID NOS: 287-296) produced by pU6.GR-17 hp (A), pU6.GR-18 hp (B), pU6.GR-19 hp (C), pU6.GR-20 hp (D), pU6.GR-21 hp (E), pU6.GR-22 hp (F), pU6.GR-23 hp (G), pU6.GR-24 hp (H), pU6.GR-25 hp (I) and pU6.GR-26 hp (J). The predicted transcripts are shown as in FIG. 2C, except the variable length GFP targeting sequences are shown in bold.

FIG. 68 shows the relative activity of the phasing constructs against Rluc (n=5; ±SD). Note the constructs pU6.GR21 hp and pU6.GR-22 hp show the highest activity.

FIG. 69 Defining constructs with higher activity by screening 2N libraries. A shows primary screening of the activity of 22 clones isolated from the pU6.GR-21-1-2N library against Rluc (n=3; ±SD). B rescreening of clones from A showing highest activity. Clone pU6.GR-21-1-2n-18 hp showed higher activity than the control pU6.GR-21 hp.

FIG. 70 shows a diagrammatic representation of the multi-target strategy. A shows a diagrammatic representation of a construct targeting 3 genes (targ. 1, targ. 2 and targ. 3 in this example). The construct contains a promoter (either pol II, pol III or any other type of promoter) and terminator (either pol II or pol III terminator or any sequence that can generate a 3′ end of the transcript): It also contains a transcribed effector sequences in sense (targ. 1, targ. 2 and targ. 3) and antisense (3.grat, 2.grat and 1.grat) orientation (arrows); loop sequences (box) and bubbles shown as black circles. B a primary transcript is produced as shown in FIG. 70A, consisting of sense and antisense effector sequences, separated by bubbles, with loop sequences separated by a loop. C The transcript then forms an hpRNA structure, presumably spontaneously. D The hp RNA transcript is then processed by Dicer to produce three different effector si RNAs. In this example the effectors can target 3 different RNAs (horizontal bars) and cleave them (vertical bars).

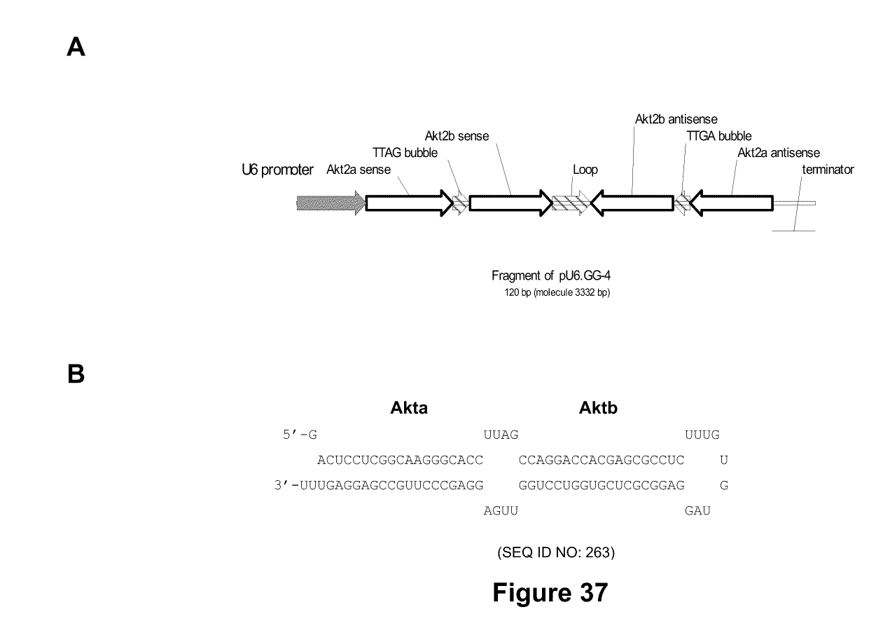

FIG. 71 shows construction of the plasmid pU6.GF-3. This plasmid contains 2 transcriptional units on a single plasmid, one designed to inactivate Akt1, the second to inactivate Akt2. A shows a map of the plasmid pU6.GL as in FIG. 2, the positions of Sma I and Kpn I restriction sites are also shown. The predicted hairpin RNA (SEQ ID NO: 297) produced from this construct as in FIG. 2C. B shows a map of the entire plasmid pU6.GG-4 (FIG. 37), the position of Hinc II and Kpn I restriction sites are shown. C shows a map of a region of the construct pU6.GF-3 which will be prepared by cloning the U6 transcriptional unit from pU6.GL as a Sma I/Hind III fragment into Hinc II/Kpn I restricted pU6.GG-4. The map shows the region of pU6.GF-3 containing the two U6 transcriptional units. The resultant plasmid is predicted to produce two hairpin RNAs, one targeting Akt2, as shown in FIG. 37B, the second targeting Akt1 as in FIG. 71B.

FIG. 72 shows a map of the construct pU6.HCVx3 hp. A shows a map of the hairpin DNA transcriptional unit as in FIG. 10A. B shows the predicted hairpin RNA (SEQ ID NO: 298) produced from this construct as in FIG. 10B.

FIG. 73 shows maps of regions of two plasmids, namely pU6.GR22-sense (A) and pU6.GR22-antisense (B). The predicted transcripts (SEQ ID NOS: 305 and 306) produced from these constructs in vivo are shown below the respective maps. The transcripts are predicted to anneal as shown in C to produce a double stranded RNA designed to inactivate both EGFP and hRluc mRNAs.

FIG. 74 shows partial maps of two DNA fragments, namely T7 GR22-sense template rc (A) and T7 GR22-antisense rc (B). The predicted transcripts (SEQ ID NOS: 307 and 308) produced from these constructs in vitro are shown below the respective maps. The transcripts are predicted to anneal as shown in C to produce a double stranded RNA designed to inactivate both EGFP and hRluc mRNAs.

### DESCRIPTION OF THE INVENTION

In one aspect, the present invention provides a ribonucleic acid (RNA) suitable for use as interfering RNA in gene silencing techniques comprising in a 5′ to 3′ direction at least a first effector sequence, a second effector sequence, a sequence substantially complementary to the second effector sequence and a sequence substantially complementary to the first effector sequence, the complementary sequences capable of forming double stranded regions with their respective effector sequences and further including one or more spacing sequences of one or more nucleotides.

In one embodiment, the first effector sequence is spaced from the second effector sequence by a first spacing sequence. In another embodiment, the sequence substantially complementary to the second effector sequence is spaced from the sequence substantially complementary to the first effector sequence by a second spacing sequence. Accordingly, RNA according to this aspect of the present invention can fold so that at least double stranded RNA region is spaced from an adjacent double stranded RNA region by spacing sequences, the spacing sequences being non-annealing and forming a so-called bubble. The terms “hybridising” and “annealing” refer to nucleotide sequences capable of forming Watson-Crick base pairs between complementary bases, as discussed further below.

In a further aspect the present invention provides a ribonucleic acid (RNA) suitable for use as interfering RNA in gene silencing techniques comprising at least a first effector sequence, a second effector sequence, a sequence substantially complementary to the second effector sequence and a sequence substantially complementary to the first effector sequence, the complementary sequences capable of forming double stranded regions with their respective effector sequences. Accordingly, at least one double stranded RNA region is directly adjacent to at least one other double stranded RNA region thereby producing at least two effector regions suitable for use in producing interfering RNA in the gene silencing technique, without intervening spacing sequences. In one preferred embodiment, the RNA further includes a spacing sequence between the second effector sequence and the sequence substantially complementary to it, the spacing sequence forming a loop about which the RNA folds to form the double-stranded regions.

In another aspect the present invention provides a ribonucleic acid (RNA) for use as interfering RNA in gene silencing techniques to silence a target gene comprising in a 5′ to 3′ direction at least a first effector sequence, a second effector sequence, a sequence substantially complementary to the second effector sequence and a sequence substantially complementary to the first effector sequence, wherein the complementary sequences are capable of forming double stranded regions with their respective effector sequences and wherein at least one of these sequences is substantially identical to the predicted transcript of a region of the target gene. Preferably, the RNA further comprises a spacer sequence of one or more nucleotides, wherein any two of the sequences are spaced by the spacing sequence. More preferably, the RNA further comprises an additional spacer sequence of one or more nucleotides.

In another aspect the present invention provides a ribonucleic acid (RNA) suitable for use as interfering RNA in gene silencing techniques comprising in a 5′ to 3′ direction at least a first effector sequence, a second effector sequence, a sequence substantially complementary to the second effector sequence and a sequence substantially complementary to the first effector sequence, the complementary sequences capable of forming double stranded regions with their respective effector sequences, the sequence substantially complementary to the second effector sequence being spaced from the sequence substantially complementary to the first effector sequence by one spacing sequence of one or more nucleotides, and the first effector sequence being spaced from the second effector sequence by another spacing sequence of one or more nucleotides. In one embodiment of this aspect of the present invention, both spacing sequences are included and do not anneal.

In a further aspect the present invention provides a ribonucleic acid (RNA) suitable for use as interfering RNA in gene silencing techniques comprising in a 5′ to 3′ direction at least a first effector sequence, a second effector sequence, a sequence substantially complementary to the second effector sequence and a sequence substantially complementary to the first effector sequence, the complementary sequences capable of forming double stranded regions with their respective effector sequences, the first effector sequence being spaced from the second effector sequence by a first spacing sequence of one or more nucleotides. In one embodiment, the sequence substantially complementary to the second effector sequence is spaced from the sequence substantially complementary to the first effector sequence by a second spacing sequence of one or more nucleotides, the second spacing sequence not being hybridisable with the first spacing sequence. Accordingly, the RNA according to this aspect of the present invention can fold so that at least one strand of at least one double stranded RNA region is spaced from an adjacent double stranded RNA region by a spacing (non-pairing) sequence, the spacing sequence forming a so-called bubble.

By an RNA “suitable for use as interfering RNA” is meant an RNA that may directly act as interfering RNA or that may be processed to produce RNA molecules that are active in RNA interference. Such RNA is suitable for genetic silencing techniques.

In another embodiment, there is provided a nucleic acid construct comprising at least a first effector sequence, a first complementary sequence that is substantially complementary to the first effector sequence, a second effector sequence and a second complementary sequence that is substantially complementary to the second effector sequence, wherein both first and second effector sequences form double stranded portions with their corresponding complementary sequences, the double stranded regions being spaced by a spacer sequence, usually a shorter sequence than the first effector sequence.

In preferred embodiments, one double stranded portion will have its two strands connected by a loop sequence forming the bend in the so-called hairpin structure. In this embodiment, the double stranded portion has this loop at one end, i.e. the loop is formed by a spacing sequence between one of the effector sequences and its substantially complementary sequence. Preferably, the nucleic acid also has a pair of spacing sequences between the double stranded portions, forming a “bubble”.

Preferably, the spacer sequence is shorter than either effector sequence. The spacer sequence is preferably 1 to 20, more preferably 1 to 10, more preferably 1 to 7 and most preferably 2 to 7 nucleotides long. Even more preferably, in one embodiment one spacer sequence is 2 nucleotides long and another spacer sequence is four nucleotides long.

As the ribonucleic acid or nucleic acid construct contains at least two effector sequences, the invention extends to such constructs containing three or more effector sequences, each with corresponding complementary sequences. The effector sequences and corresponding complementary sequences may be spaced from each other by spacing (non-pairing) sequences with the spacing sequence forming a bubble when the effector sequences base pair with the complementary sequences. In preferred embodiments, the ribonucleic acid or nucleic acid construct contains three effector sequences and three corresponding complementary sequences, each separated by a spacing sequence forming a bubble; four effector sequences and four corresponding complementary sequences, each separated by a spacing sequence forming a bubble; or five effector sequences and five corresponding complementary sequences, each separated by a spacing sequence forming a bubble. In further preferred embodiments, the ribonucleic acid or nucleic acid construct contains three effector sequences and three corresponding complementary sequences; four effector sequences and four corresponding complementary sequences; or five effector sequences and five corresponding complementary sequences without intervening spacing sequences between adjacent effector and complementary sequences. There may similarly be six, seven, eight, nine, ten or more effector sequences and complementary sequences in an RNA or nucleic acid construct of the invention. The effector sequences may be the same or different and directed to the same or different target genes, different regions of the same target gene or a combination of these.

In another embodiment, there is provided a ribonucleic acid suitable for use as interfering RNA in gene silencing techniques comprising in a 5′ to 3′ direction at least a first effector sequence, a second effector sequence, a sequence substantially complementary to the second effector sequence and a sequence substantially complementary to the first effector sequence, the complementary sequences capable of forming double stranded regions with their respective effector sequences, the second effector sequence being spaced from the sequence substantially complementary to the second effector sequence by a spacing sequence of one or more nucleotides.

In the context of the present invention, “target gene” refers to a gene which is targeted for silencing by RNA interference techniques. The RNA product of the gene may be a messenger RNA (mRNA) capable of being translated to form an amino acid sequence, or it may be a non-translated RNA, such as a ribosomal RNA, small uracil-rich RNA, or ribozyme.

Reference herein to a “gene” or “genes” is to be taken in its broadest context and includes:

(i) a classical genomic gene consisting of transcription and/or translational regulatory sequences and/or coding region and/or non-translated sequences (i.e. introns, 5′- and 3′-untranslated sequences); and/or

(ii) DNA and RNA viral genes; and/or

(iii) cDNA corresponding to the coding regions (i.e. exons) and/or 5′- and 3′-untranslated sequences,

whether naturally occurring or synthesised. Furthermore, “gene” includes within its scope both a nucleic acid coding for an amino-acid encoding RNA (i.e. mRNA) as well as a nucleic acid encoding a RNA that does not code for an amino acid sequence.

By “substantially identical” is meant about 70% identical to a portion of the target gene. Preferably, it is at least 80-90%, more preferably at least 95-100% identical, and includes 100% identity. Thus a sequence substantially identical to a region of a target gene has this degree of sequence similarity. Generally, a double-stranded RNA region of the invention may be subjected to mutagenesis to produce single or several nucleotide substitutions, deletions or additions without substantially affecting its ability to modify gene expression.

It is known that RNAi is generally optimised by identical sequences between the target and the RNAi construct, but that the RNA interference phenomenon can be observed with less than 100% homology. As is understood by those skilled in the art, the strands comprising the double-stranded regions must be sufficiently homologous to each other to form the specific double stranded regions. The precise structural rules to achieve a double-stranded region effective to result in RNA interference have not been fully identified, but approximately 70% identity is generally sufficient. Greater identity in the central portion of the effector sequence as opposed to the end portions is required as explained below. Another consideration is that base-pairing in RNA is subtly different from DNA in that G will pair with U, although not as strongly as it does with C, in RNA duplexes.

By “substantially complementary” is meant that the sequences are hybridisable or annealable. Moreover, it is know that hybridisation is affected by the conditions of the solution. In general, substantially complementary sequences will have at least 70% Watson-Crick base pairing.

The two sequences of an RNA duplex or double-stranded region are referred to as the “sense” strand and “antisense” strand, even though they may be different portions of one polynucleotide (eg. where it forms a hairpin). The “sense” strand is the one where the sequence is broadly related to the relevant region of the target gene (ie, one that is substantially the predicted transcription product), and the sequence annealing to the sense strand sequence is termed “antisense”. For RNAi efficacy, it is more important that the antisense strand be homologous (ie, exactly complementary) to the target sequence. In some circumstances, it is known that 17 out of 21 nucleotides is sufficient to initiate RNAi, but in other circumstances, identity of 19 or 20 nucleotides out of 21 is required. It is believed, at a general level, that greater homology is required in the central part of a double stranded region (i.e. duplex) than at its ends. Some predetermined degree of lack of perfect homology may be designed into a particular construct so as to reduce its RNAi activity which would result in a partial silencing or repression of the target gene's product, in circumstances in which only a degree of silencing was sought. In such a case, it is envisaged that only one or two bases of the antisense strand of the RNA construct would be changed. On the other hand, the other, sense strand of the RNA construct is more tolerant of mutations. It is believed this is due to the antisense strand being the one that is catalytically active. Thus, less identity between the sense strand and the transcript of a region of a target gene will not necessarily reduce RNAi activity, particularly where the antisense strand perfectly hybridises with that transcript. Mutations in the sense strand (such that it is not identical to the transcript of the region of the target gene) may be useful to assist sequencing of hairpin constructs and potentially for other purposes, such as modulating dicer processing of a hairpin transcript or other aspects of the RNAi pathway.

The terms “hybridising” and “annealing” (and grammatical equivalents) are used interchangeably in this specification in respect of nucleotide sequences and refer to nucleotide sequences that are capable of forming Watson-Crick base pairs due to their complementarity. The person skilled in the art would understand that non-Watson-Crick base-pairing is also possible, especially in the context of RNA sequences. For example a so-called “wobble pair” can form between guanosine and uracil residues in RNA. “Complementary” is used herein in its usual way to indicate Watson-Crick base pairing, and “non-complementary” is used to mean non-Watson-Crick base pairing, even though such non-complementary sequences may form wobble pairs or other interactions. However, in the context of the present invention, reference to “non-pairing” sequences relates specifically to sequences between which Watson-Crick base pairs do not form. Accordingly, embodiments of spacing or bubble sequences according to the present invention are described and illustrated herein as non-pairing sequences, regardless of whether non-Watson-Crick base pairing could theoretically or does in practice occur.

The term “effector sequence” and “effector” in the context of this specification relates to either DNA or RNA, depending on the context, and the term is used to denote a sequence that anneals to form a double-stranded region, due to complementarity of bases in the annealed region. The double-stranded region may determine the region of the target gene to which the construct is directed where the effector sequence, or the sequence substantially complementary to the effector sequence, is substantially identical to a region of the target gene.

In several preferred embodiments, the double stranded regions are interfering RNA (RNAi) sequences. Preferably, at least one of the effector sequences is substantially identical to at least a region of a target gene in the case of an RNA gene, or substantially identical to the predicted transcript of at least a region of a target gene in the case of a DNA gene. Preferably, the first effector sequence has this characteristic. In another preferred embodiment, the effector sequences are each separately substantially identical to different regions of a single target gene, or their predicted transcripts, as the case may be. In another preferred embodiment, the effector sequences are each separately substantially identical to regions of different target genes. In this context, “transcript” includes RNA which could theoretically be encoded by a DNA sequence, also called a “predicted transcript” regardless of the actual method of generation of that RNA sequence. In the DNA described in the embodiments below, at least one of the effector sequences is substantially identical or complementary to a region of the target gene (where the target gene is DNA). In this context, such a sequence may be called the “targeting sequence” where it is directed to a region of the gene to be silenced. Such a sequence may also be referred to structurally as an “intramolecular self-complementary targeting sequence”.

Alternatively, a double-stranded region may form a so-called “stem” sequence. In some embodiments, one or more of the effector sequences will have a different length to the sequence substantially complementary to it. In such a case, the unpaired portion may function as a spacer sequence. For example, where the effector sequence is generated by identity (or substantial identity) to a region of a target gene and the sequence substantially complementary to it is longer or shorter, the unpaired sequence will still be substantially identical to the corresponding region of the target gene, but may function as a spacer (e.g. loop or bubble) in the RNA, rather than as part of the effector sequence. In one embodiment, the effector sequence and the sequence substantially complementary to it are adjacent on the polynucleotide, in which case the region between these two sequences forms a loop comprised by either:

(i) the 3′ end of the effector sequence and the 5′ end of the complementary sequence; or

(ii) an unpaired sequence.

Similarly, where the effector and complementary sequences are not adjacent, but separated by one or more other double-stranded regions, the unpaired sequence may form a bubble.

The effector sequences may be of the same or different lengths. Preferably, effector sequences are at least 10 nucleotides in length, preferably 10-200 nucleotides in length. More preferably, they are 17 to 30 and most preferably 21 to 23 nucleotides in length. In different embodiments, the effector sequences are 17, 18, 19, 20, 21, 22, 23, 24, 25, 26, 27, 28, 29 or 30 nucleotides in length, respectively, or any combination of two or more of these lengths.

It will also be understood that the term “comprises” (or its grammatical variants) as used in this specification is equivalent to the term “includes” and should not be taken as excluding the presence of other elements or features.

Where the first effector sequence is longer than a second effector sequence, it has been found that the activity of the double-stranded sequence may be enhanced. In such a situation, the second effector sequence (which usually is not designed to be substantially identical to any particular target) can be called a “stem”. Preferably, the stem sequence is 1 to 50 nucleotides in length. A suitable stem sequence is GACUGAA and its complement.

Bubbles are formed by two unpaired, or partially unpaired, strands (which may also be spacing sequences) containing at least a single unpaired base that bridge or link the double stranded regions on the nucleic acid. Further, a bubble may form where one strand of the nucleic acid includes one or more spacer nucleotides between the double stranded regions and the other strand includes no such spacer nucleotides. In this case, as the end nucleotides on that other strand near the junction of the double-stranded regions form the bubble with the one or more spacer nucleotides. Preferably the RNA according to this aspect of the present invention includes one loop region and one or more bubble regions. Preferably the bubble regions comprise 1 to 20 unpaired nucleotides per RNA strand. More preferably, the bubble regions comprise 2 to 10 unpaired nucleotides. In a preferred embodiment the bubble region includes the nucleotide sequence AA, UU, UUA, UUAG, UUACAA or N₁AAN₂, where N₁ and N₂ are any of C, G, U and A and may be the same or different. In a further preferred embodiment, the opposing sequence to each of these to form the bubble is AA, UU, UUG, UUGA, UUGUUG, and N₁AAN₂ respectively, where N₁ and N₂ are any of C, G, U or A and may be the same or different.

In a preferred embodiment, a nucleic acid according to the present invention comprises two double stranded RNA regions separated by a bubble region and a loop at one end of the double stranded RNA region. In another preferred embodiment, the nucleic acid according to the present invention comprises five double stranded RNA regions, with the first and second, second and third, third and fourth and fourth and fifth double stranded regions, respectively, being separated by a bubble region and with a loop at one end of the fifth double stranded RNA region.

In another preferred embodiment, there is provided a construct including sequence -X-A-Y-L-Y′-B-X′-, wherein:

X is a nucleotide sequence substantially identical to a first region, or a transcript of a region, of a target gene;

Y is a nucleotide sequence of one or more nucleotides;

A is a nucleotide sequence shorter than X;

B is a nucleotide sequence shorter than X and non-complementary to A;

L is a loop sequence;

X′ is substantially complementary to X; and

Y′ is substantially complementary to Y.

Additional effector sequences, with complementary sequences to form duplexes, and with or without spacer sequences like A in this embodiment may be added.

In another preferred embodiment, there is provided a construct including sequence -X-A-Y-L-Y′-X′-, wherein:

X is a nucleotide sequence substantially identical to a first region, or a transcript of a region, of a target gene;

Y is a nucleotide sequence of one or more nucleotides;

A is a nucleotide sequence shorter than X;

L is a loop sequence;

X′ is substantially complementary to X; and

Y′ is substantially complementary to Y.

In another preferred embodiment there is provided a construct including sequence -X-Y-L-Y′-X′-, wherein:

X is a nucleotide sequence substantially identical to a first region, or a transcript of a region, of a target gene;

Y is a nucleotide sequence of one or more nucleotides;

L is a loop sequence;

X′ is substantially complementary to X; and

Y′ is substantially complementary to Y.

In a further embodiment, L comprises -P-Q-R-S-T-, wherein P, Q, R, S and T each represent a nucleotide sequence of one or more nucleotides and Q and S are hybridisable with each other, P and T do not hybridise so forming a bubble and R is an unpaired loop region. P is preferably one of UU, UUA, UUAG or UUACAA. Preferably, the opposing sequence to each of these to form the bubble is UU, UUG, UUGA and UUGUUG respectively or vice versa. In one preferred embodiment, R is UUCAAGAGA.

In one embodiment, Y is substantially identical to a second region, or a transcript of a region, of a target gene, the target gene being the same or different from the gene referred to in the definition of X. Where the target genes are the same, typically different regions will be targeted by X and Y.

In another preferred embodiment, there is provided a construct further including the sequences C and D in the form -C-X-A-Y-L-Y′-B-X′-D-, wherein:

C is a nucleotide sequence shorter than X;

D is a nucleotide sequence shorter than X non-complementary to C.

In another preferred embodiment, there is provided a construct including sequence -S-A-T-A-U-A-V-A-W-L-W′-B-V′-B-U′-B-T′-B-S′-, wherein:

S, T, U, V and W are nucleotide sequences each substantially identical to a region, or a transcript of a region, of a target gene;

A is a nucleotide sequence shorter than S, T, U, V and W (each A may be the same or different);

B is a nucleotide sequence shorter than S, T, U, V and W and non-complementary to A (each B may be the same or different, but each B is non-complementary to its opposed A sequence when a double-stranded construct is formed about sequence L by annealing of S, T, U, V and W with their respective complements);

L is a loop sequence;

S′, T′, U′, V′ and W′ are nucleotide sequences substantially complementary to S, T, U, V and W.

As will be appreciated by one skilled in the art, it is not necessary that the entire construct is generated as one sequence. For example, in one embodiment of the invention, the at least first and second effector sequences, together with any spacing sequence, are generated (eg, transcribed by one DNA sequence), and the sequences substantially complementary to the effector sequences, together with any spacing sequence, are generated (eg, transcribed from a separate DNA sequence). The two or more DNA sequences may be under the control of separate promoters. Any loop sequence may be attached to either transcript or part of the loop attached to the 3′ end of one transcript and the 5′ end of the other transcript, and a ligation performed. In circumstances where the RNA construct is to be delivered by a DNA construct to a cell, in this embodiment, the two transcripts would be separately generated, and then would hybridise through annealing between the at least first and second effector sequences and their complements.

In a further aspect of the present invention there is provided a nucleic acid construct encoding any of the ribonucleic acids described above. In a preferred embodiment, this construct is a deoxyribonucleic acid (DNA) construct. In one embodiment, the DNA construct includes a sequence encoding a ribonucleic acid (RNA) suitable for use as interfering RNA in gene silencing techniques, the construct comprising in a 5′ to 3′ direction at least a first effector-encoding sequence, a second effector-encoding sequence, a sequence substantially complementary to the second effector-encoding sequence and a sequence substantially complementary to the first effector-encoding sequence, the complementary sequences' transcripts capable of forming double stranded regions with the respective effector-encoding sequences' transcripts. In an embodiment of this aspect of the invention, the first effector-encoding sequence is spaced from the second effector-encoding sequence by a first spacing sequence of one or more nucleotides. Preferably, the sequence substantially complementary to the second effector-encoding sequence is spaced from the sequence substantially complementary to the first effector-encoding sequence by a second spacing sequence of one or more nucleotides. Preferably, the second spacing sequence does not anneal with the first spacing sequence. Accordingly, the RNA of, or encoded by, the nucleic acid construct according to this embodiment can fold so that at least one double stranded RNA region is spaced from an adjacent double stranded RNA region by a spacing (non-pairing) sequence, the spacing sequence forming a so-called bubble. Preferably, the nucleic acid construct further includes a spacing sequence between the second effector sequence and the sequence substantially complementary to it, wherein the RNA of, or encoded by, the nucleic acid construct according to this embodiment forms a loop about which the RNA folds to form the double-stranded region between the second effector sequence and the sequence substantially complementary to the second effector sequence.

In a further aspect the present invention provides a nucleic acid construct including a sequence encoding a ribonucleic acid (RNA) suitable for use as interfering RNA in gene silencing techniques to silence a target gene, the construct comprising in a 5′ to 3′ direction at least a first effector-encoding sequence, a second effector-encoding sequence, a sequence substantially complementary to the second effector-encoding sequence and a sequence substantially complementary to the first effector-encoding sequence, wherein the transcripts of the complementary sequences are capable of forming double stranded regions with the transcripts of their respective effector-encoding sequences and wherein at least one of these sequences is substantially identical to a region of the target gene.

Preferably, the nucleic acid construct further comprises a spacing sequence of one or more nucleotides wherein any two of the encoding sequences are spaced by a spacing sequence. In preferred embodiments, the first effector-encoding sequence is spaced from the second effector-encoding sequence by the spacing sequence and/or the sequence substantially complementary to the first effector-encoding sequence is paced from the sequence substantially complementary to the first effector-encoding sequence by the spacing sequence.

In a further preferred embodiment the nucleic acid construct further comprises an additional spacing sequence. In a preferred embodiment, the first effector-encoding sequence is spaced from the second effector-encoding sequence or the sequence substantially complementary to the second effector-encoding sequence is spaced from the sequence substantially complementary to the first effector-encoding sequence by the additional spacing sequence and the transcript of the first spacing sequence is not annealable with the transcript of the additional spacing sequence.

The nucleic acid construct or an RNA according to the invention will usually be a recombinant or isolated molecule.

In a further preferred embodiment, the nucleic acid construct comprises a spacing sequence of one or more nucleotides between the second effector encoding sequence and the sequence substantially complementary to the second effector-encoding sequence.

Preferably, the nucleic acid construct further includes a loop coding sequence between the second effector-encoding sequence and the sequence substantially complementary to the second effector-encoding sequence. The loop forms the “hinge” of the hairpin. In one embodiment, the loop's sequence is 5′TTCAAGAGA3′. In a further embodiment, the loop sequence is 5′TTTGTGTAG3′.

Preferably the construct is derived from a DNA vector selected from the group consisting of a plasmid, a bacteriophage and a viral-based vector. Preferably the DNA construct is suitable for producing RNA suitable for use as interfering RNA in gene silencing technologies. More preferably, the construct can be introduced into a cell where gene silencing is to take place and interfering RNA can be transcribed within this cell.

Preferably the first effector sequence or its complementary sequence is substantially identical or substantially complementary to a region of a target gene. In one embodiment, the second effector sequence or its complementary sequence is substantially identical to the same or a different region of the same or a different target gene. In another embodiment, the second effector sequence or its complementary sequence is substantially identical to a region of a different target gene.

In another embodiment, the DNA construct comprises up to five effector-encoding sequences. Each of the encoded effector sequences or their complementary sequences is substantially identical to a region of a target gene. The encoded effector sequences or their complementary sequences may be substantially identical to regions of different target genes, or to different regions in the same target gene.

The construct according to the present invention may further contain one or more regulatory elements to allow transcription of the RNA to take place. Preferably at least one of the regulatory elements is a promoter, which is operably linked with the portion of the construct encoding the nucleic acid according to the present invention. A variety of promoters may be included in the polynucleotide vector. Factors influencing the choice of promoter include the desire for inducible transcription of the oligonucleotide or oligonucleotide and polynucleotide sequences, the strength of the promoter and the suitability of the promoter to induce expression in the in vivo or in vitro environment in which the transcription is to take place. In a preferred embodiment the promoter is an RNA polymerase III (pol III) promoter such as U6 or H1 promoters.

One or more of the regulatory elements of the construct according to the present invention may be a terminator sequence. Such a terminator sequence may be operably linked with the portion of the construct encoding the nucleic acid of the present invention in order to determine the sequence of the 3′ end of the transcribed nucleic acid. Terminators for the various classes of RNA polymerase as known to those skilled in the art. In one embodiment, the terminator is a pol II terminator. In another embodiment, the terminator is a pol III terminator. Preferably, the pol III terminator includes the sequences TTTTT or TTTTTT.

As will be appreciated, such constructs will often also include selection markers or sequences (eg, Ampicillin resistance) and/or restriction enzyme sites.

In a preferred embodiment, the nucleic acid construct includes a transcriptional unit comprising a promoter; at least a first effector-encoding sequence; a second effector-encoding sequence; a sequence substantially complementary to the second effector-encoding sequence; a sequence substantially complementary to the first effector-encoding sequence and a terminator sequence, the promoter, effector sequences, sequences complementary to the effector sequences and terminator being operably linked. The nucleic acid construct may include in addition to the transcriptional unit described above at least one further transcriptional unit encoding RNA suitable for use as interfering RNA for use in gene silencing techniques. By “operably linked” in the context of the present invention means that the transcription of a nucleic acid is modulated by the regulatory element with which it is connected. Preferably these are incorporated within a vector.

The DNA construct may have regulatory and other elements inserted by methods known in the art so as to optimise the transcription of the RNA suitable for use as interfering RNA in gene silencing techniques.

It will be apparent to the person skilled in the art that deoxyribonucleic acids (DNA) and ribonucleic acids (RNA) may include modified nucleotides. Thus RNA in the context of the present invention includes nucleic acid containing principally any or all of the ribonucleotides uracil (U), guanosine (G), cytosine (C) and adenosine (A), however modified or otherwise altered nucleotides and nucleotide analogues may also be included within an RNA sequence. Likewise, DNA contains principally any or all of the deoxyribonucleotides thymidine (T), guanosine (G), cytosine (C) and adenosine (A), however modified or otherwise altered nucleotides and nucleotide analogues may also be included within a DNA sequence.

In another aspect of the present invention there is provided a method of producing RNA from the construct according to the present invention. The RNA is preferably RNAi for use in gene silencing techniques. The RNA may be produced from the construct according to the present invention in vitro, or by in vivo techniques after introduction of the construct into a cell. In this specification, “silence” means reduced expression, but is not limited to prevention of expression.

In another aspect of the present invention there is provided a method of inhibiting the expression of a target gene by introducing the nucleic acid or construct of the present invention into a cell or other system or environment permitting expression permitting expression of a target gene (including for example a cell lysate, tissue, in vitro system etc) containing a target gene to be silenced using RNAi techniques. In a preferred embodiment, multiple target genes or multiple gene targets are silenced.

A variety of vectors may be used to introduce the nucleic acid or construct encoding the nucleic acid of the present invention into a cell. Virus-based vectors, such as those related to adenovirus, lentivirus or retrovirus, may be used. The expression of the nucleic acid according to the present invention may be in vitro, ex vivo or in vivo. The expression of the nucleic acid after introduction of the construct according to the present invention into a cell may be stable (that is, long-term) or transient. Adeno-associated virus is one preferred vector. Other preferred vectors are retroviral and lentiviral vectors.

The use of the method of this aspect of the present invention has applications in gene therapy strategies where multiple gene inactivation and/or complete inactivation of a gene (for example, an oncogene) would be advantageous. For example, viruses may be controlled by targeting two or more regions of a viral genome, or genes of a virus; thereby decreasing the likelihood that the virus might mutate to become resistant to the effect of a particular DNA construct. Furthermore, multiple site in a single viral gene may be targeting using the nucleic acid or construct according to the present invention. Another potential use in viral control might be to design a single construct inactivating both viral genes and also host genes involved in viral replication. Such uses and methods are within the scope of the invention. Accordingly, the method of the present invention may be used to inactivate two or more genes of the human immunodeficiency virus (HIV) or to inactivate one or more HIV genes and one or more HIV receptors on the host cell, for example the CCR4 receptor.

In cancers, mutations frequently occur in multiple genes. For gene therapy approaches, inactivation of two or more critical genes involved in tumour development are likely to prove more effective in controlling cancer cell proliferation than DNA constructs inactivating a single gene. For example, the development of a particular type of tumour may be accelerated by the cumulative effect of two signalling pathways controlled by two different genes. The simultaneous inactivation of the two genes may result in more immediate control of tumour growth. Furthermore, the tumour development may involve two alternative pathways controlled by different genes, whereby the inhibition of both pathways would be a requirement for the effective inhibition of tumour development.

The method according to this aspect of the present invention may be useful for the treatment and/or prevention of disease in plants and animals, including humans. This method has the advantage over many other treatments in that the gene can be targeted with high specificity, reducing the possibility for side-effects.

Multiple gene inactivation strategies are also likely to have uses in target definition and gene function studies. For example, DNA constructs according to the present invention may be designed whereby the construct can inactivate a single gene A by possessing a target sequence for that gene. In order to establish the phenotypic effects of inhibiting the expression of a particular gene in the environment where gene A is not expressed, other sequences can be included in the multiple target construct. For example, random shotgun library sequences can be cloned into the DNA construct already possessing the target sequence for gene A. Therefore, such a library can be used to screen for genes of unknown functions in a background where the first gene is also inactivated.

Regions of target genes targeted by RNAi techniques may be predicted, including empirically or by various algorithms. Where there is more than one optimal target sequence, all such target sequences may be included in one construct.

Different non-complementary bubble-forming or bubble-encoding sequences in the constructs or nucleic acids of the present invention may have different activity in respect of gene silencing. Accordingly, random libraries of bubble sequences may be generated to determine the optimal sequences required for gene silencing activity for any given application or system. Such a method may involve inserting one or more randomised nucleotides into specific defined positions along a bubble sequence in a DNA construct and testing the activity of the interfering RNA encoded by the adjacent double-strand forming region. Such bubble sequences may be up to ten nucleotides in length or more. Preferably the bubble sequence is four or six nucleotides in length.

Constructs inactivating multiple target genes may also be used in transgenic systems to screen directly for the effects of inactivating two known genes. Such an approach may circumvent the requirement of complex breeding programs to generate individual animals possessing multiple gene inactivation.

The nucleic acid or construct according to the present invention may be introduced into a cell in a suitable context. The carriers, excipients and/or diluents utilised in delivering the subject nucleic acid or constructs to a host cell should be acceptable for human or veterinary applications. Such carriers, excipients and/or diluents are well-known to those skilled in the art. Carriers and/or diluents suitable for veterinary use include any and all solvents, dispersion media, aqueous solutions, coatings, antibacterial and antifungal agents, isotonic and absorption delaying agents, and the like. Except insofar as any conventional media or agent is incompatible with the active ingredient, use thereof in the composition is contemplated. Supplementary active ingredients can also be incorporated into the compositions.

In another aspect of the present invention there is provided a method of inhibiting the expression of a target gene by introducing RNA produced from the construct of the present invention into a cell containing a target gene to be silenced using RNAi techniques.

A viral delivery system based on any appropriate virus may be used to deliver the RNA or nucleic acid construct of the present invention. In addition, hybrid viral systems may be of use. The choice of viral delivery system will depend on various parameters, such as the tissue targeted for delivery, transduction efficiency of the system, pathogenicity, immunological and toxicity concerns, and the like. Given the diversity of infections, diseases and other conditions that are amenable to interference by the RNA and RNA encoded by the nucleic acid constructs of the present invention, it is clear that there is no single viral system that is suitable for all applications. When selecting a viral delivery system to use in the present invention, it is important to choose a system where the interfering RNA-containing viral particles are preferably: 1) reproducibly and stably propagated; 2) able to be purified to high titres; and 3) able to mediate targeted delivery (delivery of the interfering RNA to the tissue or organ of interest without widespread dissemination).

In general, the five most commonly used classes of viral systems used in gene therapy can be categorized into two groups according to whether their genomes integrate into host cellular chromatin (oncoretroviruses and lentiviruses) or persist in the cell nucleus predominantly as extrachromosomal episomes (adeno-associated virus, adenoviruses and herpes viruses). This distinction is an important determinant of the suitability of each vector for particular applications; non-integrating vectors can, under certain circumstances, mediate persistent gene expression in non-proliferating cells, but integrating vectors are the tools of choice if stable genetic alteration needs to be maintained in dividing cells, for example where the target cells are rapidly proliferating cancer cells.

For example, in one embodiment of the present invention, viruses from the Parvoviridae family are utilized. The Parvoviridae is a family of small single-stranded, non-enveloped DNA viruses with genomes approximately 5000 nucleotides long. Included among the family members is adeno-associated virus (AAV), a dependent parvovirus that by definition requires co-infection with another virus (typically an adenovirus or herpes virus) to initiate and sustain a productive infectious cycle. In the absence of such a helper virus, AAV is still competent to infect or transduce a target cell by receptor-mediated binding and internalization, penetrating the nucleus in both non-dividing and dividing cells.

Once in the nucleus, the virus uncoats and the transgene is expressed from a number of different forms—the most persistent of which are circular monomers. AAV will integrate into the genome of 1-5% of cells that are stably transduced (Nakai, et al., J. Virol. 76:11343-349 (2002)). Expression of the transgene can be exceptionally stable and in one study with AAV delivery of Factor IX, a dog model continues to express therapeutic levels of the protein 4.5 years after a single direct infusion with the virus. Because progeny virus is not produced from AAV infection in the absence of helper virus, the extent of transduction is restricted only to the initial cells that are infected with the virus. However, unlike retrovirus, adenovirus, and herpes simplex virus, AAV appears to lack human pathogenicity and toxicity (Kay, et al., Nature 424: 251 (2003) and Thomas, et al., Nature Reviews Genetics 4:346-58 (2003)).

Typically, the genome of AAV contains only two genes. The “rep” gene codes for at least four separate proteins utilized in DNA replication. The “cap” gene product is spliced differentially to generate the three proteins that comprise the capsid of the virus. When packaging the genome into nascent virus, only the Inverted Terminal Repeats (ITRs) are obligate sequences; rep and cap can be deleted from the genome and be replaced with heterologous sequences of choice. However, in order produce the proteins needed to replicate and package the AAV-based heterologous construct into nascent virion, the rep and cap proteins must be provided in trans. The helper functions normally provided by co-infection with the helper virus, such as adenovirus or herpes virus mentioned above, also can be provided in trans in the form of one or more DNA expression plasmids. Since the genome normally encodes only two genes it is not surprising that, as a delivery vehicle, AAV is limited by a packaging capacity of 4.5 single stranded kilobases (kb). However, although this size restriction may limit the genes that can be delivered for replacement gene therapies, it does not adversely affect the packaging and expression of shorter sequences such as RNAi nucleic acids.

However, technical hurdles must be addressed when using AAV as a vehicle for nucleic acid constructs. For example, various percentages of the human population may possess neutralizing antibodies against certain AAV serotypes. However, since there are several AAV serotypes, some of which the percentage of individuals harbouring neutralizing antibodies is vastly reduced, other serotypes can be used or pseudo-typing may be employed. There are at least eight different serotypes that have been characterized, with dozens of others which have been isolated but have been less well described. Another limitation is that as a result of a possible immune response to AAV, AAV-based therapy may only be administered once; however, use of alternate, non-human derived serotypes may allow for repeat administrations. Administration route, serotype, and composition of the delivered genome all influence tissue specificity.

Another limitation in using unmodified AAV systems with a nucleic acid construct is that transduction can be inefficient. Stable transduction in vivo may be limited to 5-10% of cells. Yet, different methods are known in the art to boost stable transduction levels. One approach is utilizing pseudo typing, where AAV-2 genomes are packaged using cap proteins derived from other serotypes. One group of investigators exhaustively pseudotyped AAV-2 with AAV-1, AAV-3B, AAV-4, AAV-5, and AAV-6 for tissue culture studies. The highest levels of transgene expression were induced by virion which had been pseudotyped with AAV-6; producing nearly 2000% higher transgene expression than AAV-2. Thus, the present invention contemplates use of a pseudotyped AAV virus to achieve high transduction levels, with a corresponding increase in the expression of the interfering RNA.

Another viral delivery system useful with the nucleic acid construct of the present invention is a system based on viruses from the family Retroviridae. Retroviruses comprise single-stranded RNA animal viruses that are characterized by two unique features. First, the genome of a retrovirus is diploid, consisting of two copies of the RNA. Second, this RNA is transcribed by the virion-associated enzyme reverse transcriptase into double-stranded DNA. This double-stranded DNA or provirus can then integrate into the host genome and be passed from parent cell to progeny cells as a stably-integrated component of the host genome.

In some embodiments, lentiviruses are the preferred members of the retrovirus family for use in the present invention. Lentivirus vectors are often pseudotyped with vesicular stomatitis virus glycoprotein (VSV-G), and have been derived from the human immunodeficiency virus (HIV), the etiologic agent of the human acquired immunodeficiency syndrome (AIDS); visan-maedi, which causes encephalitis (visna) or pneumonia in sheep; equine infectious anemia virus (EIAV), which causes autoimmune hemolytic anemia and encephalopathy in horses; feline immunodeficiency virus (FIV), which causes immune deficiency in cats; bovine immunodeficiency virus (BIV) which causes lymphadenopathy and lymphocytosis in cattle; and simian immunodeficiency virus (Sly), which causes immune deficiency and encephalopathy in non-human primates. Vectors that are based on HIV generally retain <5% of the parental genome, and <25% of the genome is incorporated into packaging constructs, which minimizes the possibility of the generation of reverting replication-competent HIV. Biosafety has been further increased by the development of self-inactivating vectors that contain deletions of the regulatory elements in the downstream long-terminal-repeat sequence, eliminating transcription of the packaging signal that is required for vector mobilization.

Reverse transcription of the retroviral RNA genome occurs in the cytoplasm. Unlike C-type retroviruses, the lentiviral cDNA complexed with other viral factors—known as the pre-initiation complex—is able to translocate across the nuclear membrane and transduce non-dividing cells. A structural feature of the viral cDNA—a DNA flap—seems to contribute to efficient nuclear import. This flap is dependent on the integrity of a central polypurine tract (cPPT) that is located in the viral polymerase gene, so most lentiviral-derived vectors retain this sequence. Lentiviruses have broad tropism, low inflammatory potential, and result in an integrated vector. The main limitations are that integration might induce oncogenesis in some applications. The main advantage to the use of lentiviral vectors is that gene transfer is persistent in most tissues or cell types.

A lentiviral-based construct that may be used to express the RNA according to the present invention preferably comprise sequences from the 5′ and 3′ LTRs of a lentivirus. More preferably the viral construct comprises an inactivated or self-inactivating 3′ LTR from a lentivirus. The 3′ LTR may be made self-inactivating by any method known in the art. In a preferred embodiment, the U3 element of the 3′ LTR contains a deletion of its enhancer sequence, preferably the TATA box, Sp1 and NF-kappa B sites. As a result of the self-inactivating 3′ LTR, the provirus that is integrated into the host cell genome will comprise an inactivated 5′ LTR. The LTR sequences may be LTR sequences from any lentivirus from any species. The lentiviral-based construct may also incorporate sequences for MMLV or MSCV, RSV or mammalian genes. In addition, the U3 sequence from the lentiviral 5′ LTR may be replaced with a promoter sequence in the viral construct. This may increase the titre of virus recovered from the packaging cell line. An enhancer sequence may also be included.

Adenoviruses are non-enveloped viruses containing a linear double-stranded DNA genome. While there are over 40 serotype strains of adenovirus—most of which cause benign respiratory tract infections in humans—subgroup C serotypes 2 or 5 are predominantly used as vectors. The adenovirus life cycle normally does not involve integration into the host genome, rather it replicates as episomal elements in the nucleus of the host cell and consequently there is no risk of insertional mutagenesis. The wild type adenovirus genome is approximately 35 kb of which up to 30 kb can be replaced with foreign DNA. There are four early transcriptional units (E1, E2, E3 and E4), which have regulatory functions, and a late transcript, which codes for structural proteins. Progenitor vectors have either the E1 or E3 gene inactivated, with the missing gene being supplied in trans either by a helper virus, plasmid or by an integrated gene in a helper cell genome. Second generation vectors additionally use an E2a temperature sensitive mutant or an E4 deletion. The most recent “gutless” vectors contain only the inverted terminal repeats (ITRs) and a packaging sequence around the transgene, all the necessary viral genes being provided in trans by a helper virus.

Adenoviral vectors are very efficient at transducing target cells in vitro and in vivo, and can be produced at high titres (>1011/ml). With the exception of one study that showed prolonged transgene expression in rat brains using an E1 deletion vector, transgene expression in vivo from progenitor vectors tends to be transient. Following intravenous injection, 90% of the administered vector is degraded in the liver by a non-immune mediated mechanism. Thereafter, an MHC class I restricted immune response occurs, using CD8+ CTLs to eliminate virus infected cells and CD4+ cells to secrete IFN-alpha which results in anti-adenoviral antibody. Alteration of the adenoviral vector can remove some CTL epitopes; however, the epitopes recognized differ with the host MHC haplotype. The remaining vectors, in those cells that are not destroyed, have their promoter inactivated and persisting antibody prevents subsequent administration of the vector.

Approaches to avoid the immune response involving transient immunosuppressive therapies have been successful in prolonging transgene expression and achieving secondary gene transfer. A less interventionist method has been to induce oral tolerance by feeding the host UV inactivated vector. However, it is more desirable to manipulate the vector rather than it is to manipulate the host through immunosuppression. Although only replication deficient vectors are used, viral proteins are expressed at a very low level, which are then presented to the immune system. The development of vectors containing fewer genes—culminating in the “gutless” vectors which contain no viral coding sequences—has resulted in prolonged in vivo transgene expression in liver tissue. However, the initial delivery of DNA packaged within adenovirus proteins—the majority of which will be degraded and presented to the immune system—may still cause problems for clinical trials.

Until recently, the mechanism by which the adenovirus targeted the host cell was poorly understood. Tissue-specific expression was therefore only possible by using cellular promoter/enhancers, e.g., the myosin light chain 1 promoter or the smooth muscle cell SM22a promoter, or by direct delivery to a local area. Uptake of the adenovirus particle has been shown to be a two-stage process involving an initial interaction of a fibre coat protein in the adenovirus with a cellular receptor or receptors, which include the MHC class I molecule and the coxsackievirus-adenovirus receptor. The penton base protein of the adenovirus particle then binds to the integrin family of cell surface heterodimers allowing internalization via receptor mediated endocytosis. Most cells express primary receptors for the adenovirus fibre coat protein, however internalization is more selective. Methods of increasing viral uptake include stimulating the target cells to express an appropriate integrin and conjugating an antibody with specificity for the target cell type to the adenovirus. However, the use of antibodies increases the production difficulties of the vector and the potential risk of activating the complement system.

Another virus that may be used as a basis for a viral delivery vector in the present invention is the Herpes simplex virus-1. HSV-1 is a double-stranded DNA virus with a packaging capacity of 40 kb, or up to 150 kb (helper dependent). HSV-1 has strong tropism for neurons, but also has a high inflammatory potential. HSV-1 is maintained episomally. Replication defective HSV-1 vectors generally are produced by deleting all, or a combination, of the five immediate-early genes (ICP0, ICP4, ICP22, ICP27 and ICP47), which are required for lytic infection and expression of all other viral proteins. Unfortunately, the ICP0 gene product is both cytotoxic and required for high level and sustained transgene expression. As such, the production of non-toxic quintuple immediate-early mutant vectors is a trade-off against efficient and persistent transgene expression. An HSV-1 protein that is activated during latency has recently be shown to complement mutations in ICP0 and overcome the repression of transgene expression that occurs in the absence of ICP0. Substitution of this protein in place of ICP0 might facilitate efficient transgene expression without cytotoxicity in non-neuronal cells. Long-term expression can be achieved in the nervous system by using one of the HSV-1 neuron-specific latency-activated promoters to drive transgene expression.

Other viral or non-viral systems known to those skilled in the art may be used to deliver the RNA or nucleic acid constructs of the present invention to cells of interest, including but not limited to gene-deleted adenovirus-transposon vectors that stably maintain virus-encoded transgenes in vivo through integration into host cells (see, Yant, et al., Nature Biotech. 20:999-1004 (2002)); systems derived from Sindbis virus or Semliki forest virus (see Perri, et al, J. Virol. 74(20):9802-07 (2002)); systems derived from Newcastle disease virus or Sendai virus; or mini-circle DNA vectors devoid of bacterial DNA sequences (see Chen, et al., Molecular Therapy. 8(3):495-500 (2003)). In addition, hybrid viral systems may be used to combine useful properties of two or more viral systems.

To deliver a viral-based nucleic acid construct into target cells, the nucleic acid construct first must be packaged into viral particles. Any method known in the art may be used to produce infectious viral particles whose genome comprises a copy of the viral construct. For example, certain methods utilize packaging cells that stably express in trans the viral proteins that are required for the incorporation of the nucleic acid construct into viral particles, as well as other sequences necessary or preferred for a particular viral delivery system (for example, sequences needed for replication, structural proteins and viral assembly) and either viral-derived or artificial ligands for tissue entry. In such a method, a nucleic acid construct is ligated to a viral delivery vector and the resulting viral nucleic acid construct is used to transfect packaging cells. The packaging cells then replicate viral sequences, express viral proteins and package the viral nucleic acid constructs into infectious viral particles. The packaging cell line may be any cell line that is capable of expressing viral proteins, including but not limited to 293, HeLa, A549, PerC6, D17, MDCK, BHK, Bing cherry, phoenix, Cf2Th, or any other line known to or developed by those skilled in the art. One packaging cell line is described, for example, in U.S. Pat. No. 6,218,181.

Alternatively, a cell line that does not stably express necessary viral proteins may be co-transfected with two or more constructs to achieve efficient production of functional particles. One of the constructs comprises the nucleic acid construct of the present invention, and the other plasmid(s) comprises nucleic acids encoding the proteins necessary to allow the cells to produce functional virus (replication and packaging construct) as well as other helper functions. This method utilizes cells for packaging that do not stably express viral replication and packaging genes. In this case, the nucleic acid construct is ligated to the viral delivery vector and then co-transfected with one or more vectors that express the viral sequences necessary for replication and production of infectious viral particles. The cells replicate viral sequences, express viral proteins and package the viral nucleic acid constructs into infectious viral particles.

The packaging cell line or replication and packaging construct may not express envelope gene products. In these embodiments, the gene encoding the envelope gene can be provided on a separate construct that is co-transfected with the viral nucleic acid construct. As the envelope protein is responsible, in part, for the host range of the viral particles, the viruses may be pseudotyped. As described supra, a “pseudotyped” virus is a viral particle having an envelope protein that is from a virus other than the virus from which the genome is derived. One with skill in the art can choose an appropriate pseudotype for the viral delivery system used and cell to be targeted. In addition to conferring a specific host range, a chosen pseudotype may permit the virus to be concentrated to a very high titre. Viruses alternatively can be pseudotyped with ecotropic envelope proteins that limit infection to a specific species (e.g., ecotropic envelopes allow infection of, e.g., murine cells only, where amphotropic envelopes allow infection of, e.g., both human and murine cells). In addition, genetically-modified ligands can be used for cell-specific targeting.

After production in a packaging cell line, the viral particles containing the nucleic acid constructs are purified and quantified (titred). Purification strategies include density gradient centrifugation, or, preferably, column chromatographic methods.

In another aspect of the present invention there is provided a method of testing nucleic acid sequences for efficacy in RNAi comprising the steps of inserting DNA encoding RNAi regions to be tested into the construct according to the present invention; introducing the construct into a cell containing the target gene corresponding to the RNAi region; allowing RNA to be produced from the construct and evaluating the effect on the expression of the target gene.

In a further aspect of the present invention there is provided a method for the production of a construct according to the present invention using long range PCR techniques. In one embodiment there is provided a method of adding a predetermined oligonucleotide to a polynucleotide, the oligonucleotide being divided into a first sub-sequence and a second sub-sequence, by a polymerase chain reaction process including:

providing a first primer having at its 3′ end a fixing part hybridizable under polymerase chain reaction conditions with at least a first part of the polynucleotide and at its 5′ end an effector part identical to the first sub-sequence, and a second primer having at its 3′ end a fixing part hybridizable with at least a second part of the polynucleotide that is adjacent the first part of the polynucleotide and at its 5′ end an effector part identical to the second sub-sequence,

introducing the primers to the nucleotide under polymerase chain reaction conditions such that the fixing parts of each primer hybridizes with the polynucleotide;

conducting a multiple polymerase chain reaction to produce an amplification product which includes the effector parts of the primers at the ends of a double-stranded sequence; and

ligating the ends of the effector parts together to form a combined polynucleotide and oligonucleotide sequence.

For additional clarification, in this description of this embodiment of the invention directed towards production of a construct, the term “effector” is used for convenience and as an appropriate term, but in a different context from that in which it is used in describing the RNA an DNA constructs themselves above. It is thus used in a different context from the way in which it is described in the paragraph above that commences “The term ‘effector sequence’ and ‘effector’ in the context of . . . ”. The term “effector” in this embodiment and the related claims should be construed in context without importing the limitations of the meaning of “effector” described above. It may also be referred to as the “variable” sequence as it largely contains the sequence that will vary from construct to construct.

By “oligonucleotide” in this process is meant a nucleic acid sequence of 40 to 100, preferably less than 100 nucleotides in length. The oligonucleotide may be single or double-stranded. Preferably the oligonucleotide is DNA.

By “polynucleotide” in this process is meant a nucleic acid sequence of at least about 1000 nucleotides in length. The polynucleotide may be single or double-stranded depending on the stage of the process according to the present invention. The polynucleotide may have a double-stranded circular conformation or a linear form, or may be the linearized form of a previously circular double stranded sequence. Preferably the polynucleotide is DNA. In a preferred embodiment of the present invention, the polynucleotide is a DNA vector selected from the group consisting of a plasmid, a bacteriophage and a viral-based vector.

It will be appreciated by a person skilled in the art that the efficiency of the polymerase chain reaction (PCR) can be modified, for example by altering the denaturation, annealing and polymerisation temperatures, the timing of the cycles and the salt concentration in the reaction mixture. Variations of these and other conditions that allow the PCR reaction to take place are encompassed in the term “polymerase chain reaction conditions”. It will be further appreciated by a person skilled in the art that a range of products may be produced from a given PCR reaction. These products may be separated by size or weight by methods known in the art, such as gel electrophoresis. In a preferred embodiment of the present invention the desired PCR product is isolated from solution.

The long range PCR method of this aspect of the present invention can be used to insert a DNA oligonucleotide into a DNA polynucleotide that is a vector in order to form a construct which enables the oligonucleotide to be transcribed into a ribonucleic acid sequence (RNA). The transcription may take place from the oligonucleotide only or the RNA transcript may be the result of the transcription of a combination of oligonucleotide and polynucleotide sequences. The transcribed RNA may further be translated into protein, or may also remain as untranslated RNA. In a preferred embodiment of this aspect of the present invention the primers have a homology with a restriction enzyme site in the polynucleotide sequence. In a further preferred embodiment of this aspect of the present invention, the primers are phosphorylated and the ligation of the amplification product is catalysed by T4 DNA ligase.

The polynucleotide used in the methods according to this long-range PCR process may contain one or more regulatory elements to allow transcription to take place. Preferably at least one of the regulatory elements is a promoter. A variety of promoters may be included in the polynucleotide vector. Factors influencing the choice of promoter include the desire for inducible transcription of the oligonucleotide or oligonucleotide and polynucleotide sequences, the strength of the promoter and the suitability of the promoter to induce expression in the in vivo or in vitro environment in which the transcription is to take place. In a preferred embodiment the promoter is an RNA polymerase III (pol III) promoter such as U6 or H1 promoters

In a preferred embodiment of this aspect of this process, the oligonucleotide codes for an RNA sequence capable of forming a double-stranded hairpin structure due to the presence an inverted repeat sequence. Preferably, the first primer contains approximately one half of the inverted repeat sequence in its effector part and the second primer contains approximately the other half of the inverted repeat sequence in its effector part. More preferably, the first and second primers further contain at least one nucleotide at their 5′ ends that forms the loop region of the hairpin-loop RNA structure.

In another embodiment of this aspect of the present invention, the effector parts are at least partially complementary, such that upon transcription (following transfection of a cell by a vector which incorporates a polynucleotide as described above) their respective RNA transcripts may hybridise with each other due to the complementarity of their sequences.

In a further preferred embodiment the oligonucleotide used in the method according to this aspect of the present invention is capable of coding RNA suitable for use as interfering RNA in gene silencing techniques. Such techniques are described in the specification of PCT/AU99/00195. Preferably the RNA has a hairpin-loop structure.

In another embodiment the oligonucleotide encodes a restriction site and the addition of the oligonucleotide to the polynucleotide results in the a restriction site being inserted into the combined oligonucleotide and polynucleotide sequence. It will be appreciated by a person skilled in the art the where the polynucleotide is a vector, such as a plasmid, the insertion of a restriction site would have many advantages in the subsequent use of the plasmid, particularly for subcloning purposes.

In a further embodiment the oligonucleotide includes an intron, or non-coding, sequence of a gene. The polynucleotide may include the coding sequence of the gene. Accordingly, the addition of the oligonucleotide to the polynucleotide using the method of the present invention may allow the insertion of the intron at the appropriate site in the coding sequence of the gene. Insertion of an intron into a coding sequence of a gene has a number of practical applications. For example, insertion of introns into DNA constructs has been shown to increase transgene expression. Another possible application is to use introns as a means of delivering double stranded RNA to induce gene silencing.

In another aspect of this aspect of the present invention there is provided a DNA construct produced by the addition of an oligonucleotide to a polynucleotide according to the method of the present invention. The DNA construct may be useful for further subcloning purposes whereby a second oligonucleotide of interest may be introduced by, for example known subcloning techniques. The DNA construct may also be an expression construct for the further production of RNA and/of protein. Preferably the DNA construct is suitable for producing RNA suitable for use as interfering RNA in gene silencing technologies. More preferably, the construct can be introduced into a cell where gene silencing is to take place and interfering RNA can be transcribed within this cell.

In another aspect of the present invention there is provided primers suitable for use in the method according to the present invention. In a further aspect of the present invention there is provided a kit comprising a polynucleotide and a primer pair for producing a polynucleotide containing an additional oligonucleotide.

In a further embodiment of this aspect of the invention there is provided a method for the large scale production of large numbers of hairpin DNA plasmids using the long range PCR method of the present invention with automation procedures. The simplicity of the long range PCR method lends itself to automation, using a robotics system to amplify DNA templates and ligate these to prepare DNA vectors. Such vectors can also be used to transform bacteria to grow substantial copy numbers of the vectors. In this way large numbers of plasmids, for example targeting different regions of a single gene could be rapidly prepared.

In a further aspect of the invention, a method for preparing libraries of sequences using long range PCR techniques is provided. In this instance, portions of one or both of the forward and reverse primers are synthesised using redundant oligonucleotides. Following amplification, ligation and transformation of bacteria, individual colonies contain unique hairpin DNA constructs reflecting the particular redundancies incorporated into individual plasmid by individual amplification primers. In this way libraries with, for example, random loop sequences are prepared and individual plasmids from the library are analysed for gene silencing activity in order to define loop sequences that enhance the activity of hairpin DNA constructs.

In another aspect of the present invention there is provided a kit for constructing a nucleic acid construct using the long range PCR method of the present invention comprising the polynucleotide, a polymerase, a first primer, a second primer and a ligating enzyme in proportions suitable for the long range PCR method according to the present invention.

In another aspect of the present invention there is provided a kit for inhibiting the expression of a target gene, including a vector suitable for use in producing a construct according to the present invention. Such a vector may include regulatory elements and facility for insertion of a cassette encoding a nucleic acid designed according to the present invention.

Without being bound by any theory or mode of action, it is believed that the invention is mediated by enzymes including Dicer and Drosha. At least these two ribonucleases, both members of the RNase III class, play a central role in the processing of double stranded RNA into siRNAs.

Dicer is the best characterized component. Dicer is a thought to be a cytoplasmic protein. It can cleave double-stranded RNA to produce approx 21 nucleotide (nt) dsRNAs with a 2 nt 3′ overhang; this overhang is a characteristic of RNase III-type enzymes. The precise requirements that allow dsRNA to act as an efficient substrate for Dicer remain unclear. miRNA precursors are one such substrate—they naturally form a hpRNA structure, but typically contain regions of mismatch, ie they do not form perfect double stranded structures, in contrast to hpRNAs designed to produce siRNAs from expression constructs. Dicer appears normally to process hpRNAs from the base of the hairpin, but definitive proof of this is not yet available. Dicer probably plays other roles in the RNAi process. It has recently been shown that the enzyme plays a role in RISC, ie it might play a role in cleavage of the target mRNA.

Drosha is another RNase III enzyme implicated in RNA interference. Much less is known about its function compared to Dicer. The enzyme is nuclear and may be nucleolar, since Drosha is known to play a role in rRNA maturation, which is a nucleolar process. The precise role of Drosha in RNAi is unknown. It is known to play a role in processing of miRNAs and may play a role in processing longer dsRNAs in RNAi. Current models suggest that Drosha may recognize loop structures in RNA, bind to these, then cut hp RNAs about 19-21 nt downstream of the loop. Most RNase 111s are thought to act by recognising loop structures, although it is recognised that the model described above for Dicer processing contradicts this view.

A hp RNA expressed from a pol III promoter thus may have 2 potential pathways by which it might enter RISC, namely:
(i) direct exit from the nucleus to the cytoplasm where the hpRNA is presumably processed by Dicer from the base of the hairpin and enters RISC. This appears to be at least the major pathway operating on hpRNAs expressed from short hpRNAs of 19 nts.
(ii) processing in the nucleus (possibly nucleolus) by Drosha, followed by export to the cytoplasm. This processing may involve recognition of the loop and will result in the formation of a stem sequence carrying a 2 nt 3′ overhang. Once exported this Drosha processed RNA is probably processed by Dicer as above.

These models are currently incomplete and are possibly not mutually exclusive, ie a longer hpRNA might be processed by both pathways, some is processed by Drosha then Dicer, some only by Dicer. Moreover some hpRNAs are expressed with a 5′ leader sequence (“U6+27”) which may target the hpRNA to the nucleolus, ie it is preferentially processed by Drosha before Dicer.

Without being bound by any theory or mode of action it is believed that improved therapeutic efficacy and safety of RNAi constructs can be achieved by optimising the length of effector sequences. This may assist the cleavage enzymes, such as Dicer and Drosha, cleaving at the same, predictable position, thereby providing predictability of result and reduction of side effects and/or variability of efficacy within and between patients.

The present invention will now be more fully described with reference to the accompanying examples and drawings. It should be understood, however, that the description following is illustrative only and should not be taken in any way as a restriction on the generality of the invention described above.

### Examples

### 1. Test Constructs

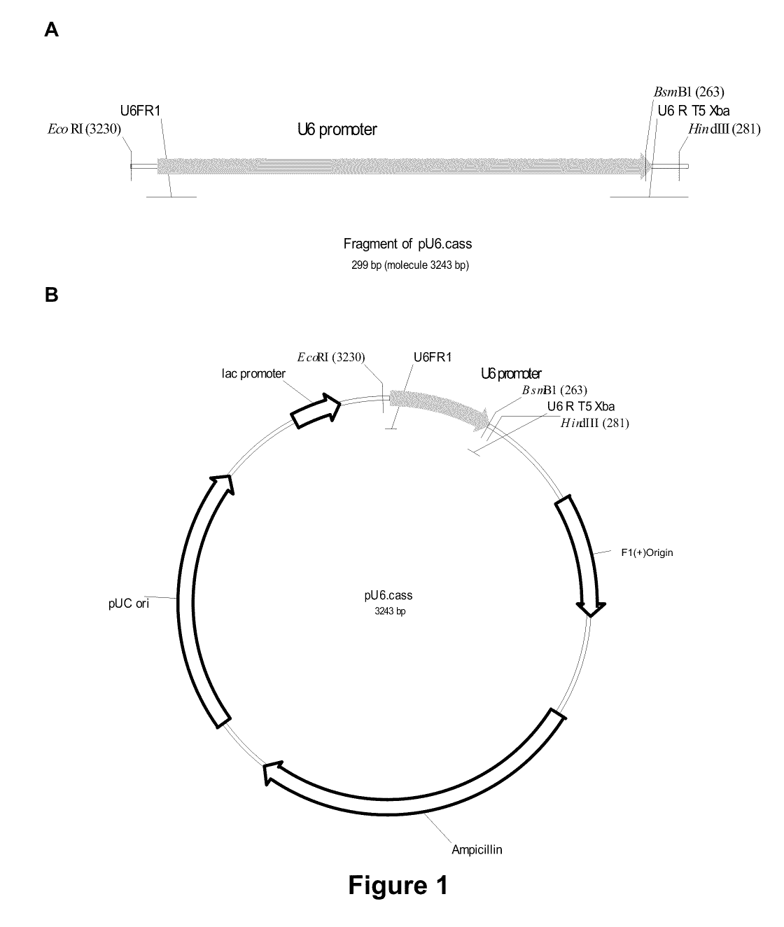

DNA constructs were prepared which were targeted to inactivate a number of genes, principally the Renilla luciferase gene because of the availability of simple rapid assays (see below). The base plasmid for all constructs was pU6.cass shown in FIG. 1. The cloning procedures used to prepare all constructs are well known to those skilled in the art. To prepare pU6.cass human genomic DNA was PCR amplified with Pfu polymerase using the primers.

U6FR1

(SEQ ID NO: 1)

GAATTCAAGGTCGGGCAGGAAGAGGG
U6T5H3

(SEQ ID NO: 2)

AAGCTTAGATCTCGTCTCACGGTGTTTCGTCCTTTCCACAAG

The resulting fragment was A-tailed using Taq polymerase and cloned into the vector pZero Blunt (pZB) using the manufacturer's protocols (Invitrogen). The human U6 promoter region was excised from this plasmid as an Eco RI/Hind III fragment and cloned into the vector pBluescript II SK+ (Stratagene), using the restriction sites introduced into the fragment by the above oligonucleotides. The resulting plasmid pU6.cass (FIG. 1) differed slightly from the predicted sequence because the particular clone chosen for subsequent manipulation had a two base pair (GA) deletion in the U6 fragment. The fragment actually cloned was an EcoRI/Hind III fragment, where the Eco RI site came from the pZB vector. pU6.cass thus had a 10 bp insertion at the 5′ end of the human U6 gene. The vector was designed to allow cloning of hairpin DNA inserts as Bsm BI/Hind III fragments, in such a fashion that hairpin RNA would be expressed from the insert.

The plasmid pU6.ACTB-A hp (FIG. 2) was prepared using annealing of four oligonucleotides, namely:

ACTB-A-hp-U6-5

(SEQ ID NO: 3)

ACCGTGTGCACCGGCACAGACATTCAAGAGA
ACTB-A-hp-U6-6

(SEQ ID NO: 4)

GCAATGATCTTGATCTTCA
ACTB-A-hp-H1-3

(SEQ ID NO: 5)

GCAATGATCTTGATCTTCATTTTTGGAAA
ACTB-A-hp-H1-4

(SEQ ID NO: 6)

AGCTTTTCCAAAAATGAAGATCAAGATCATTGCTCTCTTGAA

The partially complementary oligonucleotide pairs, ACTB-A-hp-U6-5 and ACTB-A-hp-U6-6 and ACTB-A-hp-H1-3 and ACTB-A-hp-H1-4 were annealed, and the annealed pairs themselves subsequently annealed to form a double-stranded DNA structure compatible with cloning into BsmB 1/Hind III digested pU6.cass. The annealed oligonucleotides were phosphorylated with T4 polynucleotide kinase using the manufacturer's (Promega) protocol and then cloned into the cut vector which had been dephosphoylated using Shrimp Alkaline Phosphatase (SAP) using the manufacturer's (Promega) protocol. This plasmid was expected to express a hairpin RNA, with transcription initiating in the human U6 promoter and terminating at the poly T tract in the 3′ region of the annealed sequences (SEQ ID NO: 226) as shown in FIG. 2C.

### 2. Long-Range PCR Method

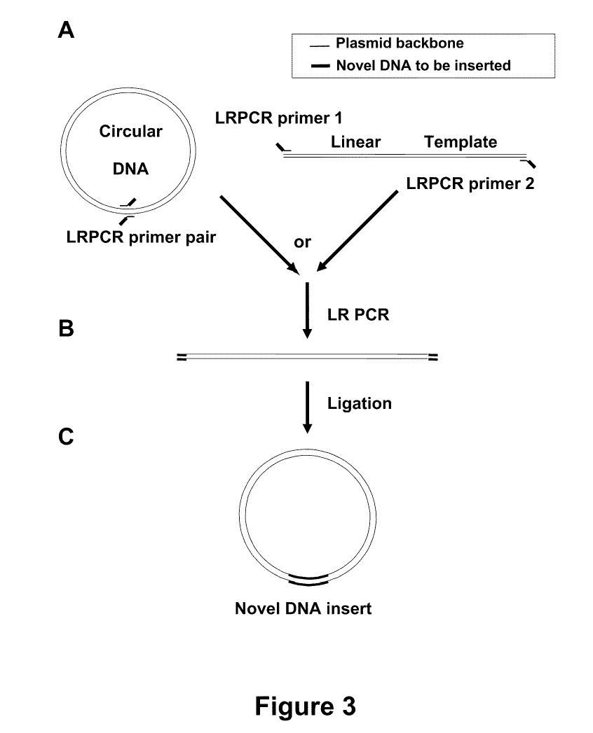

The general strategy of the long-range PCR method is shown in FIG. 3. The steps of the method are as follows:

Step 1: Long-range PCR (LPCR) primers are used to extend and amplify circular or linear templates. DNA templates are shown as two lines, denoting double stranded DNA, although single stranded DNA could be used as a template. The LPCR primers are shown as bent lines above and below the templates; thin regions represent 3′ fixing parts of primers, thick lines represent 5′ effector parts of primers.

A. Step 2: Amplify DNA molecule. PCR amplification of either of the templates in A will result in the production of linear DNA molecules, where the effector parts of the two LPCR oligonucleotides, denoted as thick lines, are incorporated into both ends of the linear DNA molecule.
C. Step 3: Circularized DNA molecule. The linear DNA can be readily recircularised using T4 DNA ligase or a similar enzyme. Note 5′ phosphorylation of at least one end of the DNA molecule is required to achieve this. This can be done by either synthesising 5′ phosphorylated oligonucleotides, or treating the linear DNA molecule with an enzyme such as T4 polynucleotide kinase; the former method is simplest.
2.1 Insertion of a Restriction Site into a Plasmid

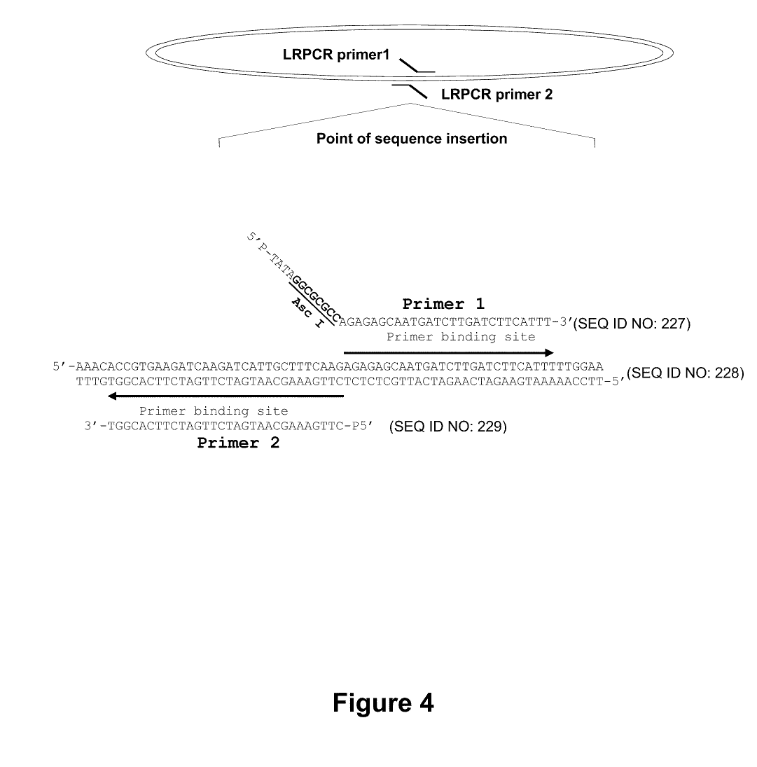

An Asc I restriction site was introduced into a plasmid as shown in FIG. 4. The addition of additional restriction sites to pre-existing DNA molecules is a widely used technique and in this instance the site was used for further manipulations.

The forward and reverse primers used in this reaction were:

(SEQ ID NO: 227)

TATAGGCGCGCCAGAGAGCAATGATCTTGATCTTCATTT
and

(SEQ ID NO: 7)

CTTGAAGCAATGATCTTGATCTTCACGGT

The substrate plasmid was amplified and ligated, and bacterial colonies were obtained and analysed as described above. In this fashion an ASC I restriction site was introduced in a single step.

The procedure is shown in FIG. 4 as follows:

A circular plasmid template is shown at the top, the two lines denote the positions at which the forward and reverse primers can anneal to the template at the point of sequence insertion. In this instance one primer contains only a 3′ fixing part, the other primer contains a 3′ fixing part as well as a 5′ effector part. The double stranded sequence (SEQ ID NO: 228) of the plasmid surrounding the point of insertion is shown below this. Above this, the sequence of the forward primer (SEQ ID NO: 227) is shown, the 3′ fixing part is shown directly above the sequence, the primer binding site is indicated by the arrow. The sequence of the 5′ effector region, which in this instance contains an Asc I restriction site, is indicated by the inclined letters. The sequence of the reverse primer (SEQ ID NO: 229) is shown below this and its primer binding site is also indicated by an arrow.

### 2.2 Long-Range PCR Strategy for Generating Hairpin DNA Constructs in U6 Expression Cassette

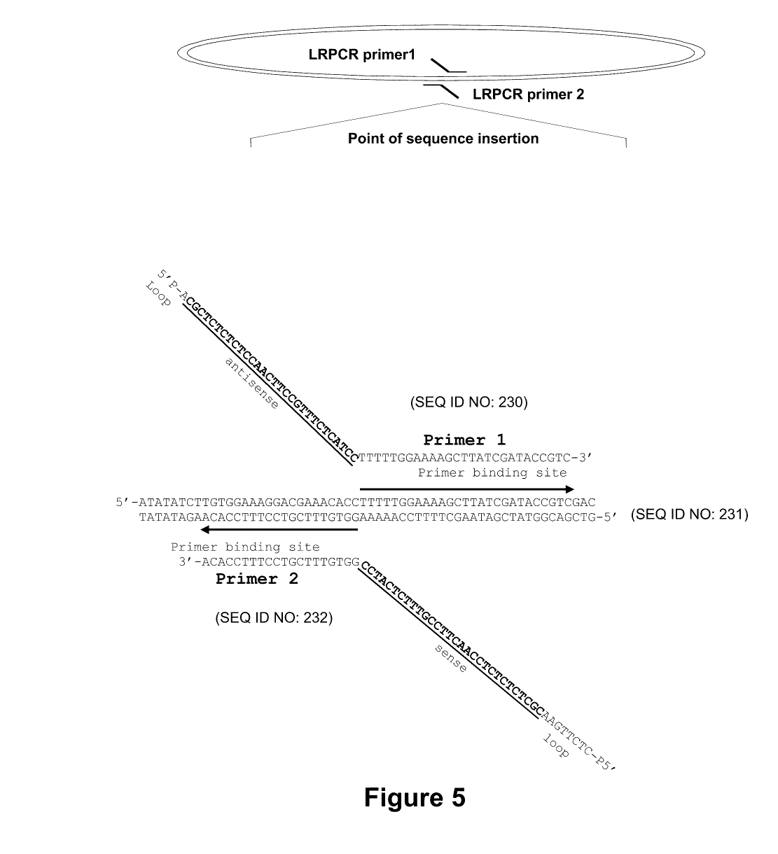

This example describes the optimised approach for generating hairpin DNA constructs using long range PCR as outlined in FIG. 5. The approach involves the use of two primers (SEQ ID NOS: 230 and 232) to generate a full copy of the expression cassette. The primers each contain approximately half of the hairpin and loop sequence (SEQ ID NO: 231), but no overlap in sequence. One primer is anchored in the U6 promoter region, the other in the pol III termination sequence and the primers are phosphorylated. The substrate used for amplification was a hairpin DNA construct (pU6.ACTB-A hp) containing both the human U6 promoter and a pol III terminator sequence; this template plasmid was prepared using conventional oligonucleotide cloning strategy similar to that described above. Following long-range amplification the PCR product is re-circularised and resultant colonies screened and a plasmid with the appropriate insert obtained.

The reverse and forward primers are designed to contain a 3′ U6 fixing part and a 3′ terminator fixing part, respectively. The 5′ sequences of each primer contain approximately half of the hairpin and loop sequences, in this instance 30 nucleotides homologous to a region of the murine GLUT4 gene separated by a 9 nucleotide loop.

The general design of the primers are shown below. U6 and terminator fixing parts are shown in bold.

The reverse primer is:
(SEQ ID NO: 8)
5′ (NNN)loop(a/s) (NNN)hairpin(a/s) GGTGTTTCGTCC
TTTCCACA 3′
The forward primer is:
(SEQ ID NO: 9)
5′(NNN)loop(s) (NNN)hairpin(a/s)
TTTTTGGAAAAGCTTATCGATACCGTC3′

In this example the sequences of the reverse and forward primers were:

G U6-A
(SEQ ID NO: 10)
CTCTTGAACGCTCTCTCTCCAACTTCCGTTTCTCATCCGGTGTTTCGT
CCTTTCCACA
G term-A
(SEQ ID NO: 11)
ACGCTCTCTCTCCAACTTCCGTTTCTCATCCTTTTTGGAAAAGCTTATC
GATACCGTC

### Long-Range PCR

To produce the linear amplification product, PCR reactions are assembled as follows:

| | |
|---|---|
| 1 μl template | 10 ng pU6.ACTB-A hp |
| 5 μl 10 x buffer | 10 x buffer (Stratagene)^{a} or Pfu |
| | Ultra ™ Buffer (Stratagene) |
| 2 μl 10 mM dNTPs | 10 mM each dNTP |
| 1 μl U6 primer | 10 μM |
| 1 μl term primer | 10 μM |
| 40 μl DDW | |
| 1 μl Pfu Turbo^{b} or Pfu Ultra ™ | Stratgene 2.5 U/μl^{b} |
| 50 μl | |

^{a}10 x cloned Pfu DNA polymerase reaction buffer (Stratagene). 200 mM Tris-HCl (pH 8.8), 20 mM MgSO₄, 100 mM KCl, 100 mM (NH₄)₂SO₄, 1% Triton X-100, 1 mg/ml BSA (nuclease free).
^{b}Pfu is added last, preferably just prior to running reaction to minimise primer degradation.

Reactions are undertaken using a “touchdown” protocol as follows:

Initial denaturation

| | |
|---|---|
| 95° C. | 2 mins |

Touch-down PCR reaction consists of 30 cycles as follows:

| | | |
|---|---|---|
| 95° C. | 30 secs | |
| 60° C./55° C. | 1 min | Decrease by 1° C. for first 5 cycles |
| 74° C. | 5 mins | |
| Final cycle | | |
| 74° C. | 10 mins | |
| Hold reaction at 4° C. | | |

### Optimising Reactions

These reaction conditions are robust. If necessary individual reactions can be optimised by:

Altering touch down and annealing conditions, e.g., use temperature ranges of 65° C./60° C., 60° C./55° C. and 55° C./50° C.

Adding MgCl₂, e.g an extra 0.5 mM MgCl₂ can dramatically effect PCR yields.

### Ligation and Transformation

PCR products are circularised using T4 DNA ligase, using a quick ligation kit according to the manufacturer's (New England Biolabs) instructions.

For Quick Ligation:

| | |
|---|---|
| 10 μl buffer | 2 x Quick Ligation Buffer |
| 10 μl DNA | Approximately 100 ng DNAl |
| 1 μl ligase | Quick T4 DNA ligase |
| 21 μl | |

Incubate for 5 mins at room temperature.

Bacteria are then transformed using standard protocols and transformed cells selected on ampicillin, since the pU6.EGFP-A hp construct encodes ampicillin resistance.

Transformed colonies were analysed using a standard “colony cracking” procedure, in which plasmids in individual colonies were amplified using M13 Forward and Reverse primers. The resultant reactions were analysed using agarose gel electrophoresis. In this instance plasmids containing the correct insert gave a larger product, since the GLUT4 hairpin was longer than the hairpin sequence in the substrate plasmid. In this example 8 colonies were analysed by colony cracking and 6 gave the correct size band. Plasmids from 3 colonies were sequenced and one gave the correct product, which was designated pU6.GA.

Both covalent closed circular or linearised templates can be used to construct hairpin plasmids in this fashion. Background levels are lower when linear templates are used. For U6 constructs the preferred template is Pu6.GA hp cut with Bsm BI, which linearises within the loop region of the construct. Treatment with shrimp alkaline phosphatise (SAP) further reduces background.

### 2.3 Increasing the Length of an Inverted Repeat in a Plasmid

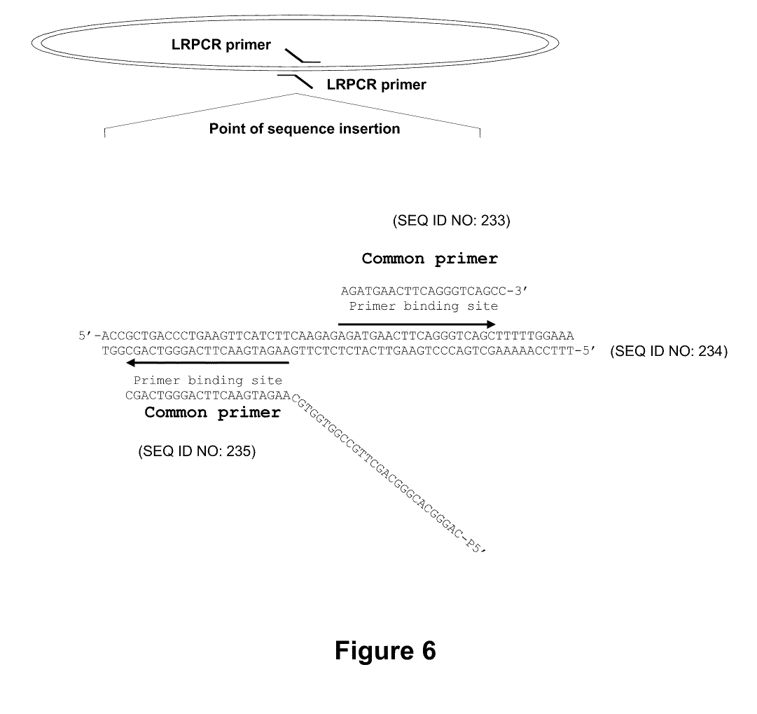

The length of an inverted repeat within a plasmid was increased as shown in FIG. 6 as follows:

The relative positions and sequences of the forward and reverse primers (SEQ ID NOS: 233 and 235) are indicated as in FIG. 4. In this instance the forward and reverse primers are identical. The primer binding site is designed to hybridise to either arm of a hairpin DNA (SEQ ID NO: 234) construct designed to target EGFP, whilst the 5′ effector sequence contains further sequences homologous to EGFP. The length of hairpin DNA constructs can be sequentially increased using this strategy.

2.4 Insertion of an Intron into a Cloned Sequence

A mouse Ige3 intron was inserted into a cloned sequence of the EGFP gene.

The reverse and forward primers were designed to contain a 3′ fixing part homologous to sequential sequences located in the EGFP gene. The 5′ effector sequences of each primer contained approximately half of the sequence of intron 3 from the mouse IgE3 gene.

The forward and reverse primers used in this reaction were:

(SEQ ID NO: 12)
GAGAACATGGTTAACTGGTTAAGTCATGTCGTCCCACAGGAGCGCACCA
TCTTCTTCAAGGA
and
(SEQ ID NO: 13)
TGAACATGAGAAGGGCTGGCCACTCTCCACCTCCTGTACTCACCTGGAC
GTAGCCTTCGGGCATGG

The substrate plasmid was amplified and ligated, and bacterial colonies were obtained and analysed as described above. In this fashion a functional intron (intron 3 from the mouse IgE gene) was inserted into the coding sequences of the EGFP gene in a single step.

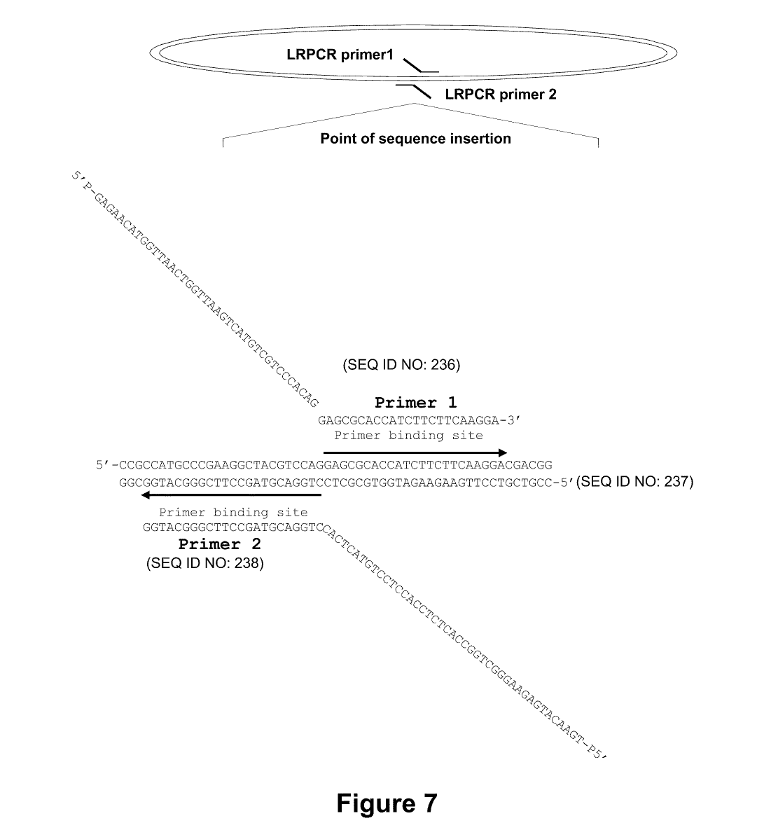

This procedure is shown in FIG. 7 as follows:

The relative positions and sequences of the forward and reverse primers (SEQ ID NOS: 236 and 238) are indicated as in FIG. 4. In this instance the forward and reverse primer binding sites bind to coding sequences of EGFP (SEQ ID NO: 237). The forward and reverse 5′ effector sequences for each primer encode approximately half of intron 3 of the mouse IgE 3 gene.

### 3. Preparation of Hairpin Constructs

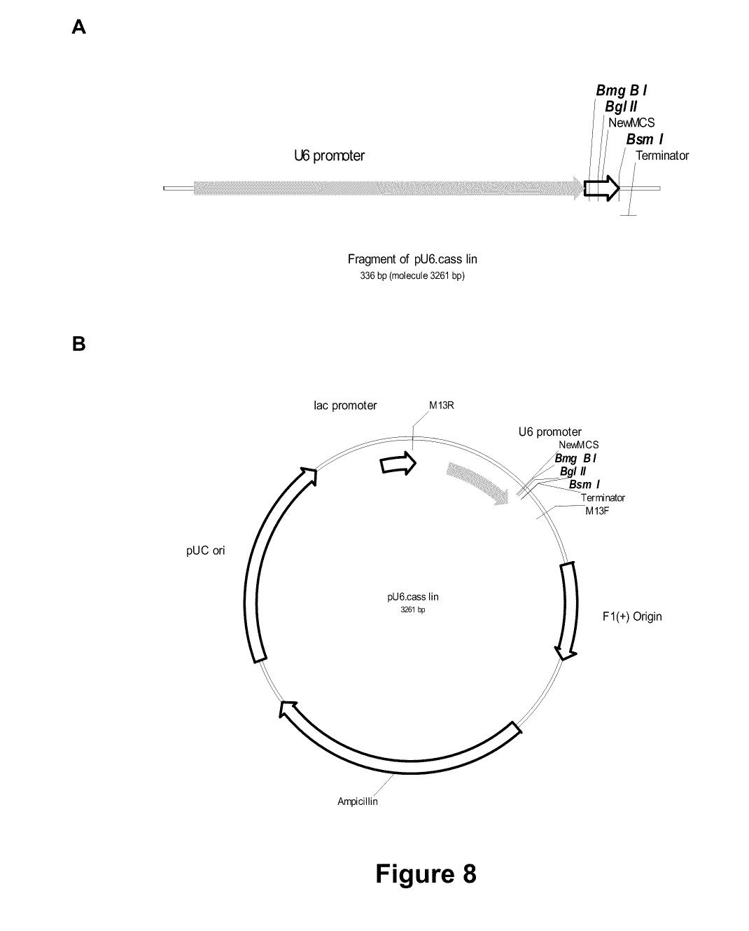

Most constructs described in this application were prepared using the long range PCR strategy described above. The plasmid pU6.cass lin (FIG. 8) may be used as a precursor construct to generate many of the constructs described below. This construct was prepared using a precursor construct pU6.GA which for this purpose is essentially identical to the plasmid pU6.ACTB-A hp (FIG. 2), except its hairpin sequences target another gene. pU6.GA contains identical U6 promoter and pol III terminator sequences to pU6.ACTB-A hp, however the new insert sequences inserted a Bsm BI restriction site which allowed linearistion of the vector prior to long range PCR amplification.

To prepare pU6.cass lin, Bsm BI linearised pU6.GA was amplified using the following primers:

U6mcs

| | |
|---|---|
| TCTTGGACGTGGGTGTTTCGTCCTTTC | (SEQ ID NO: 14) |
| termmcs | |
| TCTTGGAATGCTTTTTTGGAAAAGCTTATCG | (SEQ ID NO: 15) |

A clone of the predicted sequence was isolated, this contains a polylinker containing three unique restriction sites (BmgB I, Bgl II and Bsm I) which can be used to linearise the vector prior to long range PCR amplification to reduce background (FIG. 8A). To generate constructs using this plasmid, the plasmid (FIG. 8B) was linearised with Bgl II prior to amplification.

The constructs used in these experiments are described in Table 1. Conventional single hp DNA constructs were used as controls. Double hairpin constructs were prepared and their activity was compared to the control constructs. The control constructs targeted a single gene, the test constructs (“double hairpin” constructs) targeted two genes, using one sequence at the “base” of the hairpin sequence (furthest from the loop), and a second sequence near the loop of the hairpin structure (the “top” of the hairpin”). This terminology can extend to triple, quadruple, etc hairpins with 3, 4, etc duplex sequences. The activity of constructs where sequences targeting the Renilla luciferase gene were located at the base or the top of a double hairpin RNA, was compared with the activity of a single construct targeting only Renilla luciferase. Using this method, the ability of a construct to target two genes can be reliably inferred. This can optionally be confirmed by determining the activity of a single construct against both target genes.

TABLE 1
Control and “double hairpin” constructs used in these
experiments.

| | | “Bubble” |
|---|---|---|
| Construct designation | Target^{a} | sequence |
| Single hp constructs | | |
| pU6.ACTB-A hp | ACTB-A site (negative control | na |
| | construct) | |
| pU6.Rluc hp | Renilla luciferase (positive control | na |
| | construct) | |
| Double hp constructs | | |
| pU6.Rluc/ACTB TTA | ACTB-A site and Renilla | 5′-UUA-3′ |
| | luciferase | 3′-GUU-5′ |
| pU6.Rluc/ACTB TTAG | ACTB-A site and Renilla | 5′-UUAG-3′ |
| | luciferase | 3′-check-5′ |
| pU6.ACTB/Rluc TTA | ACTB-A site and Renilla | 5′-UUA-3′ |
| | luciferase | 3′-GUU-5′ |
| pU6.ACTB/Rluc TTAG | ACTB-A site and Renilla | 5′-UUAG-3′ |
| | luciferase | 3′-check-5′ |
| pU6.ACTB/AD1 hp | ACTB-A site and ADAR 1 site | |
| | (negative control construct) | |

^{a}mRNA targeted for inactivation
ACTB-A corresponds to positions 1047-1065 of Genbank accession NM_001101.
Rluc site corresponds to positions 1543-1561 0f Genbank accession U47298.
ADAR 1 site corresponds to positions 1477 to 1497 of GenBank sequence NM_001111.

The test constructs were prepared as follows.

pU6.Rluc hp

This construct was designed to target Renilla luciferase mRNA, present in HeLa cells stably transformed with a construct designed to express Renilla luciferase. The construct was prepared using the long range PCR strategy described above using Bgl II linearised pU6.cass lin as a substrate; this was amplified with Pfu Turbo polymerase (Stratagene) using the primers:

U6lucb

(SEQ ID NO: 16)

ACACAAAGTAGGAGTAGTGAAAGGCCGGTGTTTCGTCCTTTC
termlucb

(SEQ ID NO: 17)

AGGTAGGAGTAGTGAAAGGCCTTTTTTGGAAAAGCTTATCG

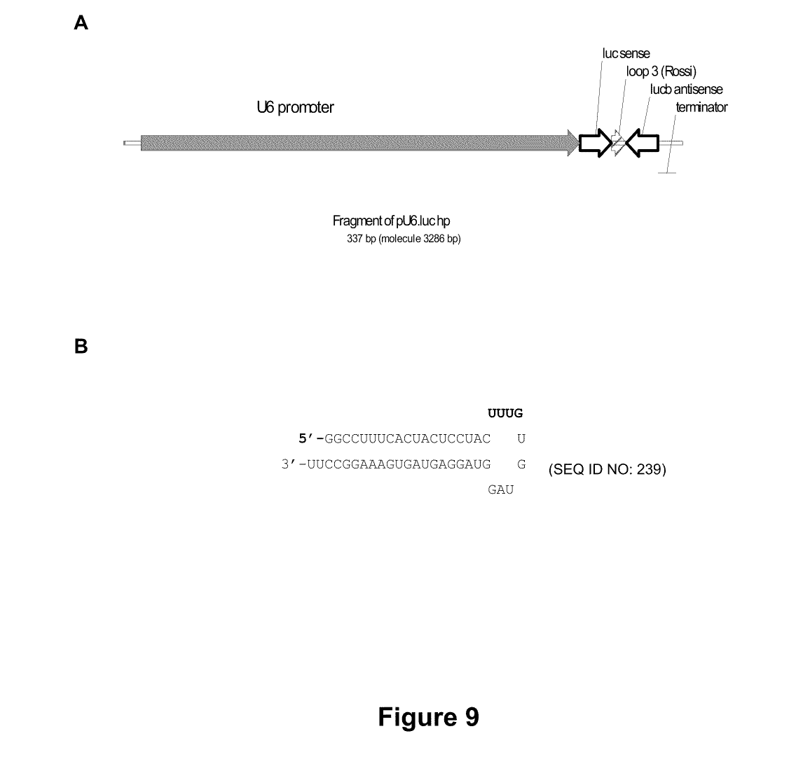

A map of this construct is shown in FIG. 9A and the sequence (SEQ ID NO: 239) and predicted structure of the RNA produced by the construct is shown in FIG. 9B.

pU6.Rluc/ACTB TTA

This construct tested whether a construct carrying a UUA bubble sequence was capable of inactivating two mRNAs, namely a Renilla luciferase transgene and β actin. The construct was prepared using the plasmid pU6.cass lin (FIG. 8) as a substrate by amplifying with the two primers:

U6lucACTB-TTA
(SEQ ID NO: 18)
ACACAAAGCAATGATCTTGATCTTCATAAGTAGGAGTAGTGAAAGGC
CGGTGTTTCGTCCTTTC
termluc-ACTB-TTG
(SEQ ID NO: 19)
AGGCAATGATCTTGATCTTCATTGGTAGGAGTAGTGAAAGGCCTTTT
TTGGAAAAGCTTATCG

A map of this construct is shown in FIG. 10A and the sequence (SEQ ID NO: 240) and predicted structure of the RNA produced by the construct is shown in FIG. 10B.

pU6.Rluc/ACTB TTAG hp

This construct tested whether a construct carrying a UUAG bubble sequence could inactivate two mRNAs, namely a Renilla luciferase transgene and β actin. The construct was prepared by annealing the following nucleotides:

Rluc/ACTB-1

(SEQ ID NO: 20)

ACCGGCCTTTCACTACTCCTACTTAGTGAAGATCAAGATCATTGC
Rluc/ACTB-2

(SEQ ID NO: 21)

TTGATCTTCACTAAGTAGGAGTAGTGAAAGGC
Rluc/ACTB-3

(SEQ ID NO: 22)

TTTGTGTAGGCAATGATCTTGATCTTCAT
Rluc/ACTB-4

(SEQ ID NO: 23)

GATCATTGCCTACACAAAGCAATGATC
Rluc/ACTB-5

(SEQ ID NO: 24)

TGAGTAGGAGTAGTGAAAGGCCTTTTTTGGAAA
Rluc/ACTB-6

(SEQ ID NO: 25)

AGCTTTTCCAAAAAAGGCCTTTCACTACTCCTACTCAATGAAGATCAA

To prepare this construct an oligo assembly strategy was used. Each oligonucleotide was resuspended at 1 ug/ml in water and 1 ul of each was added together, to create a final volume of 100 ul, containing 0.5× strength Buffer M (Roche; 10× Buffer M is 100 mM tris HCl (pH 7.5), 100 mM MgCl₂, 500 mM NaCl, 10 mM DTE). The mixture was heated to 95° C., then oligonucleotides annealed by cooling to 30° C. at 1° C. per minute; these manipulations were performed in a Corbett Palm-Cycler PCR machine (Corbett Research). 20 ul of annealed oligonucleotides were then treated with T4 polynucleotide kinase according to the manufacturer's (Promega) protocol. The annealed oligonucleotides were then purified using a Qiagen PCR purification column, according to the manufacturer's (Qiagen) protocol. 2 ul of eluted oligonucleotides (from 28 ul of eluted material) were then ligated to approximately 100 ng of BsmB I/Hind III Shrimp Alkaline Phosphatase (SAP: Promega) treated pU6.cass prepared, using procedures well known to those familiar with the art, Colonies containing the appropriate sequences were then isolated, and sequence of the construct was confirmed using well known sequencing protocols.

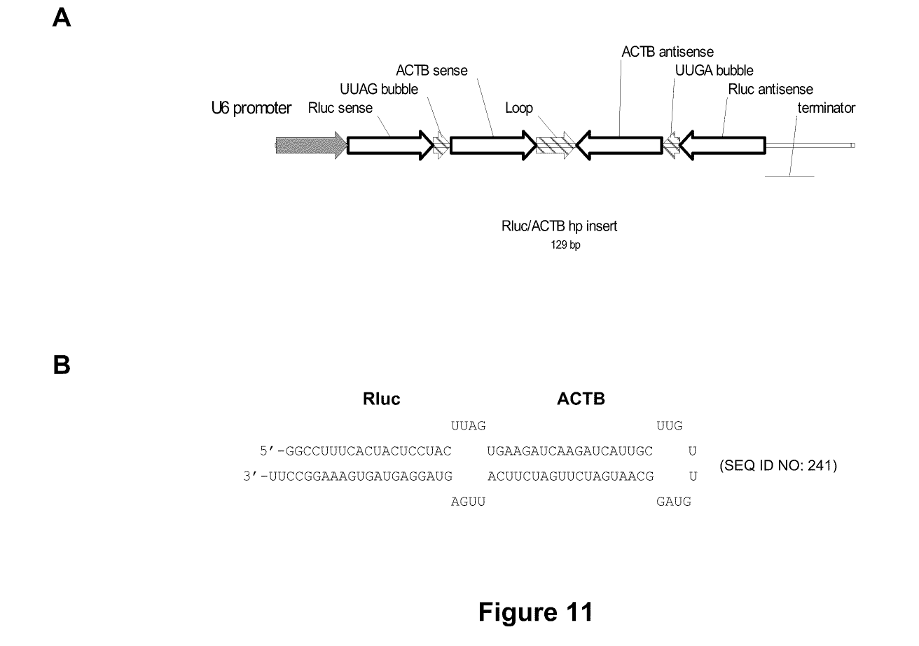

A map of this construct is shown in FIG. 11A and the sequence (SEQ ID NO: 241) and predicted structure of the RNA produced by the construct is shown in FIG. 11B.

pU6.ACTB/Rluc TTA

This construct tests whether a construct carrying a UUA bubble sequence inactivates two mRNAs, namely β actin and a Renilla luciferase transgene. The construct is prepared using the plasmid pU6.cass lin as a substrate by amplifying with the two primers:

U6ACTB-luc-TTA

(SEQ ID NO: 26)

ACACAAAGTAGGAGTAGTGAAAGGCCTAAGCAATGATCTTGATCTTCAC
GGTGTTTCGTCCTTTC
termACTB-luc-TTG

(SEQ ID NO: 27)

AGGTAGGAGTAGTGAAAGGCCTTGGCAATGATCTTGATCTTCATTTTTTG
GAAAAGCTTATCG

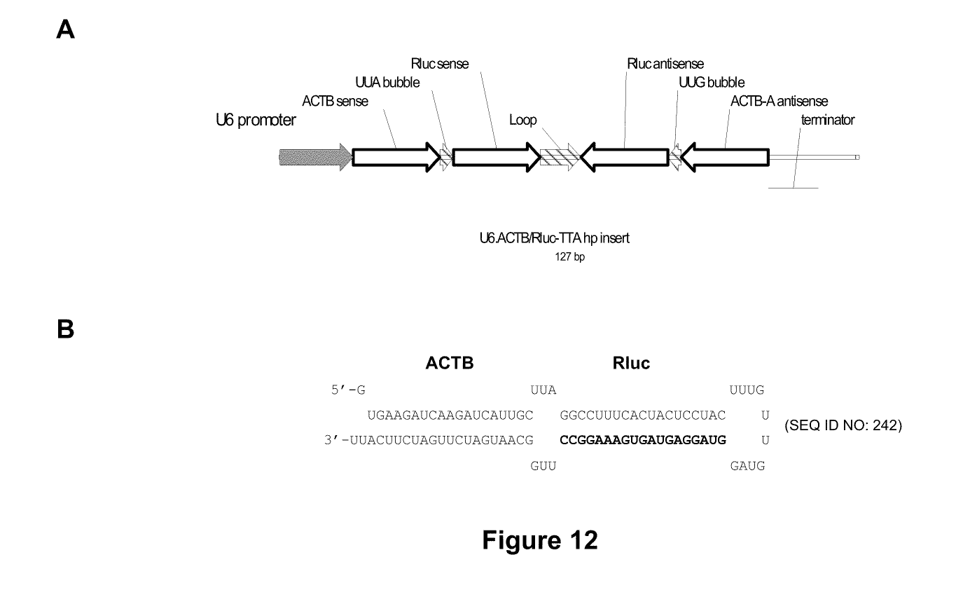

A map of this construct is shown in FIG. 12A and the sequence (SEQ ID NO: 242) and predicted structure of the predicted RNA produced by the construct is shown in FIG. 12B.

pU6.ACTB/Rluc TTAG

This construct tests whether a construct carrying a UUAG bubble sequence is capable of inactivating two mRNAs, namely β actin and a Renilla luciferase transgene. The construct is prepared using the plasmid pU6.cass lin as a substrate by amplifying with the two primers:

U6ACTB-luc-TTAG

(SEQ ID NO: 28)

ACACAAAGTAGGAGTAGTGAAAGGCCCTAAGCAATGATCTTGATCTTCAG
GTGTTTCGTCCTTTC
termACTB-luc-TTGA

(SEQ ID NO: 29)

AGGTAGGAGTAGTGAAAGGCCTTGAGCAATGATCTTGATCTTCATTTTTT
GGAAAAGCTTATCG

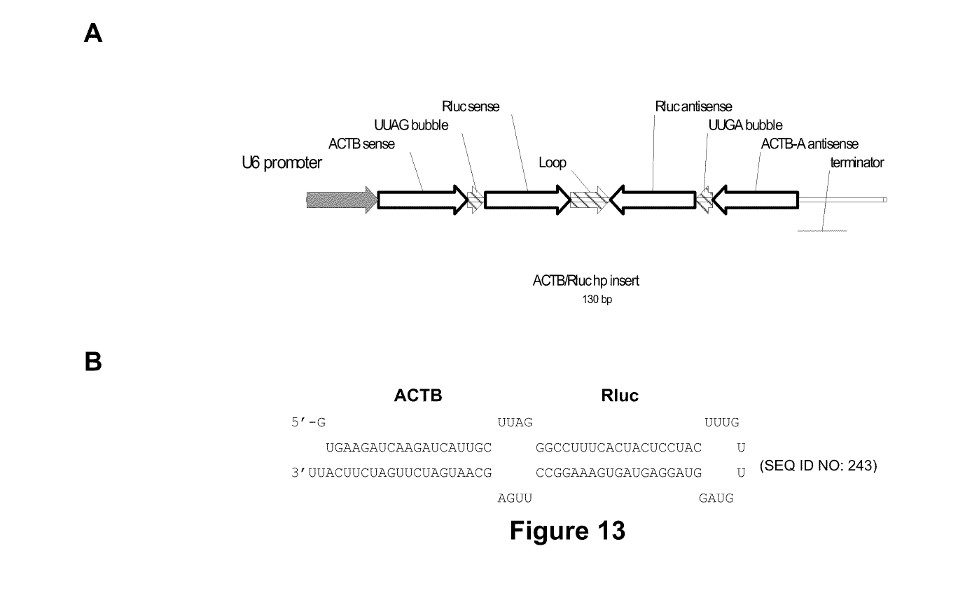

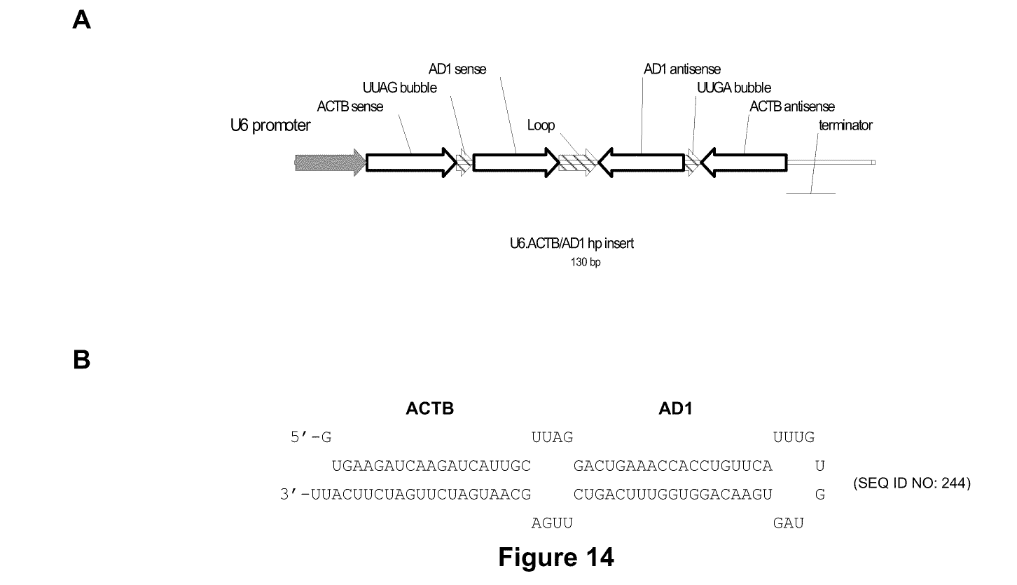

A map of this construct is shown in FIG. 13A and the sequence (SEQ ID NO: 243) and predicted structure of the RNA produced by the construct is shown in FIG. 13B.

### 4. Constructs Targeting Three Genes

Four constructs were prepared targeting Renilla luciferase and a variety of other genes. Three constructs contain Renilla luciferase-targeting sequences at three different positions, respectively, within the hairpin RNA, namely the base, middle and top of the hairpin RNAs and contain the UUAG bubble sequence. The constructs are outlined in Table 2. A fifth construct acted as a negative control.

TABLE 2
Hairpin constructs targeting three genes

| | | Bubble |
|---|---|---|
| Construct | Target^{a} | Sequence |
| pU6.Rluc/ACTB/AD1 hp | Renilla luciferase | 5′-UUAG-3′ |
| | β actin (ACTB-A) | 3′-AGUU-5′ |
| | ADAR1 | |
| pU6.ACTB/Rluc/AD1 hp | β actin (ACTB-A) | 5′-UUAG-3′ |
| | Renilla luciferase | 3′-AGUU-5′ |
| | ADAR1 | |
| pU6.ACTB/AD1/Rluc hp | β actin (ACTB-A) | 5′-UUAG-3′ |
| | ADAR1 | 3′-AGUU-5′ |
| | Renilla luciferase | |
| pU6.ACTB/AD1/GFP hp | Renilla luciferase | 5′-UUAG-3′ |
| | β actin (ACTB-A) | 3′-AGUU-5′ |
| | EGFP | |
| pU6.ACTB/AD1/GFP hp | β actin (ACTB-A) | 5′-UUAG-3′ |
| | ADAR1 | 3′-AGUU-5′ |
| | EGFP | |

^{a}mRNA targeted for inactivation
EGFP target corresponds to positions 924-942 of pEGFPN1-MCS (Invitrogen).

The constructs were prepared mainly using the long range PCR strategy described above.

pU6.Rluc/ACTB/AD1

This construct tested whether a construct carrying sequences targeting a Renilla luciferase transgene in the base of the predicted hairpin RNA inactivated Renilla luciferase. The construct was prepared using linearised plasmid pU6.cass lin as a substrate by amplifying with the two primers:

U6LBA

(SEQ ID NO: 30)

ACAAATGAACAGGTGGTTTCAGTCCTAAGCAATGATCTTGATCTTCACT
AAGTAGGAGTAGTGAAAGGCCGGTGTTTCGTCCTTTC
termLBA

(SEQ ID NO: 31)

GTAGTGAACAGGTGGTTTCAGTCTTGAGCAATGATCTTGATCTTCATTGA
GTAGGAGTAGTGAAAGGCCTTTTTTGGAAAAGCTTATCG

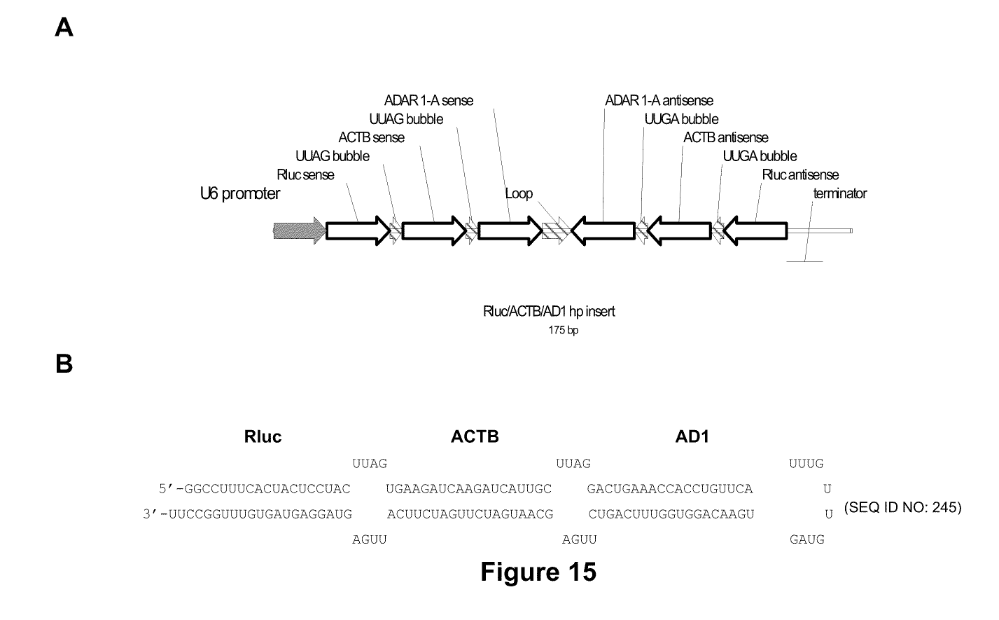

A map of this construct is shown in FIG. 15A and the sequence (SEQ ID NO: 245) and predicted structure of the predicted RNA produced by the construct is shown in FIG. 15B.

pU6.ACTB/Rluc/AD1 hp

This construct tested whether a construct carrying sequences targeting a Renilla luciferase transgene in the middle of the predicted hairpin RNA inactivated Renilla luciferase. The construct was prepared using linearised plasmid pU6.cass lin as a substrate by amplifying with the two primers:

U6BLA

(SEQ ID NO: 32)

CACAAATGAACAGGTGGTTTCAGTCCTAAGTAGGAGTAGTGAAAGGCCC
TAAGCAATGATCTTGATCTTCACCGGTGTTTCGTCCTTTC
termBLA

(SEQ ID NO: 33)

TAGTGAACAGGTGGTTTCAGTCTTGAGTAGGAGTAGTGAAAGGCCTTG
AGCAATGATCTTGATCTTCATTTTTTGGAAAAGCTTATCG

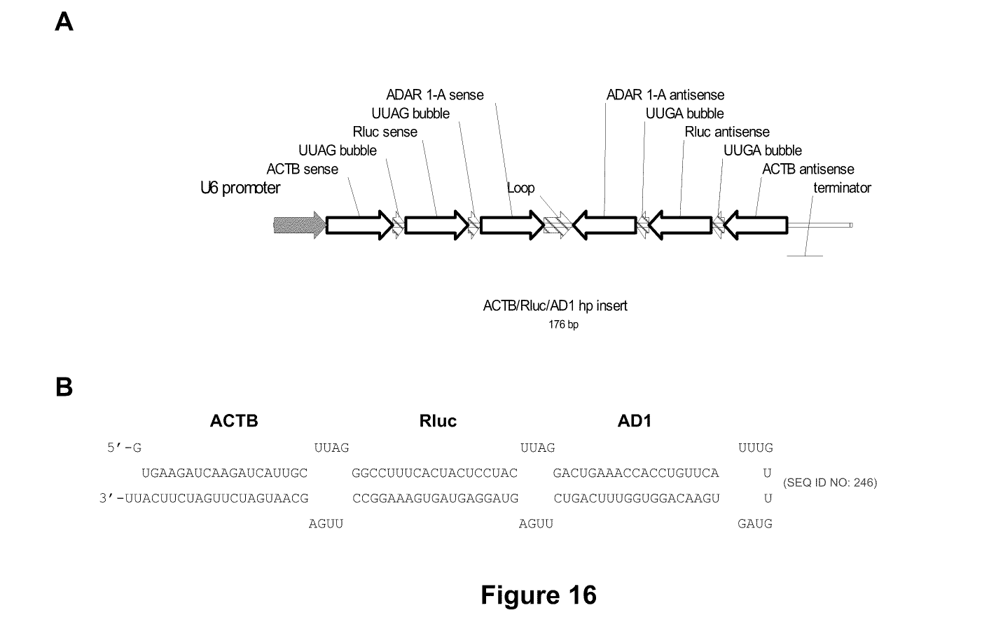

A map of this construct is shown in FIG. 16A and the sequence (SEQ ID NO: 246) and predicted structure of the RNA produced by the construct is shown in FIG. 16B.

pU6.ACTB/AD1/Rluc hp

This construct tested whether a construct carrying sequences targeting a Renilla luciferase transgene at the top of the predicted hairpin RNA inactivated Renilla luciferase. The construct was prepared using the plasmid pU6.cass lin as a substrate by amplifying with the two primers:

U6BAL

(SEQ ID NO: 34)

CACAAAGTAGGAGTAGTGAAAGGCCCTAATGAACAGGTGGTTTCAGTCC
TAAGCAATGATCTTGATCTTCACGGTGTTTCGTCCTTTC
termBAL

(SEQ ID NO: 35)

TAGGTAGGAGTAGTGAAAGGCCTTGATGAACAGGTGGTTTCAGTCTTGA
GCAATGATCTTGATCTTCATTTTTTGGAAAAGCTTATCG

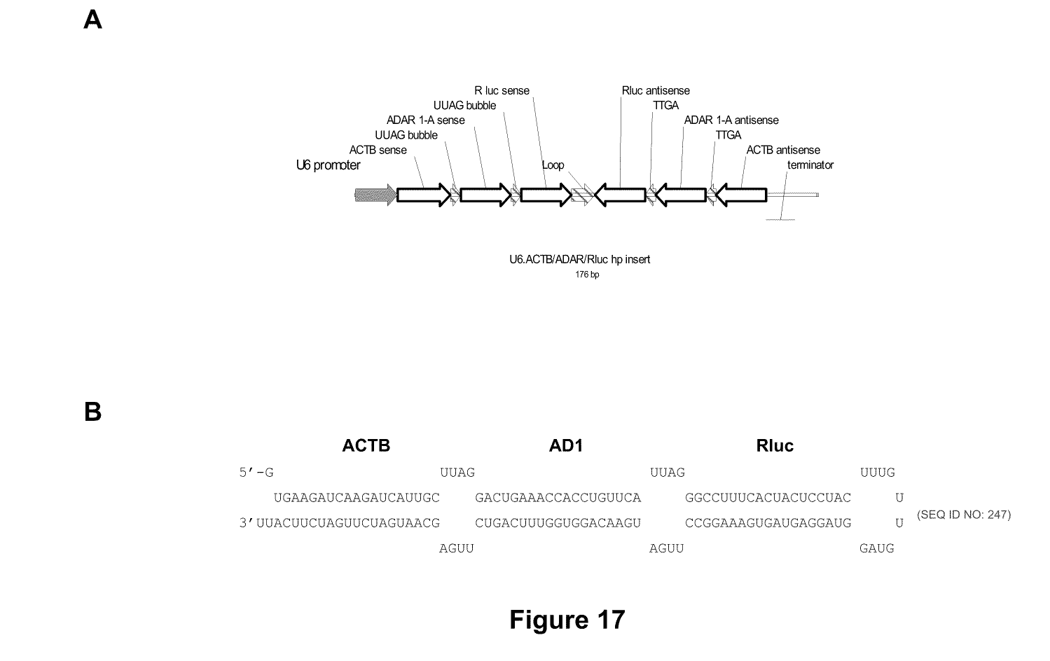

A map of this construct is shown in FIG. 17A and the sequence (SEQ ID NO: 247) and predicted structure of the RNA produced by the construct is shown in FIG. 17B.

pU6.ACTB/AD1/GFP hp

This construct acted as a negative control for the three previous constructs. The construct was prepared using linearised plasmid pU6.cass lin as a substrate by amplifying with the two primers

U6BAG

(SEQ ID NO: 36)

CACAAAGATGAACTTCAGGGTCAGCCTAATGAACAGGTGGTTTCAGTCC
TAAGCAATGATCTTGATCTTCACGGTGTTTCGTCCTTTC
termBAG

(SEQ ID NO: 37)

TAGGATGAACTTCAGGGTCAGCTTGATGAACAGGTGGTTTCAGTCTTG
AGCAATGATCTTGATCTTCATTTTTTGGAAAAGCTTATCG

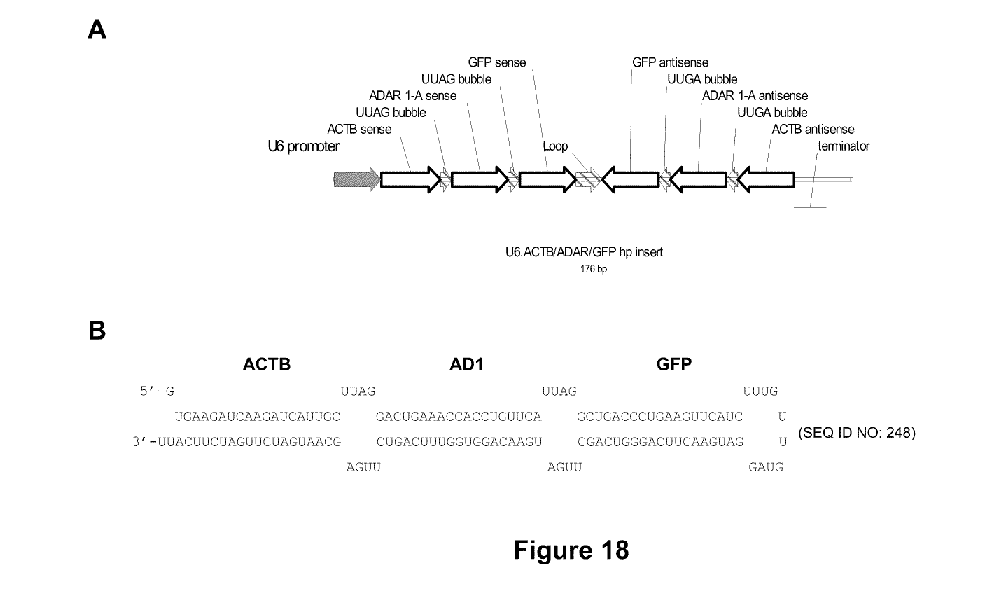

A map of this construct is shown in FIG. 18A and the sequence (SEQ ID NO: 248) and predicted structure of the RNA produced by the construct is shown in FIG. 18B.

### 5. Constructs Targeting Four Genes

To test constructs targeting four separate genes, five constructs may be prepared targeting Renilla luciferase and a variety of other genes. The four constructs each contain a sequence targeting Renilla luciferase at one of four possible positions within the predicted hairpin RNA, namely the base, next to the base, next to the top and top of the hairpin RNAs. The hairpin RNAs further contain the UUAG bubble sequence separating the various components. The constructs are outlined in Table 3.

TABLE 3
Hairpin constructs targeting four genes

| | | Bubble |
|---|---|---|
| Construct | Target^{a} | Sequence |
| pU6.Rluc/ACTB/AD1/ | Renilla luciferase | 5′-UUAG-3′ |
| GFP hp | β actin (ACTB-A) | 3′-AGUU-5′ |
| | ADAR1 | |
| | EGFP | |
| pU6.ACTB/Rluc/AD1/ | β actin (ACTB-A) | 5′-UUAG-3′ |
| GFP hp | Renilla luciferase | 3′-AGUU-5′ |
| | ADAR1 | |
| | EGFP | |
| pU6.ACTB/AD1/Rluc/ | β actin (ACTB-A) | 5′-UUAG-3′ |
| GFP hp | ADAR1 | 3′-AGUU-5′ |
| | Renilla luciferase | |
| | EGFP | |
| pU6.ACTB/AD1/GFP/ | β actin (ACTB-A) | 5′-UUAG-3′ |
| Rluc hp | ADAR1 | 3′-AGUU-5′ |
| | EGFP | |
| | Renilla luciferase | |
| pU6.ACTB/AD1/GFP/ | β actin (ACTB-A) | 5′-UUAG-3′ |
| HER2 hp | ADAR1 | 3′-AGUU-5′ |
| | EGFP | |
| | HER-2 | |

^{a}mRNA targeted for inactivation
HER2 target corresponds to positions 223-241 of Genbank accession HUMHER2A.

The constructs may be prepared using the long range PCR strategy described above.

pU6.Rluc/ACTB/AD1/GFP hp

This construct tests whether a construct carrying sequences targeting a Renilla luciferase transgene in the base of the predicted hairpin RNA inactivates Renilla luciferase. The construct is prepared using the plasmid pU6.Rluc/ACTB/AD1 hp as a substrate by amplifying with the two primers:

U6AGG4

(SEQ ID NO: 38)

GTTCATCAAGCTGACCCTGAAGTTCATCCTACACAAAGATGAACTTCAGG
GTCAGCCTAATGAACAGGTGGTTTCAGTCCTAA
ABL4

(SEQ ID NO: 39)

AGGTGGTTTCAGTCTTGAGCAATGATCTTGATCTTCATTGAGTAGGAGT
AGTGAAAGGCCTTTTTTGGAAAAGCTTATCG

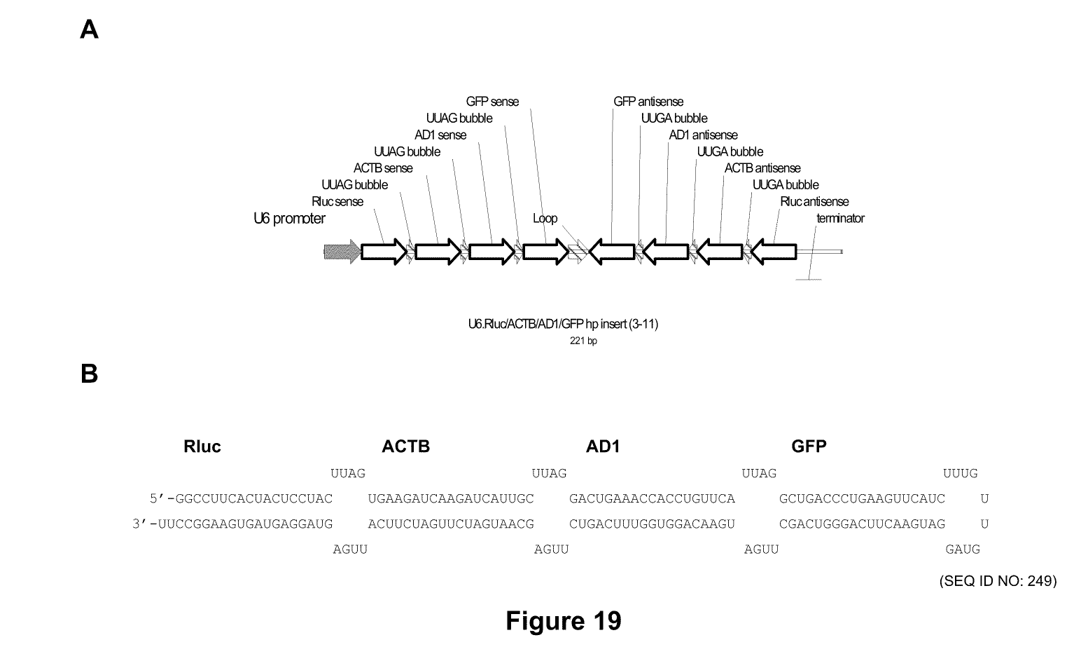

A map of the construct is shown in FIG. 19A and the sequence (SEQ ID NO: 249) and predicted structure of the RNA produced by the construct is shown in FIG. 19B.

pU6.ACTB/Rluc/AD1/GFP hp

This construct tests whether a construct carrying sequences targeting a Renilla luciferase transgene in the second position from the base of the predicted hairpin RNA inactivates Renilla luciferase. The construct is prepared using the plasmid pU6.ACTB/Rluc/AD1 hp as a substrate by amplifying with the two primers:

U6AGG4

(SEQ ID NO: 40)

GTTCATCAAGCTGACCCTGAAGTTCATCCTACACAAAGATGAACTTCAGG
GTCAGCCTAATGAACAGGTGGTTTCAGTCCTAA
termALB4

(SEQ ID NO: 41)

AGGTGGTTTCAGTCTTGAGTAGGAGTAGTGAAAGGCCTTGAGCAATGATC
TTGATCTTCATTTTTTGGAAAAGCTTATCG

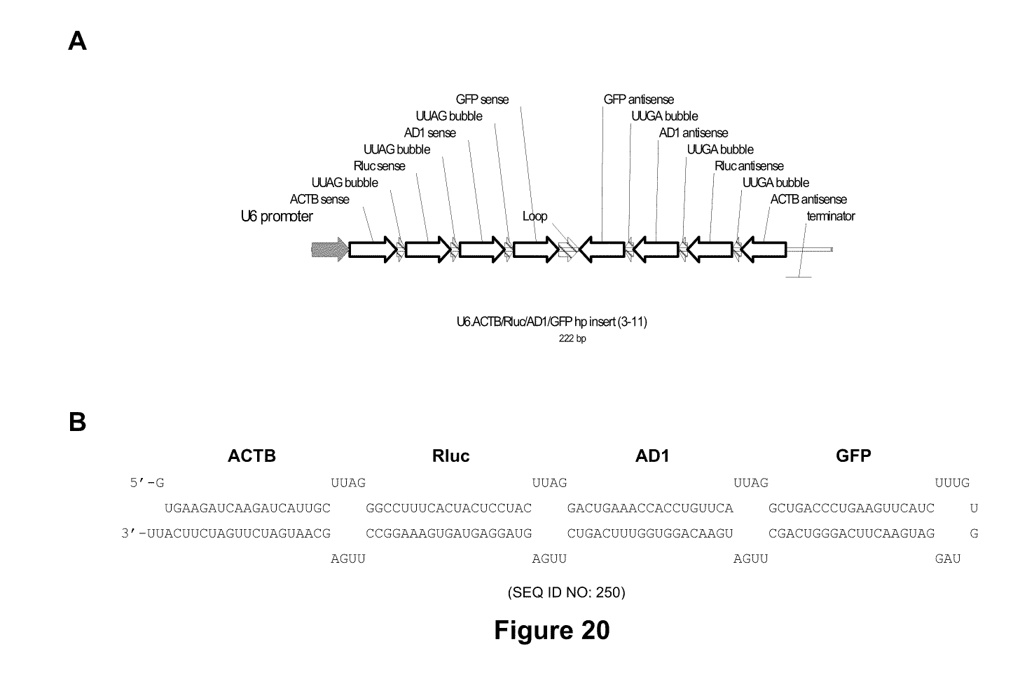

A map of the construct is shown in FIG. 20A and the sequence (SEQ ID NO: 250) and potential structure of the RNA produced by the construct is shown in FIG. 20B.
pU6.ACTB/AD1/Rluc/GFP hp

This construct tests whether a construct carrying sequences targeting a Renilla luciferase transgene in the third position from the base of the predicted hairpin RNA inactivates Renilla luciferase. The construct is prepared using the plasmid pU6.ACTB/AD1/Rluc hp as a substrate by amplifying with the two primers:

U6LGG4

(SEQ ID NO: 42)

CTACTCAAGCTGACCCTGAAGTTCATCCTACACAAAGATGAACTTCAGGG
TCAGCCTAAGTAGGAGTAGTGAAAGGCCCTAA
termLAB4

(SEQ ID NO: 43)

GAGTAGTGAAAGGCCTTGATGAACAGGTGGTTTCAGTCTTGAGCAATGA
TCTTGATCTTCATTTTTTGGAAAAGCTTATCG

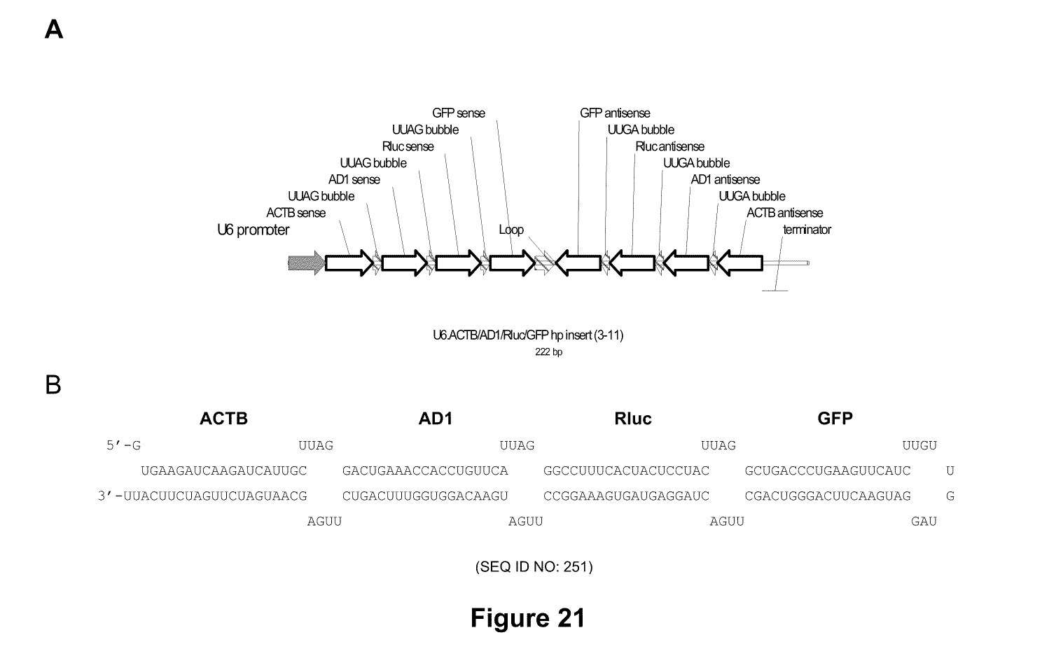

A map of the construct is shown in FIG. 21A and the sequence (SEQ ID NO: 251) and potential structure of the predicted RNA produced by the construct is shown in FIG. 21B.

pU6.ACTB/AD1/GFP/Rluc hp

This construct tested whether a construct carrying sequences targeting a Renilla luciferase transgene adjacent to the loop of the predicted hairpin RNA inactivated Renilla luciferase. The construct was prepared by annealing the following oligonucleotides:

BAGR1

(SEQ ID NO: 44)

ACCGTGAAGATCAAGATCATTGCTTAGGACTGAAACCA
BAGR2

(SEQ ID NO: 45)

ATGAACAGGTGGTTTCAGTCCTAAGCAATGATCTTGATCTTCA
BAGR3

(SEQ ID NO: 46)

CCTGTTCATTAGGCTGACCCTGAAGTTCATCTTAG
BAGR4

(SEQ ID NO: 47)

TGAAAGGCCCTAAGATGAACTTCAGGGTCAGCCTA
BAGR5

(SEQ ID NO: 48)

GGCCTTTCACTACTCCTACTTTGTGTAGGTAGGAGTAGTGAAAGGCC
BAGR6

(SEQ ID NO: 49)

TCATCTCAAGGCCTTTCACTACTCCTACCTACACAAAGTAGGAGTAG
BAGR7

(SEQ ID NO: 50)

TTGAGATGAACTTCAGGGTCAGCTTGATGAACAGGTGGTTTCAGTC
BAGR8

(SEQ ID NO: 51)

CACCTGTTCATCAAGCTGACCCTGAAGT
BAGR9

(SEQ ID NO: 52)

TTGAGCAATGATCTTGATCTTCATTTTTTGGAAA
BAGR10

(SEQ ID NO: 53)

AGCTTTTCCAAAAAATGAAGATCAAGATCATTGCTCAAGACTGAAAC

The oligonucleotides were annealed together, treated with T4 PNK according to the manufacturer's (Promega) protocol and cloning the resultant mixture into BsmB I/Hind III cleaved pU6.cass that had been treated with SAP as described above.

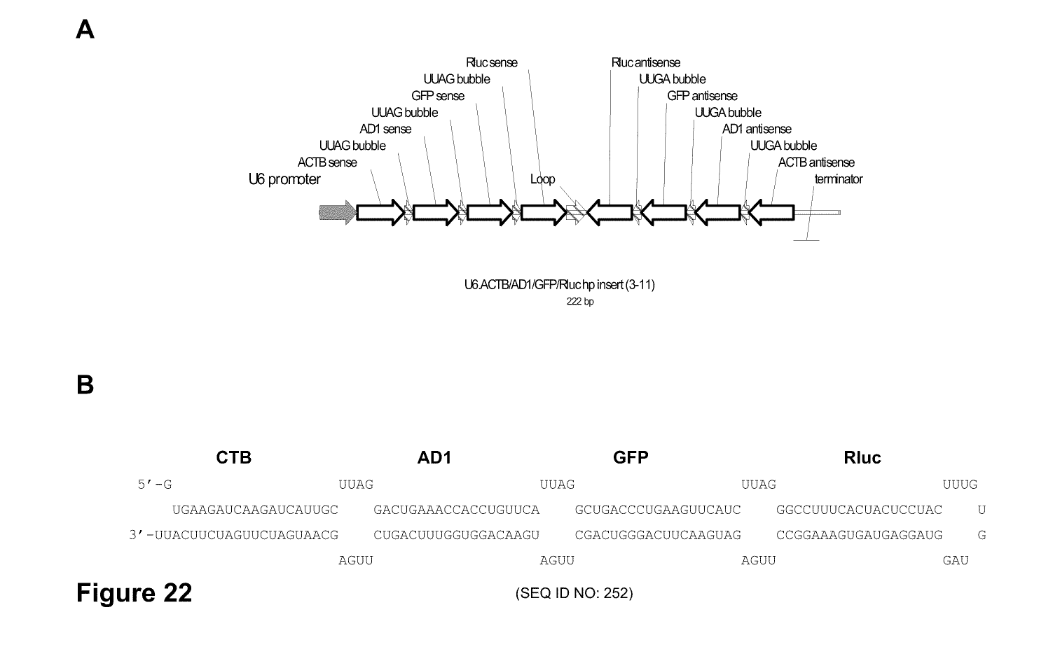

A map of the construct is shown in FIG. 22A and the sequence (SEQ ID NO: 252) and predicted structure of the RNA produced by the construct is shown in FIG. 22B.

pU6.ACTB/AD1/GFP/HER2 hp

This construct acts as a negative control for the four previous constructs. The construct is prepared using the plasmid pU6.ACTB/AD1/GFP hp as a substrate by amplifying with the two primers:

U6GHH4

(SEQ ID NO: 54)

CATCTCAAGTGTGCACCGGCACAGACACTACACAAATGTCTGTGCCGG
TGCACACCTAAGATGAACTTCAGGGTCAGCCTAA
termGAB4

(SEQ ID NO: 55)

AACTTCAGGGTCAGCTTGATGAACAGGTGGTTTCAGTCTTGAGCAATGA
TCTTGATCTTCATTTTTTGGAAAAGCTTATCG

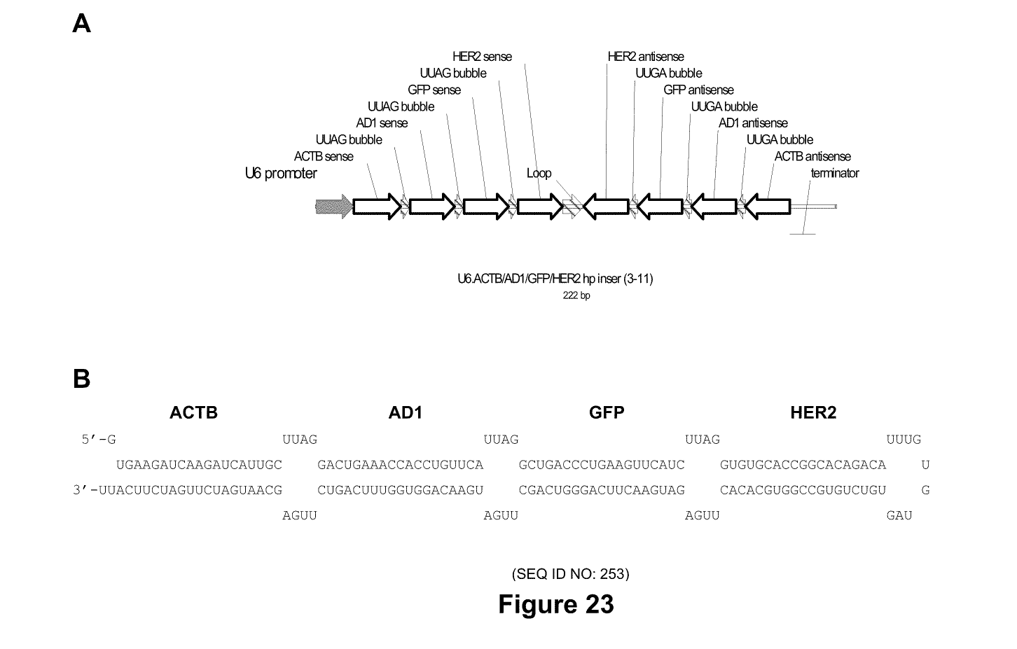

A map of the construct is shown in FIG. 23A and the sequence (SEQ ID NO: 253) and predicted structure of the RNA produced by the construct is shown in FIG. 23B.

### 6. Constructs Targeting Five Genes

To test constructs targeting five separate genes, six constructs may be prepared targeting Renilla luciferase and a variety of other genes. Each of the five constructs contains a sequence targeting Renilla luciferase at one of five possible positions within the predicted hairpin RNA, namely the base, all positions from next to the base to next to the top and the top of the hairpin RNAs. The hairpin RNAs further contain the UUAG bubble sequence separating the various components. The constructs are outlined in Table 4.

TABLE 4
Hairpin constructs targeting five genes

| | | Bubble |
|---|---|---|
| Construct | Target^{a} | Sequence |
| pU6.Rluc/ACTB/AD1/ | Renilla luciferase | 5′-UUAG-3′ |
| GFP/HER2 hp | β actin (ACTB-A) | 3′-AGUU-5′ |
| | ADAR1 | |
| | EGFP | |
| | HER2 | |
| pU6.ACTB/Rluc/AD1/ | β actin (ACTB-A) | 5′-UUAG-3′ |
| GFP/HER2 hp | Renilla luciferase | 3′-AGUU-5′ |
| | ADAR1 | |
| | EGFP | |
| | HER2 | |
| pU6.ACTB/AD1/Rluc/ | β actin (ACTB-A) | 5′-UUAG-3′ |
| GFP/HER2 hp | ADAR1 | 3′-AGUU-5′ |
| | Renilla luciferase | |
| | EGFP | |
| | HER2 | |
| pU6.ACTB/AD1/GFP/ | β actin (ACTB-A) | 5′-UUAG-3′ |
| Rluc/HER2 hp | ADAR1 | 3′-AGUU-5′ |
| | EGFP | |
| | Renilla luciferase | |
| | HER2 | |
| pU6.ACTB/AD1/GFP/ | β actin (ACTB-A) | 5′-UUAG-3′ |
| HER2/Rluc hp | ADAR1 | 3′-AGUU-5′ |
| | EGFP | |
| | HER2 | |
| | Renilla luciferase | |
| pU6.ACTB/AD1/GFP/ | β actin (ACTB-A) | 5′-UUAG-3′ |
| HER2/LAM hp | ADAR1 | 3′-AGUU-5′ |
| | EGFP | |
| | HER2 | |
| | Lamin A/C | |

^{a}mRNA targeted for inactivation
LAM target corresponds to positions 820-838 of Genbank accession NM_005572.

The constructs may be prepared using the long range PCR strategy described above.

pU6.Rluc/ACTB/AD1/GFP/HER2 hp

This construct tests whether a construct carrying sequences targeting a Renilla luciferase transgene in the base of the predicted hairpin RNA inactivates Renilla luciferase. The construct is prepared using the plasmid pU6.Rluc/ACTB/AD1/GFP hp as a substrate by amplifying with the two primers:

U6GHH5

(SEQ ID NO: 56)

TCAAGTGTGCACCGGCACAGACACTACACAAATGTCTGTGCCGGTGCA
CACCTAAGATGAACTTCAGGGTCAGCCTAA
termGABL5

(SEQ ID NO: 57)

GATGAACTTCAGGGTCAGCTTGATGAACAGGTGGTTTCAGTCTTGAGCA
ATGATCTTGATCTTCATTGAGTAGGAGTAGTGAAAGGCCTTTTTTGGAAA
AGCTTATCG

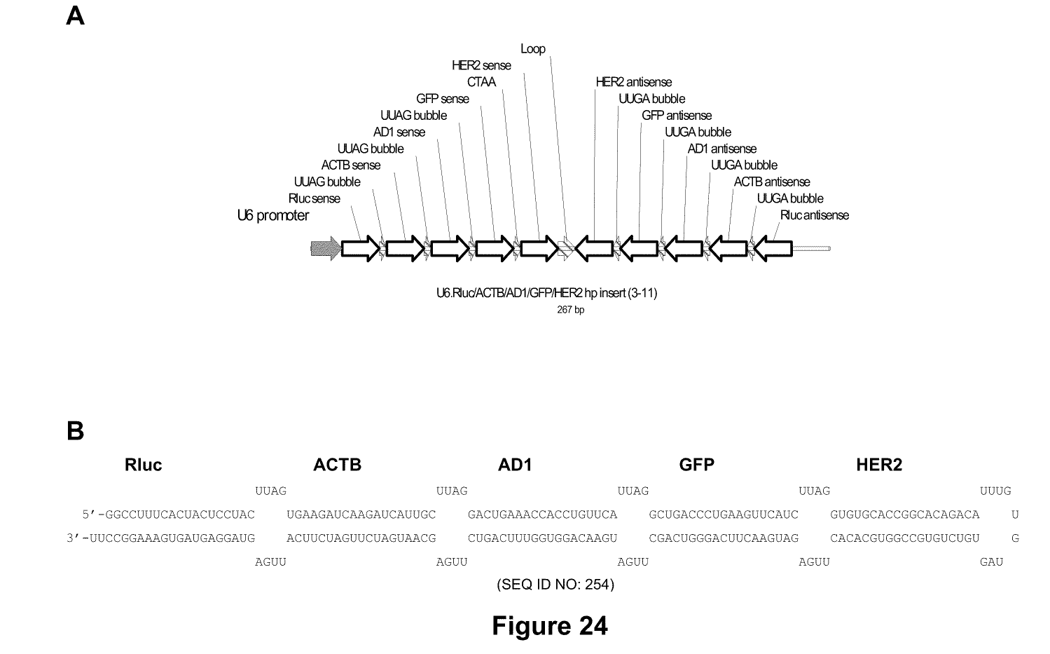

A map of the construct is shown in FIG. 24A and the sequence (SEQ ID NO: 254) and predicted structure of the RNA produced by the construct is shown in FIG. 24B.

pU6.ACTB/Rluc/AD1/GFP/HER2 hp

This construct tests whether a construct carrying sequences targeting a Renilla luciferase transgene in position two, one position up the base of the predicted hairpin RNA inactivates Renilla luciferase. The construct is prepared using the plasmid pU6.ACTB/Rluc/AD1/GFP hp as a substrate by amplifying with the two primers:

U6GHH5

(SEQ ID NO: 58)

TCAAGTGTGCACCGGCACAGACACTACACAAATGTCTGTGCCGGTGCA
CACCTAAGATGAACTTCAGGGTCAGCCTAA
termGALB5

(SEQ ID NO: 59)

GATGAACTTCAGGGTCAGCTTGATGAACAGGTGGTTTCAGTCTTGAGT
AAGGGTAGTGAAAGGCCTTGAGCAATGATCTTGATCTTCATTTTTTGGA
AAAGCTTATCG

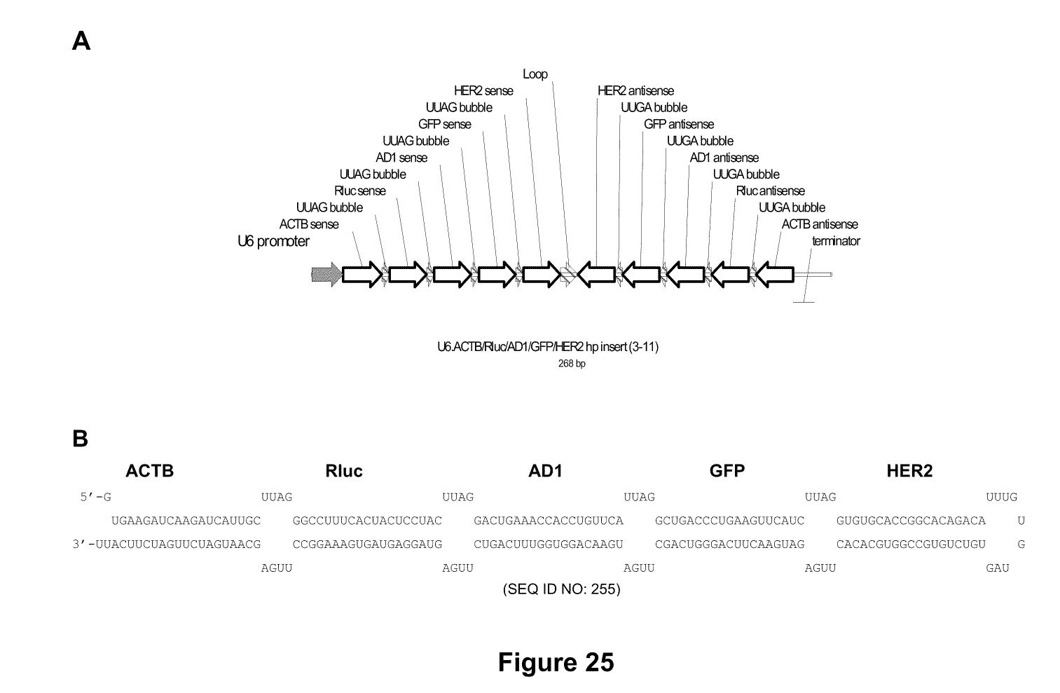

A map of the construct is shown in FIG. 25A and the sequence (SEQ ID NO: 255) and predicted structure of the RNA produced by the construct is shown in FIG. 25B.

pU6.ACTB/AD1/Rluc/GFP/HER2 hp

This construct tests whether a construct carrying sequences targeting a Renilla luciferase transgene in position three, in the middle of the predicted hairpin RNA, inactivates Renilla luciferase. The construct is prepared using the plasmid pU6.ACTB/AD1/Rluc/GFP hp as a substrate by amplifying with the two primers:

U6GHH5

(SEQ ID NO: 60)

TCAAGTGTGCACCGGCACAGACACTACACAAATGTCTGTGCCGGTGCA
CACCTAAGATGAACTTCAGGGTCAGCCTAA
termGLAB5

(SEQ ID NO: 61)

GATGAACTTCAGGGTCAGCTTGAGTAGGAGTAGTGAAAGGCCTTGATG
AACAGGTGGTTTCAGTCTTGAGCAATGATCTTGATCTTCATTTTTTGGA
AAAGCTTATCG

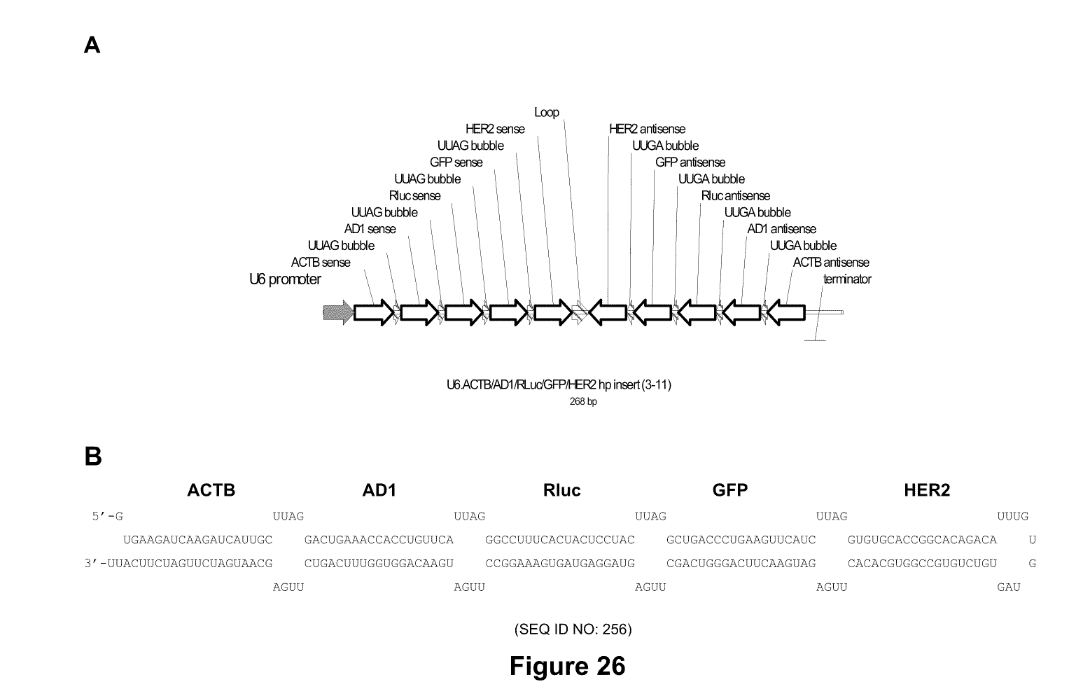

A map of the construct is shown in FIG. 26A and the sequence (SEQ ID NO: 256) and predicted structure of the predicted RNA produced by the construct is shown in FIG. 26B.

pU6.ACTB/AD1/GFP/Rluc/HER2 hp

This construct tests whether a construct carrying sequences targeting a Renilla luciferase transgene in position four, one back from the loop sequence of the predicted hairpin RNA, inactivates Renilla luciferase. The construct is prepared using the plasmid pU6.ACTB/AD1/GFP/Rluc hp as a substrate by amplifying with the two primers:

U6LHH5

(SEQ ID NO: 62)

TCAAGTGTGCACCGGCACAGACACTACACAAATGTCTGTGCCGGT
GCACACCTAAGTAGGAGTAGTGAAAGGCCCTAA
termLGAB5

(SEQ ID NO: 63)

GTAGGAGTAGTGAAAGGCCTTGAGATGAACTTCAGGGTCAGCTTGA
TGAACAGGTGGTTTCAGTCTTGAGCAATGATCTTGATCTTCATTTTTT
GGAAAAGCTTATCG

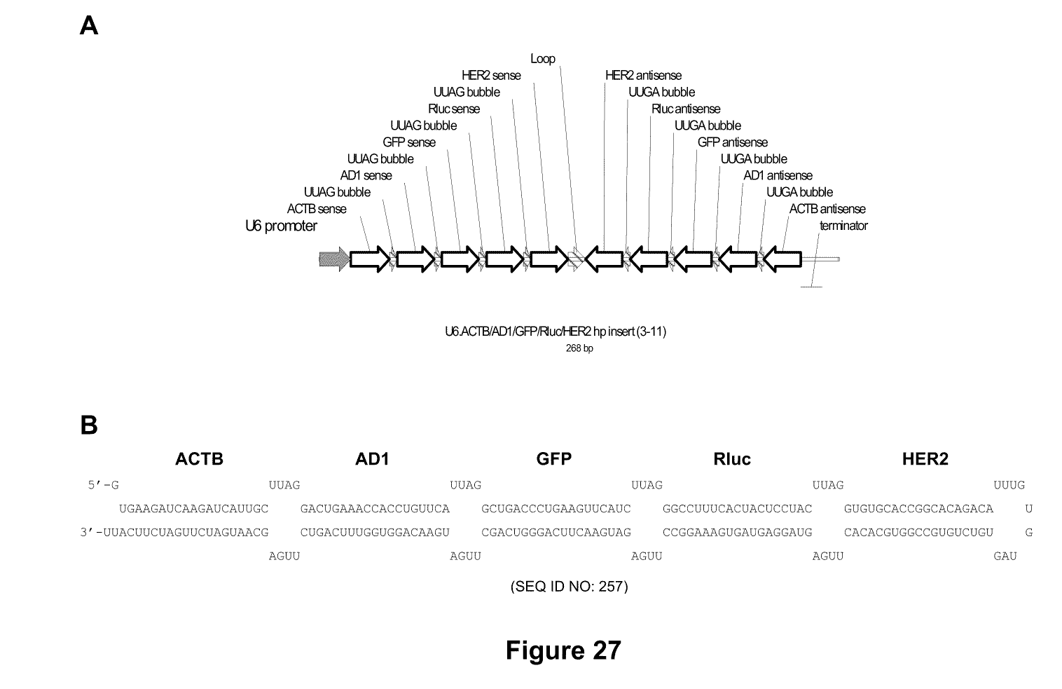

A map of the construct is shown in FIG. 27A and the sequence (SEQ ID NO: 257) and predicted structure of the predicted RNA produced by the construct is shown in FIG. 27B.

pU6.ACTB/AD1/GFP/HER2/Rluc hp

This construct tested whether a construct carrying sequences targeting a Renilla luciferase transgene in position five, adjacent to the loop sequence of the predicted hairpin RNA, inactivated Renilla luciferase. The construct was prepared by annealing the following oligonucleotides:

BAGR1

(SEQ ID NO: 64)

ACCGTGAAGATCAAGATCATTGCTTAGGACTGAAACCA
BAGR2

(SEQ ID NO: 65)

ATGAACAGGTGGTTTCAGTCCTAAGCAATGATCTTGATCTTCA
BAGR3

(SEQ ID NO: 66)

CCTGTTCATTAGGCTGACCCTGAAGTTCATCTTAG
BAGHR4

(SEQ ID NO: 67)

GGTGCACACCTAAGATGAACTTCAGGGTCAGCCTA
BAGHR5

(SEQ ID NO: 68)

GTGTGCACCGGCACAGACATTAGGGCCTTTCACTACTCCTACTTTGT
BAGHR6

(SEQ ID NO: 69)

CCTACCTACACAAAGTAGGAGTAGTGAAAGGCCCTAATGTCTGTGCC
BAGHR7

(SEQ ID NO: 70)

GTAGGTAGGAGTAGTGAAAGGCCTTGATGTCTGTGCCGGTGCACAC
BAGHR8

(SEQ ID NO: 71)

TCATCTCAAGTGTGCACCGGCACAGACATCAAGGCCTTTCACTACT
BAGR7

(SEQ ID NO: 72)

TTGAGATGAACTTCAGGGTCAGCTTGATGAACAGGTGGTTTCAGTC
BAGHR10

(SEQ ID NO: 73)

CACCTGTTCATCAAGCTGACCCTGAAGT
BAGR9

(SEQ ID NO: 74)

TTGAGCAATGATCTTGATCTTCATTTTTTGGAAA
BAGR10

(SEQ ID NO: 75)

AGCTTTTCCAAAAAATGAAGATCAAGATCATTGCTCAAGACTGAAAC

The oligonucleotides were annealed together, treated with T4 PNK according to the manufacturer's (Promega) protocol and cloning the resultant mixture into BsmB I/Hind III cleaved pU6.cass that had been treated with SAP as described above.

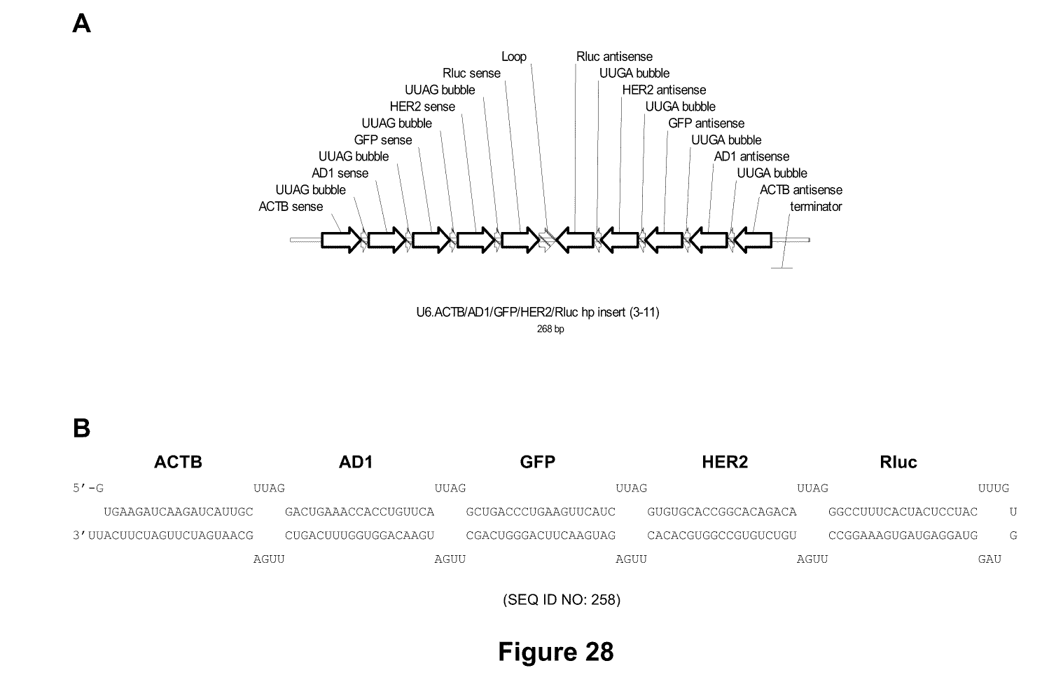

A map of the construct is shown in FIG. 28A and the sequence (SEQ ID NO: 258) and predicted structure of the RNA produced by the construct is shown in FIG. 28B.

pU6.ACTB/AD1/GFP/HER2/Lam hp

This construct acts as a control for the five previous constructs. The construct is prepared using the plasmid pU6.ACTB/AD1/GFP/HER2 hp as a substrate by amplifying with the two primers:

U6HMM5

(SEQ ID NO: 76)

TCAACTGGACTTCCAGAAGAACACTACACAAATGTTCTTCTGGAAGT
CCAGCTAATGTCTGTGCCGGTGCACACCTAA
termHGAB5

(SEQ ID NO: 77)

TGTCTGTGCCGGTGCACACTTGAGATGAACTTCAGGGTCAGCTTGATG
AACAGGTGGTTTCAGTCTTGAGCAATGATCTTGATCTTCATTTTTTGGAA
AAGCTTATCG

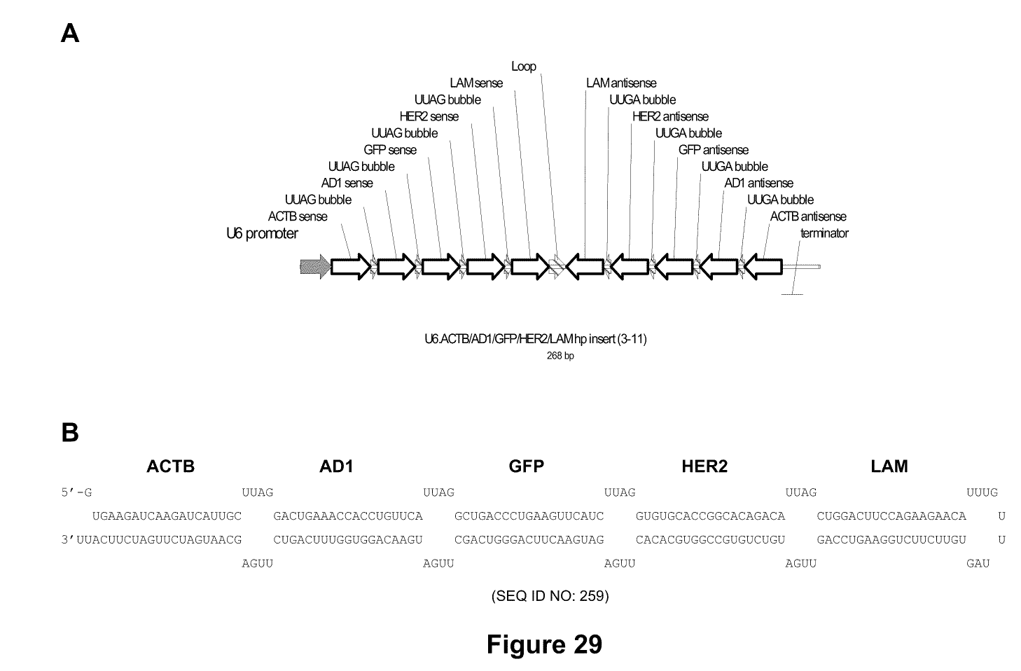

A map of the construct is shown in FIG. 29A and the sequence (SEQ ID NO: 259) of the predicted RNA produced by the construct is shown in FIG. 29B.

### 7. Testing the Activity of Constructs

To test the activity of constructs targeting Renilla luciferase, plasmids were prepared using Qiagen columns according to the manufacturer's protocol. Plasmid DNAs were then transfected into HeLa cells that had been previously stably transformed with the construct. pHRLSV40 (Promega). This was done by co-transfection of pHRLSV40 with a selectable marker plasmid encoding hygromycin resistance; techniques to obtain such stably transformed cells are well known to those familiar with the art.

3,000 cells (as determined by haemocytometer count) were plated into each well of a 96 well tissue culture plates (Costar) and incubated overnight in 100 ul of DMEM media (Gibco) supplemented with heat inactivated 10% FBS (Gibco). To transfect cells each well was treated as follows:

0.2 ul or 0.3 ul LT1 transfection reagent (Mirus Corp.) was added to 25 ul serum free media (DMEM) and incubated for 10 mins at room temperature.

100 ng of DNA was added to this mixture and complex formation allowed to proceed for a further 10 mins at room temperature. The entire mixture was then added to a well of transgenic HeLa cells.

Cells were incubated at 37° C. overnight then media removed and 100 ul fresh DMEM 10% FBS added and incubation continued.

To determine Renilla luciferase activity, media was removed and fresh media containing EnduRen was added according to the manufacturer's (Promega) protocol; cells were then incubated for 5 hrs. Renilla luciferase activity was determined using a Veritas Microplate Luminometer according to the manufacturer's (Turner Biosystems) protocols. These values were then corrected for relative cell numbers which were determined using CellTiter-Glo reagent according to the manufacturer's (Promega) protocols using a Veritas Microplate Luminometer according to the manufacturer's (Turner Biosystems) protocols.

In this fashion the relative activity of individual constructs could be easily and accurately determined, moreover these activities were then corrected using appropriate negative controls, typically pU6.cass, to determine the relative activities of constructs.

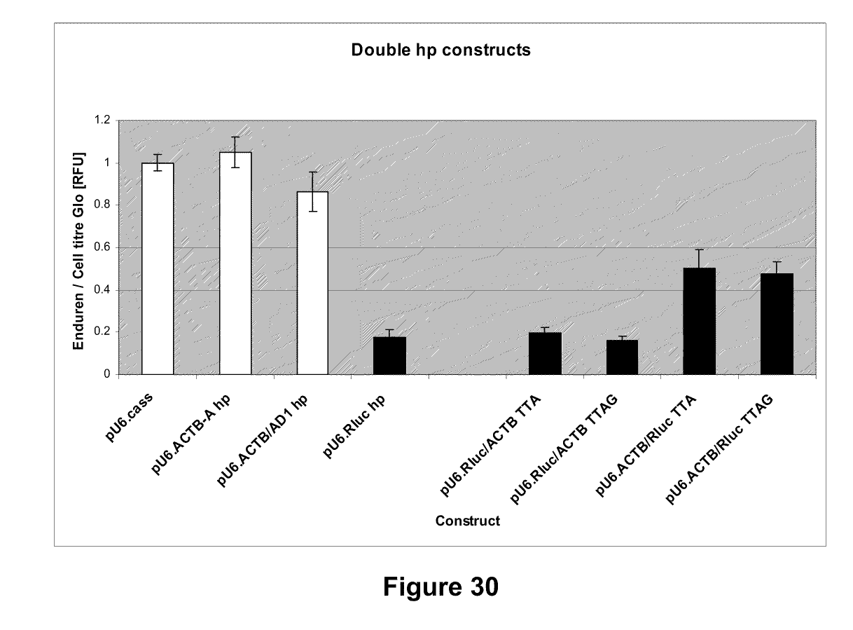

FIG. 30 shows the activity of double hairpin constructs targeting Renilla luciferase. These data demonstrate that sequences at the base or top of a double hairpin construct both significantly reduce Renilla luciferase activity, constructs were most active when the Rluc sequences were present in the base of the predicted transcript, but significant activity was retained in constructs where Rluc targeting sequences were present in the top of the predicted transcript, adjacent to the loop. This indicates that such a double hairpin construct can produce two active siRNAs and it follows necessarily that constructs may be designed to either inactivate two genes, or inactivate a single gene more effectively via the additive effect of producing two separate siRNAs targeting separate regions of an individual mRNAs.

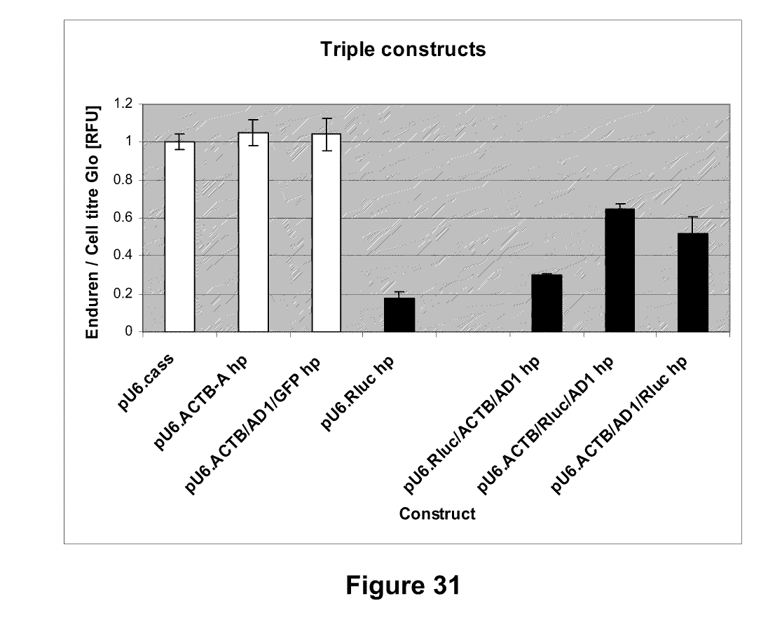

FIG. 31 shows the activity of triple hairpin constructs targeting Renilla luciferase. These data demonstrate that sequences at the base, middle or top of a triple hairpin construct significantly reduce Renilla luciferase activity. This indicates that such a triple hairpin construct can produce three active siRNAs and it follows necessarily that constructs may be designed to inactivate three genes. Moreover such constructs might be used to inactivate a single gene more effectively via the additive effect of producing three separate siRNAs targeting separate regions of an individual mRNAs.

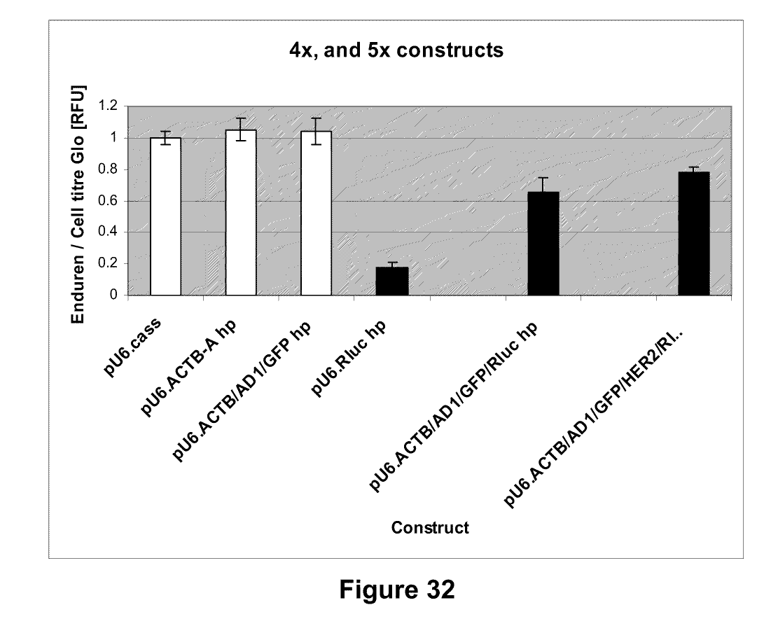

FIG. 32 shows the activity of constructs 4× and 5× constructs targeting Renilla luciferase. These data demonstrated that sequences at the top of a 4× or 5× construct significantly reduced Renilla luciferase activity. This indicates that such constructs can produce four or five active siRNAs and it follows necessarily that constructs may be designed to inactivate four or five genes. Moreover such constructs may be used to inactivate a single gene more effectively via the additive effect of producing four or five separate siRNAs targeting separate regions of an individual mRNAs.

8. Inactivating Two Genes with a Single Construct

To demonstrate that the above strategy can be used to inactivate two endogenous genes a single construct was prepared targeting the Akt1 (site a) and Akt2 (sites a and b) genes. This construct, pU6.GF-2, was designed to inactivate two genes, Akt1 and Akt2; sequences were designed based on the data of Jiang Z Y, Zhou Q L, Coleman K A, Chouinard M, Boese Q, Czech M P (2003). Insulin signalling through Akt/protein kinase B analysed by small interfering RNA-mediated gene silencing. Proc Natl Acad Sci USA. 100(13):7569-74. pU6.GF-2 was prepared using the long range PCR strategy described above using Bgl II linearised pU6.cass lin as a substrate; this was amplified with Pfu Turbo polymerase (Stratagene) using the primers:

G U6F-2

(SEQ ID NO: 78)

CACAAAGAGGCGCTCGTGGTCCTGGCTAACAGCTTCTCGTGGTCCTGGCG
GTGTTTCGTCCTTTC
and
G termF-2

(SEQ ID NO: 79)

TAGGAGGCGCTCGTGGTCCTGGTTGACAGCTTCTCGTGGTCCTGGTTTT
TTGGAAAAGCTTATCG

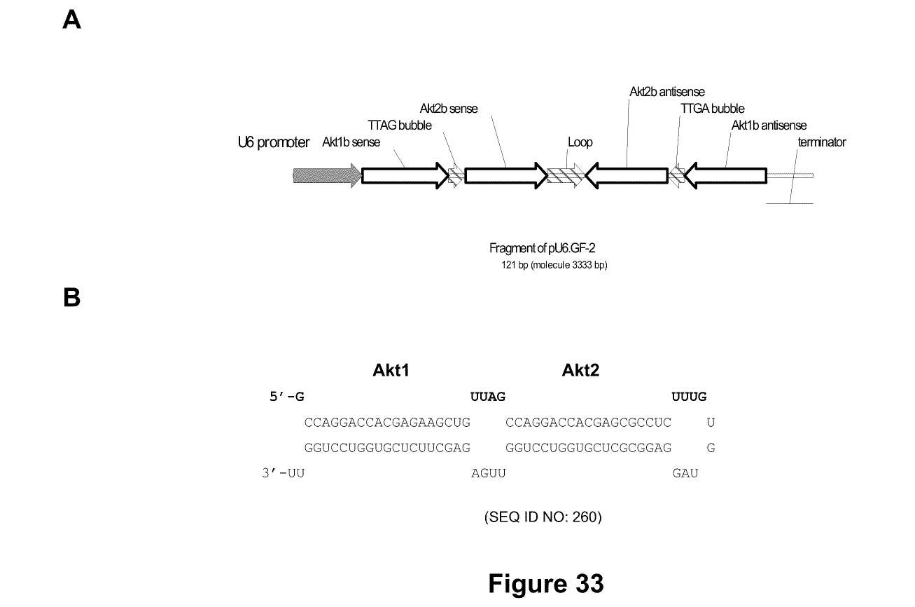

A map of the construct is shown in FIG. 33A and the sequence (SEQ ID NO: 260) of the predicted RNA produced by the construct is shown in FIG. 33B.

To assay the activity of this construct C2C12 cells were transfected with the plasmid using Lipofectamine 2000 according to the manufacturer's (Invitrogen) protocols. After 48 hrs total proteins were isolated and Akt1 and Akt2 protein levels were determined using Western blots; blots were also probed with a control antibody to ensure even loading. Procedures for these experiments are well known to those familiar with the art.

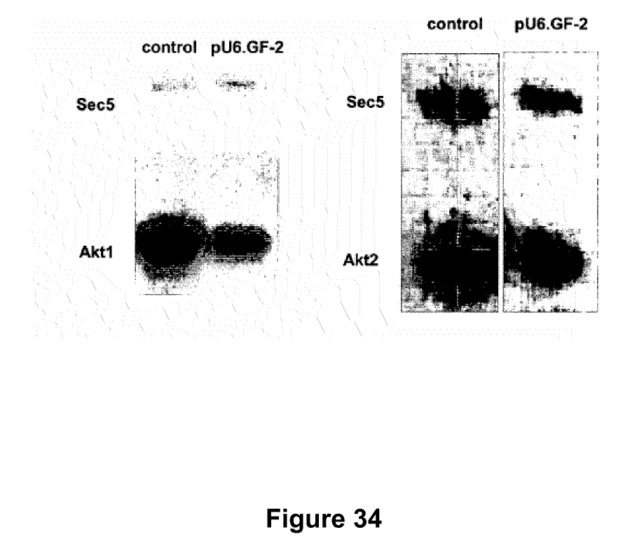

FIG. 34 shows that the levels of both Akt1 and Akt2 were reduced in cells transfected with pU6.GF-2.

### 9. Increasing the Activity of a Construct by Targeting Two Regions of a Single Gene

To demonstrate this approach can be used to increase the activity of constructs the plasmid pU6.GG-4 was prepared. This construct targets the Akt2 gene, target site selection was based on the data of Jiang et al (2003) cited above. Two sites (“a” and “b”) within the Akt2 gene were targeted and compared to the activity of the two single hp constructs targeting each sites, these single hp constructs were named pU6.GG-2 and pU6.GG-3.

The construct pU6.GG-2 was prepared using the long range PCR strategy described above using Bgl II linearised pU6.cass lin as a substrate; this was amplified with Pfu Turbo polymerase (Stratagene) using the primers:

G U6-G2

(SEQ ID NO: 80)

CACAAAGGTGCCCTTGCCGAGGAGTCGGTGTTTCGTCCTTTC
and
G term-G2

(SEQ ID NO: 81)

TAGGAGGCGCTCGTGGTCCTGGTTTTTTGGAAAAGCTTATCG

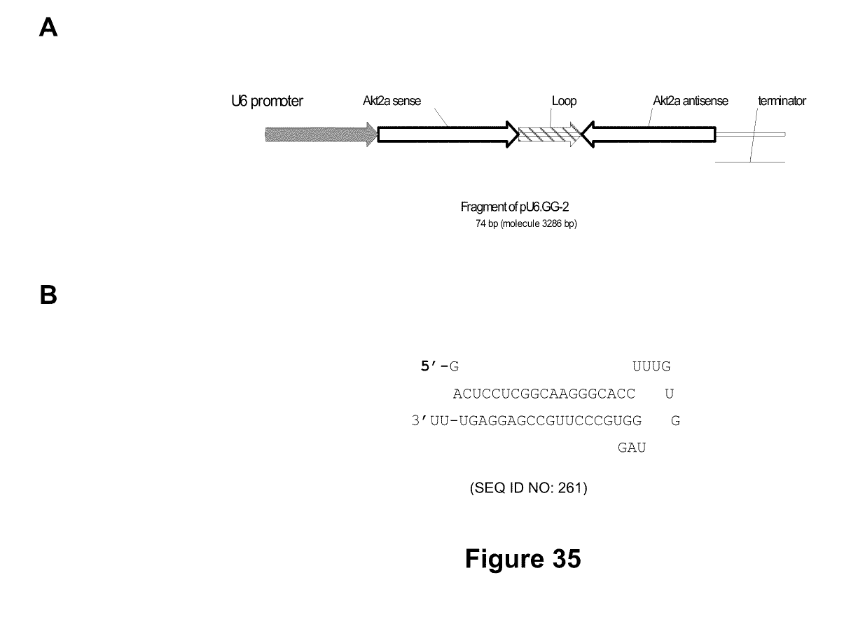

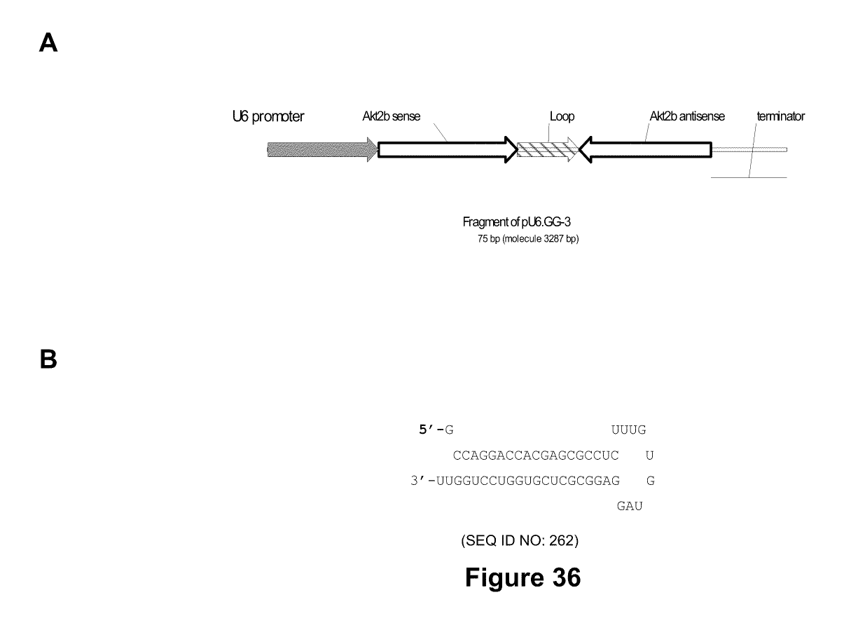

A map of the construct is shown in FIG. 35A and the sequence (SEQ ID NO: 261) of the predicted RNA produced by the construct is shown in FIG. 35B.

The construct pU6.GG-3 was prepared using the long range PCR strategy described above using Bgl II linearised pU6.cass lin as a substrate; this was amplified with Pfu Turbo polymerase (Stratagene) using the primers:

GU6-G3

(SEQ ID NO: 82)

CACAAAGAGGCGCTCGTGGTCCTGGCGGTGTTTCGTCCTTTC
and
G term-G3

(SEQ ID NO: 83)

TAGGGTGCCCTTGCCGAGGAGTTTTTTGGAAAAGCTTATCG

A map of the construct is shown in FIG. 36A and the sequence (SEQ ID NO: 262) of the predicted RNA produced by the construct is shown in FIG. 36B.

The construct pU6.GG-4 was prepared using the long range PCR strategy as described above using Bgl II linearised pU6.cass lin as a substrate; this was amplified with Pfu Turbo polymerase (Stratagene) using the primers:

G U6-G4

(SEQ ID NO: 84)

CACAAAGAGGCGCTCGTGGTCCTGGCTAAGGTGCCCTTGCCGAGGAGTC
GGTGTTTCGTCCTTTC
and
G term-G4

(SEQ ID NO: 85)

TAGGAGGCGCTCGTGGTCCTGGTTGAGGTGCCCTTGCCGAGGAGTTTTTT
GGAAAAGCTTATCG

A map of the construct is shown in FIG. 37A and the sequence (SEQ ID NO: 263) of the predicted RNA produced by the construct is shown in FIG. 37B.

To assay the activity of these constructs C2C12 myoblasts were transfected with the constructs and Akt2 protein levels were determined using quantitative Western blots as described above.

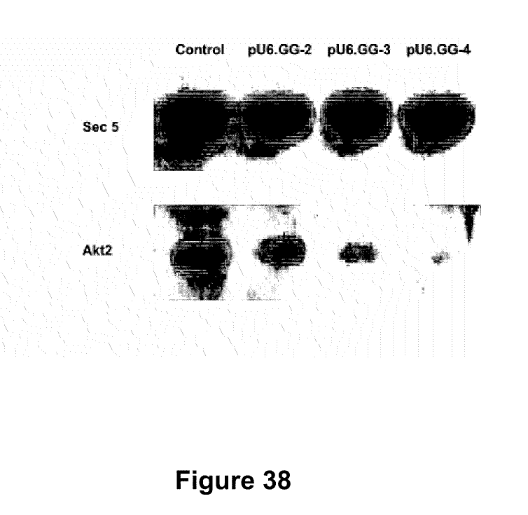

The results of these experiments are shown in FIG. 38 which demonstrates that pU6.GG-4 shows increased activity compared to either of the constructs. pU6.GG-2 or pU6.GG-3.

### 10. Double Hairpin Constructs Targeting ADAR and β Actin

### 10.1 Test Constructs

In this example DNA constructs were prepared which were targeted to inactivating the ADAR 1 and ADAR 2 genes (these are also sometimes known as ADARA and ADARB respectively).

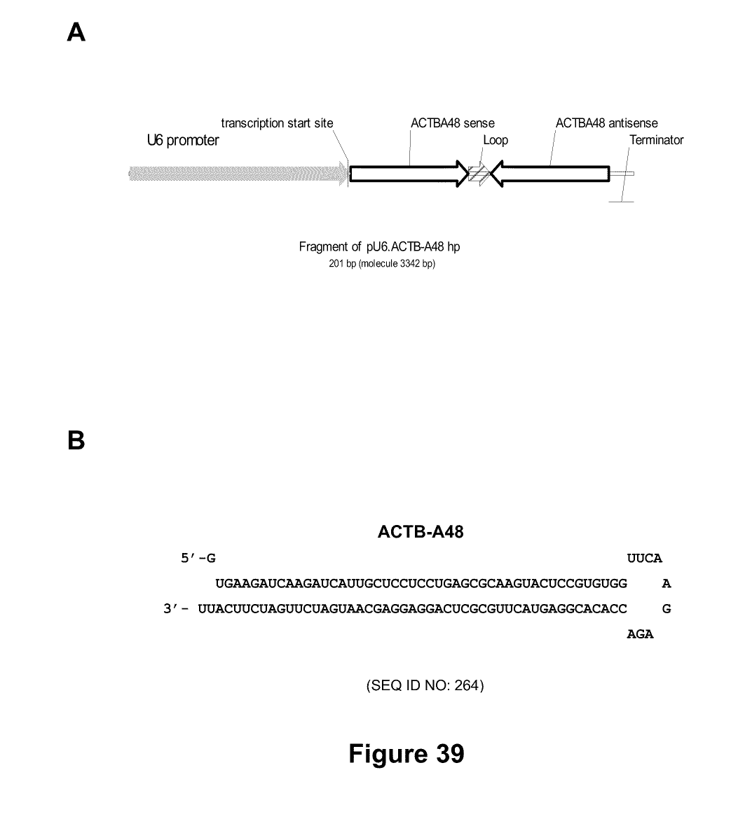

Two plasmids, pU6.ACTB-A hp (FIG. 2) and pU6.ACTB-A48 hp (FIG. 39) were used. These constructs were designed to inactivate β actin mRNA and were used as controls in the experiments described below, in addition pU6.ACTB-A hp was used as a precursor to generate many of the constructs designed below.

A similar strategy to that described above for pU6.ACTB-Ahp was used to prepare the construct pU6.ACTB-A48 hp. In this instance eight oligonucleotides were annealed, namely

ACTB48-9

(SEQ ID NO: 86)

ACCGTGAAGATCAAGATCATTGCTCCTCCTGA
ACTB48-10

(SEQ ID NO: 87)

CAATGATCTTGATCTTCA
ACTB48-3

(SEQ ID NO: 88)

GCGCAAGTACTCCGTGTGGTTCAAGAGA
ACTB48-4

(SEQ ID NO: 89)

CCACACGGAGTACTTGCGCTCAGGAGGAG
ACTB48-5

(SEQ ID NO: 90)

CCACACGGAGTACTTGCGCTCAGGAGGAGCA
ACTB48-6

(SEQ ID NO: 91)

TGAGCGCAAGTACTCCGTGTGGTCTCTTGAA
ACTB48-11

(SEQ ID NO: 92)

AATGATCTTG ATCTTCATTTTTGGAAA
ACTB48-12

(SEQ ID NO: 93)

AGCTTTTCCAAAAATGAAGATCAAGATCATTGCTCCTCC

Four partially complementary pairs of oligonucleotides (ACTB48-9 and ACTB48-10, ACTB48-3 and ACTB48-4, ACTB48-5 and ACTB48-6, and ACTB48-11 and ACTB48-12) were annealed, and annealed pairs were themselves annealed through two further cycles of annealing to produce a double-stranded DNA structure (SEQ ID NO: 264) compatible with cloning into BsmB 1/Hind III digested pU6.cass as shown diagrammatically in FIG. 39. The annealed oligonucleotides were cloned into pU6.cass as described above. This plasmid was expected to express a 48 nt hairpin RNA, with transcription initiating in the human U6 promoter and terminating at the poly T tract in the 3′ region of the annealed sequences.

The constructs used in these experiments are described in Table 5. Conventional single hp DNA constructs were used as controls. Double hairpin constructs were prepared and their activity was compared to the control constructs. The control constructs targeted a single gene, the test constructs (“double hairpin” constructs) targeted two genes, one gene targeted by the base of the hairpin sequence, the second by sequences near the loop of the hairpin structure.

TABLE 5
Control and “double hairpin constructs used in these experiments.

| Construct designation | Target^{a} | “Bubble” sequence |
|---|---|---|
| Single hp constructs | | |
| pU6.AD1-A | ADAR 1 site A | na |
| pU6.AD2-C | ADAR 2 site C | na |
| pU6.AD2-A | ADAR 2 site A | na |
| pU6.AD1/2-B | ADAR 1 site B & ADAR 2 site B | na |
| | (single 19 nt hairpin) | |
| Double hp constructs | | |
| pU6.AD1&2-A/UU | ADAR 1 site A and ADAR 2 site A | 5′-UU-3′ |
| | (19 nt hairpin targeting each | 3′-UU-5′ |
| | target) | |
| pU6.AD1&2-A/UUA | ADAR 1 site A and ADAR 2 site A | 5′-UUA-3′ |
| | (19 nt hairpin targeting each | 3′-GUU-5′ |
| | target) | |
| pU6.AD1&2-A/UUACAA | ADAR 1 site A and ADAR 2 site A | 5′-UUACAA-3′ |
| | (19 nt hairpin targeting each | 3′-GUUGUU-5′ |
| | target) | |

^{a}mRNA targeted for inactivation
ADAR 1 site A corresponds to positions 1477 to 1497 of GenBank sequence NM_001111.
ADAR 2 site C corresponds to positions 22 to 42 of GenBank sequence HSU82121.
ADAR 2 site A corresponds to positions 2134 to 2154 of GenBank sequence HSU82121.
ADAR 1 site A and ADAR 2 site A are completely different sequences.
ADAR1 site B and ADAR 2 site B are identical sequences present in both the ADAR 1 and ADAR 2 genes ADAR1 site B corresponds to positions 2906 to 2927 of GenBank sequence NM_00111. ADAR2 site B corresponds to positions 1174 to 1192 of GenBank sequence HSU82121.

The test constructs were prepared as follows.

pU6.AD1-A

This construct was designed to target ADAR 1 mRNA for inactivation at the ADAR 1 A site and acted as a control for the double hairpin constructs which all targeted ADAR1 mRNA at the A site with sequences located at the base of the hairpin (SEQ ID NO: 265). The construct was prepared using the long range PCR strategy described above. The plasmid pU6.ACTB-A hp was used as a substrate, this was amplified using Pfu Turbo polymerase (Stratagene) with the primers:

pU6 ADAR-A Fwd

(SEQ ID NO: 94)

AGAGATGAACAGGTGGTTTCAGTCTTTTTGGAAAAGCTTATCGATACC
pU6 ADAR-A Rev

(SEQ ID NO: 95)

TGAATGAACAGGTGGTTTCAGTCGGTGTTTCGTCCTTTCCACAAG

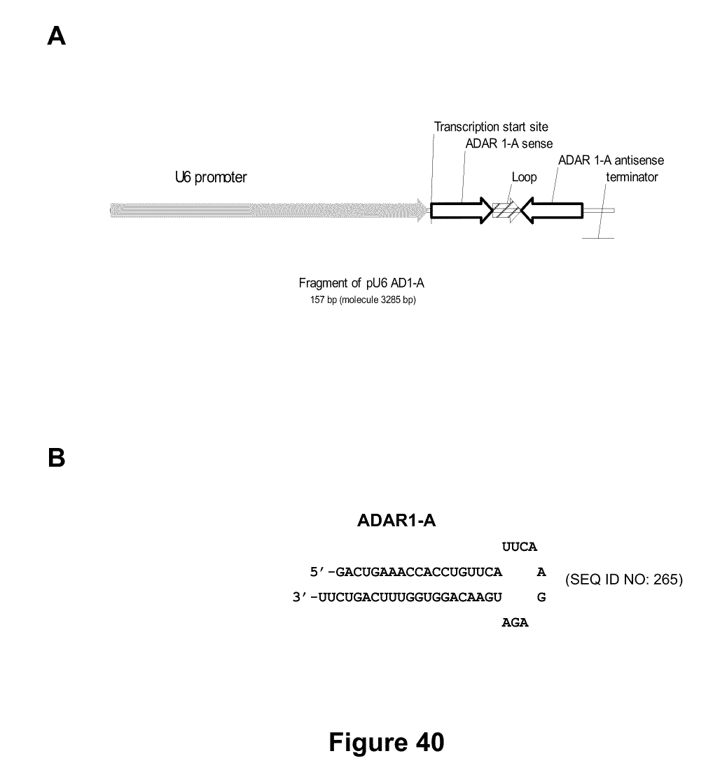

A map of this construct is shown in FIG. 40.

pU6.AD2-C

This construct was designed to target ADAR 1 mRNA for inactivation at the ADAR 2 C site and acted as a control for the double hairpin constructs (SEQ ID NO: 266) which all targeted ADAR2 mRNA at a different site. The construct was prepared using the long range PCR strategy described above. The plasmid pU6.ACTB-A hp was used as a substrate, this was amplified using Pfu Turbo polymerase (Stratagene) with the primers:

pU6 ADARB1-A Fwd

(SEQ ID NO: 96)

AGAGAGGCTGTGAACAGACGCGCCTTTTTGGAAAAGCTTATCGATACC
pU6 ADARB1-A Rev

(SEQ ID NO: 97)

TGAAGGCTGTGAACAGACGCGCCGGTGTTTCGTCCTTTCCACAAG

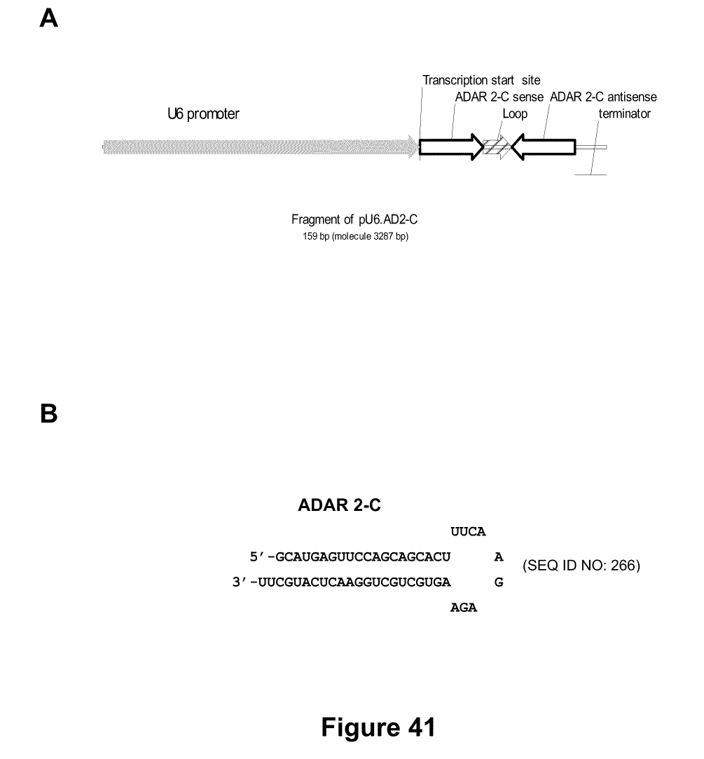

A map of this construct is shown in FIG. 41.

pU6.AD2-A

This construct was designed to target ADAR 2 mRNA for inactivation at the ADAR 2 A site, and acted as a control for the double hairpin constructs which all targeted ADAR 2 mRNA at the A site with sequences located near the loop of the hairpin structure (SEQ ID NO: 267). The construct was prepared using the long range PCR strategy described above. The plasmid pU6.ACTB-A hp was used as a substrate, this was amplified using Pfu Turbo polymerase (Stratagene) with the primers:

pU6 ADARB1-C Fwd

(SEQ ID NO: 98)

3AGAGAAGTGCTGCTGGAACTCATGCTTTTTGGAAAAGCTTATCGATAC
CG
pU6 ADARB1-C Rev

(SEQ ID NO: 99)

3TGAAAGTGCTGCTGGAACTCATGCGGTGTTTCGTCCTTTCCACAAG

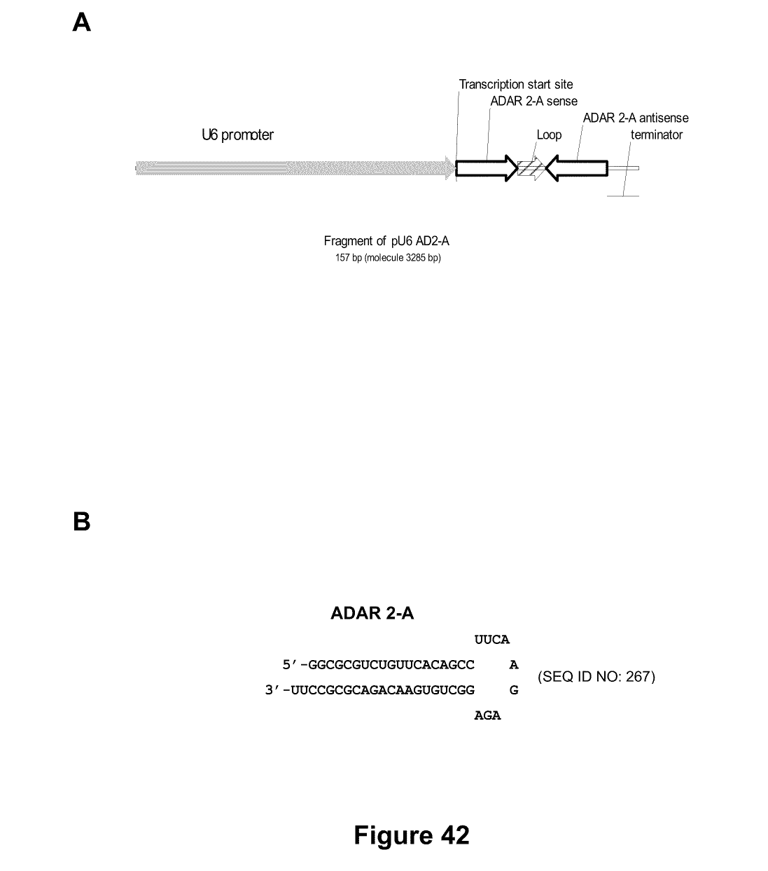

A map of this construct is shown in FIG. 42.

pU6.AD1/2-B

This construct was designed to target both ADAR 1 and ADAR 2 mRNA for inactivation at the ADAR 1 B site and the ADAR 2 B site. Both ADAR 1 mRNA and ADAR 2 mRNA contain this site, both mRNAs were therefore potentially inactivated by a single hairpin element within the construct (SEQ ID NO: 268). This construct acted as a control for the double hairpin constructs which all targeted ADAR 1 and/or ADAR 2 mRNAs at different sites. The construct was prepared using the long range PCR strategy described above. The plasmid pU6.ACTB-A hp was used as a substrate, this was amplified using Pfu Turbo polymerase (Stratagene) using the primers:

pU6 ADAR1/2-B Fwd

(SEQ ID NO: 100)

AGAGATTATTTCTGCATGGCAGTCATTTTTGGAAAAGCTTATCGATACCG
pU6 ADAR1/2-B Rev

(SEQ ID NO: 101)

3TGAATTATTTCTGCATGGCAGTCGGTGTTTCGTCCTTTCCACAAG

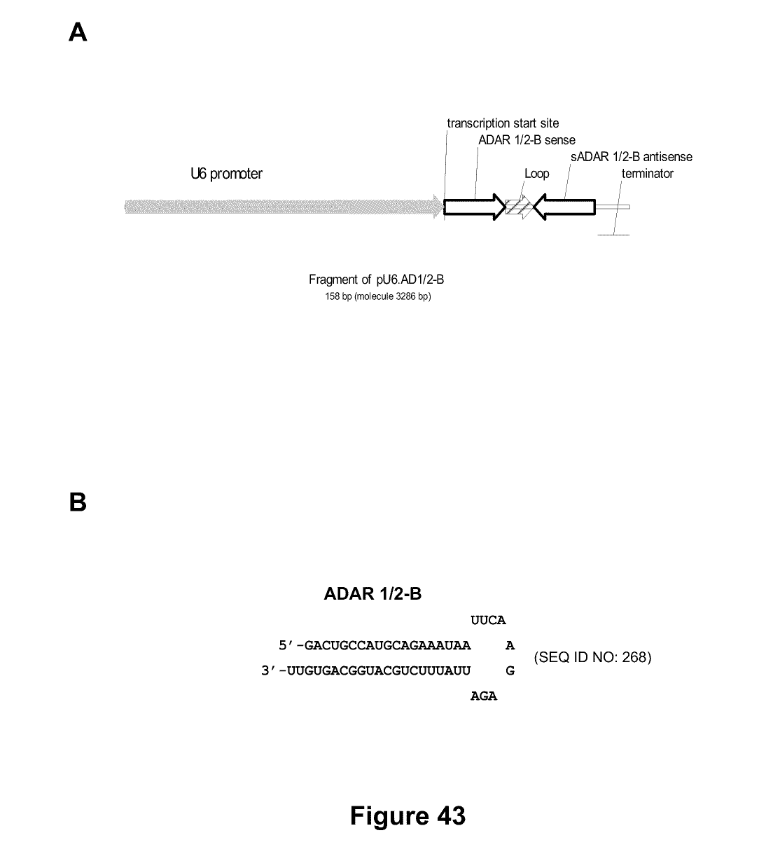

A map of this construct is shown in FIG. 43.

pU6.AD1 &2-A/UU

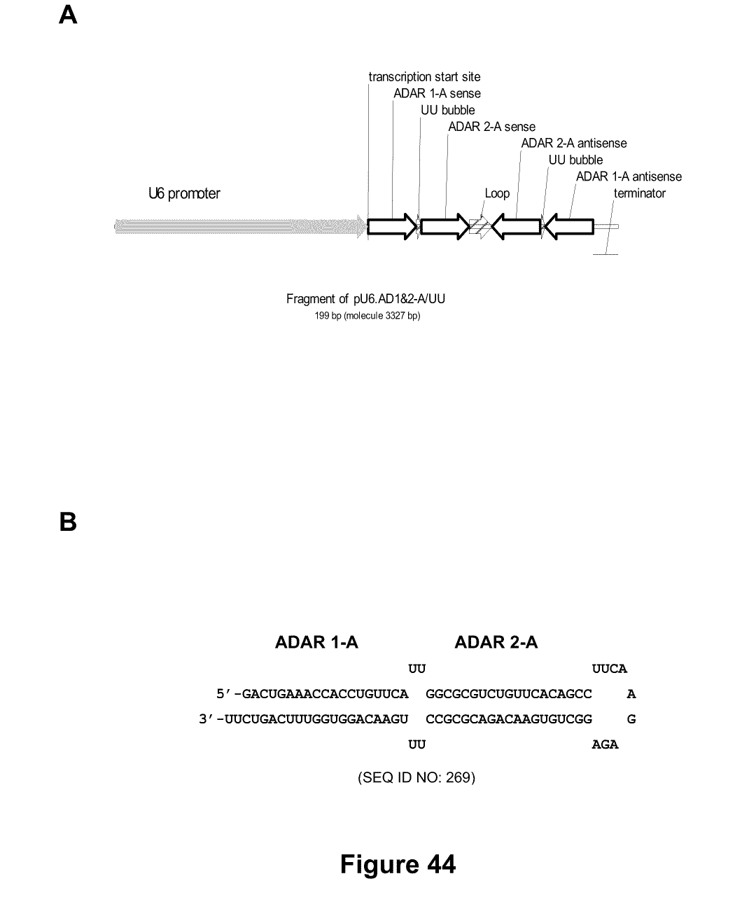

This double hairpin construct was designed to inactivate ADAR 1 mRNA at the ADAR 1 A site with sequences at the base of the hairpin DNA construct, and ADAR 2 mRNA at the ADAR 2 A site with sequences near the loop of the double hairpin structure (SEQ ID NO: 269). The two structural elements were separated by a two nucleotide “bubble” sequence UU (FIG. 44). The construct was prepared using the long range PCR strategy described above. The plasmid pU6.AD1-A was used as a substrate, this was amplified using Pfu Turbo polymerase (Stratagene) using the primers:

pU6 ADAR 1/2-AA Fwd

(SEQ ID NO: 102)

AGAGAGGCTGTGAACAGACGCGCCTTTGAACAGGTGGTTTCAGTCTTTT
TGGAAAAGC
pU6 ADAR 1/2-AA Rev

(SEQ ID NO: 103)

TGAAGGCTGTGAACAGACGCGCCAATGAACAGGTGGTTTCAGTCGGTGT
TTCGT

A map of this construct is shown in FIG. 44.

pU6.AD1&2-A/UUA

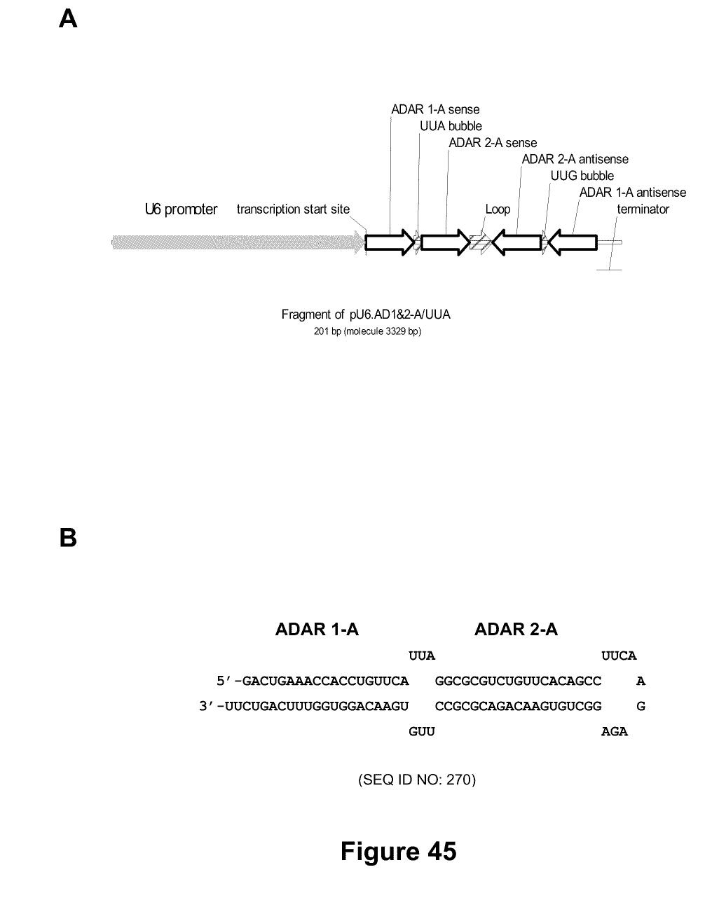

This double hairpin construct was designed to inactivate ADAR 1 mRNA at the ADAR 1 A site with sequences at the base of the hairpin DNA construct, and ADAR 2 mRNA at the ADAR 2 A site with sequences near the loop of the double hairpin structure (SEQ ID NO: 270). The two structural elements were separated by a three nucleotide “bubble” sequence UUA (FIG. 45). The construct was prepared using the long range PCR strategy described above. The plasmid pU6.AD1-A was used as a substrate, this was amplified using Pfu Turbo polymerase (Stratagene) using the primers:

pU6ADAR1/2-AA (+1) F

(SEQ ID NO: 104)

AGAGAGGCTGTGAACAGACGCGCCTTGTGAACAGGTGGTTTCAGTCTTT
TTGGAAAAGC
pU6ADAR1/2-AA(+1)R

(SEQ ID NO: 105)

TGAAGGCTGTGAACAGACGCGCCTAATGAACAGGTGGTTTCAGTCGGTG
TTTCGT

A map of this construct is shown in FIG. 45.

pU6.AD1 &2-A/UUACAA

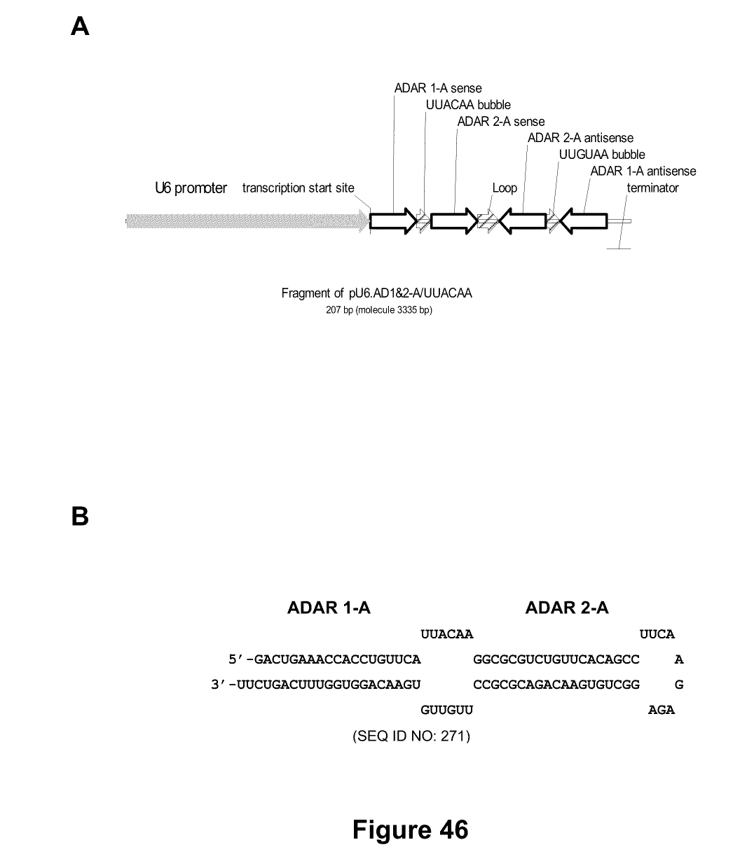

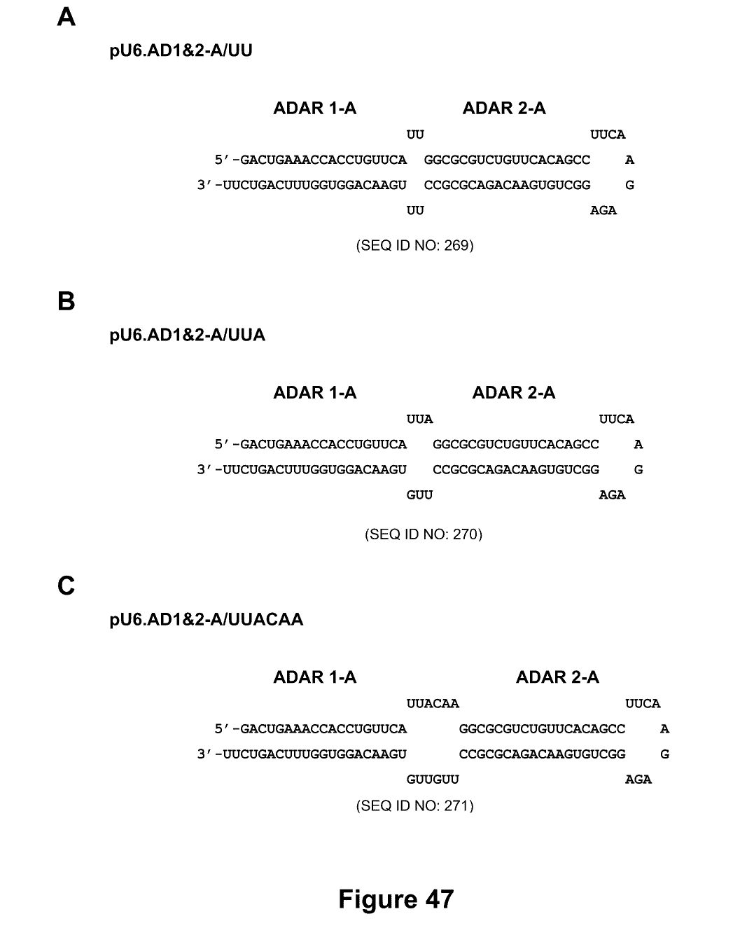

This double hairpin construct was designed to inactivate ADAR 1 mRNA at the ADAR 1 A site with sequences at the base of the hairpin DNA construct, and ADAR 2 mRNA at the ADAR 2 A site with sequences near the loop of the double hairpin structure (SEQ ID NO: 271). The two structural elements were separated by a six nucleotide “bubble” sequence UUACAA (FIG. 46). The construct was prepared using the long range PCR strategy described above. The plasmid pU6.AD1-A was used as a substrate, this was amplified using Pfx Turbo polymerase (Invitrogen) using the primers:

pU6ADAR1/2-AA ButF

(SEQ ID NO: 106)

AGAGAGGCTGTGAACAGACGCGCCTTGTAATGAACAGGTGGTTTCAGTC
TTTTTGGAAAAGC
pU6ADAR1/2-AA ButR

(SEQ ID NO: 107)

TGAAGGCTGTGAACAGACGCGCCTTGTAATGAACAGGTGGTTTCAGTCG
GTGTTTCG

A map of this construct is shown in FIG. 46.

### 10.2 Control Constructs

The two hairpin DNA constructs, pU6.ACTB-A hp (FIG. 2) and pU6.ACTB-A48 hp (FIG. 39) were used as controls for non-specific effects of expressing hairpin RNAs. The construct pU6.ACTB-A48 hp was the most appropriate control since it expresses a hairpin RNA of very similar size to that expressed by the double hairpin constructs. The ACTB-A sequence targeted by pU6.ACTB-A hp, corresponds to positions 1045-1065 of GenBank sequence NM_{—}001101. The ACTB-A sequence targeted by pU6.ACTB-A48 hp, corresponds to positions 1045-1094 of GenBank sequence NM_{—}001101. As an additional control the effects of an siRNA targeting the ADAR 1 B and ADAR 2 B sites was tested using RNA transcribed from T7 promoters. The siRNA was termed siAD1/2-B and was prepared using the oligonucleotides:

| | |
|---|---|
| ADAR1/2-B T7 S | |
| AATGACTGCCATGCAGAAATACCTGTCTC | (SEQ ID NO: 108) |
| ADAR1/2-B T7 AS | |
| AATATTTCTGCATGGCAGTCACCTGTCTC | (SEQ ID NO: 109) |

As an additional control the effects of an siRNA targeting the ACTB-A site was tested using RNA transcribed from T7 promoters. The siRNA was termed siACTB-A and the DNA encoding this siRNA was prepared using the oligonucleotides:

| | |
|---|---|
| ACTB-A T7 S | |
| AATGAAGATCAAGATCATTGCCCTGTCTC | (SEQ ID NO: 110) |
| ACTB-A T7 AS | |
| AAGCAATGATCTTGATCTTCACCTGTCTC | (SEQ ID NO: 111) |

### 10.3 Double Hairpin Constructs Targeting β Actin and Adar1

Five further constructs, targeting both β actin and ADAR 1 were prepared as outlined in the Table 6 illustrating other embodiments of the invention.

TABLE 6
Double hairpin constructs

| | | Bubble |
|---|---|---|
| Construct | Target^{a} | Sequence |
| pU6.AD1-A&ACTB-A/UU | ADAR1 site A | 5′-UU-3′ |
| | β actin (ACTB-A) | 3′-UU-5′ |
| pU6.AD1-A&ACTB-A/UUA | ADAR1 site A | 5′-UUA-3′ |
| | β actin (ACTB-A) | 3′-GUU-5 |
| pU6.AD1-A&ACTB-A/UUAG | ADAR1 site A | 5′-UUAG-3′ |
| | β actin (ACTB-A) | 3′-AGUU-5′ |
| pU6.AD1-A&ACTB-A/ | ADAR1 site A | 5′-UUACAA-3′ |
| UUACAA | β actin (ACTB-A) | 3′-UUAGUU-5 |
| pU6.ACTB-A&AD1-A/UUA | β actin (ACTB-A) | 5′-UUA-3′ |
| | ADAR1 site A | 3′-GUU-5′ |

^{a}ACTB-A site corresponds to positions 1045-1065 of NM_001101.

The constructs were prepared using the long range PCR strategy described above.

pU6.ACTB-A/UUA

This construct was designed to test whether a UUA bubble sequence (SEQ ID NO: 272) will enhance the activity of a single hairpin DNA construct having the sequence of ACTB-A (β actin). The construct is prepared using the plasmid pU6.ACTB-A hp as a substrate by amplifying with the two primers:

ACTADdelR

(SEQ ID NO: 112)

CTGAAATCTCTTGAATTTCAGTCTAAGCAATGATCTTGATCTTCACGGTG
ACTADdelF

(SEQ ID NO: 113)

TCTTGGCAATGATCTTGATCTTCATTTTTGGAAAAGCTTATCGATACCGT
C

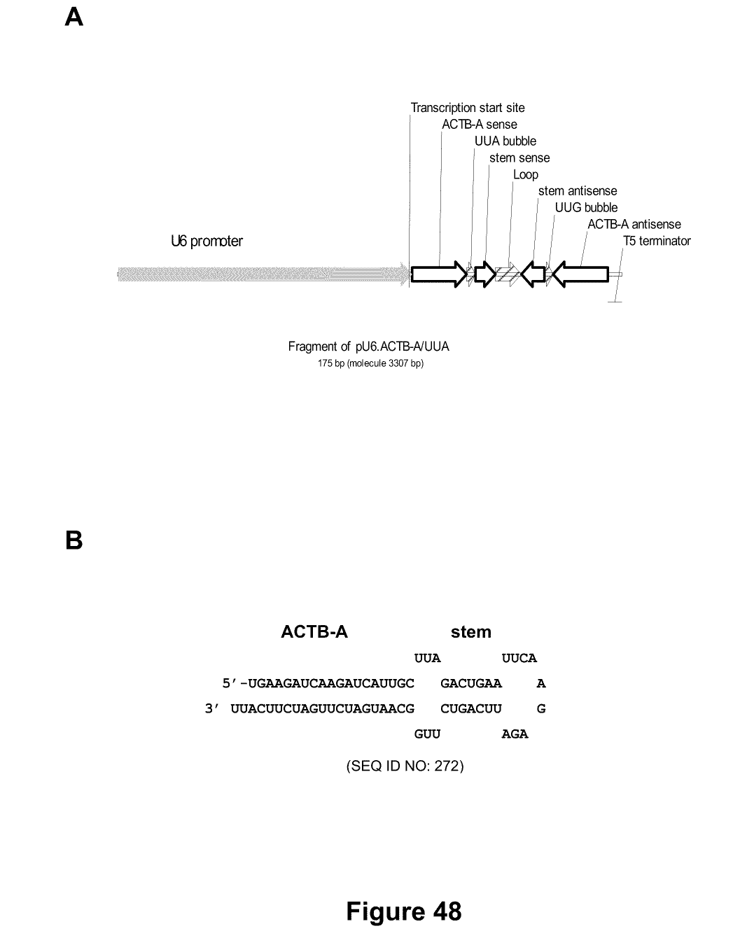

A map of this construct is shown in FIG. 48.

pU6.AD1-A&ACTB-A/UU

This construct was designed to test whether a construct carrying a UU bubble sequence (SEQ ID NO: 273) was capable of inactivating two mRNAs, namely ADAR 1 and β actin. The construct was prepared using the plasmid pU6.AD1&2-A/UU as a substrate by amplifying with the two primers:

AAR

(SEQ ID NO: 114)

TCATTGCTCTCTTGAAGCAATGATCTTGATCTTCAAATGAACAGGTGGTT
TCAGTCGGTG
AAF

(SEQ ID NO: 115)

TCTTGATCTTCATTTGAACAGGTGGTTTCAGTCTTTTTGGAAAAGCTTAT
CGATACCGTC

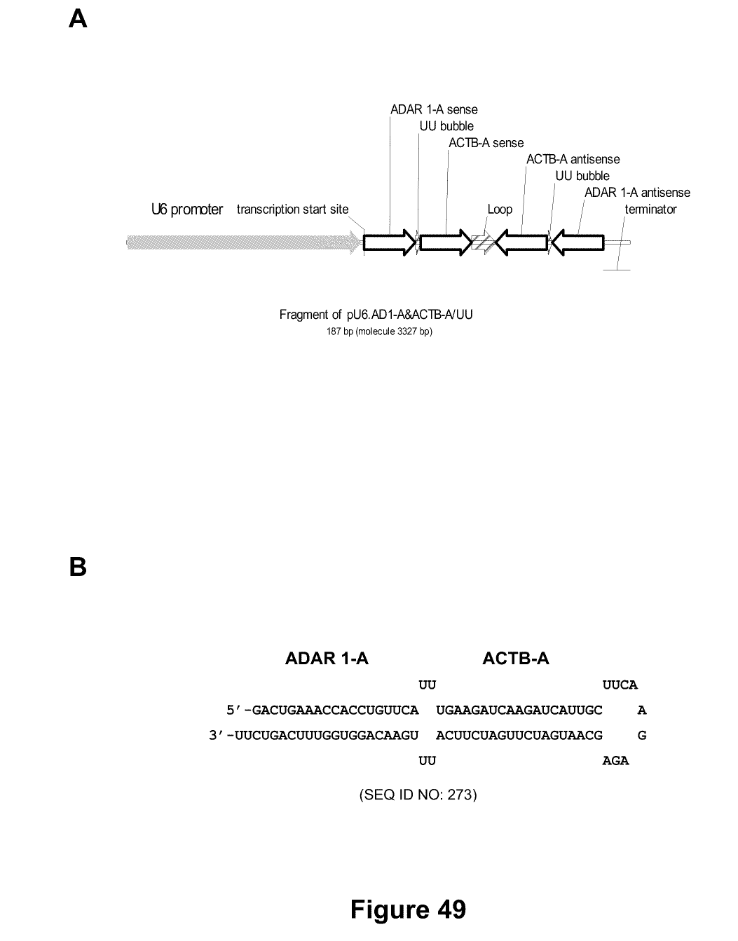

A map of this construct is shown in FIG. 49.

pU6.AD1-A&ACTB-A/UUA

This construct was designed to test whether a construct carrying a UUA bubble sequence (SEQ ID NO: 274) was capable of inactivating two mRNAs, namely ADAR 1 and β actin. The construct was prepared using the plasmid pU6.AD1&2-A/UU as a substrate by amplifying with the two primers:

AA + 1R

(SEQ ID NO: 116)

TCATTGCTCTCTTGAAGCAATGATCTTGATCTTCATAATGAACAGGTGGT
TTCAGTCGGTG
AA + 1F

(SEQ ID NO: 117)

TCTTGATCTTCATTGTGAACAGGTGGTTTCAGTCTTTTTGGAAAAGCTTA
TCGATACCGTC

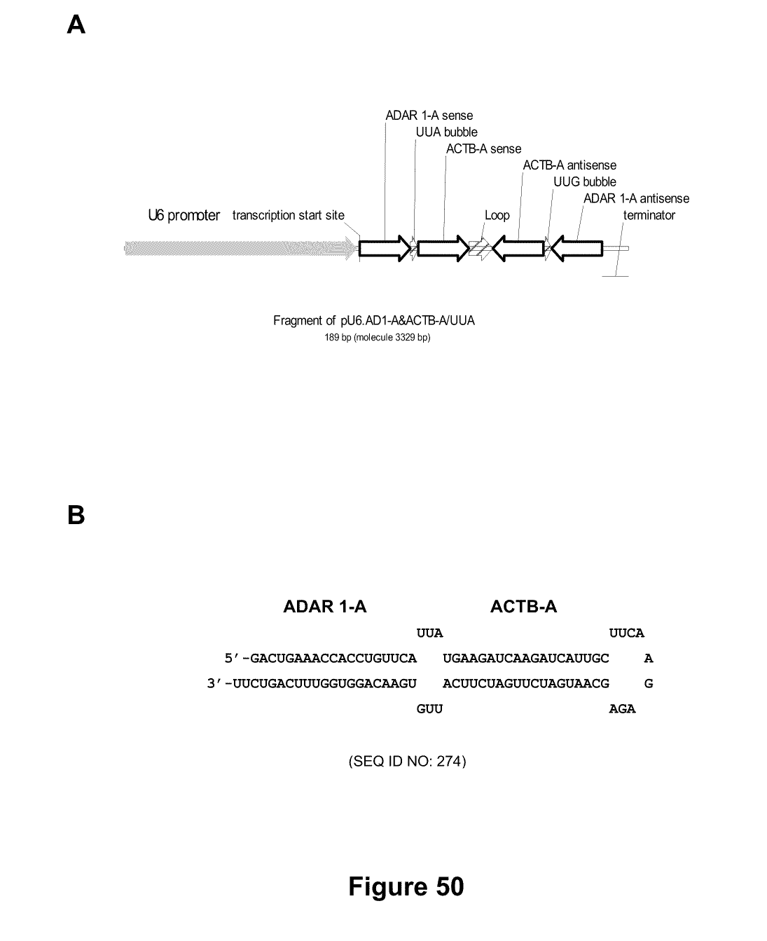

A map of this construct is shown in FIG. 50.

pU6.AD1-A&ACTB-A/UUAG

This construct was designed to test whether a construct carrying a UUAG bubble sequence (SEQ ID NO: 275) was capable of inactivating two mRNAs, namely ADAR 1 and β actin. The construct was prepared using the plasmid pU6.AD1&2-A/UU as a substrate by amplifying with the two primers:

AA + 2R

(SEQ ID NO: 118)

CATTGCTCTCTTGAAGCAATGATCTTGATCTTCACTAATGAACAGGTGGT
TTCAGTCGGTG
AA + 2F

(SEQ ID NO: 119)

ATCTTGATCTTCATTGATGAACAGGTGGTTTCAGTCTTTTTGGAAAAGCT
TATCGATACCGTC

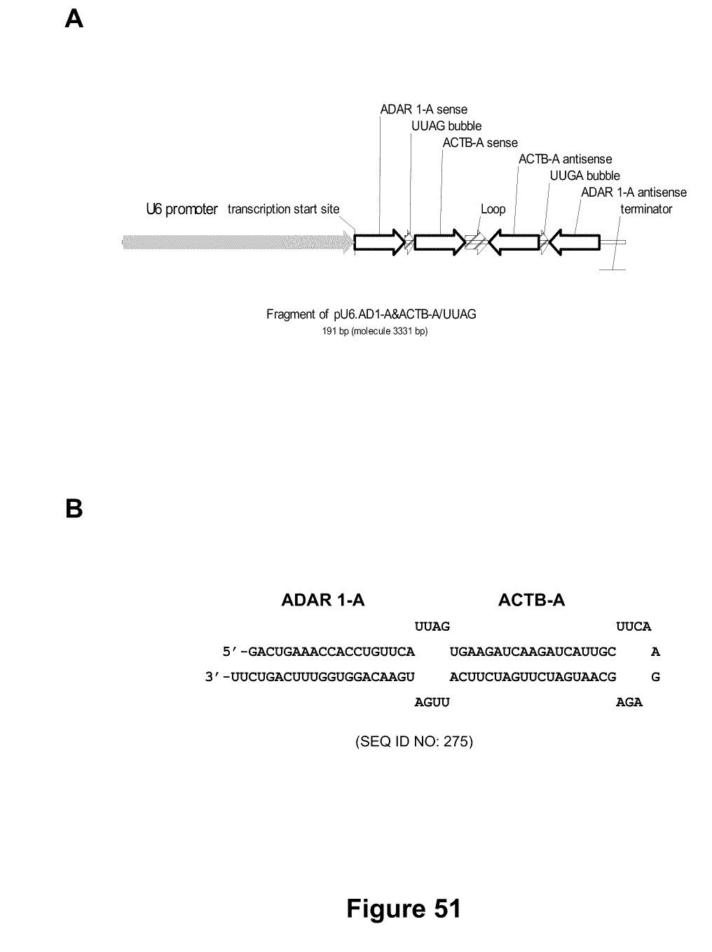

A map of this construct is shown in FIG. 51.

pU6.AD1-A&ACTB-A/UUACAA

This construct was designed to test whether a construct carrying a UUACAA bubble sequence (SEQ ID NO: 276) was capable of inactivating two mRNAs, namely ADAR 1 and β actin. The construct was prepared using the plasmid pU6.AD1&2-A/UU as a substrate by amplifying with the two primers:

AABR

(SEQ ID NO: 120)

CATTGCTCTCTTGAAGCAATGATCTTGATCTTCATTGTAATGAACAGGTG
GTTTCAGTCGGTG
AABF

(SEQ ID NO: 121)

ATCTTGATCTTCATTGTAATGAACAGGTGGTTTCAGTCTTTTTGGAAAAG
CTTATCGATACCGTC

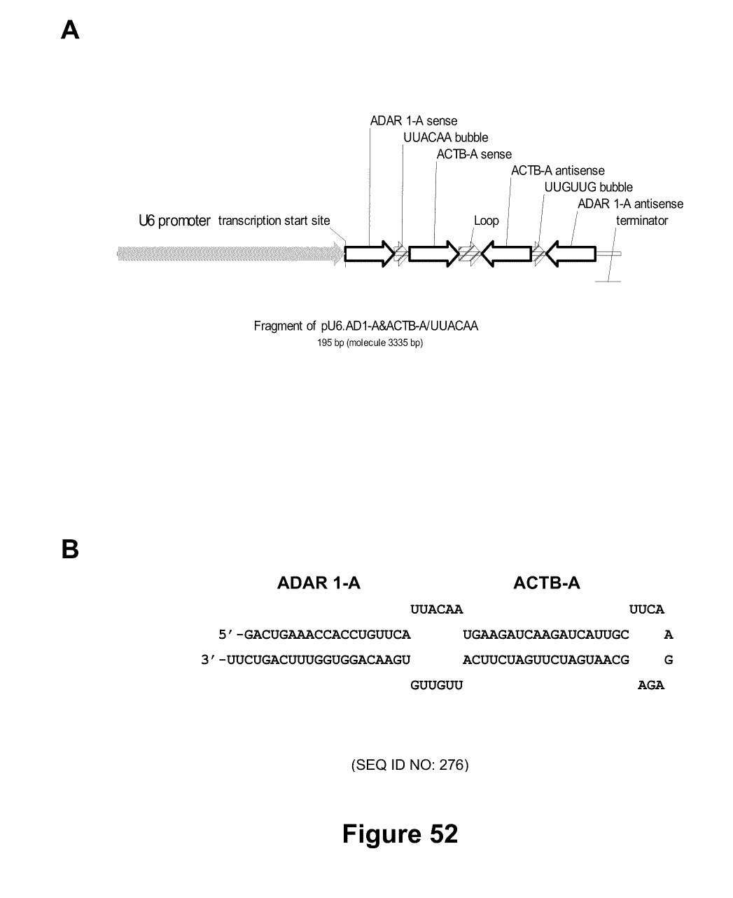

A map of this construct is shown in FIG. 52.

pU6.ACTB-A&AD1-A/UUA

This construct was designed to test whether a construct carrying a UUA bubble sequence (SEQ ID NO: 277) was capable of inactivating two mRNAs, namely 0 actin and ADAR 1. The construct differed from the construct pU6.AD1-A&ACTB-A/UUA in that the relative positions of the AD1-A and ACTB-A differed. The construct was prepared using the plasmid pU6.ACTB-A hp as a substrate by amplifying with the two primers:

ACTADR

(SEQ ID NO: 122)

TGTTCATCTCTTGAATGAACAGGTGGTTTCAGTCTAAGCAATGATCTTGA
TCTTCACGGTG
ACTADF

(SEQ ID NO: 123)

GGTGGTTTCAGTCTTGGCAATGATCTTGATCTTCATTTTTGGAAAAGCTT
ATCGATACCGTC

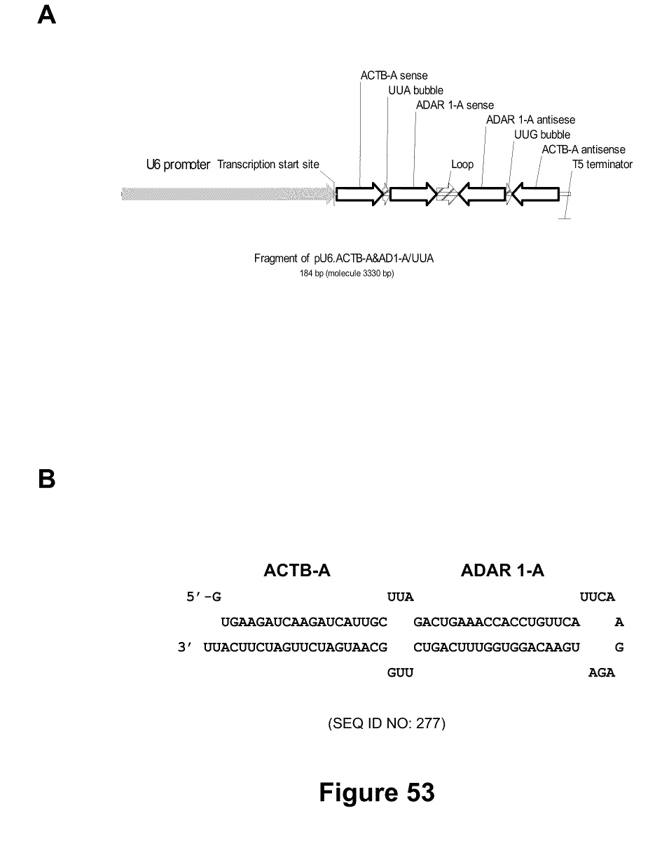

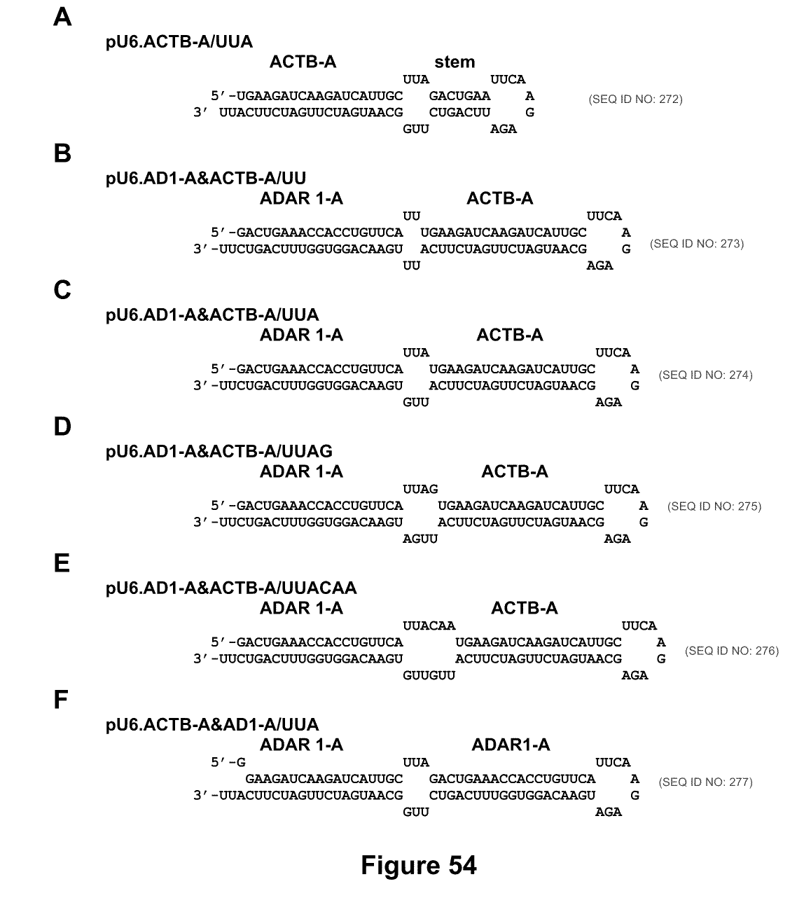

A map of this construct is shown in FIG. 53. FIG. 54 shows the predicted structure of hairpin RNAs (SEQ ID NOS: 273-277) produced by the double hairpin constructs targeting ADAR 1 and β actin.

### 10.4 Tissue Culture

HeLa cells were grown and maintained in tissue culture using known procedures. To transfect HeLa cells, 200,000 cells were plated in each well of a 6 well tissue culture plate. After overnight incubation cells were transfected with either siRNAs or plasmid DNAs. siRNAS were transfected using Oligofectamine according to manufacturer's (Invitrogen) protocol. Plasmid DNAs were transfected into cells using PolyFect according to manufacturer's (Qiagen) protocol. Cells were incubated for 48 hrs following transfection and total RNAs were isolated for analysis of ADAR1, ADAR 2 and/or β actin mRNA levels.

### 10.5 RNA Preparation and Analysis Using Quantitative Real Time PCR and Northern Blot Assays

Total RNAs were prepared using QIAGEN RNeasy mini columns according to the manufacturer's protocol. To remove DNase contamination samples were treated with DNase according to the manufacturer's (Qiagen) protocol. Poly A⁺ RNA was prepared using DYNAL Dynabeads® mRNA DIRECT™ Micro Kit according to the manufacturer's (DYNAL) protocol. Levels of ADAR 1 and ADAR 2 mRNAs were determined using Quantitative Real Time PCR assays. Three duplicate assays were performed for each RNA sample using SYBR green incorporation to determine relative mRNA levels. The reactions and analyses were performed using procedures widely known to those skilled in the art.

Quantitative Northern blot analyses were used to determine levels of 0 actin mRNA, and thereby quantify β actin inactivation. Northern blots of total RNAs isolated from cells were probed with a fragment specific to the 3′ UTR of β actin mRNA, prepared using PCR of total HeLa cell RNA, and the degree of hybridization quantified using a phosphoimager. To correct for unequal loading, Northern filters were stripped then reprobed with a PCR fragment corresponding to human GAPDH and the degree of hybridization also quantified using a phosphoimager. β actin mRNA levels in individual RNA samples were then normalized to GAPDH levels and the relative levels of β actin between experimental treatments were determined. The methodologies and procedures used for these analyses are widely known to those skilled in the art.

### 10.6 Double Hairpin Constructs can Simultaneously Inactivate Two Genes and can Show Enhanced Activity

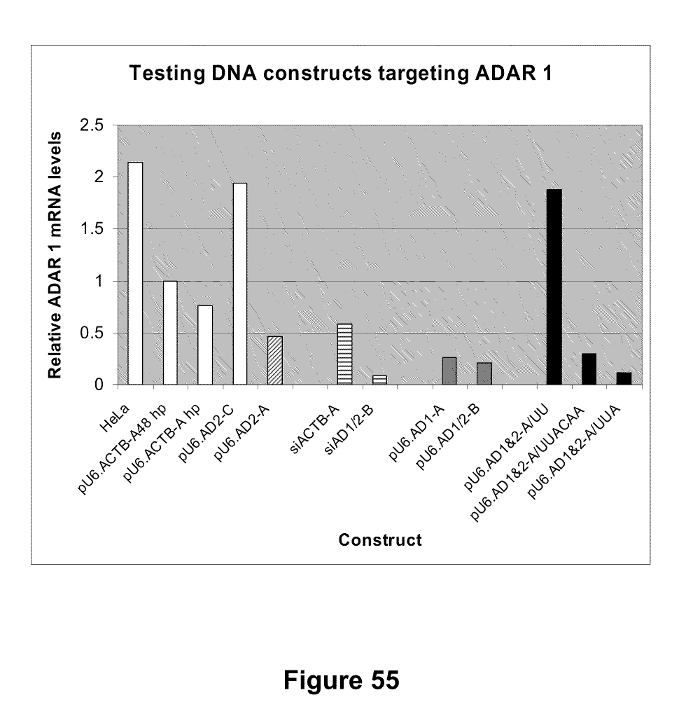

In FIG. 55, the graph shows the relative ADAR 1 mRNA levels in cells transfected with various DNA constructs and siRNAs. All data were normalized to ADAR 1 mRNA levels determined in cells transfected with pU6.ACTB-A48 hp, since this construct produced a hairpin RNA most similar to the double hp constructs. White bars represent ADAR 1 mRNA levels in HeLa cells and in HeLa cells transfected with various non-specific controls. The constructs pU6.ACTB-A hp and pU6.AD2-C had relatively minor effects on ADAR 1 mRNA levels. The construct pU6.AD2-A (the stippled box) which targeted ADAR 2 reduced ADAR 1 mRNA levels; this result might be artifactual but could reflect genuine reductions in ADAR 1 mRNA, since 17/21 nucleotides of the ADAR 2-A site are shared in the ADAR 1 sequence. The horizontally stippled bars represent relative ADAR 1 mRNA levels in HeLa cells transfected with siRNA controls. ACTB-A siRNA had a moderate, non-specific effect on ADAR 1 mRNA levels, whilst siAD1/2-B dramatically reduced ADAR 1 mRNA. The grey bars represent relative ADAR 1 mRNA levels in HeLa cells transfected with the DNA constructs pU6.AD1-A and pU6.AD1/2-B. Both constructs target ADAR 1 mRNA for degradation, and both reduced ADAR 1 mRNA levels markedly. The black bars represent relative ADAR 1 mRNA levels in HeLa cells transfected with the double hairpin DNA constructs, pU6.AD1 &2/UU, pU6.AD1 &2/UUACAA and pU6.AD1 &2/UUA. Both pU6.AD1&2/UUACAA and pU6.AD1&2/UUA markedly reduced ADAR 1 mRNA levels. Most significantly the construct pU6.AD1&2/UUA shows increased activity compared to the control pU6.AD1-A. The construct pU6.AD1 &2/UU showed no activity against ADAR 1 mRNA. These data indicated that the inclusion of a small bubble sequence in a hairpin DNA construct enhanced the activity of a DNA construct, either in the context of a single construct or a double hairpin construct.

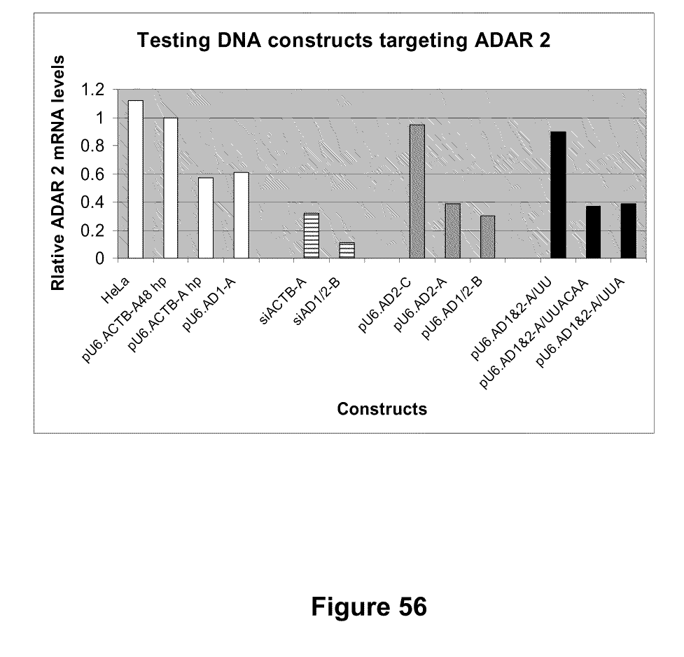

In FIG. 56, the graph shows the relative ADAR 2 mRNA levels in cells transfected with various DNA constructs and siRNAs. All data were normalized to ADAR 2 mRNA levels determined in cells transfected with pU6.ACTB-A48 hp, since this construct produced a hairpin RNA most similar to the double hp constructs. White bars represent ADAR 2 mRNA levels in HeLa cells and in HeLa cells transfected with various non-specific controls. The constructs pU6.ACTB-A hp and pU6.AD1-A have relatively minor effects on ADAR 2 mRNA levels. The horizontally stippled bars represent relative ADAR 1 mRNA levels in HeLa cells transfected with siRNA controls. ACTB-A siRNA had a moderate, non-specific effect on ADAR 1 mRNA levels, whilst siAD1/2-B dramatically reduced ADAR 1 mRNA. The grey bars represent relative ADAR 2 mRNA levels in HeLa cells transfected with the DNA constructs pU6.AD2-C, pU6.AD2-A and pU6.AD1/2-B. The construct pU6.AD2-C has no effect on ADAR 2 mRNA levels, whilst pU6.AD2-A and pU6.ACT1/2-B reduced ADAR 2 mRNA to a moderate degree. The black bars represent relative ADAR 1 mRNA levels in HeLa cells transfected with the double hairpin DNA constructs. The construct pU6.AD1&2/UU showed no activity against ADAR 2 mRNA, as was the case with ADAR 1 mRNA. Both the constructs pU6.AD1&2/UUACAA and pU6.AD1&2/UUA moderately reduced ADAR 2 mRNA levels to a similar degree to that seen for pU6.AD2-A and pU6.AD1/2-B. These data indicated that a short hairpin sequence adjacent to the loop sequence can inactivate a second gene in the context of at least two of the bubble sequences we tested.

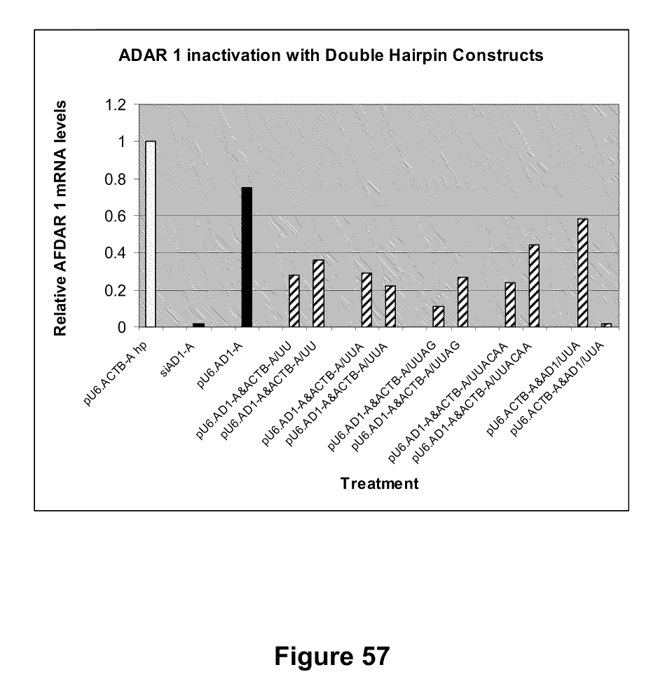

FIG. 57 shows the relative levels of ADAR1 mRNA in cells transfected with various DNA constructs. All data were normalized to cells transfected with pU6.ACTB-A hp which was used as a non-specific control in this experiment. The grey bar shows that construct pU6.AD1-A had a moderate effect on ADAR 1 mRNA levels, similar to that seen in FIG. 55. The constructs pU6.AD1-A&ACTB-A/UU, pU6.AD1-A&ACTB-A/UUA, pU6.AD1-A&ACTB-A/UUAG, pU6.AD1-A&ACTB-A/UU ACAA and pU6.ACTB-A&AD1/UUA all resulted in reductions in ADAR 1 mRNA levels, the construct pU6AD1-A&ACTB-A/UUAG showed the highest activity. These data demonstrated that ADAR 1 sequences in the context of a double hairpin can inactivate ADAR1 mRNA.

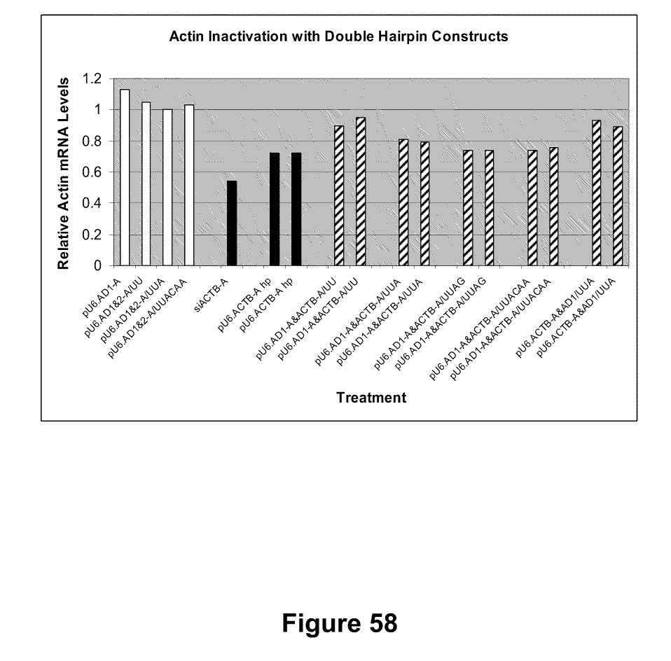

FIG. 58 shows the relative levels of β actin mRNA in cells transfected with various DNA constructs, as determined by quantitative Northern blot analyses. In this instance all data are normalized to the construct pU6.AD1&2-A/UUA. Various non-specific controls (pU6.Ad1-A, pU6.AD1&2-A/UU, pU6.AD1&2-A/UUA and pU6.AD1&2-A/UUACAA) showed essentially no effect on β actin mRNA levels. Cells transfected with the construct pU6.ACTB-A hp showed an approximately 30% reduction in β actin mRNA levels. Cells transfected with the constructs pU6.AD1-A&ACTB-A/UU, pU6.AD1-A&ACTB-A/UUA, pU6.AD1-A&ACTB-A/UUAG, pU6AD1-A&ACTB-A/UUACAA and pU6.ACTB-A&AD1/UUA all showed reductions in the levels of β actin mRNA, the construct pU6.AD1-A&ACTB-A/UUAG showed the highest activity. These data demonstrated that β actin sequences in the context of a double hairpin can inactivate β actin mRNA. These data combined with the results shown in FIG. 57 demonstrate that a double hairpin construct can simultaneously inactivate two genes.

### 11. Increasing the Activity of Double Hairpin Constructs by Screening Random Libraries of “Bubble” Sequences

To define sequences that may increase the activity of double and higher order hairpin constructs, a series of libraries are prepared containing randomised sequences in regions of the hp RNAs that might be predicted to be sites for Dicer processing.

The base construct for these experiments is the construct pU6.GR-21. This was prepared using the oligonucleotide annealing strategy described above, using the primers:

LGR-1

(SEQ ID NO: 124)

ACCGCTGACCCTGAAGTTCATCCTGGCCTTTC
LGR-2

(SEQ ID NO: 125)

GGAGTAGTGAAAGGCCAGGATGAACTTCAGGGTCAG
LGR-3

(SEQ ID NO: 126)

ACTACTCCTACTTTGTGTAGGT
LGR-4

(SEQ ID NO: 127)

ACTACTCCTACCTACACAAAGTA
LGR-5

(SEQ ID NO: 128)

AGGAGTAGTGAAAGGCCAGGATGAACTTC
LGR-6

(SEQ ID NO: 129)

CTGACCCTGAAGTTCATCCTGGCCTTTC
LGR-7

(SEQ ID NO: 130)

AGGGTCAGCTTTTTTGGAAA
LGR-8

(SEQ ID NO: 131)

AGCTTTTCCAAAAAAG

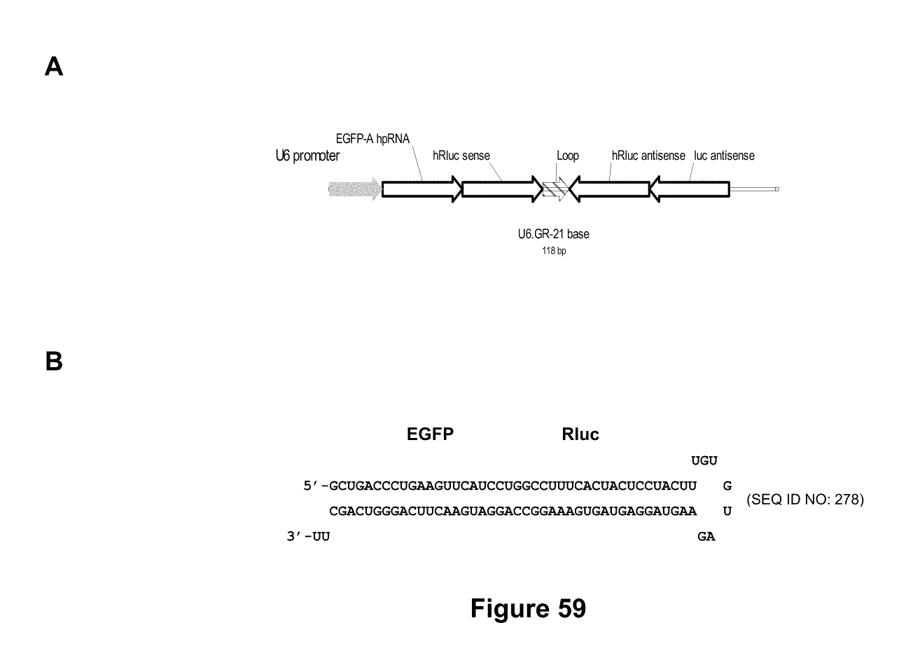

A map of the construct is shown in FIG. 59A and the sequence (SEQ ID NO: 278) of the predicted RNA produced by the construct is shown in FIG. 59B.

The library constructs described below all contain identical sequences targeting Renilla luciferase at the top position of the double hairpin construct. By comparing the activity of individual clones from the library against Renilla luciferase as described above to the activity of pU6.GR-21 sequences of bubbles showing enhanced activity might be determined. Based on such data, generalised design rules to enhance the activity of double, and higher order, hairpin constructs may be developed.

pU6.GR-21-1-2N

This construct series was prepared using the oligonucleotide assembly strategy described above. Libraries were prepared using the oligonucleotides:

LGR-1-2N

(SEQ ID NO: 132)

ACCGCTGACCCTGAAGTTCATCCNNGCCTTTC
LGR-2-2N

(SEQ ID NO: 133)

GGAGTAGTGAAAGGCNNGGATGAACTTCAGGGTCAG
LGR-3

(SEQ ID NO: 134)

ACTACTCCTACTTTCAGTAGGT
LGR-4

(SEQ ID NO: 135)

ACTACTCCTACCTACACAAAGTA
LGR-5

(SEQ ID NO: 136)

AGGAGTAGTGAAAGGCCAGGATGAACTTC
LGR-6

(SEQ ID NO: 137)

CTGACCCTGAAGTTCATCCTGGCCTTTC
LGR-7

(SEQ ID NO: 138)

AGGGTCAGCTTTTTTGGAAA
LGR-8

(SEQ ID NO: 139)

AGCTTTTCCAAAAAAG

In this instance N denotes any nucleotide. A map of such constructs is shown in FIG. 60A and the sequence of the predicted RNA (SEQ ID NO: 279) produced by the construct is shown in FIG. 60B.

pU6.GR-21-4-2N

This construct series was prepared using the oligonucleotide assembly strategy described above. Libraries were prepared using the oligonucleotides:

LGR-1

(SEQ ID NO: 140)

ACCGCTGACCCTGAAGTTCATCCTGGCCTTTC
LGR-2

(SEQ ID NO: 141)

GGAGTAGTGAAAGGCNNGGATGAACTTCAGGGTCAG
LGR-3

(SEQ ID NO: 142)

ACTACTCCTACTTTGTGTAGGT
LGR-4

(SEQ ID NO: 143)

ACTACTCCTACCTACACAAAGTA
LGR-5-2N

(SEQ ID NO: 144)

AGGAGTAGTGAAAGGCCANNATGAACTTC
LGR-6-2N

(SEQ ID NO: 145)

CTGACCCTGAAGTTCATNNTGGCCTTTC
LGR-7

(SEQ ID NO: 146)

AGGGTCAGCTTTTTTGGAAA
LGR-8

(SEQ ID NO: 147)

AGCTTTTCCAAAAAAG

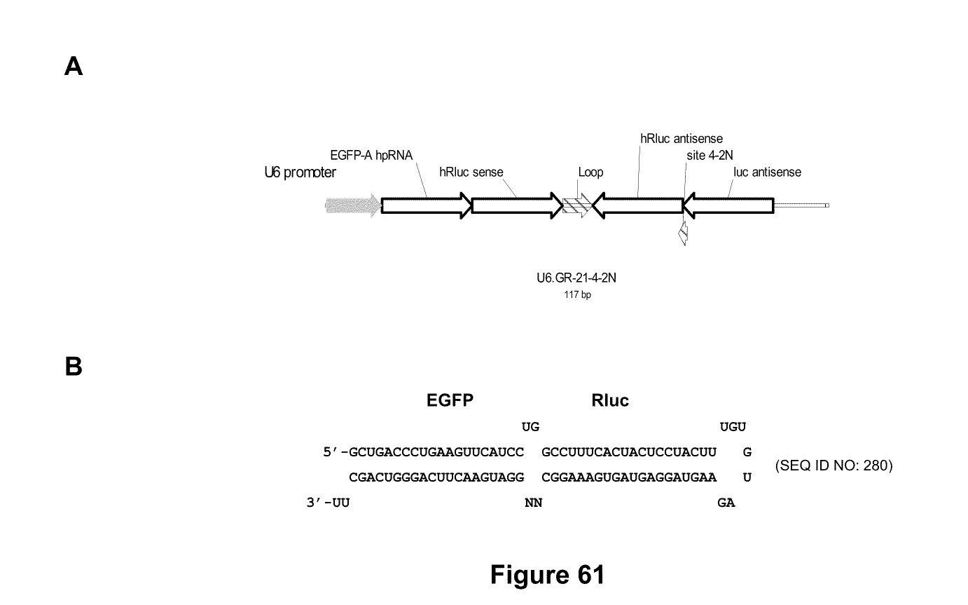

In this instance N denotes any nucleotide. A map of such constructs is shown in FIG. 61A and the sequence of the predicted RNA (SEQ ID NO: 280) produced by the construct is shown in FIG. 61B.

pU6.GR-21-1&4-2N

This construct series may be prepared using the oligonucleotide assembly strategy described above. Libraries may be prepared using the oligonucleotides:

LGR-1-2N

(SEQ ID NO: 148)

ACCGCTGACCCTGAAGTTCATCCNNGCCTTTC
LGR-2-2N

(SEQ ID NO: 149)

GGAGTAGTGAAAGGCNNGGATGAACTTCAGGGTCAG
LGR-3

(SEQ ID NO: 150)

ACTACTCCTACTTTGTGTAGGT
LGR-4

(SEQ ID NO: 151)

ACTACTCCTACCTACACAAAGTA
LGR-5-2N

(SEQ ID NO: 152)

AGGAGTAGTGAAAGGCCANNATGAACTTC
LGR-6-2N

(SEQ ID NO: 153)

CTGACCCTGAAGTTCATNNTGGCCTTTC
LGR-7

(SEQ ID NO: 154)

AGGGTCAGCTTTTTTGGAAA
LGR-8

(SEQ ID NO: 155)

AGCTTTTCCAAAAAAG

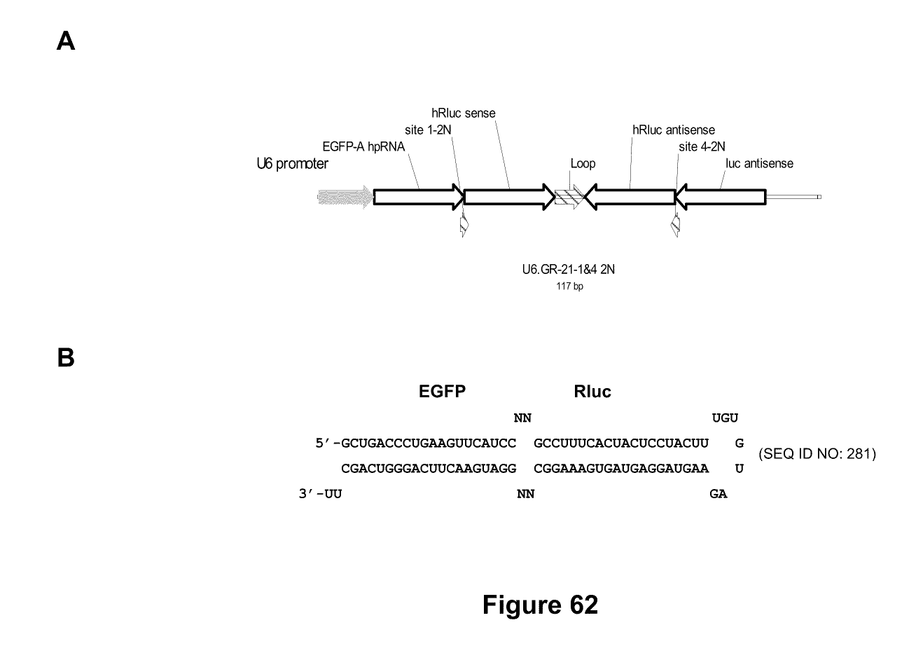

In this instance N denotes any nucleotide. A map of such constructs is shown in FIG. 62A and the sequence of the predicted RNA (SEQ ID NO: 281) produced by the construct is shown in FIG. 62B.

pU6.GR22-1-4N

This construct series may be prepared using the oligonucleotide assembly strategy described above. In this instance random oligonucleotides are not used, rather three nucleotides which are incapable of base pairing in the predicted hpRNA are incorporated synthetically. To generate the constructs, the oligonucleotides GR5-22, GR6-22, GR7 and GR8 are annealed together with:

| | |
|---|---|
| GR22-1-4N-1 | |
| ACCGCTGACCCTGAAGT | (SEQ ID NO: 156) |
| GR22-1-4N-2 | |
| AGTGAAAGGDDBHAGATGAACTTCAGGGTCAG | (SEQ ID NO: 157) |
| GR22-1-4N-3 | |
| TCATCTDVHHCCTTTCACTACTCCTACTTTGTG | (SEQ ID NO: 158) |
| GR22-1-4N-4 | |
| CTCCTACCTACACAAAGTAGGAGT | (SEQ ID NO: 159) |

In this instance, D denotes A,G or T; B denotes C,G or T; H denotes A,C or T and V denotes A,C or G. A map of such constructs is shown in FIG. 63A and the predicted sequence (SEQ ID NO: 282) and structure of hpRNAs produced from such constructs is shown in FIG. 63B.

pU6.GR22-1-4N

This construct series may be prepared using the oligonucleotide assembly strategy described above. In this instance random oligonucleotides are not used, rather three nucleotides which are incapable of base pairing in the predicted hpRNA are incorporated synthetically. To generate the constructs, the oligonucleotides GR1, GR2-22, GR3-22, GR4, GR7 and GR8 are annealed together with:

| | |
|---|---|
| GR22-4-4N-5 | |
| TAGGTAGGAGTAGTGAAAGGDDBHAGATGAA | (SEQ ID NO: 160) |
| GR22-4-4N-6 | |
| ACCCTGAAGTTCATCTDVHHCCTTTCACTA | (SEQ ID NO: 161) |

In this instance, D denotes A,G or T; B denotes C,G or T; H denotes A,C or T and V denotes A,C or G. A map of such constructs is shown in FIG. 63C and the predicted sequence (SEQ ID NO: 283) and structure of hpRNAs produced from such constructs is shown in FIG. 63D.

### PU6.GR22-1-NAAN

This construct series may be prepared using the oligonucleotide assembly strategy described above. In this instance random oligonucleotides may be incorporated to screen for sequences that may augment the optimal AA sequence identified previously. To generate the constructs, the oligonucleotides GR22-1-4N-1, GR2-1-4N-4, GR5-22, GR6-22, GR7 and GR8 are annealed together with:

| | |
|---|---|
| GR22-1-NAAN-2 | |
| AGTGAAAGGNTTNAGATGAACTTCAGGGTCAG | (SEQ ID NO: 162) |
| GR22-1-NAAN-3 | |
| TCATCTNAANCCTTTCACTACTCCTACTTTGTG | (SEQ ID NO: 163) |

In this instance N denotes any nucleotide. A map of such constructs is shown in FIG. 64A and the predicted sequence (SEQ ID NO: 284) and structure of hpRNAs produced from such constructs is shown in FIG. 64B.

### PU6.GR22-4-NAAN

This construct series may be prepared using the oligonucleotide assembly strategy described above. In this instance random oligonucleotides may be incorporated to screen for sequences that might potentially augment the optimal AA sequence defined previously.

To generate the constructs, the oligonucleotides GR1, GR2-22, GR3-22, GR4, GR7 and GR8 are annealed together with:

GR22-4-NAAN-5

| | |
|---|---|
| TAGGTAGGAGTAGTGAAAGGNAANAGATGAA | (SEQ ID NO: 164) |

GR22-4-NAAN-6

| | |
|---|---|
| ACCCTGAAGTTCATCTNTTNCCTTTCACTA | (SEQ ID NO: 165) |

In this instance N denotes any nucleotide. A map of such constructs is shown in FIG. 64C and the predicted sequence (SEQ ID NO: 285) and structure of hpRNAs produced from such constructs is shown in FIG. 64D. pU6.GR21-1&4-4N

This construct series may be prepared using the long range PCR strategy described above. In this instance random oligonucleotides are not used, rather three nucleotides which are incapable of conventional base pairing in the predicted hp RNA are incorporated. The oligonucleotides suitable for use in these experiments are:

LU6GR-21-4N
(SEQ ID NO: 166)
CACAAAGTAGGAGTAGTGAAAGGDDBHGATGAACTTCAGGGTCAGCGG
TGTTTCGTCCTTTC
and
LtermGR-21-4N
(SEQ ID NO: 167)
TAGGTAGGAGTAGTGAAAGGCCBHHBTGAACTTCAGGGTCAGCTTTTTT
GGAAAAGCTTATCG

In this instance, D denotes A,G or T; B denotes C,G or T, H denotes A,C or T and V denotes A,C or G. A map of such constructs is shown in FIG. 65A and the sequence (SEQ ID NO: 286) of the predicted RNA produced by the construct is shown in FIG. 65B.

### 12. Phasing Constructs

Based on the model whereby Dicer processes from the base of an expressed hpRNA, the actual distance (in nucleotides) between dicer cuts becomes a critical factor in designing multi-constructs to obtain maximum activity, since this “phasing” of Dicer processing will be critical in precisely defining the sequence of effector siRNAs produced from a hpRNA. To determine the optimal phasing a series of constructs were prepared which were designed to express variable lengths of EFGP effector sequences at the base of a double hairpin construct and constant sequences at the top, targeting Rluc.

The constructs were prepared using the oligonucleotide assembly strategy and cloned into BsmBI/Hind III digested pU6.cass as described above. The constructs and oligonucleotides used to prepare the constructs were:

| | | |
|---|---|---|
| pU.GR-17 hp | | |
| GR1 | ACCGCTGACCCTGAAGTTC | (SEQ ID NO: 168) |
| GR2-17 | GAAAGGCCAGAACTTCAGGGTCAG | (SEQ ID NO: 169) |
| GR3-17 | TGGCCTTTCACTACTCCTACTTTGTG | (SEQ ID NO: 170) |
| GR4 | CTCCTACCTACACAAAGTAGGAGTAGT | (SEQ ID NO: 171) |
| GR5-17 | TAGGTAGGAGTAGTGAAAGGCCAGAA | (SEQ ID NO: 172) |
| GR6-17 | ACCCTGAAGTTCTGGCCTTTCACT | (SEQ ID NO: 173) |
| GR7 | CTTCAGGGTCAGCTTTTTTGGAAA | (SEQ ID NO: 174) |
| GR8 | AGCTTTTCCAAAAAAGCTG | (SEQ ID NO: 175) |
| pU.GR-18 hp | | |
| GR1, GR4, GR7, GR8 and: | | |
| GR2-18 | GAAAGGCCATGAACTTCAGGGTCAG | (SEQ ID NO: 176) |
| GR3-18 | ATGGCCTTTCACTACTCCTACTTTGTG | (SEQ ID NO: 177) |
| GR5-18-2 | TAGGTAGGAGTAGTGAAAGGCCATGAA | (SEQ ID NO: 178) |
| GR6-18-2 | ACCCTGAAGTTCATGGCCTTTCACTA | (SEQ ID NO: 179) |
| pU6.GR-19 hp | | |
| GR1, GR4, GR7, GR8 and: | | |
| GR2-19 | GAAAGGCCAATGAACTTCAGGGTCAG | (SEQ ID NO: 180) |
| GR3-19 | ATTGGCCTTTCACTACTCCTACTTTGTG | (SEQ ID NO: 181) |
| GR5-19 | TAGGTAGGAGTAGTGAAAGGCCAGTGAA | (SEQ ID NO: 182) |
| GR6-19 | ACCCTGAAGTTCACTGGCCTTTCACTA | (SEQ ID NO: 183) |
| pU6.GR-20 hp | | |
| GR1, GR4, GR7, GR8 and: | | |
| GR2-20 | GAAAGGCCAGATGAACTTCAGGGTCAG | (SEQ ID NO: 184) |
| GR3-20 | ATCTGGCCTTTCACTACTCCTACTTTGTG | (SEQ ID NO: 185) |
| GR5-20 | TAGGTAGGAGTAGTGAAAGGCCAGATGAA | (SEQ ID NO: 186) |
| GR6-20 | ACCCTGAAGTTCATCTGGCCTTTCACTA | (SEQ ID NO: 187) |
| pU6.GR-21 hp | | |
| GR1, GR4, GR7, GR8 and: | | |
| GR2-21 | GAAAGGCCAGGATGAACTTCAGGGTCAG | (SEQ ID NO: 188) |
| GR3-21 | ATCCTGGCCTTTCACTACTCCTACTTTGTG | (SEQ ID NO: 189 |
| GR5-21 | TAGGTAGGAGTAGTGAAAGGCCAGGATGAA | (SEQ ID NO: 190) |
| GR6-21 | ACCCTGAAGTTCATCCTGGCCTTTCACTA | (SEQ ID NO: 191) |
| pU6.GR-22 hp | | |
| GR1, GR4, GR7, GR8 and: | | |
| GR2-22 | GAAAGGCCAGAGATGAACTTCAGGGTCAG | (SEQ ID NO: 192 |
| GR3-22 | ATCTCTGGCCTTTCACTACTCCTACTTTGTG | (SEQ ID NO: 193) |
| GR5-22 | TAGGTAGGAGTAGTGAAAGGCCAGAGATGAA | (SEQ ID NO: 194) |
| GR6-22 | ACCCTGAAGTTCATCTGCTGGCCTTTCACTA | (SEQ ID NO: 195) |
| pU6.GR-23 hp | | |
| GR1, GR4, GR7, GR8 and: | | |
| GR2-23 | GAAAGGCCAGCAGATGAACTTCAGGGTCAG | (SEQ ID NO: 196) |
| GR3-23 | ATCTGCTGGCCTTTCACTACTCCTACTTTGTG | (SEQ ID NO: 197) |
| GR5-23 | TAGGTAGGAGTAGTGAAAGGCCAGCAGATGAA | (SEQ ID NO: 198) |
| GR6-23 | ACCCTGAAGTTCATCTGCTGGCCTTTCACTA | (SEQ ID NO: 199) |
| pU6.GR-24 hp | | |
| GR1, GR4, GR7, GR8 and: | | |
| GR2-24 | GAAAGGCCAGGCAGATGAACTTCAGGGTCAG | (SEQ ID NO: 200) |
| GR3-24 | ATCTGCCTGGCCTTTCACTACTCCTACTTTGTG | (SEQ ID NO: 201) |
| GR5-24 | TAGGTAGGAGTAGTGAAAGGCCAGGCAGATGAA | (SEQ ID NO: 202) |
| GR6-24 | ACCCTGAAGTTCATCTGCCTGGCCTTTCACTA | (SEQ ID NO: 203) |
| pU6.GR-25 hp | | |
| GR1, GR4, GR7, GR8 and: | | |
| GR2-25 | GAAAGGCCAGTGCAGATGAACTTCAGGGTCAG | (SEQ ID NO: 204) |
| GR3-25 | ATCTGCACTGGCCTTTCACTACTCCTACTTTGTG | (SEQ ID NO: 205) |
| GR5-25 | TAGGTAGGAGTAGTGAAAGGCCAGTGCAGATGAA | (SEQ ID NO: 206) |
| GR6-25 | ACCCTGAAGTTCATCTGCACTGGCCTTTCACTA | (SEQ ID NO: 207) |
| pU6.GR-26 hp | | |
| GR1, GR4, GR7, GR8 and: | | |
| GR2-26 | GAAAGGCCAGGTGCAGATGAACTTCAGGGTCAG | (SEQ ID NO: 208) |
| GR3-26 | ATCTGCACCTGGCCTTTCACTACTCCTACTTTGTG | (SEQ ID NO: 209) |
| GR5-26 | TAGGTAGGAGTAGTGAAAGGCCAGGTGCAGATGAA | (SEQ ID NO: 210) |
| GR6-26 | ACCCTGAAGTTCATCTGCACCTGGCCTTTCACTA | (SEQ ID NO: 211) |

Examples of phasing constructs are shown in FIG. 66. The sequence (SEQ ID NO: 287-296) and predicted structure of the hpRNAs produced by these constructs are shown in FIG. 67.

These constructs were transformed into transgenic Rluc-expressing HeLa cells and Rluc activity determined as described above. Results of these experiments are shown in FIG. 68. Note that the constructs pU6.GR-21 hp show the greatest activity. Phasing of 21, or preferably 22 nt is therefore optimal for multiple hpRNAs.

### 13. Screening 2N Libraries

Plasmid DNAs from randomly picked clones from the pU6.GR-21-1-2N and pU6.GR-21-4-2N libraries were prepared and screened for activity against Rluc in transgenic HeLa cells as described above.

A total of 22 clones from the pU6.GR-21-4-2N library were screened in this fashion. None of these clones showed increased activity (Data not shown).

A total of 38 clones from the pU6.GR-21-1-2N library were screened in this fashion. Data from 22 clones are shown in FIG. 69A. In this experiment the activity of these clones was compared to a control pU6.ACTB Rluc TTA, however the activity of this clone was considered to be unusually high in this particular experiment. Consequently, the activity of the three best clones was retested, results are shown in FIG. 69B. The data demonstrate that the clone pU6.GR-21-1-2N-18 showed enhanced activity compared to the most appropriate control, pU.6GR-21 hp and these data are confirmed in FIG. 69B.

Upon sequencing it was shown that pU6.GR-21-1-2N-18 had the sequence AA between positions 21 and 22 of the predicted hpRNA.

### 14. Inactivation of Multiple Genes Using Constructs Containing Multiple Transcriptional Units

An alternative approach to inactivating multiple genes, is to express multiple transcripts from a single construct. An example of such a construct is shown in FIG. 71.

This construct pU6.GF-3 (FIG. 71D) may be prepared from two precursors, pU6.GL (FIG. 71A) and pU6 GG-4 (FIG. 33 and FIG. 71C). pU6.GL targets murine Akt1 at the same region of Akt1 as the double construct pU6.GF-2 shown in FIG. 33. pU6.GL is made using the long range PCR strategy described above; Bgl II, SAP-treated pU6.cass lin is amplified using the primers:

U6 GL

(SEQ ID NO: 212)

CACAAACAGCTTCTCGTGGTCCTGGCGGTGTTTCGTCCTTTC
term GL

(SEQ ID NO: 213)

TAGCAGCTTCTCGTGGTCCTGGTTTTTTGGAAAAGCTTATCG

A map of a portion of pU6.GL is shown in 71A, the positions of Sma I and Kpn I cloning sites in the plasmid are also shown. The predicted transcript produced (SEQ ID NO: 297) from this plasmid is shown in FIG. 71B. pU6.GF-3 may be prepared by cloning the U6 transcriptional unit from pU6.GL as a Sma I/Kpn I fragment into Hinc II/Kpn I digested pU6.GG-4 to produce pU6.GF-3. pU6.GF-3 will contain two U6 transcriptional units as shown in FIG. 71D, and is designed to express two separate hairpin RNAs, one targeting Akt1, the other targeting Akt2. The activity of this construct may be determined as described above (Example 8).

### 15. Constructs Targeting HCV

One disease state that may be treated with the multiple target interfering RNA nucleic acid constructs of the present invention is hepatitis C virus (HCV) infection. Based on statistics compiled from the Centers for Disease Control and Prevention, almost 2% of the American population (nearly 4 million people) is currently infected with HCV. Initially, the majority of the individuals infected with HCV exhibit no symptoms; however, greater than 80% will develop chronic and progressive liver disease eventually leading to cirrhosis or hepatocellular carcinomas. HCV is the leading indication for liver transplantation within the United States and results in the death of 8,000 to 10,000 Americans every year. On a global level, the World Heath Organization estimates that there are more than 170 million affected individuals, with infection rates as high as 10-30% of the general population in some countries.

HCV is a positive-sense single stranded enveloped RNA virus belonging to the Flaviviridae family. The infectious cycle of HCV typically begins with the entry of the viral particle into the cell by receptor-mediated binding and internalization. After uncoating in the cytoplasm, the positive strand of RNA that comprises the genome can interact directly with the host cell translational machinery. Lacking 5′ cap methylation, the RNA forms an extensive secondary structure in the 5′ untranslated Region (UTR) that serves as an internal ribosomal entry site (IRES) and permits the direct binding of the 40S subunit as the initiating step of the translation process.

The HCV genome, approximately 9600 nucleotides in length, encodes a single long open reading frame termed the polyprotein. Viral proteins are produced as linked precursors from the polyprotein which is subsequently cleaved into mature products by a wide variety of viral and cellular enzymes. Encoded amongst the genes are the structural proteins, including the core and envelope glycoproteins, so named because they are integral structural components in progeny virions. Non-structural proteins, which provide indispensable functions such as the RNA dependent RNA polymerase, are also produced. The viral replication machinery is established within the cytoplasm of infected cells that transcribe the positive-sense RNA into a negative strand intermediate. Thus, the HCV genomic RNA serves as both a template for its own replication and as a messenger RNA for translation of the virally encoded proteins. The negative strand is transcribed back into a positive strand of RNA, thereby amplifying the number of positive strand copies within the cell. At this stage, the positive strand can interact with the host cell translational machinery once again or, if there have been enough structural proteins accumulated, be packaged into virions. Following egress from the cell, the virus repeats its infectious cycle.

Although many of the individual steps of HCV replication are understood, until recently there was no tissue culture system that propagated the viral life cycle, making studies of the virus difficult. However, an in vitro replicon system has been developed (see, e.g., U.S. Pat. Nos. 5,585,258; 6,472,180; and 6,127,116 to Rice, et al.). A replicon is an autonomously replicating portion of HCV genomic RNA containing a marker gene for selection and verification of replication. HCV-RNA constructs are transfected into cell lines that are amenable to support continuous propagation. Following the steps of the infectious cycle, the RNA is translated by the cellular machinery and produces the appropriate viral proteins required for replication of the genome are produced, as is the selectable marker. Full-length and sub-genomic replicons have been generated and shown to be functional, although only the non-structural proteins are obligate. The autonomously replicating properties of the RNA remain independent of expression of the structural genes. Even when present in replicons expressing the full length HCV genome, the core and envelope proteins fail to effectively package the genome into infectious particles, resulting in the loss of a model system to study the packaging, egress and re-entry steps of the virus. Regardless, the replicon is able to recreate a portion of the biology and mechanisms utilized by HCV.

### Development of an AAV-2 Expression Vector for In Vivo Delivery of Interfering RNA According to the Present Invention

Before the delivery of interfering RNA nucleic acid constructs according to the present invention by infectious particles is tested, the appropriate expression plasmid is constructed and validated. AAV-2 vectors which have been gutted of rep and cap provide the backbone (hereinafter referred to as the rAAV vector) for the viral interfering RNA nucleic acid construct. This vector has been extensively employed in AAV studies and the requirements for efficient packaging are well understood. The U6 and H1 promoters may be used for the expression of interfering RNA according to the present invention, though there have been reports of vastly different levels of inhibition of an identical interfering RNA driven independently by each promoter. However, vector construction is such that promoters can be easily swapped if such variation is seen.

As with virtually any viral delivery system, the rAAV vector must meet certain size criteria in order to be packaged efficiently. In general, an rAAV vector must be 4300-4900 nucleotides in length (McCarty, et al. Gene Ther. 8: 1248-1254 (2001)). When the rAAV vector falls below the limit, a ‘stuffier’ fragment must be added (Muzyczka, et al. Curr. Top. Microbiol. Immunol. 158: 970129 (1992)). In the AAV vector embodiment described here, one or more selectable marker genes may be engineered into the rAAV interfering RNA nucleic acid construct in order to assess the transfection efficiency of the rAAV interfering RNA nucleic acid construct as well as allow for quantification of transduction efficiency of target cells by the rAAV interfering RNA nucleic acid construct delivered via infectious particles.

The initial test expression construct drives expression of interfering RNAs designed from sequences with demonstrated ability to inhibit luciferase activity from a reporter construct (see, Elbashir, et al. Embo. J. 20(23): 6877-6888 (2001)). A commercially available expression plasmid that encodes for the production of luciferase functions as the reporter to verify the ability of the various interfering RNAs to downregulate the target sequences.

Although the interfering RNAs against luciferase have been previously validated, the efficacy of rAAV-delivered interfering RNAs is assessed in vitro prior to testing the construct in vivo. The test and reporter constructs are transfected into permissive cells utilizing standard techniques. An rAAV expression construct in which the luciferase-specific RNAi agent has been replaced by an unrelated RNA sequence is utilized as a negative control in the experiments. The relative percentage of transfection efficiency is estimated directly by assessing the levels of the selective marker using fluorescence microscopy. For assessing inhibitory activity of each different RNAi agent, luciferase activity is measured utilizing standard commercial kits. Alternatively, quantitative real time PCR analysis (Q-PCR) is run on RNA that is harvested and purified from parallel experimental plates. Activity decreases greater than about 70%, relative to the activity recovered in lysates from cells treated with the unrelated RNA species, are an indication that the RNAi agent is functional.

Subsequent experiments are performed in order to assess the effects of interfering RNAs on a luciferase reporter system that is transfected into the livers of mice, similar to the work of McCaffrey et al. in Nature, 418: 38-39 (2002). Nucleic acids delivered to mice by hydrodynamic transfection methods (high pressure tail vein injection) primarily localized to the livers. Much like the principle which governs co-transfection in cell culture, simultaneous injection of multiple plasmids from a mixture often permits the penetrance of all of the expression constructs into the same cell. Thus, even though the tail vein injection procedures are well documented to only transfect 5-40% of the hepatocytes within the liver (McCaffrey, et al. Nature Biotech. 21(6): 639-644 (2003)), co-injection permits delivery of the reporter system and the expression construct into the same cells.

The rAAV nucleic acid construct bearing the interfering RNA targeted against luciferase is co-injected with the reporter construct that encodes for the luciferase gene. In animals receiving the negative control, an expression construct bearing an unrelated RNA is co-injected with the reporter construct. After seven days, the mice are sacrificed and the livers harvested. Luciferase activity is measured on lysates generated from a portion of the liver. Remaining portions of the liver are utilized for Q-PCR measurements as well as histological analysis to determine marker protein expression for normalization of the data. Alternative methods to assess transfection efficiency may include ELISA measurements of serum from mice that have been co-injected with a third marker plasmid for a secreted protein such as human α1-antitrypsin (hAAT) (Yant, et al. Nature Genetics. 25: 35-41 (2000), see also McCaffrey, et al. Nature Biotech. 21(6): 639-644 (2003)).

Once it is established that the nucleic acid construct is functional in both in vitro cell culture systems as well as in vivo mouse models by utilizing co-transfection of the naked DNA plasmids, testing is initiated on the rAAV expression construct packaged into infectious particles. The infectious particles are produced from a commercially available AAV helper-free system that requires the co-transfection of three separate expression constructs containing 1) the rAAV nucleic acid construct expressing the interfering RNA against luciferase (flanked by the AAV ITRs); 2) the construct encoding the AAV rep and cap genes; and 3) an expression construct comprising the helper adenovirus genes required for the production of high titer virus. Following standard purification procedures, the viral particles are ready for use in experiments.

Before mice can be infused with the rAAV particles, a reporter system is established in the mouse livers. Hydrodynamic transfection is employed to deliver the luciferase reporter construct as well as an expression plasmid for hAAT to control for differences in transfection efficiencies from animal to animal. The mice are permitted to recover for several days in order to establish sufficient levels of reporter activity. After luciferase reporter activity has been established in the livers, AAV particles are infused into normal C57B1/6 mice either through portal vein or tail vein injection. AAV particles bearing the expression construct of an unrelated RNA are used as a negative control. Initially, the mice are infused with relatively high doses (2×1012 vector genomes (vg)) which are reduced in follow-up experiments performed to generate dose-response curves. After seven to ten days, the mice are sacrificed, the livers harvested and samples of serum collected. The relative levels of hepatic luciferase activity and RNA are determined from the isolated livers utilizing the luciferase assay and QPCR procedures previously described. Additionally, the efficiency of transduction is assessed by measurement of the marker protein in serial slices of the hepatic tissues.

It has been estimated that hydrodynamic transfection procedures may result in the transfection of 5-40% of hepatocytes. Transduction of liver cells by AAV-2 delivery procedures have been shown to result in 5-10% transduction efficiencies. Although AAV may preferentially transduce the same pool of hepatocytes that were transfected by the initial tail vein injection procedure, it is possible that the subsets of cells that each technique affects are non-overlapping. If the former occurs, a reduction in luciferase activity relative to mice transduced with an unrelated interfering RNA is seen. If the latter occurs, then no decrease in luciferase activity is seen.

### Modifications to Enhance Efficiency of AAV Transduction of Liver Tissues

Although it has been demonstrated that AAV-based vectors can deliver desired sequences to hepatocytes, the relative level of transduction that occurs within those tissues has been rather poor. For current clinical hemophilia studies which employ AAV-2 to deliver and express blood factor IX, this is not a significant issue. For treatment of hemophilia, it is critical only to replenish levels of secreted protein to therapeutic levels. Such replenishment may occur from a small number of transduced cells able to express significant levels of the desired protein. However, because the mechanism of interfering RNA action is intracellular and the effect is not transmitted directly from cell to cell, the transduction efficiency must be increased in order for AAV expressing interfering RNAs to be utilized as a therapeutic.

McCarty et al. were able to generate a self complementary AAV vector (scAAV) that has both a plus and a minus strand of the same expression cassette within its capsid (Gene Ther. 8: 1248-1254 (2001)). This was achieved by mutating the 5′ ITR and leaving the 3′ ITR intact. By mutating or deleting the terminal resolution site other non-essential AAV sequences, thus eliminating possible recombination by wild type AAV and this construct, a DNA template is created where replication starts at the 3′ ITR. Once the replication machinery reaches the 5′ ITR, no resolution takes place and replication continues to the 3′ ITR. The resulting product has both a plus and complementary minus strand, yet is efficiently packaged. Employing the scAAV vectors, transduction of liver cells was increased to 30% of the total hepatocytes (Fu, et al. Molec Therapy. 8(6):911-917 (2003)). When delivered intercisternally, more than 50% of the Purkinje cells in the cerebellum were transduced by the scAAV particles. Thomas et al. showed that self-complementary vectors could produce 50-fold higher luciferase transgene expression levels in mouse livers than their corresponding single-stranded AAV counterparts when infused into mouse livers at equivalent doses (Thomas et al., J. Virol (2004), 78:6 p. 3110-3122). Though dropping slightly, the relative difference of expression between the vectors persisted at 20-fold nearly one year after injection.

Other modifications of AAV-delivery systems also have been used to dramatically enhance transduction efficiencies, including the production of pseudotyped viral particles by packaging rAAV-2 vector genomes with the Cap protein from other serotypes. Because they have been among the best characterized of all of the serotypes, the Cap proteins from AAV-1 through AAV-6 are used most commonly to pseudotype the AAV-2 vectors. Even with the advantages gained by these employing pseudotyping strategies, the threshold of transduction efficiency of hepatocytes may be increased only to 15% of the total population. However, dozens of other serotypes of AAV have been isolated and identified, but have not been characterized to any appreciable degree. For example, one of these is AAV-8, which was isolated originally from the heart tissue of a rhesus monkey. In an effort to determine effects novel cap proteins on transduction, pseudotyped virus in which the single stranded AAV-2 genome was pseudotyped with AAV-8 cap was created. The vectors carried the LacZ gene to assess the relative efficiency of transduction of mouse livers after infusion with increasing doses of infectious particles. A summary of the results (Thomas, et al. (2004)) is shown below in Table 1:

TABLE 1
AAV-2/2 and AAV-2/8 Dose Responses-(% beta-gal positive hepatocytes)

| | Dose (v.g./mouse) | | | | |
|---|---|---|---|---|---|
| Vector | 5 × 10¹⁰ | 3 × 10¹¹ | 1.8 × 10¹² | 3.9 × 10¹² | 7.2 × 10¹² |
| AAV-2 | 0.6 ± 0.4% | 3.0 ± 0.5% | 8.1 ± 1.0% | 8.9 ± 1.0% | NA |
| nlslac Z | | | | | |
| AAV-8 | 8.1 ± 1.8% | 14.9 ± 3.4% | 65.8 ± 9.1% | NA | 97.4 ± 0.3% |
| nlslac Z | | | | | |

As the dose of infused control AAV-2/2 particles is increased, there is a modest increase in transduction of hepatocytes; however, the upper threshold of transduction remains entrenched near the 10% limit. Surprisingly, pseudotyped AAV-2/8 particles transduced 8% of hepatocytes at the lowest dose of particles administered; doses that were 30-80 fold less than their AAV-2/2 counterparts. Additionally, the dose-dependent increase in transduction efficiency for AAV-2/8 surpassed the transduction efficiency for AAV-2/2 to greater than 97% at the highest dose. Transduction efficiencies within this range enable to efficient delivery of interfering RNA to cells within tissues.

Similar modifications of AAV are engineered into the rAAV interfering RNA nucleic acid constructs. Following incorporation of these simple modifications, stocks of virus are generated for testing in the mouse model system. The following rAAV RNAi experimental virus stocks are tested: single-strand AAV-2/2; single-strand AAV-2/8; self-complementary AAV-2/2; and self-complementary AAV-2/8.

Corresponding viral particles that harbor rAAV vectors expressing unrelated RNA sequences are produced and used as negative controls. Large decreases in relative levels of luciferase activity correlate with increases in transduction efficiency.

### Development of an AAV Interfering RNA Nucleic Acid Construct

Construction of a nucleic acid construct according to the present invention includes two or more individual interfering RNAs under the influence of a single promoter. Initially, assessment of promoter strength of various promoter sequences is conducted in vectors containing the single, individual promoters, driving expression of the same interfering RNA with demonstrated functional inhibition of luciferase activity (Elbashir, et al. Nature. 411: 494-498 (2001a)). Since there is a wealth of data demonstrating the successful utilization of the U6 promoter for the expression of interfering RNAs, it is used as the standard for assessing the relative strength of other promoters. The majority of the promoters that are tested are quite short, most in the range of 200-300 nucleotides in length. Long, overlapping oligonucleotides may be used to assemble the promoters and terminators de novo and are then cloned into multiple cloning sites that flank the sequence encoding the interfering RNA. The promoter is paired with the termination signal that occurs naturally downstream of the gene from which the promoter is taken.

The relative strength of each promoter is assessed in vitro by the decrease in activity of a co-transfected luciferase reporter. The test and reporter constructs are transfected into permissive cells utilizing standard techniques. Controls consist of a test promoter construct in which the sequence encoding the functional interfering RNA against luciferase is replaced by an unrelated RNA sequence. A third marker construct encoding for the secreted protein human α1-antitrypsin (hAAT) is co-transfected into the cells in order to assess for variations in transfection efficiencies. For assessing inhibitory activity of the interfering RNA, luciferase activity is measured utilizing standard commercial kits. The interfering RNA-mediated decrease in luciferase expression, normalized to hAAT levels, is an indirect measurement of promoter strength. Alternatively or in addition, quantitative real time PCR analysis (Q-PCR) on luciferase RNA levels is performed on RNA that is harvested and purified from parallel experimental plates.

### Testing of Highly Functional Interfering RNA Against HCV In Vivo

It must be verified that AAV particles delivered by the interfering RNA nucleic acid construct of the present invention inhibit the luciferase-HCV fusion reporter in vitro. Permissive tissue culture cells are transfected with one of the reporter constructs described supra. In addition, each co-transfection mixture is supplemented with a plasmid coding for hAAT. Following 48 hours of incubation, cells are dosed with infectious particles harboring the interfering RNA nucleic acid construct against HCV. AAV particles containing a triple promoter construct expressing three unrelated RNAs serve as the negative control. Measurement of luciferase activity is used to verify that the AAV-delivered interfering RNAs are highly functional.

Nucleic acids delivered to mice by hydrodynamic transfection methods (high pressure tail vein injection) localize primarily to the liver; thus, this technique is used to deliver the luciferase-HCV fusions to mouse livers. In order to assess the differences in transfection efficiency from animal to animal, a hAAT expression plasmid is included in the transfection mixture.

Infectious AAV particles containing constructs that express the interfering RNAs targeted against HCV sequences are delivered to normal C57B1/6 mice either by tail vein or hepatic portal vein injection. Infectious AAV particles expressing three unrelated RNAs serve as the negative control. Initially, a fairly high dose of virus, e.g. 2×1012 vector genomes, is used, though subsequent experiments are performed to establish dose-response curves. After 48-72 hours, the mice are sacrificed, the livers harvested and samples of serum collected. Luciferase activity is used as a benchmark to assess efficacy of the AAV-delivered RNA agents. In addition to monitoring the levels of hAAT, serum levels of the liver enzymes alanine aminotransferase, aspartate aminotransferase, and tumor necrosis factor alpha are measured by ELISA to ensure general hepatic toxicity is not induced by the treatment.

### 16. Constructs Targeting Three Separate Regions of HCV

Hepatitis C virus (HSV) is a small single stranded RNA virus that shows a high degree of sequence variation. The use of multiple constructs targeting HCV and other variable viruses, such as HIV, offers considerable advantages. Specifically, the use of multiple constructs may act to greatly reduce or eliminate the development of ddRNAi-resistant HCV strains. Moreover, as demonstrated by examples above, more active constructs may be obtained.

The construct pU6.HCVx3 hp (FIG. 72) is designed to target 3 separate regions of the HCV genome, (SEQ ID NO: 198) namely positions 130-151, 148-169 and 318-340 of Accession No. NC_{—}004102.

pU6.HCVx3 may be prepared using the oligonucleotide assembly strategy with the following oligonucleotides:

| | |
|---|---|
| HCV-3x-1 | |
| ACCGGAGAGCCATAGTGGTCTGGAAA | (SEQ ID NO: 214) |
| HCV-3x-2 | |
| ACCGGTTCCGTTTCCAGACCACTATGGCTCTC | (SEQ ID NO: 215) |
| HCV-3x-3 | |
| CGGAACCGGTGAGTACACGAAAAGG | (SEQ ID NO: 216) |
| HCV-3x-4 | |
| GCACGGTCTACGAGACCTTTTCGTGTACTC | (SEQ ID NO: 217) |
| HCV-3x-5 | |
| TCTCGTAGACCGTGCATTTGTGTA | (SEQ ID NO: 218) |
| HCV-3x-6 | |
| AGACCGTGCACTACACAAAT | (SEQ ID NO: 219) |
| HCV-3x-7 | |
| GTGCACGGTCTACGAGACCTCAAGGTG | (SEQ ID NO: 220) |
| HCV-3x-8 | |
| GGTGAGTACACCTTGAGGTCTCGT | (SEQ ID NO: 221) |
| HCV-3x-9 | |
| TACTCACCGGTTCCGCAAGCAGACCAC | (SEQ ID NO: 222) |
| HCV-3x-10 | |
| AGAGCCATAGTGGTCTGCTTGCGGAACC | (SEQ ID NO: 223) |
| HCV-3x-11 | |
| TATGGCTCTCCTTTTTTGGAAA | (SEQ ID NO: 224) |
| HCV-3x-12 | |
| AGCTTTTCCAAAAAAGG | (SEQ ID NO: 225) |

The activity of this construct against HCV may be determined using the assays described above.

### 17. Production of Sense and Antisense RNAs In Vivo

The interfering RNA of the present invention may be produced by two constructs in vivo. FIG. 73 shows an example of this approach. Two ddRNAi constructs, pU6.GR22-sense (A) and pU6.GR22-antisense (B) may be prepared using the long range PCR strategy described above. pU6.GR22-sense is prepared using the oligonucleotides:

U6 GR22-s

(SEQ ID NO: 299)

TGAGATGAACTTCAGGGTCAGCGGTGTTTCGTCCTTTC
term GR22-s

(SEQ ID NO: 300)

AGCCTTTCACTACTCCTACTTTTTTTTGGAAAAGCTTATCG

pU6.GR22-Antisense is Prepared Using the Oligonucleotides

U6 GR22-as

(SEQ ID NO: 301)

CTGGCCTTTCACTACTCCTACCTGGTGTTTCGTCCTTTC
term GR22-as

(SEQ ID NO: 302)

AGATGAACTTCAGGGTCAGCTTTTTTGGAAAAGCTTATCG

These constructs are designed to produce RNAs that are the reverse complement of each other (SEQ ID NOS: 305 and 306). They are predicted to (spontaneously) form double stranded RNA as shown in FIG. 73C thereby triggering degradation of EGFP and hRluc mRNAs. hRluc mRNA degradation can readily be assayed as described above. EGFP degradation can be readily assayed by monitoring reductions in expression of a EGFP in co-transfection experiments, methods for which are well known to those familiar with the art.

The constructs might be tested by co-transfecting the two plasmids into HeLa cells expressing hRluc and hRluc inactivation assayed as described above. Alternatively the two transcriptional units might be combined into a single construct as shown in FIG. 71 and this construct assayed. Similar experiments may be performed in HeLa cells to monitor for inactivation of a co-transfected EGFP expressing plasmid.

### 18. Production of Sense and Antisense RNAs In Vitro

The interfering RNA of the present invention may be produced by two constructs in vitro. FIG. 74 shows an example of this approach. In vitro transcribed RNA may be prepared from these fragments using a commercial kit (Ambion siRNA construction kit) according to the manufacturer's protocols. Transcripts from two DNA fragments, namely, T7 GR22-sense (A) and T7 GR22-antisense (B) may be prepared using the above kit.

T7 GR22-sense is prepared using the oligonucleotide:

T7 GR22-s
(SEQ ID NO: 303)
GCTGACCCTGAAGTTCATCTCAAGCCTTTCACTACTCCTACTTCCTGTC
TC

T7 GR22-antisense is prepared using the oligonucleotide:

T7 GR22-as
(SEQ ID NO: 304)
AAAGTAGGAGTAGTGAAAGGCCAGAGATGAACTTCAGGGTCAGCCTG
TCTC

The two transcripts (SEQ ID NOS: 307 and 308) are predicted to anneal and following the appropriate RNase treatment they will produce the dsRNA shown in FIG. 74C. The activity of the constructs may be determined as described above.

It will be understood that the invention disclosed and defined in this specification extends to all alternative combinations of two or more of the individual features mentioned or evident from the text or drawings. All of these different combinations constitute various alternative aspects of the invention.

1. A ribonucleic acid (RNA) for use as interfering RNA in gene silencing techniques to silence a target gene comprising in a 5′ to 3′ direction:
a first effector sequence of 17 to 21 nucleotides in length;
a second effector sequence of 17 to 21 nucleotides in length;
a sequence of one or more nucleotides capable of forming a loop;
a sequence complementary to the second effector sequence; and
a sequence complementary to the first effector sequence;
wherein at least one of the effector sequences or sequences complementary to the effector sequences is substantially identical to the predicted transcript of a region of the target gene; and
wherein the complementary sequences are capable of forming double stranded regions with their respective effector sequences with the loop between the second effector sequence and the sequence complementary to the second effector sequence.

2. An RNA according to claim 1, further comprising a spacing sequence of one or more nucleotides wherein the first effector sequence is spaced from the second effector sequence by the spacing sequence.

3. An RNA according to claim 2, further comprising an additional spacing sequence of one or more nucleotides, wherein the sequence complementary to the second effector sequence is spaced from the sequence complementary to the first effector sequence by this additional spacing sequence.

4. An RNA according to claim 1, further comprising a spacing sequence of one or more nucleotides wherein the sequence complementary to the second effector sequence is spaced from the sequence complementary to the first effector sequence by the spacing sequence.

5. An RNA according to claim 4 wherein the spacing sequences are not annealable.

6. An RNA according to claim 2, wherein the spacing sequence includes a sequence selected from the group consisting of AA, UU, UUA, UUAG, UUACAA, UUG, UUGA, UUGUUG.

7. An RNA according to claim 1, comprising three effector sequences and three sequences substantially complementary to the effector sequences wherein the sequences substantially complementary to the effector sequences are capable of forming double stranded regions with the effector sequences.

8. An RNA according to claim 1, comprising four effector sequences and four sequences substantially complementary to the effector sequences wherein the sequences substantially complementary to the effector sequences are capable of forming double stranded regions with the effector sequences.

9. An RNA according to claim 1, comprising five effector sequences and five sequences substantially complementary to the effector sequences wherein the sequences substantially complementary to the effector sequences are capable of forming double stranded regions with the effector sequences.

10. An RNA according to claim 1, comprising more than five effector sequences and the same number of sequences substantially complementary to the effector sequences wherein the sequences substantially complementary to the effector sequences are capable of forming double stranded regions with the effector sequences.

11. An RNA according to claim 4, wherein the spacing sequence includes a sequence selected from the group consisting of AA, UU, UUA, UUAG, UUACAA, UUG, UUGA, UUGUUG.

12. An RNA according to claim 3, wherein the additional spacing sequence includes a sequence selected from the group consisting of AA, UU, UUA, UUAG, UUACAA, UUG, UUGA, UUGUUG.

13. A nucleic acid construct encoding an RNA according to claim 1.

14. A double stranded nucleic acid construct including a sequence encoding a ribonucleic acid (RNA) suitable for use as interfering RNA in gene silencing techniques to silence a target gene, the construct comprising in a 5′ to 3′ direction:
a first effector-encoding sequence of 17 to 21 nucleotides in length;
a second effector-encoding sequence of 17 to 21 nucleotides in length;
a sequence encoding RNA of one or more nucleotides that is capable of forming a loop;
a sequence encoding RNA that is capable of forming double-stranded regions with RNA encoded by the second effector-encoding sequence; and
a sequence encoding RNA that is capable of forming double-stranded regions with RNA encoded by the first effector-encoding sequence;
wherein at least one of the sequences encoded by the effector-encoding sequence or encoded by the sequence encoding RNA that is capable of forming double stranded regions with the sequence encoded by the effector-encoding sequence is substantially identical to the predicted transcript of a region of the target gene.

15. A double stranded nucleic acid construct according to claim 14 further comprising a sequence encoding a spacing sequence of one or more nucleotides, the sequence encoding the spacing sequence being located between the first effector-encoding sequence and the second effector-encoding sequence.

16. A double stranded nucleic acid construct according to claim 14 further comprising a sequence encoding a spacing sequence of one or more nucleotides, the sequence encoding the spacing sequence being located between the sequence complementary to the second effector-encoding sequence and the sequence complementary to the first effector-encoding sequence.

17. A double stranded nucleic acid construct according to claim 16 wherein the first effector-encoding sequence is spaced from the second effector-encoding sequence by an additional sequence encoding a spacing sequence of one or more nucleotides.

18. A double stranded nucleic acid construct according to claim 17 wherein the transcript of the spacing sequence is not annealable with the transcript of the additional spacing sequence.

19. A nucleic acid construct according to claim 14, comprising three effector-encoding sequences and three sequences substantially complementary to the effector-encoding sequence wherein the primary transcripts of the effector-encoding sequences are capable of forming double-stranded regions with the sequences complementary to the effector-encoding sequences.

20. A nucleic acid construct according to claim 14, comprising four effector-encoding sequences and four sequences substantially complementary to the effector-encoding sequence wherein the primary transcripts of the effector-encoding sequences are capable of forming double-stranded regions with the sequences complementary to the effector-encoding sequences.

21. A nucleic acid construct according to claim 14, comprising five effector-encoding sequences and five sequences substantially complementary to the effector-encoding sequence wherein the primary transcripts of the effector-encoding sequences are capable of forming double-stranded regions with the sequences complementary to the effector-encoding sequences.

22. A nucleic acid construct according to claim 14 further comprising a promoter and a terminator operably linked to the effector-encoding and substantially complementary sequences.

23. A method of inhibiting expression of a target gene by introducing a nucleic acid construct according to claim 1 into a cell.

24. A method of inhibiting expression of a target gene by introducing a nucleic acid construct according to claim 14 into a cell.

25. A method of inhibiting expression of a target gene by introducing to the target gene an RNA according to claim 1.

26. A method of constructing a nucleic acid construct according to claim 14 comprising adding a predetermined oligonucleotide to a polynucleotide, the oligonucleotide being divided into a first sub-sequence and a second sub-sequence, by a polymerase chain reaction process including:
providing a first primer having at its 3′ end a fixing part hybridizable under polymerase chain reaction conditions with at least a first part of the polynucleotide and at is 5′ end an effector part identical to the first sub-sequence, and a second primer having at its 3′ end a fixing part hybridizable with at least a second part of the polynucleotide that is adjacent the first part of the polynucleotide and at its 5′ end an effector part identical to the second sub-sequence,
introducing the primers to the nucleotide under polymerase chain reaction conditions such that the fixing parts of each primer hybridizes with the polynucleotide;
conducting a multiple polymerase chain reaction to produce an amplification product which includes the effector parts of the primers at the ends of a double-stranded sequence; and
ligating the ends of the effector parts together to form a combined polynucleotide and oligonucleotide sequence.

27. A kit for constructing a nucleic acid construct by the method of claim 26 comprising the polynucleotide, a polymerase, a first primer, a second primer and a ligating enzyme in proportions suitable for the method of claim 26.

28. A ribonucleic acid (RNA) for use as interfering RNA in gene silencing techniques to silence a target gene comprising in a 5′ to 3′ direction:
a first effector sequence of 17 to 30 nucleotides in length;
a second effector sequence of 17 to 30 nucleotides in length;
a sequence of one or more nucleotides capable of forming a loop;
a sequence complementary to the second effector sequence; and
a sequence complementary to the first effector sequence;
wherein at least one of the effector sequences or sequences complementary to the effector sequences is substantially identical to the predicted transcript of a region of the target gene; and
wherein the complementary sequences are capable of forming double stranded regions with their respective effector sequences with the loop being between the second effector sequence and the sequence complementary to the second effector sequence.

29. A double stranded nucleic acid construct including a sequence encoding a ribonucleic acid (RNA) suitable for use as interfering RNA in gene silencing techniques to silence a target gene, the construct comprising in a 5′ to 3′ direction:
a first effector-encoding sequence of 17 to 30 nucleotides in length;
a second effector-encoding sequence of 17 to 30 nucleotides in length;
a sequence encoding RNA of one or more nucleotides that is capable of forming a loop;
a sequence encoding RNA that is capable of forming double stranded regions with RNA encoded by the second effector-encoding sequence; and
a sequence encoding RNA that is capable of forming double stranded regions with RNA encoded by the first effector-encoding sequence;
wherein at least one of the sequences encoded by the effector-encoding sequence or encoded by the sequence encoding RNA that is capable of forming double stranded regions with the sequence encoded by the effector-encoding sequence is substantially identical to the predicted transcript of a region of the target gene.

30. An RNA according to claim 1, wherein the loop forming sequence includes a sequence selected from the group consisting of UU, UUA, UUAG, UUACAA, UUG, UUGA and UUGUUG and N.sub.1AAN.sub.2, wherein N.sub.1 and N.sub.2 are any of C, G, U and A and may be the same or different.

31. An RNA according to claim 1, wherein the target gene is in a mammalian cell.

32. A double-stranded nucleic acid construct according to claim 14, wherein the target gene is in a mammalian cell.

33. An RNA according to claim 28, wherein the target gene is in a mammalian cell.

34. A double-stranded nucleic acid construct according to claim 29, wherein the target gene is in a mammalian cell.
